SEQUENCE LISTING (134 sequences)

SEQ ID NO: 1 - protein, 22 aa, Homo sapiens
MLLLVTSLLLCELPHPAFLLIP

SEQ ID NO: 2 - protein, 120 aa, synthetic construct
  REGION 1..120: note = FMC63 VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 3 - protein, 18 aa, synthetic construct
  REGION 1..18: note = Whitlow Linker
GSTSGSGKPGSGEGSTKG

SEQ ID NO: 4 - protein, 107 aa, synthetic construct
  REGION 1..107: note = FMC63 VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 5 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 6 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 245 aa, synthetic construct
  REGION 1..245: note = FMC63 scFV
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 8 - protein, 42 aa, Homo sapiens
KRGRKKLLYIFKQPFMRPVQTTQEEDGCSCRFPEEEEGGCEL

SEQ ID NO: 9 - protein, 94 aa, Homo sapiens
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 10 - protein, 62 aa, Homo sapiens
KKVAKKPTNKAPHPKQEPQEINFPDDLPGSNTAAPVQETLHGCQPVTQEDGKESRISVQERQ

SEQ ID NO: 11 - protein, 42 aa, Homo sapiens
ALYLLRRDQRLPPDAHKPPGGGSFRTPIQEEQADAHSTLAKI

SEQ ID NO: 12 - protein, 25 aa, Homo sapiens
TYCFAPRCRERRRNERLRRESVRPV

SEQ ID NO: 13 - protein, 61 aa, Homo sapiens
KWKKKKRPRNSYKCGTNTMEREESEQTKKREKIHIPERSDEAQRVFKSSKTSSCDKSDTCF

SEQ ID NO: 14 - protein, 48 aa, Homo sapiens
QRRKYRSNKGESPVEPAEPCHYSCPREEEGSTIPIQEDYRKPEPACSP

SEQ ID NO: 15 - protein, 38 aa, Homo sapiens
CWLTKKKYSSSVHDPNGEYMFMRAVNTAKKSRLTDVTL

SEQ ID NO: 16 - protein, 51 aa, Homo sapiens
MGWIRGRRSRHSWEMSEFHNYNLDLKKSDFSTRWQKQRCPVVKSKCRENAS

SEQ ID NO: 17 - protein, 24 aa, Homo sapiens
LCARPRRSPAQEDGKVYINMPGRG

SEQ ID NO: 18 - protein, 52 aa, Homo sapiens
YFLGRLVPRGRGAAEAATRKQRITETESPYQELQGQRSDVYSDLNTQRPYYK

SEQ ID NO: 19 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 20 - protein, 41 aa, Homo sapiens
RSKRSRLLHSDYMNMTPRRPGPTRKHYQPYAPPRDFAAYRS

SEQ ID NO: 21 - protein, 47 aa, Homo sapiens
TTTPAPRPPTPAPTIASQPLSLRPEACRPAAGGAVHTRGLDFACDIY

SEQ ID NO: 22 - protein, 39 aa, Homo sapiens
IEVMYPPPYLDNEKSNGTIIHVKGKHLCPSPLFPGPSKP

SEQ ID NO: 23 - protein, 21 aa, Homo sapiens
IYIWAPLAGTCGVLLLSLVIT

SEQ ID NO: 24 - protein, 27 aa, Homo sapiens
FWVLVVVGGVLACYSLLVTVAFIIFWV

SEQ ID NO: 25 - protein, 15 aa, synthetic construct
  REGION 1..15: note = (GGGGS)3
GGGGSGGGGSGGGGS

SEQ ID NO: 26 - protein, 20 aa, synthetic construct
  REGION 1..20: note = Linker 3
GGSEGKSSGSGSESKSTGGS

SEQ ID NO: 27 - protein, 8 aa, synthetic construct
  REGION 1..8: note = Linker 4
GGGSGGGS

SEQ ID NO: 28 - protein, 12 aa, synthetic construct
  REGION 1..12: note = Linker 5
GGGSGGGSGGGS

SEQ ID NO: 29 - protein, 16 aa, synthetic construct
  REGION 1..16: note = Linker 6
GGGSGGGSGGGSGGGS

SEQ ID NO: 30 - protein, 20 aa, synthetic construct
  REGION 1..20: note = Linker 7
GGGSGGGSGGGSGGGSGGGS

SEQ ID NO: 31 - protein, 20 aa, synthetic construct
  REGION 1..20: note = Linker 8
GGGGSGGGGSGGGGSGGGGS

SEQ ID NO: 32 - protein, 25 aa, synthetic construct
  REGION 1..25: note = Linker 9
GGGGSGGGGSGGGGSGGGGSGGGGS

SEQ ID NO: 33 - protein, 14 aa, synthetic construct
  REGION 1..14: note = Linker 10
IRPRAIGGSKPRVA

SEQ ID NO: 34 - protein, 15 aa, synthetic construct
  REGION 1..15: note = Linker 11
GKGGSGKGGSGKGGS

SEQ ID NO: 35 - protein, 15 aa, synthetic construct
  REGION 1..15: note = Linker 12
GGKGSGGKGSGGKGS

SEQ ID NO: 36 - protein, 15 aa, synthetic construct
  REGION 1..15: note = Linker 13
GGGKSGGGKSGGGKS

SEQ ID NO: 37 - protein, 15 aa, synthetic construct
  REGION 1..15: note = Linker 14
GKGKSGKGKSGKGKS

SEQ ID NO: 38 - protein, 15 aa, synthetic construct
  REGION 1..15: note = Linker 15
GGGKSGGKGSGKGGS

SEQ ID NO: 39 - protein, 15 aa, synthetic construct
  REGION 1..15: note = Linker 16
GKPGSGKPGSGKPGS

SEQ ID NO: 40 - protein, 20 aa, synthetic construct
  REGION 1..20: note = Linker 17
GKPGSGKPGSGKPGSGKPGS

SEQ ID NO: 41 - protein, 20 aa, synthetic construct
  REGION 1..20: note = Linker 18
GKGKSGKGKSGKGKSGKGKS

SEQ ID NO: 42 - protein, 14 aa, synthetic construct
  REGION 1..14: note = Linker 19
STAGDTHLGGEDFD

SEQ ID NO: 43 - protein, 15 aa, synthetic construct
  REGION 1..15: note = Linker 20
GEGGSGEGGSGEGGS

SEQ ID NO: 44 - protein, 15 aa, synthetic construct
  REGION 1..15: note = Linker 21
GGEGSGGEGSGGEGS

SEQ ID NO: 45 - protein, 15 aa, synthetic construct
  REGION 1..15: note = Linker 22
GEGESGEGESGEGES

SEQ ID NO: 46 - protein, 15 aa, synthetic construct
  REGION 1..15: note = Linker 23
GGGESGGEGSGEGGS

SEQ ID NO: 47 - protein, 20 aa, synthetic construct
  REGION 1..20: note = Linker 24
GEGESGEGESGEGESGEGES

SEQ ID NO: 48 - protein, 18 aa, synthetic construct
  REGION 1..18: note = Linker 25
GSTSGSGKPGSGEGSTKG

SEQ ID NO: 49 - protein, 19 aa, synthetic construct
  REGION 1..19: note = Linker 26
PRGASKSGSASQTGSAPGS

SEQ ID NO: 50 - protein, 19 aa, synthetic construct
  REGION 1..19: note = Linker 27
GTAAAGAGAAGGAAAGAAG

SEQ ID NO: 51 - protein, 19 aa, synthetic construct
  REGION 1..19: note = Linker 28
GTSGSSGSGSGGSGSGGGG

SEQ ID NO: 52 - protein, 20 aa, synthetic construct
  REGION 1..20: note = Linker 29
GKPGSGKPGSGKPGSGKPGS

SEQ ID NO: 53 - protein, 4 aa, synthetic construct
  REGION 1..4: note = Linker 30
GSGS

SEQ ID NO: 54 - protein, 10 aa, synthetic construct
  REGION 1..10: note = Linker 31
APAPAPAPAP

SEQ ID NO: 55 - protein, 20 aa, synthetic construct
  REGION 1..20: note = Linker 32
APAPAPAPAPAPAPAPAPAP

SEQ ID NO: 56 - protein, 32 aa, synthetic construct
  REGION 1..32: note = Linker 33
AEAAAKEAAAKEAAAAKEAAAAKEAAAAKAAA

SEQ ID NO: 57 - protein, 15 aa, Homo sapiens
EPKSCDKTHTCPPCP

SEQ ID NO: 58 - protein, 12 aa, Homo sapiens
ERKCCVECPPCP

SEQ ID NO: 59 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 60 - protein, 12 aa, Homo sapiens
ESKYGPPCPSCP

SEQ ID NO: 61 - DNA, 63 bp, Mus musculus
  source 1..63: mol_type = genomic DNA
atggccaggagccccgcccagctgctgggcctgctgctgctgtggctgagcggcgccaggtgc

SEQ ID NO: 62 - protein, 21 aa, Mus musculus
MARSPAQLLGLLLLWLSGARC

SEQ ID NO: 63 - DNA, 36 bp, synthetic construct
  misc_feature 1..36: note = Short Hinge
  source 1..36: mol_type = other DNA
gagagcaagtatgggcccccttgtcctccttgtccg

SEQ ID NO: 64 - protein, 12 aa, synthetic construct
  REGION 1..12: note = Short Hinge
ESKYGPPCPPCP

SEQ ID NO: 65 - DNA, 357 bp, synthetic construct
  misc_feature 1..357: note = Medium Hinge
  source 1..357: mol_type = other DNA
gagagcaagtatgggcccccttgtcctccttgtccggggcagccccgagagccacaggtgtacactctgccaccaagtcaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagagcctctccctgtctctgggaaag

SEQ ID NO: 66 - protein, 119 aa, synthetic construct
  REGION 1..119: note = Medium Hinge
ESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 67 - DNA, 684 bp, synthetic construct
  misc_feature 1..684: note = Long Hinge
  source 1..684: mol_type = other DNA
gagtctaagtatgggcccccttgtcctccttgtccggcacctcccgtggctggaccaagtgtattcttatttcccccaaaacccaaagatactctcatgatttcccggacccctgaggttacatgcgtggtggtggatgtgagccaggaagaccccgaagtccagtttaactggtacgtggatggagtggaggtgcataatgcaaagacaaagcctcgggaagaacagtttcagagcacataccgtgtggttagtgtcctcacagttctgcaccaggactggctgaacggcaaggagtataagtgtaaggtctccaataaaggcctcccgtcatcgatcgaaaaaaccatcagtaaagccaaagggcagccaagggagccacaggtgtatactttaccaccaagtcaggaggaaatgaccaagaaccaggtatctctgacctgcctagtcaaaggcttttaccccagcgatatcgctgtggagtgggagtctaatgggcagccagagaacaactacaagaccacacctcctgtgctggactccgatggctccttctttctatacagcaggttaaccgtggataagagcaggtggcaggaggggaatgtcttctcatgctctgtgatgcatgaggctctgcacaaccactacacacagaagagcctctccctgtctctgggaaag

SEQ ID NO: 68 - protein, 228 aa, synthetic construct
  REGION 1..228: note = Long Hinge
ESKYGPPCPPCPAPPVAGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 69 - DNA, 735 bp, synthetic construct
  misc_feature 1..735: note = Herceptin scFv
  source 1..735: mol_type = other DNA
gacatccagatgactcagtcaccatcaagcctgagtgcatccgtgggcgatcgagtgacaataacatgtagagcgagccaggatgtaaatacggcagtagcgtggtaccaacagaaacccggcaaggctcctaagctgttaatctacagcgccagcttcctttatagtggagtgccttcaaggttctcaggatctaggtccggtactgacttcacgctgacaatctcgagcctacaacccgaggacttcgccacttattactgccagcagcattacactactcctcccacattcggacagggaaccaaagtcgagatcaaaggatcaacctctggatctggcaagcccgggagcggggaaggctctactaagggtgaggtgcaactagtggagagtggcggagggctcgtccagccaggaggttccctgaggctgagttgcgctgcaagcggattcaatatcaaggacacgtacatacactgggtgcgccaggcccccggaaagggactggagtgggtcgcccgaatctatcctactaatggctacaccaggtatgctgattcagtgaaaggaaggtttacaatctctgccgatacttcaaagaatacagcttatctacagatgaattcacttagagccgaggatacagccgtgtattattgctcccgatggggaggagatgggttctacgctatggactactggggtcaaggaaccctggtgaccgttagttca

SEQ ID NO: 70 - protein, 245 aa, synthetic construct
  REGION 1..245: note = Herceptin scFv
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGSTSGSGKPGSGEGSTKGEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 71 - protein, 107 aa, synthetic construct [sequence omitted]

SEQ ID NO: 72 - protein, 120 aa, synthetic construct [sequence omitted]

SEQ ID NO: 73 - DNA, 732 bp, synthetic construct
  misc_feature 1..732: note = scFv69 (Herceptin anti-ID)
  source 1..732: mol_type = other DNA
tcttctgagctgactcaggaccctgctgtgtctgtggccttgggacagacagtcaggatcacatgccaaggagacagcctcagaagctattatgcaagctggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaacagcagtgaaccaaccccaccaagagtggtcttcggcggcggaacaaaactgacagtgctgggctctacaagcggcagcggcaaacctggatctggcgagggatctaccaagggcgaggtgcaactattggaaagtggtggcgggctggtccaaccgggcgggtccttgaggctgtcctgtgcagccagcgggtttactttttcttcctacgccatgtcttgggtacgacaggctcccggaaaagggctcgagtgggtgagtgcaatctccgggagtgggggctctacctactacgccgattctgtcaagggtaggttcactatctccagggataattcaaagaatactttatacctgcagatgaattcactgcgagcggaagatacagcagtgtactattgtgccaagaacgtgcacatccagccctttgattactggggccagggcaccagcgtgaccgtgtctagc

SEQ ID NO: 74 - protein, 244 aa, synthetic construct
  REGION 1..244: note = scFv69 (Herceptin anti-ID)
SSELTQDPAVSVALGQTVRITCQGDSLRSYYASWYQQKPGQAPVLVIYGKNNRPSGIPDRFSGSSSGNTASLTITGAQAEDEADYYCNSSEPTPPRVVFGGGTKLTVLGSTSGSGKPGSGEGSTKGEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKNVHIQPFDYWGQGTSVTVSS

SEQ ID NO: 75 - protein, 108 aa, synthetic construct
  REGION 1..108: note = scFv69 VL
SSELTQDPAVSVALGQTVRITCQGDSLRSYYASWYQQKPGQAPVLVIYGKNNRPSGIPDRFSGSSSGNTASLTITGAQAEDEADYYCNSSEPTPPRVVFGGGTKLTVL

SEQ ID NO: 76 - protein, 118 aa, synthetic construct
  REGION 1..118: note = scFv69 VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKNVHIQPFDYWGQGTSVTVSS

SEQ ID NO: 77 - DNA, 381 bp, synthetic construct
  misc_feature 1..381: note = 9G8 VHH (anti-EGFR)
  source 1..381: mol_type = other DNA
gaagtgcagcttgtggagagtggcggtggtttagttcaaccagggggcagcctgcgcttgagttgtgccgcatcgggtcgtaccttttctagctacgcaatggggtggtttcgtcaagctcctgggaaggaacgtgagtttgtggtcgccattaattggtcatcagggagtacatactacgctgattccgtcaaagggcgctttacaatctcacgcgataacagcaagaataccctttatttacaaatgaatagtctgcgtgcagaagatacggctgtgtattactgcgctgcggggtaccaaatcaactctgggaattacaactttaaggactacgagtatgattattggggccagggcactcaggttacagtctcgagc

SEQ ID NO: 78 - protein, 127 aa, synthetic construct
  REGION 1..127: note = 9G8 VHH (anti-EGFR)
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSS

SEQ ID NO: 79 - DNA, 66 bp, synthetic construct
  misc_feature 1..66: note = T2A Sequence
  source 1..66: mol_type = other DNA
ggatccggcgccacaaacttcagcctgctgaaacaggccggcgacgtggaggaaaacccaggccca

SEQ ID NO: 80 - protein, 22 aa, synthetic construct
  REGION 1..22: note = T2A Sequence
GSGATNFSLLKQAGDVEENPGP

SEQ ID NO: 81 - DNA, 714 bp, synthetic construct
  misc_feature 1..714: note = GFP
  source 1..714: mol_type = other DNA
gtgtccaagggcgaagaactgttcaccggcgtggtgcccattctggtggaactggacggggatgtgaacggccacaagttcagcgttagaggcgaaggcgaaggggatgccacaaacggcaagctgaccctgaagttcatctgcaccaccggaaagctgcccgtgccttggcctacactggtcaccacactgacatacggcgtgcagtgcttcagcagataccccgaccatatgaagcagcacgacttcttcaagagcgccatgcctgagggctacgtgcaagagagaaccatcaccttcaaggacgacggcacctacaagaccagagccgaagtgaagttcgagggcgacaccctggtcaaccggatcgagctgaagggcatcgacttcaaagaggacggcaacatcctgggccacaaacttgagtacaacttcaacagccacaacgtgtatatcaccgccgacaagcagaagaacggcatcaaggccaacttcaagatccggcacaacgtggaagatggcagcgtgcagctggccgatcactaccagcagaacacacccatcggagatggccctgtgctgctgcccgataaccactacctgagcacccagagcaagctgagcaaggaccccaacgagaagcgggaccacatggtgctgctggaatttgtgacagccgccggaatcacccacggcatggatgagctgtacaag

SEQ ID NO: 82 - protein, 238 aa, synthetic construct
  REGION 1..238: note = GFP
VSKGEELFTGVVPILVELDGDVNGHKFSVRGEGEGDATNGKLTLKFICTTGKLPVPWPTLVTTLTYGVQCFSRYPDHMKQHDFFKSAMPEGYVQERTITFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSKLSKDPNEKRDHMVLLEFVTAAGITHGMDELYK

SEQ ID NO: 83 - DNA, 354 bp, synthetic construct
  misc_feature 1..354: note = Listeria internalin 6dyx
  source 1..354: mol_type = other DNA
caggtgaagttggaagagtctggtggtggcctcgtccaggctggggggagtcttcgccttagttgcgcagcttcaggtcggacatacagcacttacgcgatggggtggtttcgccagactccggggaaggagagagaattggtggcagcaataaattggtcagggggcaacacgcactatgcagactctgtaaaaggccggttcacgatcagtagagataacgccaagagcaccgtctacctccagatgaattccctgaaacctgaggacacagcagtttattactgtgccgctccgaaaggccacacaggggatcattactggggacccggcacccaagtgactgtgagctcg

SEQ ID NO: 84 - protein, 118 aa, synthetic construct [sequence omitted]

SEQ ID NO: 85 - DNA, 372 bp, synthetic construct
  misc_feature 1..372: note = Naja kaouthia phospholipase A2 P3-3
  source 1..372: mol_type = other DNA
caggtgcaactcgttgaaagtggaggcggtagcgttcaggcaggtggaagcctcaggctgtcctgtgcggccagtagagacacgtatgattcacactgtatggggtggttccggcaagcgcccggaaaagagagggaacaggtggcggcacataacggtggccgaaacacatattacgcagatagcgttaaaggacgatttacaatatctcaggacaatgctaagaatacgatgtatctccaaatgaatagccttaaacctgaagatacagccatttactactgcgccgcggacatgtccgcgagaagggtcgcaaacacaggatgcagatacaattattggggccaaggcactttggtaactgtgagctcg

SEQ ID NO: 86 - protein, 124 aa, synthetic construct
  REGION 1..124: note = Naja kaouthia phospholipase A2 P3-3
QVQLVESGGGSVQAGGSLRLSCAASRDTYDSHCMGWFRQAPGKEREQVAAHNGGRNTYYADSVKGRFTISQDNAKNTMYLQMNSLKPEDTAIYYCAADMSARRVANTGCRYNYWGQGTLVTVSS

SEQ ID NO: 87 - DNA, 345 bp, synthetic construct
  misc_feature 1..345: note = Ebola nucleoprotein anti-Z C
  source 1..345: mol_type = other DNA
caggtgcaacttcaacaaagcggcgggggatccgtaacgccaggtggatcactgcggctttcttgtgctgcttccggatccatttcagactttgctgctatggcatggtatcggcaggcccctggcaaagaaagggattgggtttttggaaccatattctcagcaggtgctttgttgtacgcagaaccagttaaaggtcggtttaccatatcaagggataacgctaagaatactgtatatcttcaaatgaacagtttgaaaccagaggatactgccgtatactattgtcgcctctatgctaaggcaatttattggggtcagggcacacaggtaacggtgagctcg

SEQ ID NO: 88 - protein, 115 aa, synthetic construct
  REGION 1..115: note = Ebola nucleoprotein anti-Z C
KVQLQQSGGGSVTPGGSLRLSCAASGSISDFAAMAWYRQAPGKERDWVFGTIFSAGALLYAEPVKGRFTISRDNAKNTVYLQMNSLKPEDTAVYYCRLYAKAIYWGQGTQVTVSS

SEQ ID NO: 89 - DNA, 372 bp, synthetic construct
  misc_feature 1..372: note = Herpes simplex virus: Glycoprotein D
  source 1..372: mol_type = other DNA
caggtgcaacttcaggctagtggagggggactggtgcaagctggcgggtctttgcgactgtcctgtgcagcctcaggccgagctacaggcaattatcccatgggatggttccgccaggcccctggaaaagaacgcgagttcgttgcagccatcagtcgggacggcgacagtacatactaccgggatagtgttaaaggccgatttaccatatcccgagacaatacgaaaaatacggcatatcttcagatgaacagtcttaagccggaggacacagccgtctactattgtgcagctgaccggctgacagcatatcgatacaatccagggcagattgactattggggacagggtacacaagttacggtgagctcg

SEQ ID NO: 90 - protein, 124 aa, synthetic construct
  REGION 1..124: note = Herpes simplex virus: Glycoprotein D
EVQLQASGGGLVQAGGSLRLSCAASGRATGNYPMGWFRQAPGKEREFVAAISRDGDSTYYRDSVKGRFTISRDNTKNTAYLQMNSLKPEDTAVYYCAADRLTAYRYNPGQIDYWGQGTQVTVSS

SEQ ID NO: 91 - DNA, 369 bp, synthetic construct
  misc_feature 1..369: note = Lacotococcal phage: RBP
  source 1..369: mol_type = other DNA
caggtgcaactggtcgaaagtggtggggggctcgttcaagccggtggcagtttgcgcttgtcatgcgccgctagtgaaagtaccttctctaactacgcgatgggatggttcaggcaagcaccaggccctgaaagggaatttgtggctacgatttctcaaacagggtcccacacctactaccgcaattctgtgaagggacgcttcacgattagtcgggataacgccaagaacacagtgtaccttcaaatgaacaatatgaagcctgaagacacggccgtgtattattgtgcagccggagacaactattactataccagaacttatgagtacgactactggggccagggtactcaggtcactgtgagctcg

SEQ ID NO: 92 - protein, 123 aa, synthetic construct
  REGION 1..123: note = Lacotococcal phage: RBP
QVQLVESGGGLVQAGGSLRLSCAASESTFSNYAMGWFRQAPGPEREFVATISQTGSHTYYRNSVKGRFTISRDNAKNTVYLQMNNMKPEDTAVYYCAAGDNYYYTRTYEYDYWGQGTQVTVSS

SEQ ID NO: 93 - DNA, 375 bp, synthetic construct
  misc_feature 1..375: note = RSV: F-protein
  source 1..375: mol_type = other DNA
caggtgcagttggtcgagtcaggtggagggtcagtgcagccaggggggtcccttcgacttagttgtgcagccagtggttttacactggattactattacattgggtggtttcgacaggcccccgggaaggaacgcgagggtgtttcttgtatttccagctcacatggctcaacctattatgctgactcagtaaaaggtcggtttacgataagccgggataatgcaaagaataccgtgtatcttcaaatgaatagccttaaaccagaagataccgctgtgtactattgtgccactatacgctctagctcatgggggggctgtgtccactacgggatggattattgggggaagggcacgcaagtcacggtgagctcg

SEQ ID NO: 94 - protein, 125 aa, synthetic construct
  REGION 1..125: note = RSV: F-protein
QVQLVESGGGSVQPGGSLRLSCAASGFTLDYYYIGWFRQAPGKEREGVSCISSSHGSTYYADSVKGRFTISRDNAKNTVYLQMNSLKPEDTAVYYCATIRSSSWGGCVHYGMDYWGKGTQVTVSS

SEQ ID NO: 95 - DNA, 372 bp, synthetic construct
  misc_feature 1..372: note = Geobacillus stearothermophilus: SbsB
  source 1..372: mol_type = other DNA
caggtgcagttgcaagaaagtggagggggattggtgcaggcaggtggatctttgaggctgtcctgtgcagcctctggtcgcactagctctgcgtacgctatgggttggtttcgacaggcccctgggaaagaacgcgagttcgttgccggcatttcaagcaaaggcggtagcacgtattatggtgccagcatgaaaggacgctttacgatatcacgggataacgcgaaaaatacggtctacttgcagatgaacggtctggccccagaagacacggcagtgtactactgcgctgcgagcgacaagtataatttcgacaccagccatgcgggatacggctattggggccaagggacccaggttacagtgagctcg

SEQ ID NO: 96 - protein, 124 aa, synthetic construct
  REGION 1..124: note = Geobacillus stearothermophilus: SbsB
QVQLQESGGGLVQAGGSLRLSCAASGRTSSAYAMGWFRQAPGKEREFVAGISSKGGSTYYGASMKGRFTISRDNAKNTVYLQMNGLAPEDTAVYYCAASDKYNFDTSHAGYGYWGQGTQVTVSS

SEQ ID NO: 97 - DNA, 360 bp, synthetic construct
  misc_feature 1..360: note = Ricin (V5E4)
  source 1..360: mol_type = other DNA
caggtgcaactcgtggagacgggtggaggacttgtgcaagcgggcggaagccttaggttgagctgtgctgcgtctggatttacatttagtagctatgcaatgggctggtttcgccaggcgccggggaaggaacgcgacttcgttgcgggtatctcacttagcggcgccgggacgtactatgtaaaaggaaggttcaccatttcacgcgataacgctaaaaacactgtctatttgcagatgaacagcctcaaaccagaggatactgcagtatattactgtaaggccacaggagaaagggggtatggagatcagggatatcttgaagtctgggggagagggacgctggttaccgtgagctcg

SEQ ID NO: 98 - protein, 120 aa, synthetic construct
  REGION 1..120: note = Ricin (V5E4)
QVQLVETGGGLVQAGGSLRLSCAASGFTFSSYAMGWFRQAPGKERDFVAGISLSGAGTYYVKGRFTISRDNAKNTVYLQMNSLKPEDTAVYYCKATGERGYGDQGYLEVWGRGTLVTVSS

SEQ ID NO: 99 - DNA, 384 bp, synthetic construct
  misc_feature 1..384: note = Lyso CW P01 B10
  source 1..384: mol_type = other DNA
caggtgcaacttgtggagagcgggggtggtctcgtccaggctggtggctcacttcgcctctcctgcgcggctagcggcgggatattctcaggtggaagaatgggctggttcagacaagctccaggtaaagagagagagttcgtagcggcggtaatcactcgcggggggtctacttattatgcggactctgtgaagggtagatttacaatttcacgggacaacgcaaagaatactgtgtaccttcaaatgaactcacttaaacctgaggacacggcggtttattactgcgctgcaagcgaggtaacctatgacgagggacattacatcggaaccaaatccacttacgacacttggggacagggcacgcaggtaactgtgagctcg

SEQ ID NO: 100 - protein, 128 aa, synthetic construct
  REGION 1..128: note = Lyso CW P01 B10
QVQLVESGGGLVQAGGSLRLSCAASGGIFSGGRMGWFRQAPGKEREFVAAVITRGGSTYYADSVKGRFTISRDNAKNTVYLQMNSLKPEDTAVYYCAASEVTYDEGHYIGTKSTYDTWGQGTQVTVSS

SEQ ID NO: 101 - DNA, 384 bp, synthetic construct
  misc_feature 1..384: note = Lyso CW P01 B11
  source 1..384: mol_type = other DNA
caggtgcagctcgtagagtccgggggaggtctggtgcaagctggaggttccttgcgattgtcatgtgcggcaagcggaggaattttttctggaggacgaatggggtggttccgacaggcaccgggaaaagagagggagttcgtagctgccgtgattacaaggggtggtagcacatactatgcagatagcgtaaagggtaggtttacgatatccagggataacgcaaagaacacggtctacctgcagatgaactcccttaaaccagaagatactgccgtttattattgcgccgcatcagaggtaacttacgatgaaggtcgatacattgggacgaagagcacctatgacacatgggggcagggtactcaagtgaccgtgagctcg

SEQ ID NO: 102 - protein, 128 aa, synthetic construct
  REGION 1..128: note = Lyso CW P01 B11
QVQLVESGGGLVQAGGSLRLSCAASGGIFSGGRMGWFRQAPGKEREFVAAVITRGGSTYYADSVKGRFTISRDNAKNTVYLQMNSLKPEDTAVYYCAASEVTYDEGRYIGTKSTYDTWGQGTQVTVSS

SEQ ID NO: 103 - protein, 351 aa, synthetic construct
  REGION 1..351: note = Lyso CW P01 D04
CAGGTGCAACTGGTGGAATCCGGGGGTGGACTGGTACAAGCCGGAGGGAGTTTGAGACTCTCTTGCGCTGCCTCCGGTAGAATTTTTTCAATATACGACACCATAGGATGGTTTCGACAAGCGCCTGGAAAGGAAAGAGAGTTTGTAGCGGCCACTATCACAACGGCGGGTATTACGACATATGATGATAGCGTTAAAGGACGGTTCACGATAAGCCGCGACAATGCCAAGAACACAGTGTACCTCCAAATGAATAGCCTTAAGCCCGAGGATACAGCCGTTTACTATTGTTACGTCCGAGTTGGTCGCGGTGACTACTGGGGTCAAGGTACTCAGGTGACTGTAAGCTCG

SEQ ID NO: 104 - protein, 117 aa, synthetic construct
  REGION 1..117: note = Lyso CW P01 D04
QVQLVESGGGLVQAGGSLRLSCAASGRIFSIYDTIGWFRQAPGKEREFVAATITTAGITTYDDSVKGRFTISRDNAKNTVYLQMNSLKPEDTAVYYCYVRVGRGDYWGQGTQVTVSS

SEQ ID NO: 105 - DNA, 351 bp, synthetic construct
  misc_feature 1..351: note = Lyso CW P01 F11
  source 1..351: mol_type = other DNA
caggtgcaactggttgaatctggtggtgggctggtccaagcgggaggcagtcttcgactttcctgcgcagcctcagggagtatattcagcttttacgacgtaggttggttccgccaagcgcccggtaaagaacgagagttcgtcgctgccagtataacgaagggaggcgggacgtactacgtagattcagtaaaagggagatttaccataagtagggacaatgcaaagaacacggtctacctccagatgaatagccttaaaccagaagatacggcagtctattactgcgccctggcaaccccccacggatatgacttttggggccaaggtacgcaagtcacggtgagctcg

SEQ ID NO: 106 - protein, 117 aa, synthetic construct
  REGION 1..117: note = Lyso CW P01 F11
QVQLVESGGGLVQAGGSLRLSCAASGSIFSFYDVGWFRQAPGKEREFVAASITKGGGTYYVDSVKGRFTISRDNAKNTVYLQMNSLKPEDTAVYYCALATPHGYDFWGQGTQVTVSS

SEQ ID NO: 107 - DNA, 354 bp, synthetic construct
  misc_feature 1..354: note = Lyso CW P01 F09
  source 1..354: mol_type = other DNA
caggtgcagctcgttgaaagcgggggtggcttggtccaagcgggaggttccctgagactgtcctgcgctgcgtcaggatcaataatcacgatttatgtagtcggatggttccgacaagccccaggtaaagagcgggaatttgtggcctccgacataggctctggtgggtcaacttattacagtgactccgtaaaaggtcggttcacaatctcaagggataacgcaaagaatacagtctacttgcaaatgaattcattgaagcctgaggatacagcagtgtactactgcgttactggagatccctctactccgtattcatactggggtcaaggcacacaggttacagtgagctcg

SEQ ID NO: 108 - protein, 118 aa, synthetic construct
  REGION 1..118: note = Lyso CW P01 F09
QVQLVESGGGLVQAGGSLRLSCAASGSIITIYVVGWFRQAPGKEREFVASDIGSGGSTYYSDSVKGRFTISRDNAKNTVYLQMNSLKPEDTAVYYCVTGDPSTPYSYWGQGTQVTVSS

SEQ ID NO: 109 - DNA, 363 bp, synthetic construct
  misc_feature 1..363: note = Lyso CW P01 C04
  source 1..363: mol_type = other DNA
caggtgcaactggtcgagagtggtggtggactggttcaagctgggggcagcctgagattgtcctgcgccgcatctggctcctccttctcaatttacgacgtgggctggttccgccaagcacctggaaaggaaagagagtttgttgctgcgacaattgagactgggggacacacgtcttacgccgactcagtgaaaggtagatttacaatctcaagggataacgctaaaaacaccgtttatctgcaaatgaactccctgaaaccggaggatacagctgtgtactattgttatgcgaagattgtctacgaccagggcccgagctactactattggggccaggggacacaggttaccgtgagctcg

SEQ ID NO: 110 - protein, 121 aa, synthetic construct
  REGION 1..121: note = Lyso CW P01 C04
QVQLVESGGGLVQAGGSLRLSCAASGSSFSIYDVGWFRQAPGKEREFVAATIETGGHTSYADSVKGRFTISRDNAKNTVYLQMNSLKPEDTAVYYCYAKIVYDQGPSYYYWGQGTQVTVSS

SEQ ID NO: 111 - protein, 128 aa, synthetic construct
  REGION 1..128: note = huLYSO_CW_P01_B11
EVQLLESGGGLVQPGGSLRLSCAASGGIFSGGRMGWFRQAPGKEREFVAAVITRGGSTYYADSVKGRFTISRDNSKNTLYLQMNSLRAEDTAVYYCAASEVTYDEGRYIGTKSTYDTWGQGTQVTVSS

SEQ ID NO: 112 - protein, 117 aa, synthetic construct
  REGION 1..117: note = huLYSO_CW_P01_D04
EVQLLESGGGLVQPGGSLRLSCAASGRIFSIYDTIGWFRQAPGKEREFVAATITTAGITTYDDSVKGRFTISRDNSKNTLYLQMNSLRAEDTAVYYCYVRVGRGDYWGQGTQVTVSS

SEQ ID NO: 113 - protein, 130 aa, synthetic construct
  REGION 1..130: note = huD04_CNTY2_F09
EVQLLESGGGLVQPGGSLRLSCAASGRTFSSGAMGWYRQAPGKERELVSAISNRGSTVYADSVKGRFTISRDNSKNTLYLQMNSLRAEDTAVYYCAADRNPSSAGAGVAAYRLIARFNYWGQGTQVTVSS

SEQ ID NO: 114 - protein, 131 aa, synthetic construct
  REGION 1..131: note = huD04_CNTY2_F08
EVQLLESGGGLVQPGGSLRLSCAASGFTLSSYAMGWYRQAPGKEREGVSSISYAGVTNYADSVKGRFTISRDNSKNTLYLQMNSLRAEDTAVYYCAAALTGYYAYRRLWSYRIGSQAYDYWGQGTQVTVSS

SEQ ID NO: 115 - protein, 133 aa, synthetic construct
  REGION 1..133: note = huD04_CNTY2_E03
EVQLLESGGGLVQPGGSLRLSCAASGLTYTGYAMGWYRQAPGKERELVSAISSRSSIYYADSVKGRFTISRDNSKNTLYLQMNSLRAEDTAVYYCAAGRPVALGSWRRTATWSAGLGAEYAYWGQGTQVTVSS

SEQ ID NO: 116 - protein, 132 aa, synthetic construct
  REGION 1..132: note = huD04_CNTY2_F04
EVQLLESGGGLVQPGGSLRLSCAASGRTFSSYGMGWYRQAPGKERELVSAISNRGSTYYADSVKGRFTISRDNSKNTLYLQMNSLRAEDTAVYYCAASPARVGVSGHSSSRRSYYGYSYDYWGQGTQVTVSS

SEQ ID NO: 117 - protein, 130 aa, synthetic construct
  REGION 1..130: note = huD04_CNTY2_H12
EVQLLESGGGLVQPGGSLRLSCAASGSIFSSDVMGWYRQAPGKERELVSAISSGGSTRYADSVKGRFTISRDNSKNTLYLQMNSLRAEDTAVYYCAATMGKSKTNRRNYGTWRYGAYAYWGQGTQVTVSS

SEQ ID NO: 118 - protein, 133 aa, synthetic construct
  REGION 1..133: note = huD04_CNTY2_F12
EVQLLESGGGLVQPGGSLRLSCAASGFTFSSNAMGWYRQAPGKEREFVSSISSGRSTTYADSVKGRFTISRDNSKNTLYLQMNSLRAEDTAVYYCAAHGTKYKWTRARLRSARQKQLETYRYWGQGTQVTVSS

SEQ ID NO: 119 - protein, 117 aa, synthetic construct
  REGION 1..117: note = huD04_CNTY1_A11
EVQLLESGGGLVQPGGSLRLSCAASGFTSSNYVMGWYRQAPGKEREFVSAIGSGGSTYYADSVKGRFTISRDNSKNTYLQMNSLRAEDTAVYYCAAEIYTSGARDYWGQGTQVTVSS

SEQ ID NO: 120 - protein, 129 aa, synthetic construct
  REGION 1..129: note = huD04_CNTY2_C03
EVQLLESGGGLVQPGGSLRLSCAASGLTFSRYAMGWYRQAPGKERELVSAISSRRSTYYADSVKGRFTISRDNSKNTLYLQMNSLRAEDTAVYYCAADYRLTSLERARYASASITYDYWGQGTQVTVSS

SEQ ID NO: 121 - protein, 131 aa, synthetic construct
  REGION 1..131: note = huD04_CNTY2_B06
EVQLLESGGGLVQPGGSLRLSCAASGFTFNRYSMGWYRQAPGKERELVSAISSGGSGYYADSVKGRFTISRDNSKNTLYLQMNSLRAEDTAVYYCAIQDYSRIWEYIALDRRRSYYGMDYWGQGTQVTVSS

SEQ ID NO: 122 - protein, 130 aa, synthetic construct
  REGION 1..130: note = huD04_CNTY2_D02
EVQLLESGGGLVQPGGSLRLSCAASGFTFRSYAMGWYRQAPGKEREGVSVISSGGTPSYADSVKGRFTISRDNSKNTLYLQMNSLRAEDTAVYYCDAIMGLVEADYVSTGTYEYTAYAYWGQGTQVTVSS

SEQ ID NO: 123 - protein, 130 aa, synthetic construct
  REGION 1..130: note = huD04_CNTY2_D04
EVQLLESGGGLVQPGGSLRLSCAASGLTFSGVCMGWFRQAPGKEREGVSAIYSSGSTYYADSVKGRFTISRDNSKNTLYLQMNSLRAEDTAVYYCAADRIPDLGEPCIGTTNLARTYNYWGQGTQVTVSS

SEQ ID NO: 124 - protein, 119 aa, synthetic construct
  REGION 1..119: note = huD04_CNTY1_B12
EVQLLESGGGLVQPGGSLRLSCAASGRTSSNYVMGWYRQAPGKEREGVSAITSGGSTYYADSVKGRFTISRDNSKNTLYLQMNSLRAEDTAVYYCAANLYSRTGAYDYWGQGTQVTVSS

SEQ ID NO: 125 - protein, 129 aa, synthetic construct
  REGION 1..129: note = huD04_CNTY2_D05
EVQLLESGGGLVQPGGSLRLSCAASGYIYSSYSMGWYRQAPAKNASLSAITSSGETYYADSVKGRFTISRDNSKNTLYLQMNSLRAEDTAVYYCAADALDAPIAGDRYYRGSGAGYAYWGQGTQVTVSS

SEQ ID NO: 126 - protein, 133 aa, synthetic construct
  REGION 1..133: note = huD04_CNTY2_D12
EVQLLESGGGLVQPGGSLRLSCAASGSAFSSNAMGWYRQAPGKERELVSAISSGGSTNYADSVKGRFTISRDNSKNTLYLQMNSLRAEDTAVYYCAARNPLTYTALVSNAPSGDYYLFEYRLWGQGTQVTVSS

SEQ ID NO: 127 - protein, 119 aa, synthetic construct
  REGION 1..119: note = huD04_CNTY1_D12
EVQLLESGGGLVQPGGSLRLSCAASGFTSSNYVMGWYRQAPGKEREGVSAISSSGRTYYADSVKGRFTISRDNSKNTLYLQMNSLRAEDTAVYYCAAAYYSGYGETDYWGQGTQVTVSS

SEQ ID NO: 128 - protein, 118 aa, synthetic construct
  REGION 1..118: note = huD04_CNTY1_D11
EVQLLESGGGLVQPGGSLRLSCAASGSTFSYYYMGWYRQAPGKERELVSAISSGGSIYYADSVKGRFTISRDNSKNTLYLQMNSLRAEDTAVYYCARRTYYGDEADYWGQGTQVTVSS

SEQ ID NO: 129 - protein, 119 aa, synthetic construct
  REGION 1..119: note = huD04_CNTY1_D10
EVQLLESGGGLVQPGGSLRLSCAASGRTSSNYVIGWYRQAPGKEREFVSAISGGGSTYYADSVKGRFTISRDNSKNTLYLQMNSLRAEDTAVYYCATRIYTARGAGDYWGQGTQVTVSS

SEQ ID NO: 130 - protein, 128 aa, synthetic construct
  REGION 1..128: note = huB11_CNTY3+4_A02
EVQLLESGGGLVQPGGSLRLSCAASGRTYTGSAMGWYRQAPGKERELVSAISTGGETYYADSVKGRFTISRDNSKNTLYLQMNSLRAEDTAVYYCAAGYVGLPYTYRPATSRRGYTYWGQGTQVTVSS

SEQ ID NO: 131 - protein, 127 aa, synthetic construct
  REGION 1..127: note = huB11_CNTY3+4_A01
EVQLLESGGGLVQPGGSLRLSCAASGFTFSSYSIGWYRQAPGKERELVSVISSGGSTYYADSVKGRFTISRDNSKNTLYLQMNSLRAEDTAVYYCAASDWSYSWITYTGTWRLYEYWGQGTQVTVSS

SEQ ID NO: 132 - protein, 118 aa, synthetic construct
  REGION 1..118: note = huB11_CNTY1_E11
EVQLLESGGGLVQPGGSLRLSCAASGFAFSDYVMGWYRQAPGKEREFVSAIRSTTSTYYADSVKGRFTISRDNSKNTLYLQMNSLRAEDTAVYYCAADVWSGYEYDYWGQGTQVTVSS

SEQ ID NO: 133 - protein, 118 aa, synthetic construct
  REGION 1..118: note = huB11_CNTY1_B11
EVQLLESGGGLVQPGGSLRLSCAASGLAFSSYAMGWYRQAPGKEREFVSVISGTGETLYADSVKGRFTISRDNSKNTLYLQMNSLRAEDTAVYYCAADVWSGYEYDYWGQGTQVTVSS

SEQ ID NO: 134 - protein, 125 aa, synthetic construct
  REGION 1..125: note = Consensus Sequence
  VARIANT 31: note = misc_feature - Xaa can be any naturally occurring amino acid
  VARIANT 33: note = misc_feature - Xaa can be any naturally occurring amino acid
  VARIANT 51: note = misc_feature - Xaa can be any naturally occurring amino acid
  VARIANT 54: note = misc_feature - Xaa can be any naturally occurring amino acid
  VARIANT 61: note = misc_feature - Xaa can be any naturally occurring amino acid
  VARIANT 99..100: note = misc_feature - Xaa can be any naturally occurring amino acid
  VARIANT 102: note = misc_feature - Xaa can be any naturally occurring amino acid
  VARIANT 104: note = misc_feature - Xaa can be any naturally occurring amino acid
  VARIANT 106..111: note = misc_feature - Xaa can be any naturally occurring amino acid
  VARIANT 113..114: note = misc_feature - Xaa can be any naturally occurring amino acid
QVQLVESGGGLVQAGGSLRLSCAASGRIFSXYXMGWFRQAPGKEREFVAAXITXGGSTYYXDSVKGRFTISRDNAKNTVYLQMNSLKPEDTAVYYCAVXXDXGXYXXXXXXYXXWGQGTQVTVSS

(19) United States
(12) Patent Application Publication
(10) Pub. No.: US 2024/0216429 A1
(43) Pub. Date: Jul. 4, 2024

(54) CHIMERIC ANTIGEN RECEPTOR SYSTEM WITH ADAPTABLE RECEPTOR SPECIFICITY

(71) Applicant: CENTURY THERAPEUTICS, INC., Philadelphia, PA (US)

(72) Inventors: Jill Marinari CARTON, Philadelphia, PA (US); Michael Francis NASO, Philadelphia, PA (US); Luis Ghira BORGES, Philadelphia, PA (US); John WHEELER, Philadelphia, PA (US); Andrew DEVANEY, Philadelphia, PA (US)

(73) Assignee: CENTURY THERAPEUTICS, INC., Philadelphia, PA (US)

(21) Appl. No.: 18/409,590

(22) Filed: Jan. 10, 2024

Related U.S. Application Data
(62) Division of application No. 17/554,062, filed on Dec. 17, 2021, now Pat. No. 11,883,432.
(60) Provisional application No. 63/127,587, filed on Dec. 18, 2020.

(51) Int. Cl. A61K 35/17; C12N 5/0783

(52) U.S. Cl. CPC A61K 35/17; C12N 5/0636

(57) ABSTRACT

The present disclosure provides chimeric antigen receptors (CARs), particularly CARs that have adaptable receptor specificity (arCARs). Also provided are polypeptides of the CARs and other related molecules, polynucleotides, vectors, and cell compositions comprising the same. Pharmaceutical compositions comprising the polypeptides, polynucleotides, vectors, or cells of the present disclosure, and their uses in treating a disease in a subject are also provided.

### CROSS REFERENCE TO RELATED APPLICATIONS

This application is a divisional of U.S. application Ser. No. 17/554,062, filed Dec. 17, 2021, which claims the benefit of U.S. Provisional Patent Application No. 63/127,587 filed Dec. 18, 2020. The entire contents of these applications are incorporated herein by reference in their entirety.

### FIELD OF THE INVENTION

The application relates to chimeric antigen receptors (CARs), particularly CARs with an adaptable receptor specificity (arCARs), and their uses in immunotherapy (e.g., adoptive cell therapy).

### REFERENCE TO SEQUENCE LISTING SUBMITTED ELECTRONICALLY

This application contains a sequence listing, which is submitted electronically via Patent Center as an XML formatted sequence listing with a file name “SequenceListing_ST26.xml” and a creation date of Mar. 4, 2024 and having a size of 178,177 bytes. The sequence listing submitted via Patent Center is part of the specification and is herein incorporated by reference in its entirety.

### BACKGROUND

Adoptive cell therapy (ACT) typically involves isolating cells from a donor, culturing and/or manipulating cells in vitro, and then transferring the cells to a patient for the treatment of a disease. To enable the cells to target a specific antigen, the cells are often engineered with a chimeric antigen receptor (CAR). A conventional CAR has a fixed design; thus, one type of CAR T cell usually can only target one antigen epitope. This rigid design limits clinical application and leads to exceptionally high manufacturing cost. While there are various approaches for switch-CAR platforms, these antigen-specific CARs are typically generated on a custom-made basis.

Accordingly, there remains a need for an improved universal CAR platform with a built-in and convenient mechanism for modulating the CAR to easily adapt the CAR's specificity to improve therapies and methods for treating diseases using adoptive cell therapy.

### SUMMARY OF THE INVENTION

In one aspect, provided herein is a universal chimeric antigen receptor system having an adaptable receptor specificity component (arCAR) comprising:
(i) an immune effector cell having a chimeric antigen receptor comprising a first polypeptide comprising (a) an extracellular tag-binding domain, (b) a transmembrane domain, and (c) at least one intracellular signaling domain; and
(ii) a second polypeptide comprising (a) an antigen-binding domain that binds to at least one antigen on a target cell, and (b) a tag that is recognized by the tag-binding domain of the first polypeptide of the chimeric antigen receptor;
   wherein: (i) the tag comprises an antibody, or antigen-binding fragment thereof, or an alternative scaffold and the tag-binding domain comprises an anti-idiotype molecule that binds to the tag, or
   (ii) the tag comprises an anti-idiotype molecule that binds to the tag-binding domain and the tag-binding domain comprises an antibody, or antigen-binding fragment thereof, or an alternative scaffold.

In some embodiments, the anti-idiotype molecule binds to at least one antigen binding region of the antibody, antigen-binding fragment thereof or alternative scaffold. In some embodiments, the anti-idiotype molecule binds to at least one complementarity determining region (CDR) of the antibody, or antigen-binding fragment thereof.

In some embodiments, the antigen-binding domain of the second polypeptide comprises an antibody, or antigen-binding fragment thereof, or an alternative scaffold. In some embodiments, the anti-idiotype molecule is an anti-idiotype antibody, or antigen-binding fragment thereof, or an anti-idiotype alternative scaffold.

In some embodiments, the antigen-binding fragment is an Fab fragment, an Fab′ fragment, an F(ab′)2 fragment, an scFv fragment, an Fv fragment, a dsFv diabody, a VHH, a VNAR, a single-domain antibody (sdAb) or nanobody, a dAb fragment, a Fd′ fragment, a Fd fragment, a heavy chain variable region, an isolated complementarity determining region (CDR), a diabody, a triabody, or a decabody.

In some embodiments, at least one of the extracellular tag-binding domain, the antigen-binding domain, or the tag comprises a single-domain antibody or nanobody. In some embodiments, at least one of the extracellular tag-binding domain, the antigen-binding domain, or the tag comprises a VHH. In some embodiments, the extracellular tag-binding domain and the tag each comprise a VHH. In some embodiments, the extracellular tag-binding domain, the tag, and the antigen-binding domain each comprise a VHH.

In some embodiments, one or more of the antigen-binding fragment, the extracellular tag-binding domain, the antigen-binding domain, and the tag comprise at least in part a polypeptide sequence having at least 90%, 91%, 92%, 93%, 94%, 95%, 96%, 97%, 98%, 99% or 100% sequence identity to a sequence selected from the group consisting of SEQ ID NOs.: 84, 86, 88, 90, 92, 94, 96, 98, 100, 102, 104, 106, 108, and 110-133.

In some embodiments, at least one of the extracellular tag-binding domain, the antigen-binding domain, or the tag comprises an scFv.

In some embodiments, the alternative scaffold is Affilin or Centyrin.

In some embodiments, the tag comprises an antibody, or antigen-binding fragment thereof, or an alternative scaffold. In some embodiments, the antibody, or antigen-binding fragment thereof, or alternative scaffold binds to a polypeptide from a non-human source. In some embodiments, the polypeptide derived from a non-human source is respiratory syncytial virus (RSV) F-protein, Listeria internalin, Cobra phospholipase A2, Ebola nucleoprotein, Herpes simplex virus (HSV) glycoprotein D, lactococcal phage receptor binding protein (RBP), Geobacillus stearothermophilus, ricin, or chicken egg white lysozyme.

In some embodiments, the antigen-binding domain binds to at least one tumor antigen or autoimmune antigen. In some embodiments, the at least one antigen are associated with the same tumor or autoimmune disease. In some embodiments, the tumor antigen is associated with glioblastoma, ovarian cancer, cervical cancer, head and neck cancer, liver cancer, prostate cancer, pancreatic cancer, renal cell carcinoma, bladder cancer, or hematologic malignancy. In some embodiments, the tumor antigen associated with glioblastoma is HER2, EGFRVIII, EGFR, CD133, PDGFRA, FGFR1, FGFR3, MET, or IL13Rα2. In some embodiments, the tumor antigen associated with ovarian cancer is FOLR1, FSHR, MUC16, MUC1, Mesothelin, CA125, EpCAM, EGFR, PDGFRα, or B7H4. In some embodiments, the tumor antigen associated with cervical cancer or head and neck cancer is GD2, MUC1, Mesothelin, HER2, or EGFR. In some embodiments, the tumor antigen associated with liver cancer is Claudin 18.2, GPC-3, EpCAM, cMET, or AFP.

In some embodiments, the transmembrane domain further comprises a hinge domain. In some embodiments, the transmembrane domain and/or hinge domain is derived from CD8 or CD28.

In some embodiments, the at least one intracellular signaling domain comprises a CD3ζ signaling domain.

In some embodiments, the at least one intracellular signaling domain comprises one or more co-stimulatory signaling domains. In some embodiments, the one or more co-stimulatory signaling domains are derived from CD28, 41BB, IL2Rb, CD40, OX40, CD80, CD86, CD27, ICOS, NKG2D, DAP10, DAP12, or 2B4 (CD244).

In various embodiments, the second polypeptide comprises the antigen-binding domain at the N-terminus and the tag at the C-terminus. In some embodiments, the second polypeptide comprises the antigen-binding domain at the C-terminus and the tag at the N-terminus.

In various embodiments, the second polypeptide is a soluble polypeptide.

In various embodiments of the arCAR described herein, the immune effector cell is a T cell, a Natural Killer (NK) cell, a cytotoxic T lymphocyte (CTL), a regulatory T cell or a tumor-infiltrating lymphocyte (TIL), a dendritic cell or a macrophage. In some embodiments, the immune effector cell is derived from an induced pluripotent stem cell (iPSC). In some embodiments, the immune effector cell is a T cell or NK cell derived from an induced pluripotent stem cell (iPSC).

In various embodiments of the arCAR described herein, the arCAR further comprises one or more polypeptides each comprising (a) an antigen-binding domain that binds to a unique antigen and (b) a tag that is recognized by the tag-binding domain of the first polypeptide.

In another aspect, provided herein is an arCAR system comprising two or more arCARs described herein, wherein each arCAR comprises a unique pair of tag and tag-binding domain.

In another aspect, provided herein is a polynucleotide encoding the first polypeptide of the arCAR system described herein.

In another aspect, provided herein is a polynucleotide encoding the second polypeptide of the arCAR system described herein.

In another aspect, provided herein is a polynucleotide encoding the first polypeptide and the second polypeptide of the arCAR system described herein.

In some embodiments of the polynucleotide described herein, one or more of the antigen-binding fragment, the extracellular tag-binding domain, the antigen-binding domain, and the tag are encoded at least in part by a polynucleotide sequence having at least 90%, 91%, 92%, 93%, 94%, 95%, 96%, 97%, 98%, 99% or 100% sequence identity to a sequence selected from the group consisting of SEQ ID NOs.: 83, 85, 87, 89, 91, 93, 95, 97, 99, 101, 103, 105, 107, and 109.

In another aspect, provided herein is a recombinant vector comprising the polynucleotide described herein. In some embodiments, the vector is a plasmid.

In another aspect, provided herein is a host cell comprising a polynucleotide that expresses the first polypeptide of the arCAR system described herein.

In another aspect, provided herein is a host cell comprising a polynucleotide that expresses the second polypeptide of the arCAR system described herein.

In another aspect, provided herein is a pharmaceutical composition comprising the immune effector cell of the arCAR system described herein and a pharmaceutically acceptable carrier and/or excipient.

In another aspect, provided herein is a pharmaceutical composition comprising the second polypeptide of the arCAR system described herein and a pharmaceutically acceptable carrier and/or excipient.

In another aspect, provided herein is a kit comprising the pharmaceutical composition comprising the immune effector cell of the arCAR system described herein and the pharmaceutical composition comprising the second polypeptide of the arCAR system in combination.

In another aspect, provided herein is a method of preparing the host cell comprising a polynucleotide that expresses the first polypeptide, comprising introducing the polynucleotide encoding the first polypeptide of the arCAR system, or a recombinant vector comprising the polynucleotide, into the cell.

In another aspect, provided herein is a method of preparing the host cell comprising a polynucleotide that expresses the second polypeptide, comprising introducing the polynucleotide encoding the second polypeptide of the arCAR system, or a recombinant vector comprising the polynucleotide, into the cell.

In another aspect, provided herein is a method of treating a disease in a subject in need thereof, comprising administering to the subject a therapeutically effective amount of
(i) an immune effector cell comprising a chimeric antigen receptor comprising the first polypeptide of the arCAR described herein, and
(ii) the second polypeptide of said arCAR, or a polynucleotide encoding said second polypeptide, or a host cell comprising said second polypeptide.

In some embodiments, the second polypeptide is administered before, after or in conjunction with the immune effector cell.

In another aspect, provided herein is a method of treating a disease in a subject in need thereof, comprising administering to the subject an therapeutically effective amount of
(i) An immune effector cell comprising the first polypeptides of two or more arCARs, wherein the two or more arCARs each comprise a unique pair of tag and tag-binding domain, and
(ii) the second polypeptides of said two or more arCARs, or one or more polynucleotide encoding said second polypeptides, or one or more host cells comprising said second polypeptides.

In some embodiments of the treatment methods described herein, the immune effector cell is a T cell, a Natural Killer (NK) cell, a cytotoxic T lymphocyte (CTL), a regulatory T cell or a tumor-infiltrating lymphocyte (TIL), dendritic cell or macrophage. In some embodiments, the immune effector cell is derived from an iPSC. In some embodiments, the immune effector cell constitutively expresses the first polypeptide.

In some embodiments, the disease is a cancer or autoimmune disease. In some embodiments, the cancer is glioblastoma, ovarian cancer, cervical cancer, head and neck cancer, liver cancer, prostate cancer, pancreatic cancer, renal cell carcinoma, bladder cancer, or hematologic malignancy.

In some embodiments, the immune effector cell and the second polypeptide(s) are administered simultaneously. In some embodiments, the immune effector cell and the second polypeptide(s) are administered sequentially.

In some embodiments, the immune effector cell is an autologous cell. In some embodiments, the immune effector cell is an allogeneic cell.

In some embodiments, the subject is a mammal. In some embodiments, the subject is a human.

### BRIEF DESCRIPTION OF THE DRAWINGS

FIG. 1 depicts a schematic representation of an exemplary CAR having an adaptable receptor specificity (arCAR) of the present disclosure. The CAR binds to a “tag” on a soluble drug. The soluble drug can be an antibody or antibody fragment (e.g., VHH, scFv, Fab, mAb) or an alternative scaffold. Tumor specificity and CAR activation is driven by the soluble drug.

FIG. 2A depicts a schematic representation of another exemplary arCAR of the present disclosure. A VHH is used in both the antigen-binding domain and the tag in the soluble drug, as well as the tag-binding domain (α-tag) of the arCAR.

FIG. 2B illustrates the structural characteristics of an anti-idiotype mAb.

FIG. 2C illustrates the structural characteristics of an exemplary VHH structure.

FIG. 2D depicts a schematic representation of another exemplary arCAR of the present disclosure. A non-human primate (NHP) VHH and an anti-idiotype NHP VHH are used in the soluble drug, as well as the tag-binding domain of the arCAR.

FIG. 3A depicts a schematic representation of another exemplary arCAR of the present disclosure.

FIG. 3B depicts a schematic representation of another exemplary arCAR of the present disclosure.

FIG. 4A depicts a schematic representation of another exemplary arCAR of the present disclosure. Herceptin scFv is fused to the CAR and a Herceptin anti-ID scFv is fused to an EGFR-targeting VHH (9G8).

FIG. 4B shows that a T cell expressing the Herceptin scFv CAR, coupled with the 9G8-scFv69 fusion protein, is capable of targeting an EGFR+ cell.

FIG. 5 depicts a vector map of the lentiviral vector used to deliver a CAR construct of the present disclosure.

FIG. 6A depicts a vector map of the plasmid used to express a bridge construct of the present disclosure.

FIG. 6B shows a gel electrophoresis image demonstrating the purity of the bridge proteins.

FIG. 7A depicts a schematic representation of the scFv69-CAR and two variants of the bridge protein: 9G8_Herceptin scFv and Herceptin scFv 9G8.

FIG. 7B demonstrates binding of the scFv69-CAR to the bridge proteins. The samples in the graph appear in the following order: 9G8_Herceptin, Herceptin_9 G8.

FIG. 8A demonstrates expression of EGFR on the EGFR transduced CHO cells.

FIG. 8B demonstrates CAR expression in T cells.

FIGS. 9A-9B show that universal CAR-T cells demonstrated CD25 activation when paired with the correct bridge protein (FIG. 9A) but not when paired with a mis-matched bridge protein (FIG. 9B). The samples in the graphs appear in the order as specified in the legend.

FIGS. 10A, 10B show that universal CAR-T cells demonstrated cytotoxic activity when paired with the correct bridge protein (FIG. 10A) but not when paired with a mis-matched bridge protein (FIG. 10B). The samples in the graphs appear in the order as specified in the legend.

FIG. 11A depicts a vector map of the lentiviral vector used to deliver a VHH CAR of the present disclosure.

FIG. 11B shows expression for VHH CAR on Jurkat cells.

FIG. 11C shows lack of tonic signaling on the surface of Jurkat cells transduced VHH CAR.

FIG. 11D shows the FACs gating strategy used for CD69 detection in FIG. 11C.

FIG. 11E shows binding of anti-Chicken Hen Egg Lysozyme-VHH CARs to lysozyme from chicken or human.

FIG. 11F shows alignment of the amino acid sequences of VHHs targeting Geobacillus stearothermophilus (SEQ ID NO: 96), LYSO_CW_P01_B11 (SEQ ID NO: 102) and LYSO_CW_P01_D04 (SEQ ID NO: 104). A consensus sequence is shown above the aligned sequences (SEQ ID NO: 134).

FIGS. 12A-1-12K show plasmid maps p510 (FIGS. 12A-1-12A-4), p511 (FIGS. 12B-1-12B-4), p514 (FIGS. 12C-1-12C-4), p515 (FIGS. 12D-1-12D-4), p516 (FIGS. 12E-1 12E-4), p517 (FIG. 12F), p518 (FIG. 12G), p519 (FIG. 12H), p520 (FIG. 12I), p521 (FIG. 12J), and p522 (FIG. 12K) for the arCAR proof-of-concept studies.

FIG. 13 shows CAR and GFP expression of Nurkat cells (Nur77 reporter line of Jurkat cells) transduced with lentivirus (LV) containing the humanized B11 (p1649) and D04 (p1648) CARs. Also shown are results for untransduced (UTD) Nurkats. CAR expression was measured by the polyclonal anti-camelid VHH cocktail from Genscript (e.g., sufficient homology between the humanized and camelid versions of VHH for robust detection).

FIG. 14 shows B11 (p1649) and D04 (p1648) CAR Nurkat cells stained with biotinylated HEL detected with streptavidin-APC via flow cytometry. UTD are untransduced Nurkat cells used as a negative control.

FIG. 15 shows an overview of the phage display panning schema. The schema for anti-idiotype discovery against D04 is shown as an example. Likewise, for anti-idiotype discovery against B11, the two VHH would swap places in the diagram.

FIGS. 16A-16D show periplasmic extract (PPE) ELISA outputs (absorbance at 450 nm) the colonies achieved for each CNTY library. Black bars represent ELISA responses against the B11 protein, while gray bars represent responses against the D04 protein.

FIGS. 17A-17B show FACS binding of anti-Idiotype VHH-Fc fusion constructs to (FIG. 17A) B11 and (FIG. 17B) D04 CAR-expressing Nurkat cells without an IgG-based linker. The dashed outline identifies select proteins from Table 15 that were observed in ELISA to bind D04. The dotted outline identifies select proteins from Table 16 that were observed in ELISA to bind B11. Each protein was tested on both cell lines in a 3-fold dilution series from 50 nM down to 0.02 pM (as shown in the legend). Fc-fusion protein was detected via anti-Fc APC, and the percentage of cells that were positive for this detection method are shown. Proteins that showed specific binding down the dilution series to their target cell line are starred below the protein ID. Controls include (i) no VHH-Fc added and (ii) the anti-VHH cocktail detection reagent (far right). The samples shown in the graphs for each protein appear in the order as specified in the legend.

FIG. 18 shows binding of the lead anti-idiotype biotinylated VHH-Fc to target and off target CAR Nurkat cells detected via FACS with streptavidin-APC. The cells lines that were tested include B11, D04, P711, P712, P713, P716, and parental cells. B11 and D04 cells are CAR Nurkats expressing B11 and D04 VHH CARs, respectively. P711, P712, P713, P716 cells are off-target CAR Nurkats. Parental cells are untransduced, non-CAR-expressing Nurkats. Controls include biotinylated Protein A (which nonspecifically binds most VHH), PROT786 (an unrelated biotinylated VHH-Fc protein), and no biotinylated protein added. The samples shown in the graphs for each protein appear in the order as specified in the legend.

FIGS. 19A-19B show results of an overnight stimulation assay with Nurkat cells incubated with 10 nM anti-idiotype VHH Fc-fusion via FACS. (FIG. 19A) GFP expression of B11 and D04 CAR Nurkat cells, as well as parental Nurkat cells after overnight coculture with anti-idiotype Fc-fusion proteins. (FIG. 19B) Detection of biotinylated protein via streptavidin-APC after the overnight incubation. Controls include PMA/Ionomycin (a TCR-crosslinker that results in robust Nurkat cell activation), biotinylated protein A, and a sample with no protein added. The samples shown in the graphs for each protein appear in the order as specified in the legend.

FIGS. 20A-20B show results of identical T cell killing assays in which either bridge protein (FIG. 20A) AMD109 or (FIG. 20B) AMD112 was added at 5 nM. Percent dead targets are shown for either U87 target cells, or K562 target cells.

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

The present application provides, among other things, universal chimeric antigen receptor systems having an adaptable receptor specificity component (arCARs) and their uses in immunotherapy (e.g., adoptive cell therapy). This arCAR platform provides a built-in and convenient mechanism for modulation of the receptor using tag polypeptide affinity and allowing for multiple receptors to be present on a cell therapy. Such arCARs can enable fit-for-purpose cell therapy.

### Definitions

The term “chimeric antigen receptor” or “CAR” as used herein generally refers to a cell-surface receptor comprising an extracellular target-binding domain, a transmembrane domain, and a cytoplasmic domain comprising a signaling domain and optionally at least one costimulatory signaling domain, all in a combination that is not naturally found together on a single protein. This particularly includes receptors wherein the extracellular domain and the cytoplasmic domain are not naturally found together on a single receptor protein.

The term “chimeric antigen receptor having an adaptable receptor specificity component” or “arCAR” as used herein refers to a two-component CAR system wherein the extracellular target-binding domain of the receptor can be coupled with a variety of different antigen-binding moieties. Except as otherwise indicated, the term “chimeric antigen receptor” or “CAR” used herein is meant to encompass the “chimeric antigen receptor having an adaptable receptor specificity component” or “arCAR” described herein. The arCAR system of the present disclosure may be used with immune effector cells such as lymphocytes including T cells and natural killer (NK) cells, which may be derived from stem cells such as induced pluripotent stem cells (iPSCs).

The term “immune effector cell” as used herein means a cell that has differentiated into a form capable of modulating or effecting a specific immune response. Such cells may include mature lymphocytes suitable for therapy, including, but not limited to, cytotoxic T-cells, helper T-cells, natural killer cells, and tumor-infiltrating lymphocytes (TILs), and may also include dendritic cells or macrophages.

The term “antibody” herein is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, scFv fragments, recombinant IgG (rIgG) fragments, single chain antibody fragments, including single chain variable fragments (scFv), and single domain antibodies (e.g., VHH, VNAR, sdAb, sdFv) fragments or nanobodies, Fd′ fragments, Fd fragments, heavy chain variable regions, or isolated complementarity determining regions (CDRs). The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, tetrabodies, decabodies, tandem di-scFv, and tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof (e.g., IgG1, IgG2, IgG3, IgG4), IgM, IgY, IgE, IgA, and IgD.

The term “anti-idiotype molecule” refers to a molecule (e.g., peptide, protein, antibody or antibody fragment, alternative scaffold) that specifically recognizes, is specifically targeted to, and/or specifically binds to an idiotope of an antibody, such as an antigen-binding fragment. The idiotopes of an antibody may include, but are not necessarily limited to, residues within one or more of complementarity determining region(s) (CDRs) of the antibody, variable regions of the antibody, and/or partial portions or portions of such variable regions and/or of such CDRs, and/or any combination of the foregoing. The CDR may be one or more selected from the group consisting of CDR-H1, CDR-H2, CDR-H3, CDR-L1, CDR-L2, and CDR-L3. The variable regions of the antibody may be heavy chain variable regions, light chain variable regions, or a combination of the heavy chain variable regions and the light chain variable regions. The partial fragments or portions of the heavy chain variable regions and/or the light chain variable regions of the antibody may be fragments including 2 or more, 5 or more, or 10 or more contiguous amino acids, for example, from about 2 to about 100, from about 5 to about 100, from about 10 to about 100, from about 2 to about 50, from about 5 to about 50, or from about 10 to about 50 contiguous amino acids within the heavy chain variable regions or the light chain variable regions of the antibody; the idiotope may include multiple non-contiguous stretches of amino acids. The partial fragments of the heavy chain variable regions and the light chain variable regions of the antibody may be fragments including 2 or more, 5 or more, or 10 or more contiguous amino acids, for example, from about 2 to about 100, from about 5 to about 100, from about 10 to about 100, from about 2 to about 50, from about 5 to about 50, or from about 10 to about 50 contiguous amino acids within the variable regions, and in some embodiments contain one or more CDRs or CDR fragments. The CDR fragments may be consecutive or non-consecutive 2 or more, or 5 or more amino acids within the CDR. Therefore, the idiotopes of the antibody may be from about 2 to about 100, from about 5 to about 100, from about 10 to about 100, from about 2 to about 50, from about 5 to about 50, or from about 10 to about 50 contiguous amino acids containing one or more CDR or one or more CDR fragments within the heavy chain variable regions or the light chain variable regions of the antibody. In another embodiment, the idiotopes may be a single amino acid which is located at the variable regions of the antibody, for example, CDR sites.

In some embodiments, the idiotope is any single antigenic determinant or epitope within the variable portion of an antibody. In some cases, it can overlap the actual antigen-binding site of the antibody, and in some cases, it may comprise variable region sequences outside of the antigen-binding site of the antibody. The set of individual idiotopes of an antibody is in some embodiments referred to as the “idiotype” of such antibody.

As used herein, the term “antigen” refers to any agent (e.g., protein, peptide, polysaccharide, glycoprotein, glycolipid, nucleic acid, portions thereof, or combinations thereof) molecule capable of being bound by an antibody or antibody fragment, T-cell receptor or alternative scaffold. An antigen is also able to provoke an immune response. An example of an immune response may involve, without limitation, antibody production, or the activation of specific immunologically competent cells, or both. A skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components, organisms, subunits of proteins/antigens, killed or inactivated whole cells or lysates.

The terms “vector”, “expression vector”, and “expression construct” are used interchangeably to refer to a composition of matter which can be used to deliver a nucleic acid of interest to the interior of a cell and mediate its expression within the cell. Most commonly used examples of vectors are autonomously replicating plasmids and viruses (such as, e.g., adenoviral vectors, adeno-associated virus vectors (AAV), lentiviral vectors, Sindbis virus vectors, etc.). An expression construct can be replicated in a living cell, or it can be made synthetically. In one embodiment, an expression vector comprises a promoter operably linked to a polynucleotide (e.g., a polynucleotide encoding the first polypeptide and/or second polypeptide of an arCAR described herein) which promoter controls the initiation of transcription by RNA polymerase and expression of the polynucleotide. Typical promoters for mammalian cell expression include, e.g., SV40 early promoter, CMV immediate early promoter (see, e.g., U.S. Pat. Nos. 5,168,062 and 5,385,839, both of which are incorporated herein by reference in their entirety), mouse mammary tumor virus LTR promoter, adenovirus major late promoter (Ad MLP), Herpes simplex virus promoter, murine metallothionein gene promoter, and U6 or H1 RNA pol III promoter. Non-limiting examples of promoters useful for expressing first polypeptide and/or second polypeptide of an arCAR described herein in the methods of the present disclosure include, e.g., Synapsin promoter (neuron specific), CamKIIa promoter (specific for excitatory neurons), ubiquitin promoter, CAG promoter, CMV promoter, and β-actin promoter. These and other promoters can be obtained from commercially available plasmids, using techniques well known in the art. See, e.g., Sambrook et al., supra. Enhancer elements may be used in association with promoters to increase expression levels of the vectors. Examples include the SV40 early gene enhancer, as described in Dijkema et al., EMBO J. (1985) 4:761, the enhancer/promoter derived from the long terminal repeat (LTR) of the Rous Sarcoma Virus, as described in Gorman et al., Proc. Natl. Acad. Sci. USA (1982b) 79:6777, which is incorporated herein by reference in its entirety, and elements derived from human CMV, as described in Boshart et al., Cell (1985) 41:521, which is incorporated herein by reference in its entirety, such as elements included in the CMV intron A sequence.

Transcription terminator/polyadenylation signals may also be present in the expression vector. Examples of such sequences include, but are not limited to, those derived from SV40, as described in Sambrook et al., supra, as well as a bovine growth hormone terminator sequence (see, e.g., U.S. Pat. No. 5,122,458, which is incorporated herein by reference in its entirety). Additionally, 5′-UTR sequences can be placed adjacent to the coding sequence in order to enhance expression of the same. Such sequences include UTRs which include, e.g., an Internal Ribosome Entry Site (IRES) present in the leader sequences of picornaviruses such as the encephalomyocarditis virus (EMCV) UTR (Jang et al. J. Virol. (1989) 63:1651-1660, which is incorporated herein by reference in its entirety). Other useful picornavirus UTR sequences include, e.g., the polio leader sequence, hepatitis A virus leader and the hepatitis C IRES.

The term “host cell” means any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5α, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, CHO, Saos, and PC12.

Host cells of the present disclosure include T cells and natural killer cells that contain the DNA or RNA sequences encoding the CAR and/or express the CAR on the cell surface. Host cells may be used for enhancing T cell activity, natural killer cell activity, treatment of cancer, and treatment of autoimmune disease.

The terms “T cell” and “T lymphocyte” are interchangeable and used synonymously herein. As used herein, T cell includes thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+CD8+ T cell, or any other subset of T cells. Other illustrative populations of T cells suitable for use in particular embodiments include naive T cells and memory T cells. Also included are “NKT cells”, which refer to a specialized population of T cells that express a semi-invariant αβ T-cell receptor, but also express a variety of molecular markers that are typically associated with NK cells, such as NK1.1. NKT cells include NK1.1+ and NK1.1−, as well as CD4+, CD4−, CD8+ and CD8− cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain. γδ T cells can play a role in immunosurveillance and immunoregulation, and were found to be an important source of IL-17 and to induce robust CD8+ cytotoxic T cell response. Also included are “regulatory T cells” or “Tregs” refers to T cells that suppress an abnormal or excessive immune response and play a role in immune tolerance. Tregs cells are typically transcription factor Foxp3-positive CD4+ T cells and can also include transcription factor Foxp3-negative regulatory T cells that are IL-10-producing CD4+ T cells.

The terms “natural killer cell” and “NK cell” are used interchangeable and used synonymously herein. As used herein, NK cell refers to a differentiated lymphocyte with a CD 16+CD56+ and/or CD57+ TCR− phenotype. NKs are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

In certain embodiments of the disclosure, the cells containing nucleic acid constructs of the present disclosure may be identified in vitro or in vivo by including a marker in the expression vector. Such markers would confer an identifiable change to the cell permitting easy identification of cells containing the expression vector. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as Herpes simplex virus thymidine kinase (tk) or chloramphenicol acetyltransferase (CAT) may be employed. Fluorescent markers (e.g., green fluorescent protein (GFP), EGFP, or Dronpa), or immunologic markers can also be employed. Further examples of selectable markers are well known to one of skill in the art.

As used herein, the term “variant” in the context of proteins or polypeptides (e.g., arCAR polypeptides or domains thereof) refer to: (a) a polypeptide that has at least 40%, 45%, 50%, 55%, 60%, 65%, 70%, 75%, 80%, 85%, 90%, 95%, 98% or 99% sequence identity to the polypeptide it is a variant of; (b) a polypeptide encoded by a nucleotide sequence that has at least 40%, 45%, 50%, 55%, 60%, 65%, 70%, 75%, 80%, 85%, 90%, 95%, 98% or 99% sequence identity to a nucleotide sequence encoding the polypeptide it is a variant of; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to the polypeptide it is a variant of; (d) a polypeptide encoded by nucleic acids can hybridize under high, moderate or typical stringency hybridization conditions to nucleic acids encoding the polypeptide it is a variant of; (e) a polypeptide encoded by a nucleotide sequence that can hybridize under high, moderate or typical stringency hybridization conditions to a nucleotide sequence encoding a fragment of the polypeptide, it is a variant of, of at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, or at least 150 contiguous amino acids; or (f) a fragment of the polypeptide it is a variant of.

Percent sequence identity can be determined using any method known to one of skill in the art. In a specific embodiment, the percent identity is determined using the “Best Fit” or “Gap” program of the Sequence Analysis Software Package (Version 10; Genetics Computer Group, Inc., University of Wisconsin Biotechnology Center, Madison, Wisconsin). Information regarding hybridization conditions (e.g., high, moderate, and typical stringency conditions) have been described, see, e.g., U.S. Patent Application Publication No. US 2005/0048549 (e.g., paragraphs 72-73).

In the context of the present disclosure insofar as it relates to any of the disease conditions recited herein, the terms “treat”, “treatment”, and the like mean to prevent, relieve or alleviate at least one symptom associated with such condition, or to slow or reverse the progression of such condition, or to arrest, delay the onset (i.e., the period prior to clinical manifestation of a disease) and/or reduce the risk of developing or worsening a disease.

As used herein the term “therapeutically effective” applied to dose or amount refers to that quantity of a compound or pharmaceutical composition that is sufficient to result in a desired activity (e.g., alleviation of symptoms associated with cancer or autoimmune disease) upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like. An appropriate “effective” amount in any individual case may be determined by one of ordinary skill in the art using routine experimentation, based upon the information provided herein.

The phrase “pharmaceutically acceptable”, as used in connection with compositions of the disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

As used herein, the term “combination” of a composition of the disclosure and at least an additional therapeutic agent means at least two, but any desired combination of agents can be delivered simultaneously or sequentially. It is contemplated that when used to treat various diseases, the compositions and methods of the present disclosure can be utilized with other therapeutic methods/agents suitable for the same or similar diseases. Such other therapeutic methods/agents can be co-administered (simultaneously or sequentially) to generate additive or synergistic effects. Suitable therapeutically effective dosages for each agent may be lowered due to the additive action or synergy.

The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

An “individual” or “subject” or “animal”, as used herein, refers to humans, veterinary animals (e.g., cats, dogs, cows, horses, sheep, pigs, etc.) or experimental animal models of a disease or disorder (e.g., cancer or autoimmune disease). In a preferred embodiment, the subject is a human.

The term “associated with” is used to encompass any correlation, co-occurrence and any cause-and-effect relationship.

The term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean within an order of magnitude, preferably within 50%, more preferably within 20%, still more preferably within 10%, even more preferably within 5%, and most preferably within 1% of a given value or range. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

It must be noted that, as used in this specification and the appended claims, the singular forms “a,” “an” and “the” include plural referents unless the content clearly dictates otherwise.

In accordance with the present disclosure there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition. Cold Spring Harbor, NY: Cold Spring Harbor Laboratory Press, 1989 (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); Ausubel, F. M. et al. (eds.). Current Protocols in Molecular Biology. John Wiley & Sons, Inc., 1994. These techniques include site directed mutagenesis as described in Kunkel, Proc. Natl. Acad. Sci. USA 82: 488-492 (1985), U.S. Pat. No. 5,071,743, Fukuoka et al., Biochem. Biophys. Res. Commun. 263: 357-360 (1999); Kim and Maas, BioTech. 28: 196-198 (2000); Parikh and Guengerich, BioTech. 24: 4 28-431 (1998); Ray and Nickoloff, BioTech. 13: 342-346 (1992); Wang et al., BioTech. 19: 556-559 (1995); Wang and Malcolm, BioTech. 26: 680-682 (1999); Xu and Gong, BioTech. 26: 639-641 (1999), U.S. Pat. Nos. 5,789,166 and 5,932,419, Hogrefe, Strategies 14. 3: 74-75 (2001), U.S. Pat. Nos. 5,702,931, 5,780,270, and 6,242,222, Angag and Schutz, Biotech. 30: 486-488 (2001), Wang and Wilkinson, Biotech. 29: 976-978 (2000), Kang et al., Biotech. 20: 44-46 (1996), Ogel and McPherson, Protein Engineer. 5: 467-468 (1992), Kirsch and Joly, Nucl. Acids. Res. 26: 1848-1850 (1998), Rhem and Hancock, J. Bacteriol. 178: 3346-3349 (1996), Boles and Miogsa, Curr. Genet. 28: 197-198 (1995), Barrenttino et al., Nuc. Acids. Res. 22: 541-542 (1993), Tessier and Thomas, Meths. Molec. Biol. 57: 229-237, and Pons et al., Meth. Molec. Biol. 67: 209-218.

Chimeric Antigen Receptors Having an Adaptable Receptor Specificity Component (arCARs)

In one aspect, provided herein is a universal chimeric antigen receptor system having an adaptable receptor specificity component (arCAR) comprising:
(i) an immune effector cell having a chimeric antigen receptor comprising a first polypeptide comprising (a) an extracellular tag-binding domain, (b) a transmembrane domain, and (c) at least one intracellular signaling domain; and
(ii) a second polypeptide comprising (d) an antigen-binding domain that binds to at least one antigen on a target cell, and (e) a tag that is recognized by the tag-binding domain of the first polypeptide of the chimeric antigen receptor;
   wherein: (i) the tag comprises an antibody, or antigen-binding fragment thereof, or an alternative scaffold and the tag-binding domain comprises an anti-idiotype molecule that binds to the tag, or
   (ii) the tag comprises an anti-idiotype molecule that binds to the tag-binding domain and the tag-binding domain comprises an antibody, or antigen-binding fragment thereof, or an alternative scaffold.
In some embodiments, the anti-idiotype molecule binds to at least one antigen binding region or non-framework region of the antibody, or antigen-binding fragment thereof.

In some embodiments, the tag comprises an antibody, or antigen-binding fragment thereof, or an alternative scaffold that binds to a polypeptide derived from a non-human source. Alternatively, the tag-binding domain comprises an antibody, or antigen-binding fragment thereof, or an alternative scaffold that binds to a polypeptide derived from a non-human source.

In some embodiments, the anti-idiotype molecule binds to at least one complementarity determining region (CDR) of the antibody, or antigen-binding fragment thereof.

In some embodiments, the anti-idiotype molecule is an anti-idiotype antibody, or antigen-binding fragment thereof, or an alternative scaffold.

In some embodiments, the antigen-binding domain of the second polypeptide comprises an antibody, or antigen-binding fragment thereof, or an alternative scaffold.

In various embodiments, antibodies or antibody fragments suitable for use in the arCAR system of the present disclosure include, but are not limited to, monoclonal antibodies, bispecific antibodies, multispecific antibodies, chimeric antibodies, polypeptide-Fc fusions, single-chain Fvs (scFv), single chain antibodies, Fab fragments, F(ab′) fragments, disulfide-linked Fvs (sdFv), masked antibodies (e.g., Probodies®), Small Modular ImmunoPharmaceuticals (“SMIPs™”), intrabodies, minibodies, single domain antibody variable domains, nanobodies, VHHs, diabodies, tandem diabodies (TandAb®), and epitope-binding fragments of any of the above. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains.

In some embodiments, the antigen-binding fragment is an Fab fragment, an Fab′ fragment, an F(ab′)2 fragment, an scFv fragment, an Fv fragment, a dsFv diabody, a VHH, a VNAR, a single-domain antibody (sdAb) or nanobody, a dAb fragment, a Fd′ fragment, a Fd fragment, a heavy chain variable region, an isolated complementarity determining region (CDR), a diabody, a triabody, or a decabody. In some embodiments, the antigen-binding fragment is an scFv fragment. In some embodiments, the antigen-binding fragment is a VHH.

In some embodiments, at least one of the extracellular tag-binding domain, the antigen-binding domain, or the tag comprises a single-domain antibody or nanobody.

In some embodiments, at least one of the extracellular tag-binding domain, the antigen-binding domain, or the tag comprises a VHH.

In some embodiments, the extracellular tag-binding domain and the tag each comprise a VHH.

In some embodiments, the extracellular tag-binding domain, the tag, and the antigen-binding domain each comprise a VHH.

In some embodiments, at least one of the extracellular tag-binding domain, the antigen-binding domain, or the tag comprises an scFv.

In some embodiments, the extracellular tag-binding domain and the tag each comprise an scFv.

In some embodiments, the extracellular tag-binding domain, the tag, and the antigen-binding domain each comprise a scFv.

In some embodiments, the antibodies or antigen-binding fragments (e.g., VHH, scFv) used herein are humanized. Humanized proteins have the potential to reduce the risk of immunogenicity.

Alternative scaffolds to immunoglobulin domains that exhibit similar functional characteristics, such as high-affinity and specific binding of target biomolecules, may also be used in the arCARs of the present disclosure. Such scaffolds have been shown to yield molecules with improved characteristics, such as greater stability or reduced immunogenicity. Non-limiting examples of alternative scaffolds that may be used in the arCAR system of the present disclosure include engineered, tenascin-derived, tenascin type III domain (e.g., Centyrin™); engineered, gamma-B crystallin-derived scaffold or engineered, ubiquitin-derived scaffold (e.g., Affilins); engineered, fibronectin-derived, 10th fibronectin type III (10Fn3) domain (e.g., monobodies, AdNectins™, or AdNexins™); engineered, ankyrin repeat motif containing polypeptide (e.g., DARPins™); engineered, low-density-lipoprotein-receptor-derived, A domain (LDLR-A) (e.g., Avimers™); lipocalin (e.g., anticalins); engineered, protease inhibitor-derived, Kunitz domain (e.g., EETI-II/AGRP, BPTI/LACI-D1/ITI-D2); engineered, Protein-A-derived, Z domain (Affibodies™); Sac7d-derived polypeptides (e.g., Nanoffitins® or affitins); engineered, Fyn-derived, SH2 domain (e.g., Fynomers®); CTLD3 (e.g., Tetranectin); thioredoxin (e.g., peptide aptamer); KALBITOR®; the β-sandwich (e.g., iMab); miniproteins; C-type lectin-like domain scaffolds; engineered antibody mimics; and any genetically manipulated counterparts of the foregoing that retains its binding functionality (Wörn A, Pluckthun A, J Mol Biol 305: 989-1010 (2001); Xu L et al., Chem Biol 9: 933-42 (2002); Wikman M et al., Protein Eng Des Sel 17: 455-62 (2004); Binz H et al., Nat Biolechnol 23: 1257-68 (2005); Hey T et al., Trends Biotechnol 23:514-522 (2005); Holliger P, Hudson P, Nat Biotechnol 23: 1126-36 (2005); Gill D, Damle N, Curr Opin Biotech 17: 653-8 (2006); Koide A, Koide S, Methods Mol Biol 352: 95-109 (2007); Skerra, Current Opin. in Biotech., 2007 18: 295-304; Byla P et al., J Biol Chem 285: 12096 (2010); Zoller F et al., Molecules 16: 2467-85 (2011), each of which is incorporated by reference in its entirety).

In some embodiments, the alternative scaffold is Affilin or Centyrin.

The molecule derived from a non-human source may be derived from any non-human organisms, including but not limited to, viruses, bacteria, yeast, fungi, plants, and non-human animals.

In some embodiments, the molecule derived from a non-human source is a polypeptide. In some embodiments, the source of the protein is from a non-human polypeptide that does not have a human homolog.

In some embodiments, the polypeptide derived from a non-human source is respiratory syncytial virus (RSV) F-protein, Listeria internalin, Cobra phospholipase A2, Ebola nucleoprotein, Herpes simplex virus (HSV) glycoprotein D, lactococcal phage receptor binding protein (RBP), Geobacillus stearothermophilus, ricin, or chicken egg white lysozyme.

In some embodiments, the first polypeptide of the arCARs of the present disclosure comprises a leader sequence. The leader sequence may be positioned at the N-terminus of the extracellular tag-binding domain. The leader sequence may be optionally cleaved from the extracellular tag-binding domain during cellular processing and localization of the CAR to the cellular membrane. Any of various leader sequences known to one of skill in the art may be used as the leader sequence. Non-limiting examples of peptides from which the leader sequence may be derived include granulocyte-macrophage colony-stimulating factor (GMCSF), FcεR, human immunoglobulin (IgG) heavy chain (HC) variable region, CD8a, mouse Ig-kappa signal peptide, or any of various other proteins secreted by T cells. In various embodiments, the leader sequence is compatible with the secretory pathway of a T cell. In certain embodiments, the leader sequence is derived from human immunoglobulin heavy chain (HC).

In some embodiments, the leader sequence is derived from GMCSF. In one embodiment, the GMCSF leader sequence comprises the amino acid sequence set forth in SEQ ID NO: 1, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 1.

In some embodiments, the leader sequence is derived from the mouse Ig-kappa signal peptide. In one embodiment, the mouse Ig-kappa signal peptide comprises the amino acid sequence set forth in SEQ ID NO: 61, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 61.

In some embodiments, the first polypeptide of the arCARs of the present disclosure comprise a transmembrane domain, fused in frame between the extracellular tag-binding domain and the cytoplasmic domain.

The transmembrane domain may be derived from the protein contributing to the extracellular tag-binding domain, the protein contributing the signaling or co-signaling domain, or by a totally different protein. In some instances, the transmembrane domain can be selected or modified by amino acid substitution, deletions, or insertions to minimize interactions with other members of the arCAR complex. In some instances, the transmembrane domain can be selected or modified by amino acid substitution, deletions, or insertions to avoid binding of proteins naturally associated with the transmembrane domain. In certain embodiments, the transmembrane domain includes additional amino acids to allow for flexibility and/or optimal distance between the domains connected to the transmembrane domain.

The transmembrane domain may be derived either from a natural or from a synthetic source. Where the source is natural, the domain may be derived from any membrane-bound or transmembrane protein. Non-limiting examples of transmembrane domains of particular use in this disclosure may be derived from (i.e. comprise at least the transmembrane region(s) of) the α, β or ζ chain of the T-cell receptor (TCR), NKG2D, CD28, CD3 epsilon, CD45, CD4, CD5, CD8, CD8α, CD9, CD16, CD22, CD33, CD37, CD40, CD64, CD80, CD86, CD134, CD137, or CD154. Alternatively, the transmembrane domain may be synthetic, in which case it will comprise predominantly hydrophobic residues such as leucine and valine. For example, a triplet of phenylalanine, tryptophan and/or valine can be found at each end of a synthetic transmembrane domain.

In some embodiments, it will be desirable to utilize the transmembrane domain of the ζ, η or FcεR1γ chains which contain a cysteine residue capable of disulfide bonding, so that the resulting chimeric protein will be able to form disulfide linked dimers with itself, or with unmodified versions of the ζ, η or FcεR1γ chains or related proteins. In some instances, the transmembrane domain will be selected or modified by amino acid substitution to avoid binding of such domains to the transmembrane domains of the same or different surface membrane proteins to minimize interactions with other members of the receptor complex. In other cases, it will be desirable to employ the transmembrane domain of ζ, η or FcεR1γ and -β, MB1 (Igα.), B29 or CD3-γ, ζ, or η, in order to retain physical association with other members of the receptor complex.

In some embodiments, the transmembrane domain is derived from CD8 or CD28. In one embodiment, the CD8 transmembrane domain comprises the amino acid sequence set forth in SEQ ID NO: 23, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 23. In one embodiment, the CD28 transmembrane domain comprises the amino acid sequence set forth in SEQ ID NO: 24, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 24.

In some embodiments, the first polypeptide of the arCAR system of the present disclosure comprises a spacer region between the extracellular tag-binding domain and the transmembrane domain, wherein the tag-binding domain, linker, and the transmembrane domain are in frame with each other.

The term “spacer region” as used herein generally means any oligo- or polypeptide that functions to link the tag-binding domain to the transmembrane domain. A spacer region can be used to provide more flexibility and accessibility for the tag-binding domain. A spacer region may comprise up to 300 amino acids, preferably 10 to 100 amino acids and most preferably 25 to 50 amino acids. A spacer region may be derived from all or part of naturally occurring molecules, such as from all or part of the extracellular region of CD8, CD4 or CD28, or from all or part of an antibody constant region. Alternatively, the spacer region may be a synthetic sequence that corresponds to a naturally occurring spacer region sequence, or may be an entirely synthetic spacer region sequence. Non-limiting examples of spacer regions which may be used in accordance to the disclosure include a part of human CD8α chain, partial extracellular domain of CD28, FcyRllla receptor, IgG, IgM, IgA, IgD, IgE, an Ig hinge, or functional fragment thereof. In some embodiments, additional linking amino acids are added to the spacer region to ensure that the antigen-binding domain is an optimal distance from the transmembrane domain. In some embodiments, when the spacer is derived from an Ig, the spacer may be mutated to prevent Fc receptor binding.

In some embodiments, the spacer region comprises a hinge domain. The hinge domain may be derived from CD8α, CD28, or an immunoglobulin (IgG). For example, the IgG hinge may be from IgG1, IgG2, IgG3, IgG4, IgM1, IgM2, IgA1, IgA2, IgD, IgE, or a chimera thereof.

In certain embodiments, the hinge domain comprises an immunoglobulin IgG hinge or functional fragment thereof. In certain embodiments, the IgG hinge is from IgG1, IgG2, IgG3, IgG4, IgM1, IgM2, IgA1, IgA2, IgD, IgE, or a chimera thereof. In certain embodiments, the hinge domain comprises the CH1, CH2, CH3 and/or hinge region of the immunoglobulin. In certain embodiments, the hinge domain comprises the core hinge region of the immunoglobulin. The term “core hinge” can be used interchangeably with the term “short hinge” (a.k.a “SH”). Non-limiting examples of suitable hinge domains are the core immunoglobulin hinge regions include EPKSCDKTHTCPPCP (SEQ ID NO: 57) from IgG1, ERKCCVECPPCP (SEQ ID NO: 58) from IgG2, ELKTPLGDTTHTCPRCP(EPKSCDTPPPCPRCP)₃ (SEQ ID NO: 59) from IgG3, and ESKYGPPCPSCP (SEQ ID NO: 60) from IgG4 (see also Wypych et al., JBC 2008 283(23): 16194-16205, which is incorporated herein by reference in its entirety for all purposes). In certain embodiments, the hinge domain is a fragment of the immunoglobulin hinge.

In some embodiments, the hinge domain is derived from CD8 or CD28. In one embodiment, the CD8 hinge domain comprises the amino acid sequence set forth in SEQ ID NO: 21, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 21. In one embodiment, the CD28 hinge domain comprises the amino acid sequence set forth in SEQ ID NO: 22, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 22.

In some embodiments, the transmembrane domain and/or hinge domain is derived from CD8 or CD28. In some embodiments, both the transmembrane domain and hinge domain are derived from CD8. In some embodiments, both the transmembrane domain and hinge domain are derived from CD28.

In certain aspects, the first polypeptide of arCARs of the present disclosure comprise a cytoplasmic domain, which comprises at least one intracellular signaling domain. In some embodiments, cytoplasmic domain also comprises one or more co-stimulatory signaling domains.

The cytoplasmic domain is responsible for activation of at least one of the normal effector functions of the host cell (e.g., T cell) in which the arCAR has been placed in. The term “effector function” refers to a specialized function of a cell. Effector function of a T-cell, for example, may be cytolytic activity or helper activity including the secretion of cytokines. Thus, the term “signaling domain” refers to the portion of a protein which transduces the effector function signal and directs the cell to perform a specialized function. While usually the entire signaling domain is present, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the signaling domain sufficient to transduce the effector function signal.

Non-limiting examples of signaling domains which can be used in the arCARs of the present disclosure include, e.g., signaling domains derived from DAP10, DAP12, Fc epsilon receptor I γ chain (FCER1G), FcR β, NKG2D, CD3δ, CD3ε, CD3γ, CD3ζ, CD5, CD22, CD226, CD66d, CD79A, or CD79B.

In some embodiments, the cytoplasmic domain comprises a CD3ζ signaling domain. In one embodiment, the CD3ζ signaling domain comprises the amino acid sequence set forth in SEQ ID NO: 6, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 6.

In some embodiments, the cytoplasmic domain further comprises one or more co-stimulatory signaling domains. In some embodiments, the one or more co-stimulatory signaling domains are derived from CD28, 41BB, IL2Rb, CD40, OX40 (CD134), CD80, CD86, CD27, ICOS, NKG2D, DAP10, DAP12, 2B4 (CD244), BTLA, CD30, GITR, CD226, CD79A, or HVEM.

In one embodiment, the co-stimulatory signaling domain is derived from 41BB. In one embodiment, the 41BB co-stimulatory signaling domain comprises the amino acid sequence set forth in SEQ ID NO: 8, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 8.

In one embodiment, the co-stimulatory signaling domain is derived from IL2Rb. In one embodiment, the IL2Rb co-stimulatory signaling domain comprises the amino acid sequence set forth in SEQ ID NO: 9, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 9.

In one embodiment, the co-stimulatory signaling domain is derived from CD40. In one embodiment, the CD40 co-stimulatory signaling domain comprises the amino acid sequence set forth in SEQ ID NO: 10, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 10.

In one embodiment, the co-stimulatory signaling domain is derived from OX40. In one embodiment, the OX40 co-stimulatory signaling domain comprises the amino acid sequence set forth in SEQ ID NO: 11, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 11.

In one embodiment, the co-stimulatory signaling domain is derived from CD80. In one embodiment, the CD80 co-stimulatory signaling domain comprises the amino acid sequence set forth in SEQ ID NO: 12, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 12.

In one embodiment, the co-stimulatory signaling domain is derived from CD86. In one embodiment, the CD86 co-stimulatory signaling domain comprises the amino acid sequence set forth in SEQ ID NO: 13, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 13.

In one embodiment, the co-stimulatory signaling domain is derived from CD27. In one embodiment, the CD27 co-stimulatory signaling domain comprises the amino acid sequence set forth in SEQ ID NO: 14, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 14.

In one embodiment, the co-stimulatory signaling domain is derived from ICOS. In one embodiment, the ICOS co-stimulatory signaling domain comprises the amino acid sequence set forth in SEQ ID NO: 15, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 15.

In one embodiment, the co-stimulatory signaling domain is derived from NKG2D. In one embodiment, the NKG2D co-stimulatory signaling domain comprises the amino acid sequence set forth in SEQ ID NO: 16, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 16.

In one embodiment, the co-stimulatory signaling domain is derived from DAP10. In one embodiment, the DAP10 co-stimulatory signaling domain comprises the amino acid sequence set forth in SEQ ID NO: 17, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 17.

In one embodiment, the co-stimulatory signaling domain is derived from DAP12. In one embodiment, the DAP12 co-stimulatory signaling domain comprises the amino acid sequence set forth in SEQ ID NO: 18, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 18.

In one embodiment, the co-stimulatory signaling domain is derived from 2B4 (CD244). In one embodiment, the 2B4 (CD244) co-stimulatory signaling domain comprises the amino acid sequence set forth in SEQ ID NO: 19, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 19.

In one embodiment, the co-stimulatory signaling domain is derived from CD28. In one embodiment, the CD28 co-stimulatory signaling domain comprises the amino acid sequence set forth in SEQ ID NO: 20, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 20.

In some embodiments, the arCAR of the present disclosure comprises one costimulatory signaling domains. In some embodiments, the arCAR of the present disclosure comprises two or more costimulatory signaling domains. In certain embodiments, the arCAR of the present disclosure comprises two, three, four, five, six or more costimulatory signaling domains.

In some embodiments, the signaling domain(s) and costimulatory signaling domain(s) can be placed in any order. In some embodiments, the signaling domain is upstream of the costimulatory signaling domains. In some embodiments, the signaling domain is downstream from the costimulatory signaling domains. In the cases where two or more costimulatory domains are included, the order of the costimulatory signaling domains could be switched.

Non-limiting exemplary CAR regions and sequences are provided in Table 1.

TABLE 1

| | | | |
|---|---|---|---|
| CAR regions | Sequence | UniProt Id | SEQ ID NO |

CD19 CAR:

| | | | |
|---|---|---|---|
| GMCSF | MLLLVTSLLLCELPHPAFLLIP | | 1 |
| Signal Peptide | | | |
| FMC63 VH | EVKLQESGPGLVAPSQSLSVTCTVSGVSLPDYGVSW | | 2 |
| | IRQPPRKGLEWLGVIWGSETTYYNSALKSRLTIIKD | | |
| | NSKSQVFLKMNSLQTDDTAIYYCAKHYYYGGSYAMD | | |
| | YWGQGTSVTVSS | | |
| Whitlow Linker | GSTSGSGKPGSGEGSTKG | | 3 |
| FMC63 VL | DIQMTQTTSSLSASLGDRVTISCRASQDISKYLNWY | | 4 |
| | QQKPDGTVKLLIYHTSRLHSGVPSRFSGSGSGTDYS | | |
| | LTISNLEQEDIATYFCQQGNTLPYTFGGGTKLEIT | | |
| CD28 | IEVMYPPPYLDNEKSGNTIIHVKGKHLCPSPLFPGP | P10747-1 | 5 |
| (AA 114-220) | SKPFWVLVVVGGVLACYSLLVTVAFIIFWVRSKRSR | | |
| | LLHSDYMNMTPRRGPTRKHYQPYAPPRDFAAYRS | | |
| CD3-zeta | RVKFSRSADAPAYQQGQNQLYNELNLGRREEYDVLD | P20963-3 | 6 |
| isoform 3 | KRRGRDPEMGGKPRRKNPQEGLYNELQKDKMAEAYS | | |
| (AA 52-163) | EIGMKGERRRGKGHDGLYQGLSTATKDTYDALHMQA | | |
| | LPPR | | |
| FMC63 scFV | EVKLQESGPGLVAPSQSLSVTCTVSGVSLPDYGVSW | | 7 |
| | IRQPPRKGLEWLGVIWGSETTYYNSALKSRLTIIKD | | |
| | NSKSQVFLKMNSLQTDDTAIYYCAKHYYYGGSYAMD | | |
| | YWGQGTSVTVSSGSTSGSGKPGSGEGSTKGDIQMTQ | | |
| | TTSSLSASLGDRVTISCRASQDISKYLNWYQQKPDG | | |
| | TVKLLIYHTSRLHSGVPSRFSGSGSGTDYSLTISNL | | |
| | EQEDIATYFCQQGNTLPYTFGGGTKLEIT | | |

Signaling Domains:

| | | | |
|---|---|---|---|
| 41BB | KRGRKKLLYIFKQPFMRPVQTTQEEDGCSCRFPEEE | Q07011 | 8 |
| (AA 214-255) | EGGCEL | | |
| IL2Rb | NCRNTGPWLKKVLKCNTPDPSKFFSQLSSEHGGDVQ | P14784 | 9 |
| (AA 266-551) | KWLSSPFPSSSFSPGGLAPEISPLEVLERDKVTQLL | | |
| | PLNTDAYLSLQELQGQDPTHLV | | |
| CD40 | KKVAKKPTNKAPHPKQEPQEINFPDDLPGSNTAAPV | P25942 | 10 |
| (AA 216-277) | QETLHGCQPVTQEDGKESRISVQERQ | | |
| OX40 | ALYLLRRDQRLPPDAHKPPGGGSFRTPIQEEQADHS | P43489 | 11 |
| (AA 236-277) | TLAKI | | |
| CD80 | TYCFAPRCRERRRNERLRRESVRPV | P33681 | 12 |
| (AA 264-288) | | | |
| CD86 | KWKKKKRPRNSYKCGTNTMEREESEQTKKREKIHIP | P42081 | 13 |
| (AA269-329) | ERSDEAQRVFKSSKTSSCDKSDTCF | | |
| CD27 | QRRKYRSNKGESPVEPAEPCHYSCPREEEGSTIPIQ | P26842 | 14 |
| (AA 213-260) | ESYRKPEPACSP | | |
| ICOS | CWLTKKKYSSSVHDPNGEYMFMRAVNTAKKSRLTDV | Q9Y6W8 | 15 |
| (AA 162-199) | TL | | |
| NKG2D | MGWIRGRRSR HSWEMSEFHN YNLDLKKSDF | P26718 | 16 |
| (AA 1-51) | STRWQKQRCP VVKSKCRENAS | | |
| DAP10 | LCARPRRSPAQEDGKVYINMPGRG | Q9UBK5 | 17 |
| (AA 70-93) | | | |
| DAP12 | YFLGRLVPRGRGAAEAATRKQRITETESPYQELQGQ | O54885 | 18 |
| (AA 62-113) | RSDVYSDLNTQRPYYK | | |
| 2B4/CD244 | WRRKRKEKQSETSPKEFLTIYEDVKDLKTRRNHEQE | Q9BZW8 | 19 |
| (AA 251-370) | QTFPGGGSTIYSMIQSQSSAPTSQEPAYTLYSLIQP | | |
| | SRKSGSRKRNHSPSFNSTIYEVIGKSQPKAQNPARL | | |
| | SRKELENFDVYS | | |
| CD3-zeta | RVKFSRSADAPAYQQGQNQLYNELNLGRREEYDVLD | P20963-3 | 6 |
| isoform 3 | KRRGRDPEMGGKPRRKNPQEGLYNELQKDKMAEAYS | | |
| (AA 52-163) | EIGMKGERRRGKGHDGLYQGLSTATKDTYDALHMQA | | |
| | LPPR | | |
| CD28 | RSKRSRLLHSDYMNMTPRRPGPTRKHYQPYAPPRDF | A10747-1 | 20 |
| (AA 180-220) | AAYRS | | |

Spacer/Hinge:

| | | | |
|---|---|---|---|
| CD8 | TTTPAPRPPTPAPTIASQPLSLRPEACRPAAGGAVH | P01732 | 21 |
| (AA 136-182) | TRGLDFACDIY | | |
| CD28 | IEVMYPPPYLDNEKSNGTIIHVKGKHLCPSPLFPGP | P10747-1 | 22 |
| (AA 114-151) | SKP | | |
| Short Hinge | GAGAGCAAGTATGGGCCCCCTTGTCCTCCTTGTCCG | | 63 |
| (nucleotide | | | |
| sequence) | | | |
| Short Hinge | ESKYGPPCPPCP | | 64 |
| (amino acid | | | |
| sequence) | | | |
| Medium Hinge | GAGAGCAAGTATGGGCCCCCTTGTCCTCCTTGTCCG | | 65 |
| (nucleotide | GGGCAGCCCCGAGAGCCACAGGTGTACACtCTGCCa | | |
| sequence) | CCAAGTCAGGAGGAGATGACCAAGAACCAGGTCAGC | | |
| | CTGACCTGCCTGGTCAAAGGCTTCTACCCCAGCGAC | | |
| | ATCGCCGTGGAGTGGGAGAGCAATGGGCAGCCGGAG | | |
| | AACAACTACAAGACCACGCCTCCCGTGCTGGACTCC | | |
| | GACGGCTCCTTCTTCCTCTACAGCAGGCTCACCGTG | | |
| | GACAAGAGCAGGTGGCAGGAGGGGAATGTCTTCTCA | | |
| | TGCTCCGTGATGCATGAGGCTCTGCACAACCACTAC | | |
| | ACACAGAAGAGCCTCTCCCTGTCTCTGGGAAAG | | |
| Medium Hinge | ESKYGPPCPPCPGQPREPQVYTLPPSQEEMTKNQVS | | 66 |
| (amino acid | LTCLVKGFYPSDIAVEWESNGQPENNYKTTPPVLDS | | |
| sequence) | DGSFFLYSRLTVDKSRWQEGNVFSCSVMHEALHNHY | | |
| | QTKSLSLSLGK | | |
| Long Hinge | GAGTCTAAGTATGGGCCCCCTTGTCCTCCTTGTCCG | | 67 |
| (nucleotide | GCACCTCCCGTGGCTGGACCAAGTGTATTCATTTCC | | |
| sequence) | CCCAAAACCCAAAGATACTCTCATGATTTCCCGGAC | | |
| | CCCTGAGGTTACATGCGTGGTGGTGGATGTGAGCCA | | |
| | GGAAGACCCCGAAGTCCAGTTTAACTGGTACGTGGA | | |
| | TGGAGTGGAGGTGCATAATGCAAAGACAAAGCCTCG | | |
| | GGAAGAACAGTTTCAGAGCACATACCGTGTGGTTAG | | |
| | TGTCCTCACAGTTCTGCACCAGGACTGGCTGAACGG | | |
| | CAAGGAGTATAAGTGTAAGGTCTCCAATAAAGGCCT | | |
| | CCCGTCATCGATCGAAAAAACCATCAGTAAAGCCAA | | |
| | AGGGCAGCCAAGGGAGCCACAGGTGTATACTTTACC | | |
| | ACCAAGTCAGGAGGAAATGACCAAGAACCAGGTATC | | |
| | TCTGACCTGCCTAGTCAAAGGCTTTTACCCCAGCGA | | |
| | TATCGCTGTGGAGTGGGAGTCTAATGGGCAGCCAGA | | |
| | GAACAACTACAAGACCACACCTCCTGTGCTGGACTC | | |
| | CGATGGCTCCTTCTTTCTATACAGCAGGTTAACCGT | | |
| | GGATAAGAGCAGGTGGCAGGAGGGGAATGTCTTCTC | | |
| | ATGCTCTGTGATGCATGAGGCTCTGCACAACCACTA | | |
| | CACACAGAAGAGCCTCTCCCTGTCTCTGGGAAAG | | |
| Long Hinge | ESKYGPPCPPCPAPPVAGPSVFLFPPKPKDLTMLIS | | 68 |
| (amino acid | RTPEVTCVVVDVSQEDPEVQFNWYVDGVEVHNAKTK | | |
| sequence) | PREEQFQSTYRVVSVLTVLHQDWLNGKEYKCKVSNK | | |
| | GLPSSIEKTISKAKGQPREPQVYTLPPSQEEMTKNQ | | |
| | VSLTCLVKGFYPSDIAVEWESNGQPENNYKTTPPVL | | |
| | DSDGSFFLYSRLTVDKSRWQEGNVFSCSVMHEALHN | | |
| | HYTQKSLSLSLGK | | |

Leader Sequence/Signal Peptide:

| | | |
|---|---|---|
| GMCSF | MLLLVTSLLLCELPHPAFLLIP | 1 |
| Signal Peptide | | |
| Mouse Ig- | | |
| Kappa Signal | ATGGCCAGGAGCCCCGCCCAGCTGCTGGGCCTGCTG | 61 |
| Peptide | CTGCTGTGGCTGAGCGGCGCCAGGTGC | |
| (nucleotide | | |
| sequence) | | |
| Mouse Ig- | MARSPAQLLGLLLLWLSGARC | 62 |
| Kappa Signal | | |
| Peptide | | |
| (amino acid | | |
| sequence) | | |

Transmembrane:

| | | | |
|---|---|---|---|
| CD8 | IYIWAPLAGTGCGVLLLSLVIT | P10732 | 23 |
| (AA 183-203) | | | |
| CD28 | FWVLVVVGGVLACYSLLVTVAFIIFWV | P10747-1 | 24 |
| (AA 153-179) | | | |

Linkers:

| | | |
|---|---|---|
| Whitlow | GSTSGSGKPGSGEGSTKG | 3 |
| Linker | | |
| (G₄S)₃ | GGGGSGGGGSGGGGS | 25 |
| Linker 3 | GGSEGKSSGSGSESKSTGGS | 26 |
| Linker 4 | GGGSGGGS | 27 |
| Linker 3 | GGGSGGGSGGGS | 28 |
| Linker 4 | GGGSGGGSGGGSGGGS | 29 |
| Linker 5 | GGGSGGGSGGGSGGGSGGGS | 30 |
| Linker 6 | GGGGSGGGGSGGGGSGGGGS | 31 |
| Linker 7 | GGGGSGGGGSGGGGSGGGGSGGGGS | 32 |
| Linker 8 | IRPRAIGGSKPRVA | 33 |
| Linker 9 | GKGGSGKGGSGKGGS | 34 |
| Linker 10 | GGKGSGGKGSGGKGS | 35 |
| Linker 11 | GGGKSGGGKSGGGKS | 36 |
| Linker 12 | GKGKSGKGKSGKGKS | 37 |
| Linker 13 | GGGKSGGKGSGKGGS | 38 |
| Linker 14 | GKPGSGKPGSGKPGS | 39 |
| Linker 15 | GKPGSGKPGSGKPGSGKPGS | 40 |
| Linker 16 | GKGKSGKGKSGKGKSGKGKS | 41 |
| Linker 17 | STAGDTHLGGEDFD | 42 |
| Linker 18 | GEGGSGEGGSGEGGS | 43 |
| Linker 19 | GGEGSGGEGSGGEGS | 44 |
| Linker 20 | GEGESGEGESGEGES | 45 |
| Linker 21 | GGGESGGEGSGEGGS | 46 |
| Linker 22 | GEGESGEGESGEGESGEGES | 47 |
| Linker 23 | GSTSGSGKPGSGEGSTKG | 48 |
| Linker 24 | PRGASKSGSASQTGSAPGS | 49 |
| Linker 25 | GTAAAGAGAAGGAAAGAAG | 50 |
| Linker 26 | GTSGSSGSGSGGSGSGGGG | 51 |
| Linker 27 | GKPGSGKPGSGKPGSGKPGS | 52 |
| Linker 28 | GSGS | 53 |
| Linker 29 | APAPAPAPAP | 54 |
| Linker 30 | APAPAPAPAPAPAPAPAPAP | 55 |
| Linker 31 | AEAAAKEAAAKEAAAAKEAAAAKEAAAAKAAA | 56 |

In some embodiments, the antigen-binding domain of the second polypeptide binds to an antigen. The antigen-binding domain of the second polypeptide may bind to more than one antigen or more than one epitope in an antigen. For example, the antigen-binding domain of the second polypeptide may bind to two, three, four, five, six, seven, eight or more antigens. As another example, the antigen-binding domain of the second polypeptide may bind to two, three, four, five, six, seven, eight or more epitopes in the same antigen.

The choice of antigen-binding domain may depend upon the type and number of antigens that define the surface of a target cell. For example, the antigen-binding domain may be chosen to recognize an antigen that acts as a cell surface marker on target cells associated with a particular disease state. In certain embodiments, the arCARs of the present disclosure can be genetically modified to target a tumor antigen of interest by way of engineering a desired antigen-binding domain that specifically binds to an antigen (e.g., on a tumor cell). Non-limiting examples of cell surface markers that may act as targets for the antigen-binding domain in the arCAR of the disclosure include those associated with tumor cells or autoimmune diseases.

In some embodiments, the antigen-binding domain binds to at least one tumor antigen or autoimmune antigen.

In some embodiments, the antigen-binding domain binds to at least one tumor antigen. In some embodiments, the antigen-binding domain binds to two or more tumor antigens. In some embodiments, the two or more tumor antigens are associated with the same tumor. In some embodiments, the two or more tumor antigens are associated with different tumors.

In some embodiments, the antigen-binding domain binds to at least one autoimmune antigen. In some embodiments, the antigen-binding domain binds to two or more autoimmune antigens. In some embodiments, the two or more autoimmune antigens are associated with the same autoimmune disease. In some embodiments, the two or more autoimmune antigens are associated with different autoimmune diseases.

In some embodiments, the tumor antigen is associated with glioblastoma, ovarian cancer, cervical cancer, head and neck cancer, liver cancer, prostate cancer, pancreatic cancer, renal cell carcinoma, bladder cancer, or hematologic malignancy. Non-limiting examples of tumor antigen associated with glioblastoma include HER2, EGFRVIII, EGFR, CD133, PDGFRA, FGFR1, FGFR3, MET, CD70, ROBO1 and IL13Rα2. Non-limiting examples of tumor antigens associated with ovarian cancer include FOLR1, FSHR, MUC16, MUC1, Mesothelin, CA125, EpCAM, EGFR, PDGFRα, Nectin-4 and B7H4. Non-limiting examples of the tumor antigens associated with cervical cancer or head and neck cancer include GD2, MUC1, Mesothelin, HER2, and EGFR. Non-limiting examples of tumor antigen associated with liver cancer include Claudin 18.2, GPC-3, EpCAM, cMET, and AFP. Non-limiting examples of tumor antigens associated with hematological malignancies include CD19, CD22, CD79, BCMA, GPRC5D, SLAM F7, CD33, CLL1, CD123, and CD70. Non-limiting examples of tumor antigens associated with bladder cancer include Nectin-4 and SLITRK6. Non-limiting examples of tumor antigens associated with renal cancer include CD70 and FOLR1.

Additional examples of antigens that may be targeted by the antigen-binding domain include, but are not limited to, alpha-fetoprotein, A3, antigen specific for A33 antibody, Ba 733, BrE3-antigen, carbonic anhydrase EX, CD1, CD1a, CD3, CD5, CD15, CD16, CD19, CD20, CD21, CD22, CD23, CD25, CD30, CD33, CD38, CD45, CD74, CD79a, CD80, CD123, CD138, colon-specific antigen-p (CSAp), CEA (CEACAM5), CEACAM6, CSAp, EGFR, EGP-I, EGP-2, Ep-CAM, EphA1, EphA2, EphA3, EphA4, EphA5, EphA6, EphA7, EphA8, EphA10, EphB1, EphB2, EphB3, EphB4, EphB6, FIt-I, Flt-3, folate receptor, HLA-DR, human chorionic gonadotropin (HCG) and its subunits, hypoxia inducible factor (HIF-I), Ia, IL-2, IL-6, IL-8, insulin growth factor-1 (IGF-I), KC4-antigen, KS-1-antigen, KS1-4, Le-Y, macrophage inhibition factor (MIF), MAGE, MUC2, MUC3, MUC4, NCA66, NCA95, NCA90, antigen specific for PAM-4 antibody, placental growth factor, p53, prostatic acid phosphatase, PSA, PSMA, RS5, S100, TAC, TAG-72, tenascin, TRAIL receptors, Tn antigen, Thomson-Friedenreich antigens, tumor necrosis antigens, VEGF, ED-B fibronectin, 17-1A-antigen, an angiogenesis marker, an oncogene marker or an oncogene product.

In one embodiment, the antigen targeted by the antigen-binding domain is CD19. In one embodiment, the antigen-binding domain comprises an anti-CD19 scFv. In one embodiment, the anti-CD19 scFv comprises a heavy chain variable region (VH) comprising the amino acid sequence set forth in SEQ ID NO: 2, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 2. In one embodiment, the anti-CD19 scFv comprises a light chain variable region (VL) comprising the amino acid sequence set forth in SEQ ID NO: 4, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 4. In one embodiment, the anti-CD19 scFv comprises the amino acid sequence set forth in SEQ ID NO: 7, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 7.

In some embodiments, the antigen is associated with an autoimmune disease or disorder. Such antigens may be derived from cell receptors and cells which produce “self”-directed antibodies. In some embodiments, the antigen is associated with an autoimmune disease or disorder such as Rheumatoid arthritis (RA), multiple sclerosis (MS), Sjögren's syndrome, Systemic lupus erythematosus, sarcoidosis, Type 1 diabetes mellitus, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Myasthenia gravis, Hashimoto's thyroiditis, Graves' disease, chronic inflammatory demyelinating polyneuropathy, Guillain-Barre syndrome, Crohn's disease or ulcerative colitis.

In some embodiments, autoimmune antigens that may be targeted by the arCAR disclosed herein include but are not limited to platelet antigens, myelin protein antigen, Sm antigens in snRNPs, islet cell antigen, Rheumatoid factor, and anticitrullinated protein, citrullinated proteins and peptides such as CCP-1, CCP-2 (cyclical citrullinated peptides), fibrinogen, fibrin, vimentin, filaggrin, collagen I and II peptides, alpha-enolase, translation initiation factor 4G1, perinuclear factor, keratin, Sa (cytoskeletal protein vimentin), components of articular cartilage such as collagen II, IX, and XI, circulating serum proteins such as RFs (IgG, IgM), fibrinogen, plasminogen, ferritin, nuclear components such as RA33/hnRNP A2, Sm, eukaryotic translation elongation factor 1 alpha 1, stress proteins such as HSP-65, -70, -90, BiP, inflammatory/immune factors such as B7-H1, IL-1 alpha, and IL-8, enzymes such as calpastatin, alpha-enolase, aldolase-A, dipeptidyl peptidase, osteopontin, glucose-6-phosphate isomerase, receptors such as lipocortin 1, neutrophil nuclear proteins such as lactoferrin and 25-35 kD nuclear protein, granular proteins such as bactericidal permeability increasing protein (BPI), elastase, cathepsin G, myeloperoxidase, proteinase 3, platelet antigens, myelin protein antigen, islet cell antigen, rheumatoid factor, histones, ribosomal P proteins, cardiolipin, vimentin, nucleic acids such as dsDNA, ssDNA, and RNA, ribonuclear particles and proteins such as Sm antigens (including but not limited to SmD's and SmB′/B), UIRNP, A2/B1 hnRNP, Ro (SSA), and La (SSB) antigens.

Non-limiting exemplary antigen targets are provided in Tables 2-4.

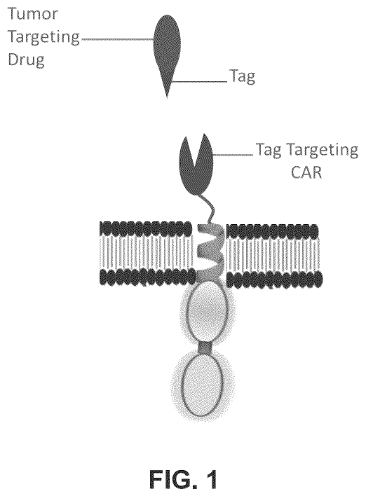

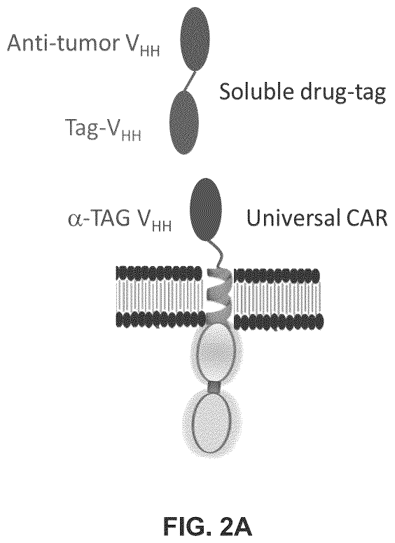

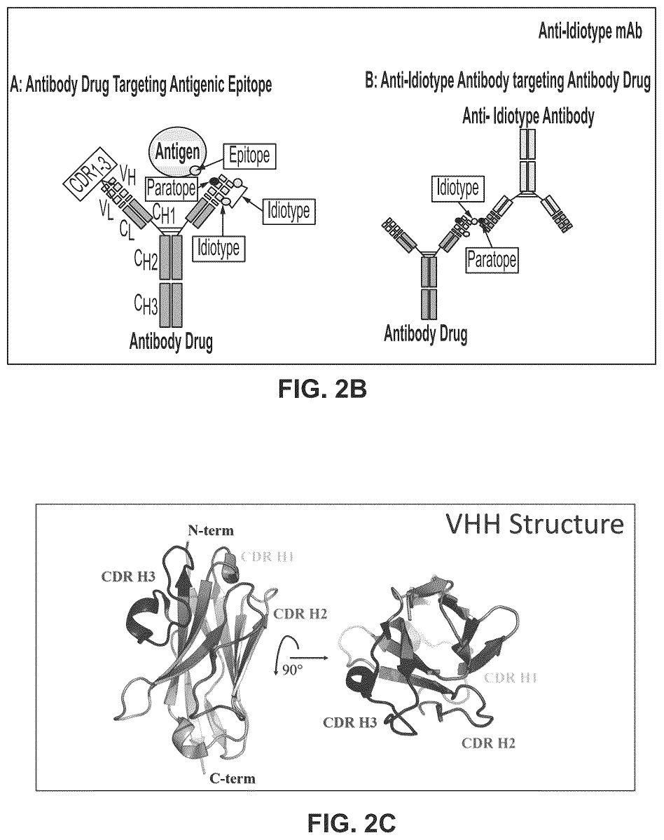

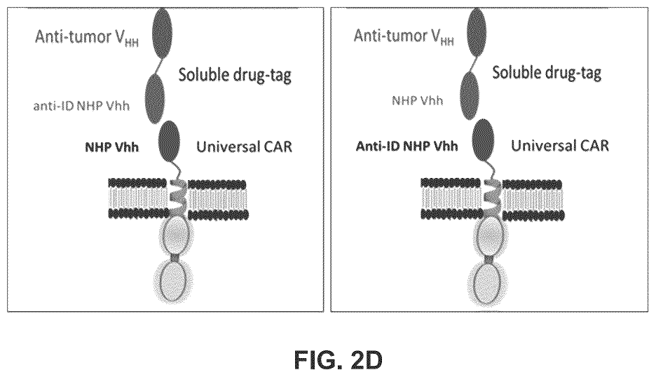

TABLE 2
The antigen-binding domain may comprise a VH sequence, a VL sequence, and/or CDRs
thereof, such as those described in the cited publications, the contents of each
publication are incorporated herein by reference in their entirety for all purposes.

| Antigen target | Type | Examples of Source | Type | Examples of Source |
|---|---|---|---|---|
| 5T4 | VH | Identifier 2, 4 in | VL | Identifier 1, 3 in |
| | | WO2016022939 | | WO2016022939 |
| AGR2 | VH | Identifier 10, 18 in | VL | Identifier 11, 19 in |
| | | WO2016040321 | | WO2016040321 |
| ALK | VH | Identifier 1, 11, 13, 15, 3, 5, | VL | Identifier 10, 12, 14, 16, 2, |
| | | 7, 9 in US20160280798A1; | | 4, 6, 8 in |
| | | Identifier 9, 1, 3, 5, 11, 13, | | US20160280798A1; |
| | | 15, 7, 9 in WO2015069922 | | Identifier 10, 12, 14, 16, 8 |
| | | | | in WO2015069922; |
| | | | | Identifier 2, 4, 6 in |
| | | | | WO2015069922 |
| AMC | VH | Identifier 17, 18, 19, 20, 21, | VL | Identifier 27, 28, 29, 31, |
| | | 22, 23, 24, 25, 26 in | | 32, 33, 34, 35, 36 in |
| | | WO2016161390 | | WO2016161390 |
| ANG2 | VH | Identifier 1, 3 in | VL | Identifier 2, 4 in |
| | | WO2015091655 | | WO2015091655 |
| APCDD1 | VH | Identifier 10, 102, 106, 110, | VL | Identifier 136, 100, 104, |
| | | 114, 118, 122, 126, 130, | | 108, 112, 116, 12, 120, |
| | | 134, 14, 6, 98 in | | 124, 128, 132, 16, 8 in |
| | | WO2012019061 | | WO2012019061 |
| APRIL | VH | Identifier 12, 14, 16. 18. 3. | VL | Identifier 20, 22, 24, 26, |
| | | 32, 34, 36, 38, 40, 42, 44, | | 28, 30, 4, 50 in |
| | | 46, 48, 52 in | | US2016026467 |
| | | US20160264674 | | |
| AXL | VH | Identifier 21, 3, 45 in | VL | Identifier 22, 4 in |
| | | WO2016097370 | | WO2016097370 |
| B2MG | VH | Identifier 28 in | VL | Identifier 29 in |
| | | WO2016126213A1 | | WO2016126213A1 |
| B7H1 | VH | Identifier 12, 32, 42, 52, 72, | VL | Identifier 17, 37, 47, 57, 7, |
| | | 2, 62 in US20130034559 | | 77, 27, 67 in |
| | | | | US20130034559 |
| B7H3 | VH | Identifier 10, 11, 12, 13, 14, | VL | Identifier 1, 2, 3, 4, 5, 6, 7, |
| | | 15, 16, 9 in WO2016033225 | | 8 in WO2016033225 |
| B7H3 (CD276) | VH | Identifier 17, 26, 7 in | VL | Identifier 18, 27, 8 in |
| | | WO2016044383 | | WO2016044383 |
| B7H4 | VH | Identifier 100, 101, 102, | VL | Identifier 104, 11, 126, |
| | | 103, 107, 108, 109, 110, | | 134, 138, 19, 27, 3, 35, 55, |
| | | 111, 112, 113, 114, 12, 127, | | 93, 95, 97, 98, 145, 146, |
| | | 130, 131, 132, 133, 137, 2, | | 147, 148 in |
| | | 20, 28, 36, 37, 38, 4, 56, 99, | | US20160159910; Identifier |
| | | 144 in US20160159910; | | 29, 31, 33 in |
| | | Identifier 13, 15, 17 in | | WO2016160620 |
| | | WO2016160620 | | |
| BAT1 | VH | Identifier 5, 6, 7, 8, 9 in | VL | Identifier 1, 2, 3, 4 in |
| | | WO2013014668 | | WO2013014668 |
| BCMA | VH | Identifier 26 in | VL | Identifier 25 in |
| | | WO2016168773 A3; | | WO2016168773 A3; |
| | | Identifier 142, 148, 154, | | Identifier 42 in |
| | | 160, 166, 172, 178, 184, | | WO2016097231; Identifier |
| | | 190, 196, 202, 208, 214, | | 143, 149, 155, 161, 167, |
| | | 220, 226, 232, 238, 244, | | 173, 179, 185, 191, 197, |
| | | 250, 256, 262, 268, 274, | | 203, 209, 215, 221, 227, |
| | | 280, 286, 292, 298, 304, | | 233, 239, 245, 251, 257, |
| | | 310, 316, 322, 328, 334, | | 263, 269, 275, 281, 287, |
| | | 340, 346, 352 in | | 293, 299, 305, 311, 317, |
| | | WO2016168595A1; | | 323, 329, 335, 341, 347, |
| | | Identifier 8 in | | 353 in WO2016168595 |
| | | WO2016094304A3; | | A1; Identifier 192, 193, |
| | | Identifier 171, 172, 173, | | 194, 195, 196, 197, 198, |
| | | 174, 175, 176, 177, 178, | | 199, 200, 201, 204, 205, |
| | | 179, 180, 181, 182, 183, | | 207, 208, 211, 259, 260, |
| | | 184, 185, 186, 187, 190, | | 84, 85, 86, 87, 88, 89, 90, |
| | | 255, 257, 258, 69, 70, 71, | | 91, 92, 93, 94, 95, 96, 97, |
| | | 72, 73, 74, 75, 76, 77, 78, | | 98 in WO2016014565; |
| | | 79, 80, 81 WO2016014565; | | Identifier 53 in |
| | | Identifier 38 in | | WO2016187349A1; |
| | | EP3057994A1; Identifier 55 | | Identifier 7 in |
| | | in WO2016187349A1; | | WO2016094304 A3; |
| | | Identifier 1, 13, 17, 21, 25, | | Identifier 10, 14, 18, 2, 22, |
| | | 29, 33, 37, 41, 45, 49, 5, 53, | | 26, 30, 34, 38, 42, 46, 50, |
| | | 57, 61, 65, 9 in | | 54, 58, 6, 62, 66 in |
| | | WO2016090320; Identifier | | WO2016090320; Identifier |
| | | 101, 743, 174, 758, 95, 759, | | 100, 102, 175, 96, 98 in |
| | | 97, 760, 99 in | | WO2016120216; Identifier |
| | | WO2016120216; Identifier | | 12, 14, 16, 18 in |
| | | 11, 741, 17 in | | WO2015158671A1; |
| | | WO2015158671A1; | | Identifier 7, 8, 9 in |
| | | Identifier 10, 11, 12, 13, 14 | | WO2016014789; Identifier |
| | | in WO2016014789; | | 14 in WO2016168766A1 |
| | | Identifier 15 in | | |
| | | WO2016168766A1 | | |
| BMPR1A | VH | Identifier 12 in | — | — |
| | | WO2011116212 | | |
| CA19.9 | VH | Identifier 117 in | VL | Identifier 118 in |
| | | US20160333114A1 | | US20160333114A1 |
| Campath 1 | VH | Identifier 34 in | VL | Identifier 31, 33 in |
| | | US20160333114A1 | | US20160333114A1 |
| CD105 | VH | Identifier 13, 14, 16 in | VL | Identifier 1, 17, 20, 22, 23 |
| | | WO2014039682 | | in WO2014039682 |
| CD123 | VH | Identifier 11, 13, 14, 21 in | VL | Identifier 9, 11, 18, 19, 20, |
| | | WO2015140268A1; | | 21, 22, 23 in |
| | | Identifier 113, 115, 57, 59, | | WO2016120220; Identifier |
| | | 63 in WO2016120216; | | 12, 16, 18, 19, 22 in |
| | | Identifier 12, 123, 24, 25, | | WO2015140268A1; |
| | | 26, 27, 28, 29, 30, 9 in | | Identifier 275, 276, 277, |
| | | WO2016120220; Identifier | | 278, 307, 308, 309, 310 in |
| | | 216, 217, 218, 219, 274 in | | WO2016028896; Identifier |
| | | WO2016028896 | | 5 in US20160333108A1; |
| | | | | Identifier 114, 116, 58, 60, |
| | | | | 64 in WO2016120216 |
| CD148 | VH | Identifier 10, 14, 18, 2, 22, | VL | Identifier 12, 16, 20, 24, |
| | | 26, 30, 6 in WO2005118643 | | 28, 32, 4, 8 in |
| | | | | WO2005118643 |
| CD16 | VH | Identifier 25 in | VL | Identifier 26 in |
| | | WO2015158868 | | WO2015158868 |
| CD19 | VH | Identifier 53, 55 in | VL | Identifier 27, 31 in |
| | | WO2016120216 | | WO2016168773 A3; |
| | | | | Identifier 49 in |
| | | | | WO2016187349A1; |
| | | | | Identifier 11 in |
| | | | | WO2016134284; Identifier |
| | | | | 194 in US20140134142A1; |
| | | | | Identifier 54, 56 in |
| | | | | WO2016120216; Identifier |
| | | | | 13, 14, 15, 16, 17, 186, |
| | | | | 187, 188, 189, 192, 196, |
| | | | | 197, 198, 199, 200, 201, |
| | | | | 202, 203, 204, 205, 64, 66, |
| | | | | 67, 68, 69, 70, 71, 91 |
| | | | | US20160152723; Identifier |
| | | | | 22 in US20160039942; |
| | | | | 3361 Identifier 63 in |
| | | | | WO2016097231; Identifier |
| | | | | 3 in US20160145337A1; |
| | | | | Identifier 112 in |
| | | | | US20160333114A1; |
| | | | | Identifier 114 in |
| | | | | US20160333114A1; |
| | | | | Identifier 13, 6 |
| | | | | US20160319020 |
| CD19H 803 | VH | Identifier 28, 29, 32, 33, 34, | — | — |
| | | 35 in WO2016168773 A3; | | |
| | | Identifier 51 in | | |
| | | WO2016187349A1; | | |
| | | Identifier 20 in | | |
| | | US20160039942; Identifier | | |
| | | 1 in WO2014184143; | | |
| | | Identifier 5 in | | |
| | | US20160145337A1; | | |
| | | Identifier 166, 167, 168, | | |
| | | 172, 176, 177, 181, 183, | | |
| | | 184, 185, 62 in | | |
| | | US20160152723; Identifier | | |
| | | 15 US20160319020; | | |
| | | Identifier 17, 33, 34, 35 in | | |
| | | EP3057994A1; Identifier 62 | | |
| | | in WO2016097231; | | |
| | | Identifier 12 in | | |
| | | WO2016134284; Identifier | | |
| | | 111, 113 in | | |
| | | US20160333114A1 | | |
| CD2 | VH | Identifier 103, 117, 119 in | VL | Identifier 102, 116 in |
| | | WO2016122701 | | WO2016122701 |
| CD20 | VH | Identifier 45 in | VL | Identifier 46 in |
| | | WO2016097231; Identifier | | WO2016097231; Identifier |
| | | 11, 13, 14, 15, 16, 17, 18, | | 10, 12, 8 in |
| | | 19, 20, 21, 22, 23, 24, 25, | | WO2017004091; Identifier |
| | | 26, 27, 28, 29, 30, 31, 32, | | 51 in US20160333114A1 |
| | | 33, 7, 9 in WO2017004091; | | |
| | | Identifier 26 in | | |
| | | US20170000900; Identifier | | |
| | | 54 in US20160333114A1; | | |
| | | Identifier 25 in | | |
| | | US20170000900; Identifier | | |
| | | 24 in US20170000900; | | |
| | | Identifier 23 in | | |
| | | US20170000900 | | |
| CD22 | VH | Identifier 3 in | VL | Identifier 17, 8, 14, 15 in |
| | | WO2013059593; Identifier | | US20150239974; Identifier |
| | | 10, 11, 12, 7, 9, 8 in | | 7 in US20150299317; |
| | | US20150299317; Identifier | | Identifier 681 in |
| | | 201 in WO2016164731; | | WO2016164731; Identifier |
| | | CD22 VH 869 Identifier | | 682 in WO2016164731; |
| | | 671 in WO2016164731; | | Identifier 683, 2020 in |
| | | Identifier 672 in | | WO2016164731; Identifier |
| | | WO2016164731; Identifier | | 684 in WO2016164731; |
| | | 673 in WO2016164731; | | Identifier 685 in |
| | | Identifier 676 in | | WO2016164731; Identifier |
| | | WO2016164731; Identifier | | 686 in WO2016164731; |
| | | 678 in WO2016164731; | | Identifier 687 in |
| | | Identifier 679 in | | WO2016164731; Identifier |
| | | WO2016164731; Identifier | | 688 in WO2016164731; |
| | | 680 in WO2016164731; | | Identifier 690 in |
| | | Identifier 700 in | | WO2016164731; Identifier |
| | | WO2016164731; Identifier | | 740 in WO2016164731; |
| | | 701 in WO2016164731; | | Identifier 741 in |
| | | Identifier 702 in | | WO2016164731; Identifier |
| | | WO2016164731; Identifier | | 742 in WO2016164731; |
| | | 703 in WO2016164731; | | Identifier 743 in |
| | | Identifier 704 in | | WO2016164731; Identifier |
| | | WO2016164731; Identifier | | 744 in WO2016164731; |
| | | 705 in WO2016164731; | | Identifier 745 in |
| | | Identifier 706 in | | WO2016164731; Identifier |
| | | WO2016164731; Identifier | | 746 in WO2016164731; |
| | | 707 in WO2016164731; | | Identifier 747 in |
| | | Identifier 708 in | | WO2016164731; Identifier |
| | | WO2016164731, Identifier | | 748 in WO2016164731; |
| | | 709 in WO2016164731; | | Identifier 749 in |
| | | Identifier 711 in | | WO2016164731; Identifier |
| | | WO2016164731; Identifier | | 750 in WO2016164731; |
| | | 712 in WO2016164731; | | Identifier 752 in |
| | | Identifier 713 in | | WO2016164731; Identifier |
| | | WO2016164731; Identifier | | 753 in WO2016164731; |
| | | 714 in WO2016164731; | | Identifier 754 in |
| | | Identifier 715 in | | WO2016164731; Identifier |
| | | WO2016164731; Identifier | | 755 in WO2016164731; |
| | | 716 in WO2016164731; | | Identifier 756 in |
| | | Identifier 717 in | | WO2016164731; Identifier |
| | | WO2016164731; Identifier | | 757 in WO2016164731; |
| | | 718 in WO2016164731; | | Identifier 758 in |
| | | Identifier 719 in | | WO2016164731; Identifier |
| | | WO2016164731; Identifier | | 759 in WO2016164731; |
| | | 720 in WO2016164731; | | Identifier 760 in |
| | | Identifier 721 in | | WO2016164731; Identifier |
| | | WO2016164731; Identifier | | 761 in WO2016164731; |
| | | 722 in WO2016164731; | | Identifier 762 in |
| | | Identifier 723 in | | WO2016164731; Identifier |
| | | WO2016164731; Identifier | | 763 in WO2016164731; |
| | | 724 in WO2016164731; | | Identifier 764 in |
| | | Identifier 725 in | | WO2016164731; Identifier |
| | | WO2016164731; Identifier | | 765 in WO2016164731; |
| | | 726 in WO2016164731; | | Identifier 766 in |
| | | Identifier 727 in | | WO2016164731; Identifier |
| | | WO2016164731; Identifier | | 767 in WO2016164731; |
| | | 728 in WO2016164731; | | Identifier 768 in |
| | | Identifier 729 in | | WO2016164731; Identifier |
| | | WO2016164731; Identifier | | 769 in WO2016164731; |
| | | 730 in WO2016164731; | | Identifier 770 in |
| | | Identifier 731 in | | WO2016164731; Identifier |
| | | WO2016164731; Identifier | | 771 in WO2016164731; |
| | | 732 in WO2016164731; | | Identifier 772 in |
| | | Identifier 733 in | | WO2016164731; Identifier |
| | | WO2016164731; Identifier | | 773 in WO2016164731; |
| | | 734 in WO2016164731; | | Identifier 774 in |
| | | Identifier 735 in | | WO2016164731; Identifier |
| | | WO2016164731; Identifier | | 775 in WO2016164731; |
| | | 736 in WO2016164731; | | Identifier 776 in |
| | | Identifier 737 in | | WO2016164731; Identifier |
| | | WO2016164731; Identifier | | 777 in WO2016164731; |
| | | 738 in WO2016164731 | | Identifier 124 in |
| | | | | WO2016122701 |
| CD276 | VH | Identifier 17, 26, 7 in | VL | Identifier 18, 27 in |
| | | US20160053017 | | US20160053017 |
| CD28 | VH | Identifier 19 in | VL | Identifier 20 in |
| | | WO2015158868 | | WO2015158868 |
| CD3 | VH | Identifier 108, 112, 115 in | VL | Identifier 104 in |
| | | WO2016122701; Identifier | | WO2016122701; Identifier |
| | | 29 in WO2014144722 A2; | | 13 in WO2016126213A1 |
| | | Identifier 12 in | | |
| | | WO2016126213A1 | | |
| CD30 | VH | Identifier 14, 16 in | VL | Identifier 13, 15 in |
| | | WO2016134284 | | WO2016134284 |
| CD324 | VH | Identifier 21, 23, 25, 27, 29, | VL | Identifier 20, 22, 24, 26, |
| | | 31, 33, 35, 37, 39, 41, 43, | | 28, 30, 32, 34, 36, 38, 40, |
| | | 45, 47, 49, 51, 53, 55, 57, | | 42, 44, 46, 48, 50, 52, 54, |
| | | 59, 61, 63, 65, 67, 69, 71 in | | 56, 58, 60, 62, 64, 66, 68, |
| | | U.S. Pat. No. 9,534,058 | | 70 in U.S. Pat. No. 9,534,058 |
| CD32B | VH | Identifier 127 in | VL | Identifier 126 in |
| | | WO2016122701 | | WO2016122701 |
| CD33 | VH | Identifier 65, 67, 69, 71, 77, | VL | Identifier 12, 14, 16, 18 in |
| | | 79, 81, 83, 84 in | | WO2015150526A2; |
| | | WO2016120216; Identifier | | Identifier 66, 68, 70, 72, |
| | | 11, 13, 15; 17 in | | 78, 80, 82 in |
| | | WO2015150526A2; | | WO2016120216; Identifier |
| | | Identifier 57, 58, 59, 60, 61, | | 66, 67, 68, 69, 70, 71, 72, |
| | | 62, 63, 64, 65 in | | 73, 74 in WO2016014576 |
| | | WO2016014576 | | |
| CD37 | VH | Identifier 11, 12, 18 in | VL | Identifier 14, 15 in |
| | | US20170000900 | | US20170000900 |
| CD38 | VH | Identifier 2 in | VL | Identifier 1, 11 in |
| | | WO2009080830; Identifier | | WO2009080830 |
| | | 10 in WO2015121454 | | |
| CD3s | VH | Identifier 7 in | VL | Identifier 8 in |
| | | WO2014144722A2 | | WO2014144722A2 |
| CD40 | VH | Identifier 1 in | VL | Identifier 2 in |
| | | WO2016069919; Identifier | | WO2016069919; Identifier |
| | | 5, 7, 8 in WO2015091655 | | 6 in WO2015091655 |
| CD45 | VH | Identifier 24 in | VL | Identifier 25 in |
| | | WO2016126213A1 | | WO2016126213A1 |
| CD46 | VH | Identifier 39, 47, 59, 15, 19, | VL | Identifier 41, 61, 21, 25, |
| | | 23, 27, 31, 35, 43, 51, 55, | | 29, 33, 37, 45, 49, 53, 57, |
| | | 63, 67, 71, 75, 79, 83, 69, | | 65, 69, 73, 77, 81, 85, 17, |
| | | 71, 83 in WO2012031273; | | 73, 77 in WO2012031273; |
| | | Identifier 1, 10, 11, 12, 13, | | Identifier 23, 24, 25, 26, |
| | | 14,15,16,17,3,5, 6, 7, 9, | | 27, 28, 29, 30, 31, 32, 33, |
| | | 18, 19, 20, 21 in | | 34, 35, 36, 37, 38, 39, 40, |
| | | WO2016040683 | | 41,42 in WO2016040683 |
| CD4BS | VH | Identifier 15, 3 in | VL | Identifier 14, 2 in |
| | | US20160194375A1 | | US20160194375A1 |
| CD4i | VH | Identifier 5 in | VL | Identifier 4 in |
| | | US20160194375A1 | | US20160194375A1 |
| CD52 | VH | Identifier 103, 136, 137 in | VL | Identifier 102, 138 in |
| | | WO2010132659 | | WO2010132659 |
| CD64 | VH | Identifier 129 in | VL | Identifier 128 in |
| | | WO2016122701 | | WO2016122701 |
| CD7 | VH | Identifier 16, 20 in | VL | Identifier 17, 21 in |
| | | WO2016126213A1 | | WO2016126213A1 |
| CD70 | VH | Identifier 81, 85, 89 in | VL | Identifier 83, 87, 91 in |
| | | WO2015121454 | | WO2015121454; Identifier |
| | | | | 83 in WO2015121454; |
| | | | | Identifier 87 in |
| | | | | WO2015121454; Identifier |
| | | | | 91 in WO2015121454 |
| CD71 | VH | Identifier 1, 3, 325, 4, 5, 699 | VL | Identifier 2, 327, 329, 331, |
| | | in US20160355599 | | 333, 335, 337, 6, 650, 652, |
| | | | | 654, 656, 658, 660, 670, |
| | | | | 671, 672, 673, 7, 701, 702, |
| | | | | 703, 704, 705, 706, 707, |
| | | | | 708, 709, 710, 711, 712, |
| | | | | 721, 722, 723, 724, 725, |
| | | | | 726, 727, 728, 729, 730, |
| | | | | 731, 732, 733, 734, 735, |
| | | | | 736, 737, 738, 739, 740, |
| | | | | 741, 742, 743, 744, 745, |
| | | | | 746, 747, 748, 749, 750, |
| | | | | 751, 752, 753, 754, 755, |
| | | | | 756, 757, 758, 759, 760, |
| | | | | 761, 762, 763, 764, 765, |
| | | | | 766, 767, 768, 769, 770, |
| | | | | 771, 772, 773, 774, 775, |
| | | | | 776, 777, 778, 779, 780, |
| | | | | 781, 782, 783, 784 785, |
| | | | | 786, 787, 788, 8, 810, 811, |
| | | | | 812, 813, 814, 815, 816, |
| | | | | 817, 818, 819, 820, 821, |
| | | | | 822, 823, 824, 825, 826, |
| | | | | 827, 828, 829, 830, 831, |
| | | | | 832, 833, 834, 835, 836, |
| | | | | 810, 842, 843, 844, 845, |
| | | | | 846, 847, 848, 849, 850, |
| | | | | 851, 852, 853, 854, 855, |
| | | | | 856, 857, 858, 859, 860, |
| | | | | 861, 862, 863, 864, 865, |
| | | | | 866, 867, 868, 869, 870, |
| | | | | 871, 872, 873, 874, 875, |
| | | | | 876, 877, 879, 879, 880, |
| | | | | 881, 882, 883, 884, 885, |
| | | | | 886, 887, 888, 889, 890, |
| | | | | 891, 892, 893, 894, 895, |
| | | | | 896, 897, 898, 899, 900, |
| | | | | 901, 902, 903, 904, 905, |
| | | | | 906, 907, 908 in |
| | | | | US20160355599 |
| CD73 | VH | Identifier 100, 103, 107, | VL | Identifier 12. 20, 44, 72, |
| | | 109, 112, 114, 116, 119, | | 76, 8, 84, 92 in |
| | | 121, 16, 32, 4, 52, 60, 68, | | US20160145350; Identifier |
| | | 80, 88 in US20160145350; | | 22, 29, 37, 4 in |
| | | Identifier 135, 40, 21, 3, 28, | | WO2016055609A1; |
| | | 36 in WO2016055609A1 | | Identifier 101, 102, 104, |
| | | | | 106, 110, 117, 118, 120, |
| | | | | 122 in US20160145350 |
| CD74 | VH | Identifier 6 in | VL | Identifier 25, 29, 31, 35 in |
| | | US20100284906A1; | | US20130171064; Identifier |
| | | Identifier 10, 11, 9 in | | 12, 13, 14, 11, 4 in |
| | | US20040115193A1; | | US20040115193A1 |
| | | Identifier 23, 27, 30, 33 in | | |
| | | US20130171064 | | |
| CD76b | VH | Identifier 15, 17, 19, 23, 27, | VL | Identifier 16, 18, 22, 38, |
| | | 29, 37, 57, 59, 61 in | | 58, 60, 62 in |
| | | US20160159906 | | US20160159906 |
| CD79 | VH | Identifier 131 in | VL | Identifier 130 in |
| | | WO2016122701 | | WO2016122701 |
| CDIM | VH | Identifier 1, 10, 11, 12, 13, | VL | Identifier 28, 29, 30, 31, |
| | | 14, 15, 16, 17, 18, 19, 2, 20, | | 32, 33, 34, 35, 36, 37, 38, |
| | | 21, 22, 3, 4, 5, 6, 7, 8, 9 in | | 39, 40, 41, 42, 43, 44, 45, |
| | | WO2013120012 | | 46, 47, 48, 49 in |
| | | | | WO2013120012 |
| CEA | VH | Identifier 8 in U.S. Pat. No. 8,287,865 | VL | Identifier 10, 38, 39, 7, 9 in |
| | | | | U.S. Pat. No. 8,287,865 |
| Claudin | VH | Identifier 101, 103, 105, | VL | Identifier 114, 116, 118, |
| | | 107, 109, 111, 113, 115, | | 120, 22, 25, 29, 33, 37, 41, |
| | | 117, 119, 121, 122, 123, | | 45, 49, 53, 57, 61, 65, 69, |
| | | 124, 125, 126, 127, 128, | | 73, 77 in |
| | | 129, 130, 131, 132, 133, | | WO2016073649A1 |
| | | 134, 135, 136, 137, 138, | | |
| | | 139, 140, 141, 142, 143, | | |
| | | 144, 145, 146, 147, 148, | | |
| | | 149, 150, 23, 27, 31, 35, 39, | | |
| | | 43, 47, 51, 55, 59, 63, 67, | | |
| | | 71, 75, 79, 81, 83, 85, 87, | | |
| | | 89, 91, 93, 95, 97, 99 in | | |
| | | WO2016073649A1 | | |
| CLDN18.2 | VH | Identifier 12, 2 in | VL | Identifier 13, 3 in |
| | | US20160347815A1 | | US20160347815A1 |
| CLL1 | VH | Identifier 13, 14, 15, 17, 19, | VL | Identifier 16, 18, 20, 22, |
| | | 21, 23, 25, 27, 29, 31, 33, 35 | | 24, 26, 28, 30, 32, 24, 36 in |
| | | in WO2016120219; | | WO2016120219; Identifier |
| | | Identifier 195, 65, 66, 67, | | 196, 78, 79, 80, 81, 82, 83, |
| | | 68, 69, 70, 71, 72, 73, 74, | | 84, 85, 86, 87, 88, 89, 90 in |
| | | 75, 76, 77 in | | WO2016014535; Identifier |
| | | WO2016014535; Identifier | | 30, 32, 35, 37, 39, 41 in |
| | | 31, 33, 34, 36, 38, 40, 42, 46 | | US20160075787; Identifier |
| | | in US20160075787; | | 152, 104, 106, 108, 110, |
| | | Identifier 150, 103, 105, | | 112, 114, 116, 118 in |
| | | 107, 109, 111, 113, 115, 117 | | WO2016179319A1 |
| | | in WO2016179319A1 | | |
| CLL3 | VH | Identifier 101, 103, 105, | VL | Identifier 100, 102, 104, |
| | | 107, 109, 111, 113, 115, | | 106, 108, 110, 112, 114, |
| | | 117, 119, 121, 123, 125, | | 116, 118, 120, 122, 124, |
| | | 127, 129, 131, 133, 135, | | 126, 128, 130, 132, 134, |
| | | 137, 139, 141, 145, 147, | | 136, 138, 140, 144, 146, |
| | | 149, 151, 153, 155, 157, | | 148, 150, 152, 154, 156, |
| | | 159, 161, 163, 165, 167, | | 158, 160, 162, 164, 166, |
| | | 169, 171, 173, 175, 177, | | 170, 172, 174, 176, 178, |
| | | 179, 181, 183, 185, 187, | | 180, 182, 184, 186, 190, |
| | | 191, 193, 195, 197, 199, | | 192, 194, 196, 198, 20, |
| | | 201, 203, 205, 207, 209, 21, | | 200, 202, 204, 206, 208, |
| | | 211, 213, 23, 25, 27, 29, 31, | | 210, 212, 22, 24, 26, 28, |
| | | 33, 35, 37, 39, 41, 43, 45, | | 30, 32, 34, 36, 38, 40, 42, |
| | | 47, 49, 51, 53, 55, 57, 59, | | 44, 46, 48, 50, 54, 56, 58, |
| | | 61, 63, 65, 67, 69, 71, 73, | | 60, 62, 64, 66, 68, 70, 72, |
| | | 75, 77, 79, 81, 83, 85, 87, | | 74, 76, 78, 80, 82, 84, 86, |
| | | 89, 91, 93, 95, 97, 99 in | | 88, 90, 92, 94, 96, 98 in |
| | | US20170000901 | | US20170000901 |
| collagen | VH | Identifier 21, 4, 15, 17, 18, | VL | Identifier 11, 12, 14, 23, |
| | | 19, 20, 5, 6, 7, 1, 2, 3 in | | 25, 26, 27, 8, 9 in |
| | | WO2007024921 | | WO2007024921 |
| CS1 | VH | Identifier 22 in | VL | Identifier 104, 106, 108 in |
| | | WO2016168773 A3; 103, | | WO2016120216; Identifier |
| | | 105, 107, 109 in | | 14, 16, 18, 20, 22 in |
| | | WO2016120216; Identifier | | WO2015166056A1; |
| | | 13, 15, 17, 19 in | | Identifier 39, 41, 43, 45, 47 |
| | | WO2015166056A1; | | in WO2015121454; |
| | | Identifier 38, 40, 42, 44, 46 | | Identifier 110, 112 in |
| | | in WO2015121454; | | WO2016120216 |
| | | Identifier 26 in | | |
| | | US20160075784A1 | | |
| CSF | VH | Identifier 10, 102, 14, 18, 2, | VL | Identifier 12, 32, 44, 48, 60 |
| | | 22, 26, 30, 34, 38, 46, 50, | | in US20050059113A1 |
| | | 54, 58, 6, 62, 66, 70, 74, 78, | | |
| | | 82, 86, 90, 94, 98 in | | |
| | | US20050059113A1 | | |
| CSPG4 | VH | Identifier 10, 16, 18, 4, 6, 8 | VL | Identifier 7 in |
| | | in WO2016077638; | | WO2016164429; Identifier |
| | | Identifier 8 in | | 12, 14 in WO2016077638 |
| | | WO2016164429 | | |
| CTLA4 | VH | Identifier 3, 31, 32, 33, 34, | VL | Identifier 36, 37, 38, 39, |
| | | 35, 41, 42, 43, 44, 45, 7 in | | 40, 46, 47, 48, 49, 50, 8, 4 |
| | | US20140105914; Identifier | | in US20140105914; |
| | | 4 in U.S. Pat. No. 8,697,845; Identifier | | Identifier 2 in U.S. Pat. No. 8,697,845; |
| | | 19 in US20150283234; | | Identifier 20 in |
| | | Identifier 17 in | | US20150283234; Identifier |
| | | WO2014066532 | | 18 in WO2014066532 |
| CXCR4 | VH | Identifier 72, 73, 74, 75, 84 | VL | Identifier 76, 77, 78, 79, |
| | | in US20110020218 | | 80, 81, 82, 87, 88, 90, 91, |
| | | | | 92, 93 in US20110020218 |
| Daclizumab | VH | Identifier 44, 46 in | VL | Identifier 43, 45 in |
| | | US20160333114A1 | | US20160333114A1 |
| DR5 | VH | Identifier 18, 82, 90, 98, 8 in | VL | Identifier 13, 23, 25, 27, 3, |
| | | WO2016122701 | | 78, 86, 94, 29 in |
| | | | | WO2016122701; Identifier |
| | | | | 62 in WO2016122701; |
| | | | | Identifier 54 in |
| | | | | WO2016122701; Identifier |
| | | | | 70 in WO2016122701 |
| E7MC | VH | Identifier 15, 16, 17, 18, 19, | VL | Identifier 238, 239, 240, |
| | | 20, 21, 22, 23, 233, 234, | | 241, 242, 243, 36, 37, 38, |
| | | 235, 236, 237, 24, 25, 26, | | 39, 41, 42, 43, 44, 45, 46, |
| | | 27, 28, 29, 30, 31, 32, 33, | | 47, 48, 49, 50, 51, 52, 53, |
| | | 34, 35 in | | 54, 55, 56 in |
| | | WO2016182957A1 | | WO2016182957A1 |
| EFNA | VH | Identifier 149, 153, 157, 161 | VL | Identifier 151, 155, 159, |
| | | in US20150125472 | | 163 in WO2012118547; |
| | | | | Identifier 27, 53 in |
| | | | | US20150125472 |
| EFNA4 | VH | Identifier 13, 39 in | — | — |
| | | US20150125472 | | |
| EGFR | VH | Identifier 14, 50, 9 in | VL | Identifier 15 in |
| | | WO2015143382; Identifier | | WO2015143382; Identifier |
| | | 12, 14, 15, 21 in | | 14 in WO2014143765; |
| | | US20100008978A1; | | Identifier 4051, 4052, |
| | | Identifier 2123 in | | 4053, 4054, 4055, 4056, |
| | | WO2018231759 | | 4057, 4058, 4059, 4060, |
| | | | | 4061, 4062, 4063, 4064, |
| | | | | 4065, 4066, 4067, 4068, |
| | | | | 4069, 4070, 4071, 4072, |
| | | | | 4073, 4074, 4075, 4076, |
| | | | | 4077, 4078, 4079, 4080, |
| | | | | 4081, 4082, 4083, 4084, |
| | | | | 4085, 4086, 4087, 4088, |
| | | | | 4089. 4090, 4091, 4092, |
| | | | | 4093, 4094, 4095, 4096, |
| | | | | 4097, 4098, 4099, 4100, |
| | | | | 4101, 4102, 4103, 4104, |
| | | | | 4105, 4106, 4107, 4108, |
| | | | | 4109, 4110, 4111, 4112, |
| | | | | 4113, 4114, 4115, 4116, |
| | | | | 4117, 4118, 4119, 4120, |
| | | | | 4121, 4122, 4123, 4124, |
| | | | | 4125, 4126, 4127, 4128, |
| | | | | 4129, 4130, 4131, 4132, |
| | | | | 4133, 4134, 4135, 4136, |
| | | | | 4137, 4138, 4139, 4140, |
| | | | | 4141, 4142, 4143, 4144, |
| | | | | 4145, 4146, 4147, 4148, |
| | | | | 4149, 4150, 4151, 4152, |
| | | | | 4153, 4154, 4155, 4156, |
| | | | | 4157, 4158, 4159, 4160, |
| | | | | 4161, 4162, 4163, 4164, |
| | | | | 4165, 4166, 4167, 4168, |
| | | | | 4169, 4170, 4171, 4172, |
| | | | | 4173, 4174, 4175, 4176, |
| | | | | 4177, 4178, 4179, 4180, |
| | | | | 4181, 4182, 4183, 4184, |
| | | | | 4185, 4186, 4187, 4188, |
| | | | | 4189, 4190, 4191, 4192, |
| | | | | 4193, 4194, 4195, 4196, |
| | | | | 4197, 4198, 4199, 4200, |
| | | | | 4201, 4202, 4203, 4204, |
| | | | | 4205, 4206, 4207, 4208, |
| | | | | 4209, 4210, 4211, 4212, |
| | | | | 4213, 4214, 4215, 4216, |
| | | | | 4217, 4218, 4219, 4220, |
| | | | | 4221, 4222, 4223, 4224, |
| | | | | 4225, 4226, 4227, 4228, |
| | | | | 4229, 4230, 4231, 4232, |
| | | | | 4233, 4234, 4235, 4236, |
| | | | | 4237, 4238, 4239, 4240, |
| | | | | 4241, 4242, 4243, 4244, |
| | | | | 4245, 4246, 4247, 4248, |
| | | | | 4249, 4250, 4251, 4252, |
| | | | | 4253, 4254, 4255, 4256, |
| | | | | 4257, 4258, 4259, 4260, |
| | | | | 4261, 4262, 4263, 4264, |
| | | | | 4265, 4266, 4267, 4268, |
| | | | | 4269, 4270, 4271, 4272, |
| | | | | 4273, 4274, 4275, 4276, |
| | | | | 4277, 4278, 4279, 42870, |
| | | | | 4281, 4282, 42834284, |
| | | | | 4285, 4286, 4287, 4288, |
| | | | | 4289, 4290, 4291, 4292, |
| | | | | 4293, 4294, 4295, 4296, |
| | | | | 4297, 4298, 4299, 4300, |
| | | | | 4301, 4302, 4303, 4304, |
| | | | | 4305, 4306, 4307, 4308, |
| | | | | 4309, 4310, 4311, 4312, |
| | | | | 4313, 4314, 4315, 4316, |
| | | | | 4317, 4318, 4319, 4320, |
| | | | | 4321, 4322, 4323, 4324, |
| | | | | 4325, 4326, 4327, 4328, |
| | | | | 4329, 4330, 4331, 4332, |
| | | | | 4333, 4334, 4335, 4336, |
| | | | | 4337, 4338, 4339, 4340, |
| | | | | 4341, 4342, 4343, 4344, |
| | | | | 4345, 4346, 4347, 4348, |
| | | | | 4349, 4350, 4351, 4352, |
| | | | | 4353, 4354, 4355, 4356, |
| | | | | 4357, 4358, 4359, 4360, |
| | | | | 4361, 4362, 4363, 4364, |
| | | | | 4365, 4366, 4367, 4368, |
| | | | | 4369, 4370, 4371, 4372, |
| | | | | 4373, 4374, 4375, 4376, |
| | | | | 4377, 4378, 4379, 4380, |
| | | | | 4381, 4382, 4383, 4384, |
| | | | | 4385, 4386, 4387, 4388, |
| | | | | 4389, 4390, 4391, 4392, |
| | | | | 4393, 4394, 4395, 4396, |
| | | | | 4397, 4398, 4399, 4400, |
| | | | | 4401, 4402, 4403, 4404, |
| | | | | 4405, 4406, 4407, 4408, |
| | | | | 4409, 4410, 4411, 4412, |
| | | | | 4413, 4414, 4415, 4416, |
| | | | | 4417, 4418, 4419, 4420, |
| | | | | 4421, 4422, 4423, 4424, |
| | | | | 4425, 4426, 4427, 4428, |
| | | | | 4429, 4430, 4431, 4432, |
| | | | | 4433, 4434, 4435, 4436, |
| | | | | 4437, 4438, 4439, 4440, |
| | | | | 4441, 4442, 4443, 4444, |
| | | | | 4445, 4446, 4447, 4448, |
| | | | | 4449, 4450, 4451, 4452, |
| | | | | 4453, 4454, 4455, 4456, |
| | | | | 4457, 4458, 4459, 4460, |
| | | | | 4461, 4462, 4463, 4464, |
| | | | | 4465, 4466, 4467, 4468, |
| | | | | 4469, 4470, 4471, 4472, |
| | | | | 4473, 4474, 4475, 4476, |
| | | | | 4477, 4478, 4479, 4480, |
| | | | | 4481, 4482, 4483, 4484, |
| | | | | 4485, 4486, 4487, 4488, |
| | | | | 4489, 4490, 4491, 4492, |
| | | | | 4493, 4494, 4495, 4496, |
| | | | | 4497, 4498, 4499, 4500, |
| | | | | 4501, 4502, 4503, 4504, |
| | | | | 4505, 4506, 4507, 4508, |
| | | | | 4509, 4510, 4511, 4512, |
| | | | | 4513, 4514, 4515, 4516, |
| | | | | 4517, 4518, 4519, 4520, |
| | | | | 4521, 4522, 4523, 4524, |
| | | | | 4525, 4526, 4527, 4528, |
| | | | | 4529, 4530, 4531, 4532, |
| | | | | 4533, 4534, 4535, 4536, |
| | | | | 4537, 4538, 4539, 4540, |
| | | | | 4541, 4542, 4543, 4544, |
| | | | | 4545, 4546, 4547, 4548, |
| | | | | 4549, 4550, 4551, 4552, |
| | | | | 4553, 4554, 4555, 4556, |
| | | | | 4557, 4558, 4559, 4560, |
| | | | | 4561, 4562, 4563, 4564, |
| | | | | 4565, 4566, 4567, 4568, |
| | | | | 4569, 4570, 4571, 4572, |
| | | | | 4573, 4574, 4575, 4576, |
| | | | | 4577, 4578, 4579, 4580, |
| | | | | 4581, 4582, 4583, 4584, |
| | | | | 4585, 4586, 4587, 4588, |
| | | | | 4589, 4590, 4591, 4592, |
| | | | | 4593, 4594, 4595, 4596, |
| | | | | 4597, 4598, 4599, 4600, |
| | | | | 4601, 4602, 4603, 4604, |
| | | | | 4605, 4606, 4607, 4608, |
| | | | | 4609, 4610, 4611, 4612, |
| | | | | 4613, 4614, 4615, 4616, |
| | | | | 4617, 4618, 4619, 4620, |
| | | | | 4621, 4622, 4623, 4624, |
| | | | | 4625, 4626, 4627, 4629, |
| | | | | 4629, 4630, 4631, 4632, |
| | | | | 4633, 4634, 4635, 4636, |
| | | | | 4637, 4638, 4639 in |
| | | | | WO2018231759 |
| EGFR | VH | Identifier 2124 in | VL | 4640, 4641, 4642, 4643, |
| (EGFRvIII) | | WO2018231759; Identifier | | 4644, 4645, 4646, 4647, |
| | | 13 in WO2016016341; | | 4648, 4649, 4650, 4651, |
| | | Identifier 24 in | | 4652, 4653, 4654, 4655, |
| | | WO2016168773 A3; | | 4656, 4657, 4658 in |
| | | Identifier 34 in | | WO2018231759; Identifier |
| | | US20160304615; Identifier | | 14 in WO2016016341; |
| | | 2 in US20160200819A1; | | Identifier 23 in |
| | | Identifier 91, 93 in | | WO2016168773 A3; |
| | | WO2016120216 | | Identifier 42 in |
| | | | | US20160304615; Identifier |
| | | | | 1 in US20160200819A1; |
| | | | | Identifier 92, 94 in |
| | | | | WO2016120216 |
| Endoglin | VH | Identifier 41, 42, 43, 71, 73, | VL | Identifier 103, 88, 89, 90, |
| | | 75, 88, 89, 90, 91, 92 in | | 91, 92, 93, 94, 95, 96, 97, |
| | | US20160009811 | | 102, 100 in |
| | | | | WO2011041441; Identifier |
| | | | | 100, 102, 103, 3, 4, 5, 70, |
| | | | | 72, 74, 93, 94, 95, 96, 97 in |
| | | | | US20160009811 |
| EphA2 receptor | VH | Identifier 20, 22, 24, 32, 34, | VL | Identifier 26, 28, 30, 47, |
| | | 36, 37, 38, 40, 42, 43, 45, | | 48, 49, 50, 52, 78, 80 in |
| | | 74, 76 in US20150274824 | | US20150274824 |
| ERBB2 | VH | Identifier 2, 4 in | VL | Identifier 1 in |
| | | US20110129464; Identifier | | US20110129464; Identifier |
| | | 10, 2, 26, 30, 38, 4, 40, 42, | | 12, 16, 20, 24, 32, 36, 44, |
| | | 52, 54, 56, 57, 58, 6 in | | 50, 51, 53, 8 in |
| | | US20130089544; Identifier | | US20130089544; Identifier |
| | | 8 in US20130266564; | | 7 in US20130266564; |
| | | Identifier 1 in | | Identifier 3 in |
| | | US20150104443 | | US20110129464 |
| Factor D | VH | Identifier 17, 20, 27, 29, 30, | VL | Identifier 16, 18, 19, 26, 3 |
| | | 31, 32, 33, 4 in | | in US20160017052 |
| | | US20160017052 | | |
| Factor XII | VH | Identifier 15 in | VL | Identifier 17 in |
| | | WO2014089493 | | WO2014089493 |
| FAP | VH | Identifier 1, 5 in | VL | Identifier 2, 6 in |
| | | WO2015118030; Identifier | | WO2015118030; Identifier |
| | | 170, 172 in | | 171, 173 in |
| | | WO2016120216; Identifier | | WO2016120216; Identifier |
| | | 8 in US20160326265 A1 | | 9 in US20160326265A1 |
| FcRL5 | VH | Identifier 12, 16, 20, 24, 28, | VL | Identifier 11, 15, 19, 23, |
| (FcReceptorLike | | 32, 36, 4, 40, 44, 48, 8, 915, | | 27, 3, 31, 35, 39, 43, 47, 7, |
| 5) | | 919 in WO2016090337 | | 917, 921 WO2016090337 |
| FGFR3 | VH | Identifier 132, 134, 136 in | VL | Identifier 133, 135, 137, |
| | | U.S. Pat. No. 9,499,623 | | 139 in U.S. Pat. No. 9,499,623 |
| Frizzled | VH | Identifier 10 in | VL | Identifier 12, 14 in |
| Receptor | | WO2010037041 | | WO2010037041 |
| GAH | VH | Identifier 7 in | VL | Identifier 8 in |
| | | US20060057147A1 | | US20060057147A1 |
| GCC1 | VH | Identifier 1 in | VL | Identifier 4, 2 in |
| | | US20160030595A1 | | US20160030595A1 |
| GD2 | VH | Identifier 17 in | VL | Identifier 10, 2, 5, 7, 9 in |
| | | WO2016134284; Identifier | | US20130216528; Identifier |
| | | 1 in US20130216528; | | 11, 12 in WO2015132604; |
| | | Identifier 10, 9 in | | Identifier 18 in |
| | | WO2015132604; Identifier | | WO2016134284 |
| | | 3, 4, 6, 8 in | | |
| | | US20130216528 | | |
| GD3 | VH | Identifier 11, 13, 15, 17 in | VL | Identifier 12, 14, 16, 18 in |
| | | WO2016185035A1 | | WO2016185035A1 |
| Glyco epitope | VH | Identifier 7 in | VL | Identifier 10 in |
| and ErbBB I | | WO2012007167A1 | | WO2012007167A1 |
| Specific | | | | |
| GM2 | VH | Identifier 20, 22, 23, 26, 27, | VL | Identifier 21, 24, 25, 31, |
| | | 28, 29, 30 in | | 32, 33, 34, 35 in |
| | | US20090028877 | | US20090028877 |
| GPC3 | VH | Identifier 10, 14, 2, 3, 4, 5, | VL | Identifier 10, 14, 18, 22, |
| | | 6, 7, 8, 9 in | | 24, 26 in U.S. Pat. No. 9,409,994B2; |
| | | US20160208015 A1; | | Identifier 16, 31 in |
| | | Identifier 22 in | | US20160208015A1; |
| | | WO2016049459; Identifier | | Identifier 23 in |
| | | 12, 16, 20, 37, 8 in | | WO2016049459 |
| | | U.S. Pat. No. 9,409,994B2 | | |
| GPDL1 | VH | Identifier 20 in | — | — |
| | | US20160108123 | | |
| GPRC5D | VH | Identifier 13, 17, 21, 25, 29, | VL | Identifier 10, 14, 18, 2, 22, |
| | | 314, 326, 33, 338, 350, 362, | | 26, 30, 303, 315, 327, 339, |
| | | 37, 374, 386, 41, 45, 49, 5, | | 34, 351, 363, 375, 38, 387, |
| | | 53, 57, 61, 65, 69, 73, 77, | | 42, 46, 50, 54, 58, 6, 62, |
| | | 81, 85, 89, 93, 1, 9 in | | 66, 70, 74, 78, 82, 86, 94 in |
| | | WO2016090312 | | WO2016090312 |
| Her2 | VH | Identifier 141, 262, 264, | VL | Identifier 140 in |
| | | 266, 268, 270 in | | WO2016054555A2; |
| | | WO2016168773A3; | | Identifier 261, 263, 265, |
| | | Identifier 11 in U.S. Pat. No. 9,518,118; | | 267, 269 in |
| | | Identifier 62 in | | WO2016168773A3; |
| | | US20160333114A1; | | Identifier 10, 18, 23 in |
| | | Identifier 19, 24 in | | U.S. Pat. No. 9,518,118; Identifier 3 in |
| | | U.S. Pat. No. 9,518,118 | | WO2016168769A1; |
| | | | | Identifier 59, 61 in |
| | | | | US20160333114A1 |
| Herl/her3 | VH | Identifier 8 of | VL | Identifier 4 of |
| | | WO2016073629 | | WO2016073629 |
| HLAG | VH | Identifier 10, 8 in | VL | Identifier 18, 20 in |
| | | WO2016160622A2 | | WO2016160622A2 |
| HSP70 | VH | Identifier 11, 12 in | VL | Identifier 16, 17 in |
| | | WO2016120217 | | WO2016120217 |
| Human collagen | VH | Identifier 31 in | VL | Identifier 32 in |
| VII | | WO2016112870 | | WO2016112870 |
| humanCD79b | VH | Identifier 27 in | VL | Identifier 28, 30 in |
| | | WO2016112870 | | WO2016112870 |
| | | humanCD79b VH 2304 | | |
| | | Identifier 29 in | | |
| | | WO2016112870 | | |
| humanERBB3 | VH | Identifier 19, 29, 38, 45, 55, | VL | Identifier 10, 20, 30, 39, |
| | | 61, 9 in WO2013052745 | | 46, 56, 62 in |
| | | | | WO2013052745 |
| ICOS | VH | Identifier 15, 16, 19, 23, 7 in | VL | Identifier 17, 18, 20, 24, 8 |
| | | US20160215059 | | in US20160215059 |
| IGFI | VH | Identifier 1, 3, 7 in | VL | Identifier 2, 4, 6, 8 in |
| | | WO2007118214; Identifier | | WO2007118214 |
| | | 7 in WO2015073575 | | |
| IGFR1 | VH | Identifier 7 in | VL | Identifier 8 in |
| | | WO2015073575 A2 | | WO2015073575 A2 |
| IL13 | VH | Identifier 302 in | VL | Identifier 303 in |
| | | US20160168242 | | US20160168242 |
| IL13Ra2 | VH | Identifier 7, 8 in | — | — |
| | | WO2016123143 | | |
| IL21 | VH | Identifier 2, 3 in | — | — |
| | | US20160145332 | | |
| IL33 | VH | Identifier 134, 136, 138, | VL | Identifier 135, 137, 139, |
| | | 185, 187, 189, 216, 218, | | 184, 188, 217, 219, 237, |
| | | 220, 221, 236, 246, 282, 284, | | 247, 283, 285, 287, 37, 39, |
| | | 286, 36, 38, 40, 84, 86, 88 | | 41, 87 in US20160168242 |
| | | in US20160168242 | | |
| IL3alpha | VH | Identifier 22 in | VL | Identifier 27, 37 in |
| | | WO2008127735 | | WO2008127735 |
| IL4R | VH | Identifier 10, 11, 14, 15, 9 in | VL | Identifier 13, 7, 8 in |
| | | WO2009121847 | | WO2009121847 |
| ILIRAP | VH | Identifier 1, 10, 19, 8, 9 in | VL | Identifier 14, 15, 17, 18, 2, |
| | | WO2016020502; Identifier | | 20 in WO2016020502; |
| | | 120, 122, 124 in | | Identifier 121, 123, 125 in |
| | | WO2016179319A1 | | WO2016179319A1 |
| Integrin | VH | Identifier 3, 4, 5 in US | VL | Identifier 10, 11, 8, 9 in US |
| | | 20140161794 | | 20140161794 |
| KDR | VH | Identifier 20, 24, 26, 29, 31, | VL | Identifier 22 in |
| | | 33 in WO2003075840 | | WO2003075840 |
| KIR (Lirilumab) | VH | Identifier 3 in | VL | Identifier 5 in |
| | | US20150290316; 2371 | | US20150290316; Identifier |
| | | Identifier 1 in | | 2 in WO2014055648 |
| | | WO2014055648 | | |
| KIR2DL 1 | VH | Identifier 36 in | VL | Identifier 37 in |
| | | WO2016126213A1 | | WO2016126213A1 |
| KIR2DL 2/3 | VH | Identifier 36 in | VL | Identifier 37 in |
| | | WO2016126213A1 | | WO2016126213A1 |
| Klon43 | VH | Identifier 47 in | VL | Identifier 48 in |
| | | WO2016097231 | | WO2016097231 |
| KMA | VH | Identifier 22 in | VL | Identifier 2, 21 in |
| | | WO2016172703 A2 | | WO2016172703A2 |
| LAG3 | VH | Identifier 102, 106, 110, | VL | Identifier 32, 36, 40, 44, |
| | | 113, 122, 18, 30, 66, 70, 74, | | 48, 52, 56, 60, 84, 88, 92, |
| | | 78 in US20150259420; | | 96, 134, 34, 38, 42, 46, 50, |
| | | Identifier 100, 104, 108, 28, | | 54, 58, 60, 86, 90, 94, 98 in |
| | | 64, 68, 72, 76, 8, 80 in | | US20150259420; Identifier |
| | | US20150259420; Identifier | | 2 in WO2015042246 |
| | | 1 in WO2015042246 | | |
| leukocytegenA2 | VH | Identifier 25 in | — | — |
| | | WO2010065962 A2 | | |
| leukocytegenA | VH | Identifier 9 in | VL | Identifier 24 in |
| | | WO2010065962 A2 | | WO2010065962 A2 |
| LGR4 | VH | Identifier 12, 13, 5, 9 in | VL | Identifier 10, 11, 6 in |
| | | US20160046723 | | US20160046723 |
| LGR5 | VH | Identifier 10, 12, 16, 18, 20, | VL | Identifier 15, 19, 21, 23, |
| | | 22, 24, 26, 4 in | | 25, 3 in US20160102146 |
| | | US20160102146 | | |
| LHR | VH | Identifier 1, 2, 3, 4, 5, 6, 7, 8 | — | — |
| | | in WO2016160618A3 | | |
| Lymphotoxin | VH | Identifier 10, 12, 14, 16, 2 in | VL | Identifier 1, 15, 4, 6, 8 in |
| beta receptor | | WO2004002431 | | WO2004002431 |
| Lysyloxidase- | VH | Identifier 42, 44 in | VL | Identifier 43, 45 in |
| like 2 | | WO2011097513 | | WO2011097513 |
| Malignant | VH | Identifier 1 in | VL | Identifier 5 in |
| Variable | | WO2015133817A1 | | WO2015133817A1 |
| Receptor | | | | |
| MCAM | VH | Identifier 115, 116, 117, | VL | Identifier 109, 110, 111, |
| | | 118, 119, 157, 158, 159, | | 112, 121, 122, 123 in |
| | | 160, 161, 178, 179 in | | US20150259419; Identifier |
| | | US20150259419; Identifier | | 30, 40, 50, 60, 70, 71, 72 in |
| | | 35, 45, 55, 65, 77, 89 in | | US20150239980 |
| | | US20150239980; Identifier | | |
| | | 101, 102, 103, 104, 105, | | |
| | | 106, 107 in | | |
| | | US20150259419 | | |
| MCSF | VH | Identifier 102, 10, 14, 18, 2, | VL | Identifier 8, 32, 52, 60, 28, |
| | | 22, 26, 30, 34, 38, 46, 50, | | 36, 4, 44, 48, 56, 62, 12, |
| | | 54, 58, 6, 66, 70, 74, 78, 82, | | 16, 20, 24 in |
| | | 86, 90, 94, 98 in | | WO2005030124 |
| | | WO2005030124 | | |
| Mesothelin | VH | Identifier 1, 6 in | VL | Identifier 3, 5 |
| | | WO2015188141; Identifier | | WO2015188141; Identifier |
| | | 119, 50 in | | 1, 2, 3 in WO2013142034; |
| | | US20160333114A1; | | Identifier 11, 15, 19, 23, 27 |
| | | Identifier 5, 6 in | | in US20160229919A1; |
| | | WO2013142034; Identifier | | Identifier 120, 47, 49 in |
| | | 15, 2 in U.S. Pat. No. 9,416,190B2; | | US20160333114A1 |
| | | Identifier 13, 17, 21, 25, 29, | | |
| | | 9 in US20160229919A1 | | |
| MN | VH | Identifier 133, 135, 137, | VL | Identifier 134, 136, 138, |
| | | 139, 141, 143, 145, 147, | | 140, 142, 144, 146, 148, |
| | | 149, 151 in WO2007070538 | | 150, 152 in |
| | | | | WO2007070538 |
| MPER | VH | Identifier 13 in | VL | Identifier 12 in |
| | | US20160194375A1 | | US20160194375A1 |
| MUC1 | VH | Identifier 5 in | VL | Identifier 7 in |
| | | US20160130357; Identifier | | US20160130357; Identifier |
| | | 2, 14 in WO2013023162; | | 16, 7 in WO2013023162; |
| | | Identifier 15, 19, 23, 60, 64, | | Identifier 17, 21, 25, 62, |
| | | 68 in WO2015116753 | | 66, 70 in WO2015116753 |
| MUC16 | VH | Identifier 1, 21, 41, 81, 61 in | VL | Identifier 2, 22, 42, 62, 82 |
| | | WO2016149368; Identifier | | in WO2016149368 |
| | | 11, 4, 6, 8 in | | |
| | | US20130171152 | | |
| MUC1C ECD | VH | Identifier 15, 19, 23, 60, 64, | VL | Identifier 17, 21, 25, 62, |
| | | 68, 72 in | | 66, 70, 75 in |
| | | US20160340442A1 | | US20160340442A1 |
| MUCIN1 | VH | Identifier 101, 106, 109, | VL | Identifier 148, 158, 162, |
| | | 115, 119, 123, 127, 141, 15, | | 167, 170, 174, 184, 190, |
| | | 23, 28, 33, 39, 42, 47, 5, 57, | | 193, 203, 208, 211, 220, |
| | | 66, 70, 75, 80, 83, 87, 92 in | | 225, 229, 234, 242, 246, |
| | | EP3049812A2 | | 250, 255, 261, 270, 275, |
| | | | | 279, 283, 291, 297, 303, |
| | | | | 308, 315, 319, 323, 333, |
| | | | | 340 in EP3049812A2 |
| MVR | VH | Identifier 1 in | VL | Identifier 5 in |
| | | US20160257762A1 | | US20160257762A1 |
| N Glycan | VH | Identifier 7, 9 in | VL | Identifier 6, 8 in |
| | | US20160194375A1 | | US20160194375A1 N |
| NKG2A | VH | Identifier 32 in | VL | Identifier 33 in |
| | | WO2016126213A1; | | WO2016126213A1; |
| | | Identifier 2, 3, 4, 5, 6 in | | Identifier 7 in |
| | | WO2016041947 | | WO2016041947 |
| NKG2D | VH | Identifier 135, 137 in | VL | Identifier 134, 136 in |
| | | WO2016122701 | | WO2016122701 |
| NOTCH 1 | VH | Identifier 58 in | VL | Identifier 16, 20 in |
| | | US20160333114A1; | | WO2013074596; Identifier |
| | | Identifier 12 in | | 55, 57 in |
| | | WO2013074596 | | US20160333114A1 |
| NOTCH 2/3 | VH | Identifier 29 in | VL | Identifier 31 in |
| | | WO2013074596 | | WO2013074596 |
| Notum | VH | Identifier 56, 331 in | VL | Identifier 332, 58 in |
| | | WO2012027723 | | WO2012027723 |
| NYBR1 | VH | Identifier 19 in | VL | Identifier 18 in |
| | | US20160333422A1 | | US20160333422A1 |
| OlfmB | VH | Identifier 1 in | VL | Identifier 2 in |
| | | WO2015054441A1 | | WO2015054441A1 |
| Olfml3 | VH | Identifier 19, 3 in | VL | Identifier 20, 4 in |
| | | WO2015054441A1 | | WO2015054441A1 |
| Oncofetal | — | — | VL | Identifier 1, 2, 7 in |
| fibronectin | | | | US20070202103A1 |
| Osteonectin | VH | Identifier 58 in | VL | Identifier 59 in |
| | | WO2016112870 | | WO2016112870 |
| OTK3 | VH | Identifier 17 in | VL | Identifier 18 in |
| | | WO2015158868 | | WO2015158868 |
| OX40 | VH | Identifier 101, 103, 105, | VL | Identifier 10, 45, 47, 49, 8 |
| | | 107, 109, 111, 113, 115, | | in U.S. Pat. No. 8,283,450; Identifier |
| | | 117, 119, 121, 123, 124, | | 11, 7 in U.S. Pat. No. 9,428,570; |
| | | 125, 17, 28, 318, 37, 48, 50, | | Identifier 116, 120, 122, |
| | | 58, 66, 74, 85, 93, 95, 97, | | 30, 38, 49, 57, 65, 73, 84, |
| | | 99 in WO2016196228; | | 86, 94, 98 in |
| | | Identifier 31, 34, 36, 38, 40, | | WO2016196228; Identifier |
| | | 42, 44, 46, 48, 50, 53, 54, | | 24, 26, 27, 28, 30, 60, 8, |
| | | 55, 58, 59, 61 in | | 81, 82, 83, 84, 85, 86, 87, |
| | | US20150190506; Identifier | | 88, 89 in U.S. Pat. No. 8,748,585; |
| | | 33, 35, 37, 39, 41, 43, 45, | | Identifier 30, 32 in |
| | | 47, 49, 51, 53, 55, 57, 59, | | US20160137740; Identifier |
| | | 61, 63, 65, 67, 71 in | | 32, 35, 39, 41, 43, 45, 47, |
| | | US20160137740; Identifier | | 49, 51, 52, 56, 57, 62 in |
| | | 44, 46, 48, 7, 9 in | | US20150190506; Identifier |
| | | U.S. Pat. No. 8,283,450; Identifier 9, 15 | | 29, 37 in US20160137740 |
| | | in U.S. Pat. No. 9,428,570; Identifier | | |
| | | 19, 21, 22, 23, 29, 58, 59, 7, | | |
| | | 77, 78, 79, 80 in | | |
| | | U.S. Pat. No. 8,748,585 | | |
| PD1 | VH | Identifier 19 in | VL | Identifier 2, 39, 7, 8, 9 in |
| | | US20150290316; Identifier | | US 20160159905; |
| | | 25, 26, 27, 28, 29 in | | Identifier 21 in |
| | | US20130291136; Identifier | | US20150290316; Identifier |
| | | 29, 3, 38 in US | | 30, 31, 32, 33 in |
| | | 20160159905; Identifier 38, | | US20130291136; Identifier |
| | | 50 in WO2015112900; | | 42, 46, 54 in |
| | | Identifier 4, 4, 6 in US | | WO2015112900; Identifier |
| | | 20160159905; Identifier 82, | | 58, 62, 66, 70, 74, 78 in |
| | | 86 in WO2015112900; | | WO2015112900; Identifier |
| | | Identifier 17 in | | 18 in WO2014055648; |
| | | WO2014055648; Identifier | | Identifier 5 in |
| | | 4 in WO2016040892; | | WO2016040892; Identifier |
| | | Identifier 12 in | | 13 in US20150190506 |
| | | US20150190506 | | |
| PDK1 | VH | Identifier 2 in | VL | Identifier 9 in |
| | | WO2016090365 | | WO2016090365 |
| PDl (Nivolumab) | VH | Identifier 2 in | VL | Identifier 11 in |
| | | WO2016040892; Identifier | | US20150190506 |
| | | 10 in US20150190506 | | |
| PDL1 | VH | Identifier 10, 32, 8 in | VL | Identifier 22, 26, 34, 42, |
| | | US20160319022; Identifier | | 58, 66, 74, 82, 86 in |
| | | 18, 30, 38, 46, 50, 54, 62, | | WO2016061142; Identifier |
| | | 70, 78 in WO2016061142; | | 30, 8, 9 in |
| | | Identifier 29, 7 in | | US20150190506; Identifier |
| | | US20150190506; Identifier | | 7, 9 in US20160319022; |
| | | 16, 18, 197, 247, 248, 250, | | Identifier 17, 22, 24, 249, |
| | | 251, 252, 253, 254, 255, | | 26, 28, 309, 311, 313, 320, |
| | | 256, 257, 258, 259, 260, 30, | | 325, 34, 340, 357, 359, 36, |
| | | 308, 310, 312, 319, 32, 324, | | 42, 44, 58, 60, 66, 68, 74, |
| | | 339, 356, 38, 40, 46, 48, 50, | | 76, 8, 82, 84, 86, 88 in |
| | | 52, 54, 6, 62, 70, 72, 78, 80, | | US20160108123 |
| | | 91, 96 in US20160108123; | | |
| | | Identifier 358, 56, 64 in | | |
| | | US20160108123 | | |
| PDL2 | VH | Identifier 43, 44, 56, 46 in | VL | Identifier 47, 48, 49, 50, 51 |
| | | US20130291136 | | in US20130291136 |
| PG16 | VH | Identifier 13 in | VL | Identifier 12 in |
| | | EP3074419A2 | | EP3074419A2 |
| PG9 | VH | Identifier 11 in | VL | Identifier 10 in |
| | | EP3074419A2 | | EP3074419A2 |
| PGT1 | VH | Identifier 15 in EP307441 | — | — |
| PGT2 | VH | Identifier 17 in | VL | Identifier 16 in |
| | | EP3074419A2 | | EP3074419A2 |
| PGT3 | VH | Identifier 19 in | VL | Identifier 18 in |
| | | EP3074419A2 | | EP3074419A2 |
| PGT4 | VH | Identifier 21 in | VL | Identifier 20 in |
| | | EP3074419A2 | | EP3074419A2 |
| PGT5 | VH | Identifier 23 in | VL | Identifier 22 in |
| | | EP3074419A2 | | EP3074419A2 |
| PRAME | VH | Identifier 50, 52, 54, 56, 58, | VL | Identifier 49, 51, 53, 55, |
| | | 60, 62 in | | 57, 59, 61 in |
| | | WO2016191246A2 | | WO2016191246A2 |
| PRP | VH | Identifier 42 in | VL | Identifier 39, 41 in |
| | | US20160333114A1 | | US20160333114A1 |
| PSMA | VH | Identifier 43 in | VL | Identifier 44 in |
| | | WO2016097231 | | WO2016097231; Identifier |
| | | | | 44 in WO2016097231 |
| PTK7 | VH | Identifier 1, 25, 49 in | VL | Identifier 20, 22, 24, 26, |
| | | US20150315293; Identifier | | 28, 30, 32, 34, 36, 38, 40, |
| | | 21, 23, 25, 27, 29, 31, 33, | | 42, 44, 46, 48, 50, 52, 54, |
| | | 35, 37, 39, 41, 43, 45, 47, | | 56, 58, 60, 62, 64, 66, 68 in |
| | | 49, 51, 53, 55, 57, 59, 61, | | WO2012112943A1; |
| | | 63, 65, 67, 69 in | | Identifier 15, 39, 63 in |
| | | WO2012112943A1 | | US20150315293 |
| RAS | VH | Identifier 17, 47, 57, 67, 7, | VL | Identifier 19, 49, 59, 69, |
| | | 77 in WO2016154047 | | 79, 9 in WO2016154047 |
| RHAMM | VH | Identifier 4 in | — | — |
| | | US20020127227A1 | | |
| RHAMM | VH | Identifier 2 in | VL | Identifier 4 in |
| antagonist body | | WO2000029447 | | WO2000029447 |
| Rituximab | VH | Identifier 66 in | VL | Identifier 63, 65 in |
| | | US20160333114A1 | | US20160333114A1 |
| ROR1 | VH | Identifier 12, 20, 28, 36, 44, | VL | Identifier 16, 24, 32, 40, |
| | | 60, 68 WO2016016343A1; | | 56, 64, 72, 36, 62, 23, 49, |
| | | Identifier 57, 19, 31, 45, 53, | | 58 WO2016016343A1; |
| | | 71 in WO2016016344A1; | | Identifier 86, 88, 90 in |
| | | Identifier 85, 87, 89 in | | WO2016120216; Identifier |
| | | WO2016120216; Identifier | | 126, 127, 234, 235, 236, |
| | | 122, 125, 175, 176, 179, | | 237, 238, 240, 241, 242, |
| | | 180, 181, 182, 183, 184, | | 243, 244, 245, 246, 247, |
| | | 185, 186, 187, 188, 189, | | 248 in US20160208018A1; |
| | | 190, 191, 192, 193, 194, | | Identifier 56 in |
| | | 195, 196, 197, 197, 199, | | EP3083671A1; Identifier |
| | | 200, 201, 202, 203, 204, | | 103, 111, 127, 135, 143, |
| | | 205, 206, 207, 208, 209 in | | 15, 151, 159, 167, 175, |
| | | US20160208018A1; | | 183, 191, 199, 207, 215, |
| | | Identifier 55 in | | 223, 23, 231, 239, 247, |
| | | EP3083671A1; Identifier | | 255, 263, 271, 279, 287, |
| | | 104, 112, 120, 128, 152, 16, | | 295, 303, 31, 311, 319, |
| | | 160, 168, 176, 184, 192, | | 327, 335, 343, 351, 359, |
| | | 200, 208, 216, 224, 232, 24, | | 39, 47, 55, 63, 7, 71, 79, |
| | | 240, 248, 256, 264, 272, | | 87, 95 in |
| | | 280, 288, 296, 304, 312, 32, | | WO2016187216A1 |
| | | 320, 336, 344, 352, 360, 40, | | |
| | | 48, 56, 64, 72, 8, 80, 88 in | | |
| | | WO2016187216A1 | | |
| SEMAPHORIN | VH | Identifier 10, 25, 9 in | VL | Identifier 17, 18, 29 in |
| 4D | | US20160115240A1 | | US20160115240A1 |
| TAG72 | VH | Identifier 115 in | VL | Identifier 116 in |
| | | US20160333114A1 | | US20160333114A1 |
| TCR | VH | Identifier 133 in | VL | Identifier 132 in |
| | | WO2016122701 | | WO2016122701 |
| TEM8 | VH | Identifier 1, 3, 5, 7 in | VL | Identifier 4, 6, 8 in |
| | | US20160264662A1 | | US20160264662A1 |
| Tie | VH | Identifier 723 in | VL | Identifier 724 in |
| | | US20060057138A1 | | US20060057138A1 |
| TIGIT | VH | Identifier 10, 11, 12, 124, | VL | Identifier 130, 131, 132, |
| | | 125, 126, 127, 128, 129, 13, | | 133, 137, 139, 145, 146, |
| | | 136, 138, 14, 143, 144, 149, | | 151, 152, 25, 26, 27, 28, |
| | | 15, 150, 16, 17, 18, 19, 20, | | 29, 30, 50, 51, 52, 64, 95, 8 |
| | | 21, 22, 23, 24, 37, 38, 39, | | in US20160355589 |
| | | 40, 41, 42, 43, 44, 45, 46, | | |
| | | 47, 63, 94, 7, 9 in | | |
| | | US20160355589 | | |
| TIM3 | VH | Identifier 102, 112, 12, 2, | — | — |
| | | 22, 32, 42, 52, 62, 72, 82, 91 | | |
| | | in US20150086574; | | |
| | | Identifier 82 in | | |
| | | WO2013006490; Identifier | | |
| | | 13, 21, 29, 37, 45, 5, 53, 61, | | |
| | | 69, 77, 85, 93 in | | |
| | | WO2016179319A1; | | |
| | | Identifier 7 in | | |
| | | WO2013006490; Identifier | | |
| | | 107, 117, 17, 27, 37, 47, 57, | | |
| | | 67, 7, 77, 87, 97 in | | |
| | | US20150086574; Identifier | | |
| | | 17, 25, 33, 41, 49, 57, 65, | | |
| | | 73, 81, 89, 9, 97 in | | |
| | | WO2016179319A1 | | |
| Tissue factor | VH | Identifier 10, 19, 23, 27, 29, | VL | Identifier 25. 31 in |
| | | 6 in WO2004094475; | | US20040229301A1; |
| | | Identifier 38 in | | Identifier 12, 21, 25, 31, 8 |
| | | US20160333114A1 | | in WO2004094475; |
| | | | | Identifier 35, 37 in |
| | | | | US20160333114A1 |
| Tn Glycopeptide | VH | Identifier 19, 20 in | — | — |
| | | WO2015120180 | | |
| TRBC1 | VH | Identifier 1 in | VL | Identifier 2 in |
| | | WO2015132598 | | WO2015132598 |
| Trophoblast | VH | Identifier 17, 13, 15, 11 in | VL | Identifier 18, 12, 14, 16 in |
| Glycoprotein | | WO2016034666A1 | | WO2016034666A1 |
| 5T4 | | | | |
| Upar | VH | Identifier 72 in | VL | Identifier 71, 73 in |
| | | US20160333114A1 | | US20160333114A1 |
| V2 | VH | Identifier 11 in | — | — |
| | | US20160194375A1 | | |
| VEGF | VH | Identifier 4, 8, in | VL | Identifier 2, 6 in |
| | | WO2000034337; Identifier | | WO2000034337; Identifier |
| | | 12, 20, 4, 44 in | | 9 in US20030175276A1; |
| | | WO2006012688A1; | | Identifier 11, 19, 27, 28, 3, |
| | | Identifier 7 in | | 43 in WO2006012688A1; |
| | | US20030175276A1; | | Identifier 160, 161, 162, |
| | | Identifier 152, 153, 154, | | 163, 164, 165, 166, 167 in |
| | | 155, 156, 157, 158, 159 in | | US20160090427 |
| | | US20160090427 | | |
| VEGFR2 | VH | Identifier 100, 101, 102, | VL | Identifier 107, 108, 109, |
| | | 103, 114, 115, 116, 117, | | 110, 111, 112, 113, 86, 87, |
| | | 118, 119, 120, 121, 122, | | 88, 89, 90, 91, 92, 93, 94 in |
| | | 123, 124, 95, 96, 97, 98, 99 | | WO2017004254 |
| | | in WO2017004254 | | |
| VISTA | VH | Identifier 37, 38, 39, 40 in | VL | Identifier 41, 42, 43, 44, 45 |
| | | WO2015097536 | | in WO2015097536 |
| VMS2 | VH | FIG. 1 in WO2000058363 | — | — |
| WT1/HLA | VH | Identifier 104, 111, 128, 14, | VL | Identifier 106, 112, 130, |
| Bispecific | | 32, 50, 68, 86 in | | 34, 52, 70, 88 in |
| | | WO2015070061 | | WO2015070061 |

TABLE 3
The antigen-binding domain may comprise an scFv derived from an antibody or antibody fragment
that binds to an antigen target such as those described in the cited publications, the contents
of each publication are incorporated herein by reference in their entirety for all purposes.

| Antigen Target | Examples of Source |
|---|---|
| Activated alpha v | Identifier 8, 2, 4 in US20090117096A1 |
| beta 3 | |
| Adalimunab | Identifier 41 in US20160208021; Identifier 41 in WO2016112870 |
| ALK | Identifier 17, 18, 19, 20, 21, 22, 23 in WO2015069922; Identifier 17, 18, |
| | 19, 20, 21, 22, 23, 24 in US20160280798A1; Identifier 24 in |
| | WO23015069922 |
| B7H3 | Identifier 99, 100, 101, 102, 103, 104, 102, 17, 18, 19, 20, 21, 22, 23, 24, |
| | 25, 26, 27, 87, 88, 89, 90, 91, 92, 94, 95, 96, 97, 98 in WO2016033225 |
| B7H4 | Identifier 1, 2, 3, 4 in WO2013067492; Identifier 1 in U.S. Pat. No. 9,422,351B2 |
| BCMA | Identifier 152, 158, 176, 185, 188, 200, 212, 218, 224, 284, 290, 296, 302, |
| | 314, 326, 344, 129, 130, 131, 132, 133, 134, 135, 136, 138, 139, 140, 141, |
| | 142, 143, 144, 145, 146, 147, 148, 149, 263, 264, 265, 266, 271, 273, 39, |
| | 40, 41, 42, 43, 44, 45, 46, 47, 48, 49, 50, 51, 52, 53, 64, 129, 130, 131, |
| | 132, 133, 134, 135, 136, 137, 138, 139, 140, 141, 142, 143, 144, 145, 146, |
| | 147, 148, 149, 263, 264, 265, 266, 39, 40, 41, 42, 43, 44, 45, 46, 47, 48, |
| | 49, 50, 51, 52, 53 in WO2016014565; Identifier 214, 215, 216, 217, 218, |
| | 219, 220, 221, 222, 223, 224, 225, 226, 227, 228, 229, 230, 231, 232, 233, |
| | 234, 235, 236, 237, 238, 239, 240, 241, 242, 243, 244, 245, 246, 247, 248, |
| | 249, 251 in US20160311907A1 |
| CCR4 | Identifier 7, 9 in WO2015191997 |
| CD123 | Identifier 157, 158, 159, 160, 184, 185, 186, 187, 188, 189, 190, 191, 192, |
| | 193, 194, 195, 196, 197, 198, 199, 200, 201, 202, 203, 204, 205, 206, 207, |
| | 208, 209, 210, 211, 212, 213, 214, 215, 478, 480, 483, 485 in |
| | WO2016028896; Identifier 36 in WO2015092024A2; Identifier 57 in |
| | WO2016115482A1; Identifier 36 in EP3083691A2; Identifier 157 in |
| | US20160311907A1 |
| CD124 | Identifier 158 in US20160311907A1 |
| CD125 | Identifier 159 in US20160311907A1 |
| CD126 | Identifier 160 in US20160311907A1 |
| CD127 | Identifier 161 in US20160311907A1 |
| CD128 | Identifier 162 in US20160311907A1 |
| CD129 | Identifier 163 in US20160311907A1 |
| CD130 | Identifier 164 in US20160311907A1 |
| CD131 | Identifier 165 in US20160311907A1 |
| CD138 | Identifier 36 in WO2016130598A1 |
| CD19 | Identifier 53, 54, 37 in EP3083671A1; Identifier 1, 10, 11, 12, 2 in |
| | WO2015157252; Identifier 10, 2, 206, 207, 208, 209, 210, 211, 213, 214, |
| | 215, 216, 217, 218, 219, 220, 221, 222, 223, 224, 225, 4, 45, 47, 49, 51, |
| | 53, 55, 57, 51, 53, 55, 57, 59, 6, 8, 87 in WO2016033570; Identifier 3, 4, |
| | 5, 59, 6, 7, 8, 9 in WO2015157252; 5754 Identifier 5 in |
| | WO2015155341A1; Identifier 7 in WO2014184143; Identifier 9 in |
| | WO2016139487; Identifier 10, 2, 206, 207, 208, 209, 210, 211, 212, 213, |
| | 214, 215, 216, 217, 218, 219, 220, 221, 222, 223, 224, 225, 4, 45, 47, 49, |
| | 51, 53, 55, 57, 59, 6, 8, 87, 89 in US20160152723; Identifier 32, 35, 38 in |
| | EP3083691A2; Identifier 174 in WO2016115482A1; Identifier 20 in |
| | WO2012079000; Identifier 32, 33, 35, 38 in WO2015092024A2; |
| | Identifier 39, 40, 41, 42, 43, 44, 45, 46, 47, 48, 49, 50, 51 in |
| | WO2016109410; Identifier 5, 6 in WO2015155341A1; Identifier 7, 9 in |
| | US20160145337A1; Identifier 20 in U.S. Pat. No. 9,499,629B2; Identifier 73 in |
| | WO2016164580; Identifier 10, 2, 206, 207, 209, 210, 212, 216, 218, 219, |
| | 220, 221, 222, 223, 224, 225, 4, 45, 47, 49, 51, 53, 55, 57, 59, 6, 8, 87, 89 |
| | in US20160152723; Identifier 5 in WO2016055551 |
| CD19/CD22 | Identifier 1303, 1307 in WO2016164731A2 |
| Bispecific | |
| CD20 | Identifier 691 in WO2016164731A100; Identifier 692 in |
| | WO2016164731A101; Identifier 693 in WO2016164731A102; Identifier |
| | 694 in WO2016164731A103; Identifier 695 in WO2016164731A104; |
| | Identifier 696 in WO2016164731A105; Identifier 175 in |
| | WO2016115482A1 |
| CD22 | Identifier 5, 6 in WO2013059593; Identifier 9 in US20150299317; |
| | Identifier 131, 132, 133, 134, 135, 136, 137, 138, 139, 140, 203, 209, 215, |
| | 221, 227, 232, 238, 244, 250, 256, 262, 268, 274, 280, 286, 292, 298, 304, |
| | 310, 316, 322, 328, 334, 340, 346, 353, 358, 364, 370, 376, 383, 388, 394, |
| | 400, 406, 412, 418, 423 in WO2016164731A2 |
| CD276 | Identifier 10, 19, 28 in US20160053017 |
| CD3 | Identifier 46, 47 in WO2015153912A1 |
| CD30 | Identifier 20 in WO2016116035A1; Identifier 2 in US20160200824A1 |
| CD33 | Identifier 262, 263, 264, 265, 266, 267, 268, 39, 40, 41, 42, 43, 44, 45, 46, |
| | 47 in WO2016014576; Identifier 37 in WO2015092024 A2; Identifier 37 |
| | in EP3083691A2; Identifier 153, 154, 155, 156, 157, 158, 159, 160, 161, |
| | 162, 163 in WO2016115482A1 |
| CD33/CD3s | Identifier 33, 34, 84 in WO2014144722A2 |
| bispecifc | |
| CD37 | Identifier 21, 22 in US20170000900 |
| CD44 | Identifier 17 in WO2016042461A1 |
| CD46 | Identifier 1-42 in WO2016040683 |
| CD5 | Identifier 16 in WO2016138491 |
| CD79b | Identifier 33 in US20160208021 |
| CEA | Identifier 1 in US20160303166A1; Identifier 22 in US20140242701A1 |
| Centuxiamb | Identifier 37 in WO2016112870; Identifier 37 in US20160208021 |
| Claudin | Identifier 11, 5, 7, 9 in WO2016073649A1; Identifier 17 in |
| | WO2014179759A1 |
| Claudin6 | Identifier 164 in WO2016115482A1 |
| Claudin7 | Identifier 165 in WO2016115482A1 |
| Claudin8 | Identifier 166 in WO2016115482A1 |
| CLDN6 | Identifier 2 in WO2016150400 |
| CLDN7 | Identifier 4 in WO2016150400 |
| CLDN8 | Identifier 6 in WO2016150400 |
| CLL1 | Identifier 39, 40, 41, 42, 43, 44, 45, 46, 48, 49, 50, 51 in WO2016014535; |
| | Identifier 200, 201, 202, 203, 204, 205, 206, 207, 208, 209, 210, 211, 212, |
| | 213 in US20160311907A1 |
| CMet | Identifier 11, 12, 13, 14, 15, 16, 17, 18, 19, 2, 21, 22, 23, 25, 26, 27, 28, 3, |
| | 30, 31, 33, 34, 35, 36, 37, 38, 39, 4; 40, 41, 42, 43, 44, 48, 49, 5, 50, 51, |
| | 52, 53, 54, 55, 56, 57, 58, 6, 60, 7, 9, 29 in US20040166544; Identifier 26, |
| | 27, 28, 29, 30, 32 in US20150299326; Identifier 32 in US20130034559 |
| CS1 | Identifier 1 of WO2016090369; Identifier 17 in WO2014179759A1 |
| CSPG4 | Identifier 2 in WO2015080981; Identifier 2 in EP3074025A1 |
| CXCR4 | Identifier 83, 85, 86, 89 in US20110020218 |
| E7MC | Identifier 223, 224, 225, 226, 227, 228, 229, 230, 231, 232 in |
| | WO2016182957A1 |
| EGFR | Identifier 11, 38, 41, 44, 47, 50, 53, 56, 59, 62, 65, 68, 71, 74, 77, 80, 83, |
| | 88, 91, 94 in WO2014130657 |
| EGFR VIII | Identifier 5 in US20140037628; Identifier 174 in US20160311907A1; |
| | Identifier 38 in U.S. Pat. No. 9,394,368B2; Identifier 5 in US20160200819A1 |
| END 0180 | Identifier 6 in WO2013098813 |
| ERBB2 | Identifier 26, 27 in US20110059076A1; Identifier 1, 2 in U.S. Pat. No. 7,244,826 |
| ESKAVT | Identifier 173 in WO2016115482A1 |
| FcRL | Identifier 11, 15, 19, 23, 27, 31, 35, 39, 3, 43, 7, 594, 596, 598, 600, 602, |
| | 604, 606, 608, 610, 612, 614, 616, 618, 620, 622, 624, 626, 628, 630, 632, |
| | 634, 636, 638, 640, 642, 644, 646, 648, 652, 654, 656; 658, 660, 662, 664, |
| | 666, 668, 670, 672, 674, 676, 680, 682, 684, 686, 688, 690, 692, 694, 696, |
| | 700, 702, 704, 706, 708, 710, 712, 714, 716, 718, 720, 722, 724, 726, 728, |
| | 730, 732, 734, 736, 738, 740, 742, 744, 746, 748, 750, 752, 754, 756, 758, |
| | 760, 762, 764, 766, 768, 770, 772, 774, 776, 778, 780, 782, 784, 786, 788, |
| | 790, 792, 794, 796, 798, 800, 802, 804, 806, 808, 810, 812, 814, 816, 818, |
| | 820, 822, 824, 826, 828, 830, 832, 834, 836, 838, 840, 842, 844; 846, 848, |
| | 850, 852, 854, 856, 858, 860, 862, 864, 866, 868, 870, 874, 876, 878, 880, |
| | 882, 884, 886, 888, 890, 892, 894, 896, 650, 678 in WO2016090337 |
| Folate receptor | Identifier 15 in US20170002072A1 |
| Folate receptor | Identifier 15, 23 in WO2012099973 |
| alpha | |
| FOLRl/CD3s | Identifier 90 in WO2014144722A2 |
| bispecific | |
| GCN4 | Identifier 165, 166, 167, 168, 169, 170 in WO2016168773 A3 |
| GD2 | Identifier 19, 20, 21, in WO2016134284; Identifier 8 in WO2015132604 |
| GPC3 | Identifier 1 in WO2016049459; Identifier 12 in US20160208015A1 |
| GPC4 | Identifier 24 in WO2016049459 |
| GPRC5D | Identifier 100, 101, 102, 103, 104, 105, 106, 107, 108, 109, 110, 111, 112, |
| | 113, 114, 115, 116, 117, 118, 119, 120, 121, 122, 123, 301, 313, 325, 337, |
| | 349, 361, 373, 385 in WO2016090312 |
| HER2/CD3 | Identifier 9 in WO2014144722 A2 |
| Human CD79b | Identifier 33 in WO2016112870 |
| Human collagen | Identifier 34 in WO2016112870 |
| VII | |
| IL4 | Identifier 17, 16 in WO2009121847 |
| Integrin bivalent | Identifier 2, 1 in WO2009070753 |
| Ipilimumab | Identifier 39 in US20160208021; Identifier 39 in WO2016112870 |
| Mec/CD3s | Identifier 78 in WO2014144722A2 |
| bispecific | |
| Mesothelin | Identifier 7 WO2015188141; Identifier NO 47, 46, 57, 48, 49, 50, 51, 53, |
| | 54, 55, 56, 58, 59, 62, 64, 65, 66, 67, 68, 69, 70, 52, 60, 61, 63 in |
| | WO2016090034; Identifier 10, 11, 12 in WO2013142034; Identifier 11 in |
| | WO2013063419 |
| MUC1 | Identifier 15 in US20160130357 |
| MUC2 | Identifier 17 in US20160130357 |
| MUC3 | Identifier 15 in US20160130357 |
| MUC4 | Identifier 17 in US20160130357 |
| Nivolumab | Identifier 38 in US20160208021; Identifier 38 in WO2016112870 |
| NYBR1 | Identifier 21 in US20160333422A1; Identifier 21, 18, 19 in |
| | WO2015112830 |
| O-acetylated GD2 | Identifier 29, 31 in US20150140023 |
| ganglioside | |
| OX40 | Identifier 33 in US20150190506 |
| PD1 | Identifier 39, 40, 41, 42, 43, 44, 45, 46, 47, 48, 49, 50, 51, 52, 53, 54, 55, |
| | 56, 57, 58, 59, 60, 61 in US20160311917A1 |
| PDK1 | Identifier 15 in WO2016090365 |
| PDL1 nanobody | Identifier 22, 23, 24, 25, 26, 27 in US20110129458 |
| PDL2 nanobody | Identifier 28, 29, 30, 31, 32, 33 in US20110129458 |
| 6462 | |
| PRAME | Identifier 63, 64, 65, 66, 67, 68, 69 in WO2016191246A2 |
| PSMA | Identifier 19, 21, 30, 31, 34, 35 in WO2012145714 |
| PSMA diabody | Identifier 12, 13, 14, 15 in WO2011069019 |
| Radiation | Identifier 22, 24 in WO2005042780A1 |
| inducible | |
| neoantigen | |
| Ranibizuman | Identifier 40 in US20160208021; Identifier 40 in WO2016112870 |
| RAS | Identifier 81 in WO2016154047 |
| Rituximab | Identifier 36 in US20160208021; Identifier 36 in WO2016112870 |
| RORI | Identifier 34 in EP3083691A2; Identifier 249, 250 251, 252, 253, 254, |
| | 255, 256, 257, 258, 259, 260, 261, 262, 263, 264, 265, 266, 267, 268 in |
| | US20160208018A1; Identifier 57 in EP3083671A1; Identifier 1, 2 in |
| | US20160304619A1; Identifier 34 in WO2015092024A2 |
| Teplizumab | Identifier 42 in WO2016112870 |
| Teplizumab | Identifier 42 in US20160208021 |
| (mutated) | |
| TOSO | Identifier 2 in EP3098237A1 |
| Trastuzumab | Identifier 35 in US20160208021; Identifier 35 in WO2016112870 |
| TRBC1 | Identifier 13, 14, 15, 16, 17, 18, 19, 20, 21, 22, 3 in WO2015132598 |
| TRBC2 | Identifier 23, 24, 25, 26, 27, 28, 29, 30, 31, 32 in WO2015132598 |
| TSLPR | Identifier 1, 2 in US20160311910A1; Identifier 1, 2 in WO2015084513 |
| VEGF | Identifier 168, 169, 170, 171, 172, 173, 174, 175 in US20160090427; |
| | Identifier 498, 500, 502, 504, 506, 508 in US20110177074A1 |
| VEGFR2 | Identifier 1, 2 in US20120213783 |
| WT1/HLA | Identifier 108, 113, 18, 36, 54, 72, 90 in WO2015070061 |
| bispecific | |

TABLE 4
The antigen-binding domain may comprise an antigen-binding domain derived
from a CAR that binds to an antigen target, such as those described
in the cited publications, the contents of each publication are incorporated
herein by reference in their entirety for all purposes.

| Antigen Target | Examples of Source |
|---|---|
| Acid/base leucine | Identifier 34, 35 in WO2016124930 |
| zipper | |
| ALK | Identifier 43, 44, 45, 46, 47, 48, 49, 50, 51, 52, 53, 54, 55, 56, 57, 58, |
| | 59, 60, 61, 62, 63, 64, 65, 66, 67, 68, 69, 70, 71, 72, 73, 74, 75, 76, |
| | 77, 78, 79, 80, 81, 82, 84, 85, 86, 87, 88, 89, 90 in WO2015069922 |
| APRIL-based CAR | Identifier 53 in US20160296562A1; Identifier 52 in |
| | US20160296562A1 |
| BCMA | Identifier 180, 162, 168, 174, 144, 150, 186, 192, 198, 204, 210, 156, |
| | 216, 222, 228, 234, 240, 246, 252, 258, 264, 270, 276 330, 282, 300, |
| | 306, 336, 354, 288, 312, 294, 342, 324, 318, 348 in |
| | WO2016168595A1; Identifier 48, 49, 50, 51, 52, 53, 54, 55, 56, 57, |
| | 58, 59, 19, 20, 21, 22, 23, 24, 25, 26, 27, 28, 29, 30, 31, 32, 33, 34, |
| | 35, 36, 37, 38, 39, 40, 41, 42 in WO2015158671A1; Identifier 124, |
| | 114, 115, 116, 117, 118, 119, 120, 121, 122, 123, 125, 126, 127, 128, |
| | 234, 235, 236, 237, 238, 239, 240, 241, 242, 243, 244, 245, 246, 247, |
| | 248, 249, 250, 251, 252, 253, 254, 267, 268, 269, 270 in |
| | WO2016014565; Identifier 1, 2, 3, 4, 5, 20 in WO2015052538; |
| | Identifier 1, 2, 3, 4, 5, 6 in US20160237139A1; Identifier 9 in |
| | WO2016094304 A3; Identifier 4, 5, 6, 8, 9, 10, 11, 12 in |
| | WO2013154760; Identifier 15, 16, 17, 18, 19, 20, 21, 22, 23, 24, 25, |
| | 26, 27, 28, 29, 71, 73 in WO2016014789; Identifier 125, 126, 127, |
| | 128, 129, 130, 131, 132, 133, 134, 135, 136, 137, 138, 139, 140, 141, |
| | 145, 146, 147, 148, 149, 150 in WO2016120216; Identifier 102, 106, |
| | 107, 108, 109, 110, 111, 112, 129, 130, 131, 132, 133, 134, 135, 136, |
| | 113, 114, 115, 116, 117, 118, 101, 100, 137, 119, 120, 121, 122, 123, |
| | 124, 125, 126, 127, 128, 103, 104, 105, 213 in WO2016097231 |
| CAR and gate (CD19 | Identifier 2 in US20160296562 |
| and CD33) CD148 | |
| phosphatase | |
| CAR and gate (CD19 | Identifier 43 in US20160296562 |
| and CD5) | |
| CAR and gate (CD19 | Identifier 45 in US20160296562 |
| and EGFR VIII) | |
| CAR and gate (CD19 | Identifier 41 in US20160296562 |
| and GD2) | |
| CAR and gate (CD19 | Identifier 3 in US20160296562 |
| or CD33) CD45 | |
| phosphatase | |
| CAR and not gate | Identifier 4, 5 in US20160296562 |
| (CD19 and not CD33) | |
| CAR and not gate | Identifier 6 in US20160296562A1 |
| (CD19 and not CD33) | |
| CAR and not gate 1 | Identifier 48 in US20160296562 |
| CAR and not gate 2 | Identifier 49 in US20160296562 |
| CAR and not gate 3 | Identifier 50 in US20160296562 |
| CAR or gate (DC19 or | Identifier 1 in US20160296562 |
| DC33) | |
| CAT19 CAR with | Identifier 12 in WO2016139487 |
| CD28 zeta | |
| endodomain | |
| CAT19 CAR with | Identifier 11 in WO2016139487 |
| OX40 zeta | |
| endodomain | |
| CAT19, campana | Identifier 10 in WO2016139487 |
| architecture | |
| CD123 | Identifier 69 in WO2016142532; Identifier 23, 24, 25, 26, 27, 28, 29, |
| | 30, 31, 32, 33, 34, 35, 36, 37, 38, 39, 40, 41, 42, 44, 45, 46, 47, 48 in |
| | WO2015140268A1; Identifier 9, 10, 11, 12 in US20140271582; |
| | Identifier 56, 57, 58, 59, 60, 61 in WO2016097231; Identifier 98, 99, |
| | 100, 101, 125, 126, 127, 128, 129, 130, 131, 132, 133, 134, 135, 136, |
| | 137, 138, 139, 140, 141, 142, 143, 144, 145, 146, 147, 148, 149, 150, |
| | 151, 152, 153, 154, 155, 156 in WO2016028896; Identifier 31, 32, |
| | 33, 34, 35, 36, 37, 38, 39, 40, 41, 42, 43, 44, 45, 46, 47, 48, 49, 50, |
| | 51, 52, 53, 54, 55, 56, 57, 58, 59, 60, 61, 62, 63, 64, 65, 66, 67, 68, |
| | 69, 70, 71, 72, 73, 74, 75, 76, 77, 78, 79, 80, 81, 82, 83, 84, 85, 86, |
| | 87, 88, 89, 90, 91, 92, 93, 94, 95, 96, 97, 98, 99, 100, 101, 102, 103, |
| | 104, 105, 106, 107, 108, 109, 110, 111, 112, 113, 114, 115, 116, 117, |
| | 118, 119, 120, 121, 122, 123, 124, 125, 126, 127, 128, 129, 130, 131, |
| | 132, 133, 134, 135, 136, 137, 138, 139, 140, 141, 142, 143, 144, 145, |
| | 146, 147, 148, 149, 150, 151, 152, 153, 154, 155, 156, 157, 158, 159, |
| | 160, 172, 173, 174, 175, 176, 177, 178, 179, 180, 181, 182, 183, 184, |
| | 185, 186, 187, 188, 189, 190, 191, 193, 194, 195, 196, 197 in |
| | WO2016120220; Identifier 1, 2, 3, 4, 5, 6, 7, 8, 9, 10, 142 in |
| | WO2016120216 |
| CD19 | Identifier 12 in US9499629B2; Identifier 24 in US20160333108A1; |
| | Identifier 25, 29 in US20160333108A1; Identifier 27 in |
| | US20160333108A1; Identifier 1 in EP2997134A4; Identifier 19, 20 |
| | in EP3071687A1; Identifier 181 in WO2016168773A3; Identifier 2 |
| | in WO2015157399A9; Identifier 56, 62 in WO2016174409A1; |
| | Identifier 145, 293, 294, 295, 296, 297, 298 in WO2016179319A1; |
| | Identifier 73 in WO2013176915A1; Identifier 73 in |
| | WO2013176916A1; Identifier 73 in US20130315884A1; Identifier |
| | 73 in US20140134142A1; Identifier 73 in US20150017136A1; |
| | Identifier 73 in US20150203817A1; Identifier 73 in |
| | US20160120905A1; Identifier 73 in US20160120906A1; Identifier |
| | 8, 5 in WO2015124715; Identifier 73 in WO2014184744; Identifier |
| | 73 in WO2014184741; Identifier 14, 15 in US20160145337A1; |
| | Identifier 14, 15 in WO2014184143; Identifier 15, 16 in |
| | WO2015075175; Identifier 16 in US20160145337A1; Identifier 16 in |
| | WO2014184143; Identifier 12 in WO2012079000; Identifier 31, 32, |
| | 33, 34, 35, 36, 37, 38, 39, 40, 41, 42, 58 in WO2016164580; |
| | Identifier 14, 15 in US20160296563A1; Identifier 31, 32, 33, 34, 35, |
| | 36, 37, 38, 39, 40, 41, 42 in WO2015157252; Identifier 14, 15 in |
| | WO2016139487; Identifier 53, 54, 55, 56, 57, 58 in |
| | US20160250258A1; Identifier 1, 2, 3, 4, 5, 6, 7, 8, 9, 10, 11, 12, 13 |
| | in WO2015187528; Identifier 31, 32, 33, 34, 35, 36, 37, 38, 39, 40, |
| | 41, 42, 58 in WO2015157252; Identifier 31, 32, 33, 34, 35, 36, 37, |
| | 38, 39, 40, 41, 42 in WO2014153270; WO2016134284 (no |
| | Identifier); Identifier 13 in WO2016139487 |
| CD19 or CD33 | Identifier 1 in WO2015075468 which recognizes CD19 OR CD33 |
| CD19/CD20 bispecific | Identifier 1308 in WO2016164731A2; Identifier 2, 8, 11 in |
| | US9447194B2 |
| CD19/IL13 bispecific | Identifier 10 in US20160340649A1 |
| CD2 | Identifier 10, 11 in WO2016138491 |
| CD20 | Identifier 25 in WO2015157399A9; Identifier 177, 181, 182, 183, |
| | 184, 185, 186, 187, 205, 206, 207, 208, 209, 210, 211, 188, 189, 190, |
| | 191, 192, 193, 176, 212, 194, 195, 196, 197, 198, 199, 200, 201, 202, |
| | 203, 178, 179, 180 in WO2016097231 |
| CD22 | Identifier 380, 204, 260, 266, 272, 278, 284, 290, 296, 302, 308, 341, |
| | 213, 320, 326, 332, 338, 347, 350, 356, 362, 368, 374, 219, 386, 392, |
| | 398, 404, 410, 416, 421, 427, 225, 230, 1109, 236, 242, 248, 254 in |
| | WO2016164731A2; Identifier 15, 16, 17, 18, 19, 20, 32 in |
| | WO2013059593; Identifier 22, 23, 24 in US20150299317 |
| CD22/CD19 bispecific | Identifier 29, 30 in WO2016149578; Identifier 1304 in |
| | WO2016164731A2 |
| CD276 | Identifier 39, 40, 41, 42, 43, 44, 45, 46, 47, 122, 123, 124, 125, 126, |
| | 127, 128, 129, 130 in US20160053017 |
| CD3 | Identifier 12 in WO2016138491 |
| CD30 | Identifier 20 in WO2016008973A1; Identifier 1 in |
| | WO2016116035A1; WO2016134284 (no Identifier); Identifier 2 in |
| | WO2016008973 |
| CD33 | Identifier 48, 49, 50, 51, 52, 53, 54, 55, 83 in WO2016014576; |
| | Identifier 19, 20, 21, 22, 23, 24, 25, 26, 27, 28, 29, 30, 31, 32, 33, 34, |
| | 35, 36, 37, 38, 39, 40, 41, 42, 48, 49, 50, 51, 52, 53, 54, 55, 56, 57, |
| | 58, 59, 60, 61, 62, 63, 64, 65, 66, 67, 68, 69, 70, 71 in |
| | WO2015150526A2 |
| CD38 | Identifier 70, 71, 72, 64, 65, 66, 67, 68, 69 in WO2016097231; |
| | Identifier 35, 36, 37 in WO2015121454 |
| CD4 | Identifier 13, 14 in WO2016138491 |
| CD410 | Identifier 7 in EP3074419A2 |
| CD435 | Identifier 5 in EP3074419A2 |
| CD44 | Identifier 21, 22, 23, 24, 25, 26, 27, 28, 31, 32, 33, 34, 35 in |
| | WO2016042461A1 |
| CD4-DDY3 | Identifier 9 in EP3074419A2 |
| CD5 | Identifier 15, 13 in WO2016138491 |
| CD52 | Identifier 18 in WO2016138491 |
| CD7 | Identifier 17 in WO2016138491 |
| CD70 | Identifier 99 in WO2015121454 |
| CD70 | Identifier 100, 93, 94, 96, 101, 95, 97, 98 in WO2015121454 |
| CD8 stalk APRIL | Identifier 51 in US20160296562A1 |
| CEA | Identifier 4 in WO2016008973A1; Identifier 29, 30 in |
| | US20140242701A |
| CLDN6 | Identifier 22, 23, 24 in WO2016150400 |
| CLL1 | Identifier 148 in WO2016179319A1; Identifier 35, 36, 37, 38, 39, 40, |
| | 41, 42, 43, 44, 45, 46, 47, 48, 49, 50, 51, 52, 53, 54, 55, 56, 57, 58, |
| | 59, 60, 61, 62, 63, 64, 65, 66, 67, 68, 69, 70, 71, 72, 73, 74, 75, 76, |
| | 77, 78, 79, 80, 81, 82, 83, 84, 85, 86, 87, 88, 89, 90, 91, 92, 93, 94, |
| | 95, 96, 97, 98, 99, 100, 101, 102, 103, 104, 105, 106, 107, 108, 109, |
| | 110, 111, 112 in WO2016120218; Identifier 91, 92, 93, 94, 95, 96, |
| | 97, 98, 99, 100, 101, 102, 103, 197 in WO2016014535 |
| COM22 | Identifier 358, 359, 360 in US20160297884A1 |
| CS1 | Identifier 55, 57, 60, 54, 56, 48, 49, 50, 51, 52, 53, 58, 59, 61, 62 in |
| | WO2015121454; Identifier 28 in WO2014179759A1 |
| DDD1/AD1 zip | Identifier 37 in WO2016124930 |
| DDD1/AD1-based zip | Identifier 36 in WO2016124930 |
| EGFR | Identifier 3, 2in WO2014130657; Identifier 36, 37, 38, 39, 35 in |
| | US20140242701 A; Identifier 43, 96, 49, 55, 61, 67, 73, 79, 85, 90, 1 |
| | in WO2014130657 |
| EGFR vIII | Identifier 15, 16, 17, 18, 24, 25, 26, 27 in WO2016016341; Identifier |
| | 5, 10, 12, 8, 31, 30, 3 in US20160311907A1; Identifier 10 in |
| | US20160200819A1; Identifier 43, 49, 55, 61, 67, 73, 79, 85, 90, 96 |
| | in US9394368B2; Identifier 49, 55, 61, 67, 73, 79, 85, 90, 2, 1 in |
| | US20170008963A1; Identifier 10, 11 in US20140037628 |
| FcRL5 | Identifier 11 in US20170008963A1 |
| Folate receptor | Identifier 12 in US20170008963A1 |
| FR | Identifier 22 in US20170002072A1 |
| FR beta | Identifier 13, 22 in US9402865B2; Identifier 2, 4, 6 in US9446105B2 |
| FRa | Identifier 13, 14 in US20120213783 |
| Fra | Identifier 959 in WO2016090337; Identifier 13 in |
| | US20170002072A1 |
| GCN4 | Identifier 8, 10 in US9446105B2 |
| GD2 | Identifier 12 in US9446105B2; Identifier 273, 274 in |
| | WO2016168773A3; Identifier 26, 27, 28, 29, 30, 31, 32, 33, 34, 35, |
| | 36, 37 in WO2015132604; WO2016134284 (no Identifier) |
| GD3 | Identifier 19 in WO2016185035A1; Identifier 20, 21, 22, 23, 24, 25, |
| | 26 in WO2016185035A1; WO2016134284 (no Identifier) |
| GFR alpha | Identifier 27 in WO2016185035A1 |
| GPC3 | Identifier 28, 29 in WO2016185035A1; Identifier 3, 27, 10, 29, 14, |
| | 30, 31, 18, 33 in WO2016049459; Identifier 22 in |
| | US20160215261A1 |
| HER2 | Identifier 25 in US20160215261A1; Identifier 9, 10 of |
| | WO2016073629; Identifier 17, 28, 98, 110 in US20160333114A1; |
| | Identifier 271, 272 in WO2016168773A3; Identifier 5 in |
| | WO2016168769A1 |
| Herl/Her3 bispecific | Identifier 23, 24 in US20160215261A1 |
| HERVK | Identifier 6 in WO2016168769A1 |
| HIV Env | Identifier 48, 49 in WO2016168766A1; Identifier 4 in |
| | EP2997134A4; Identifier 7, 9, 47, 49 in WO2015077789 |
| HSP70 | Identifier 51, 53, 5 in WO2015077789; Identifier 21, 22, 23, 24, 25, |
| | 26, 27, 28, 29 in WO2016120217 |
| IL13 | Identifier 30, 31, 32 in WO2016120217 |
| IL13Ra2specific | Identifier 4, 5, 6 in WO2016089916A1; Identifier 47, 49 in |
| | WO2016123143; Identifier 51, 53, 55 in WO2016123143; Identifier |
| | 1, 31, 32, 33, 34, 35, 36, 37, 38, 39, 40, 41, 42, 43, 44, 45 in |
| | US20160340649A1 |
| KMA | Identifier 46 in US20160340649A1 |
| Mesothelin | Identifier 47, 48 in US20160340649A1; Identifier 48 in |
| | US20160340649A1; Identifier 27 in WO2016172703A2; Identifier |
| | 18, 19, 20, 21, 22, 23 in WO2013142034; Identifier 3 in |
| | WO2013067492 |
| MUC1 | Identifier 5, 7 in WO2013063419; Identifier 51 in |
| | US20160340406A1; Identifier 30, 32, 34 in US20160130357; |
| | Identifier 295, 298, 301, 304, 307, 607, 609, 611, 613 in |
| | WO2016130726 |
| NCAR with RQR82 | Identifier 615 in WO2016130726 |
| ACD 19 | |
| NYBR1 | Identifier 617, 619 in WO2016130726; Identifier 218 in |
| | WO2016097231; Identifier 26, 29, 60 in WO2015112830; Identifier |
| | 1 in US20160333422A1 |
| P5A | Identifier 26, 29, 60 in US20160333422A1 |
| P5AC1 | Identifier 343, 344, 345, 346 in US20160297884A1 |
| P5AC16 | Identifier 347, 396, 348 in US20160297884A1 |
| P6AP | Identifier 349, 350, 351 in US20160297884A1 |
| P6DY | Identifier 364, 365, 366 in US20160297884A1 |
| PC1C12 | Identifier 352, 353, 354 in US20160297884A1 |
| PCI | Identifier 361, 362, 363 in US20160297884A1 |
| PD1 | Identifier 355, 356, 357 in US20160297884A1; Identifier 119 in |
| | WO2014153270; Identifier 121 in WO2014153270; Identifier 22, 24, |
| | 63, 64, 65, 66, 67, 68, 69, 70, 71, 72, 73, 74, 75, 76, 77, 78, 79, 80, |
| | 81, 82, 83, 84, 85, 86 in US20160311917A1; Identifier 26, 39 in |
| | WO2016172537A1; Identifier 40 in US20160311907A1; Identifier |
| | 121, 119 in WO2015157252; Identifier 24 in WO2016014565; |
| | Identifier 22 in WO2016014565 |
| PD1 | Identifier 23 in WO2016014565; Identifier 26 in WO2015142675 |
| PSMA | Identifier 39 in WO2015142675; Identifier 28, 29 in |
| | US20160311907A1; Identifier 140, 144, 145, 146, 147, 148, 149, |
| | 150, 167, 168, 169, 170, 171, 172, 173, 174, 151, 152, 153, 154, 155, |
| | 156, 139, 138, 175, 157, 158, 159, 160, 161, 162, 163, 164, 165, 166, |
| | 141, 142, 143, 214 in WO2016097231 |
| ROR1 | Identifier 216, 217, 215 in WO2016097231; Identifier 79, 80, 81, 82, |
| | 83, 84, 85, 86, 87, 88, 89, 90, 91, 92, 93, 94, 95, 96, 103, 104, 105, |
| | 106, 107, 108, 109, 110, 111, 112, 113, 114, 115, 116, 117, 118, 119, |
| | 120, 127, 128, 129, 130, 131, 132, 133, 134, 1335, 136, 137, 138, 97, |
| | 98, 99, 100, 101, 102, 121, 122, 123, 124, 125, 126 in |
| | WO2016016344A1; Identifier 386, 387, 388, 389, 390, 391, 392, |
| | 393, 394 in WO2016187216A1 |
| SNAP | Identifier 395 in WO2016187216A1 |
| SSEA4 | Identifier 396, 397 in WO2016187216A1 |
| Tan | Identifier 19 in US20160311907A1 recognizes CD19 AND CD33 |
| | using a CD45 phosphatase; Identifier 5 in WO2016026742A1 |
| | recognizes CD19 AND CD33 using a CD148 phosphatase; Identifier |
| | 6 in WO2016026742A1 which recognizes CD19 AND NOT CD33 |
| | and is based on an ITIM containing endodomain from LAIR1; |
| | Identifier 3 in WO2015075468 which recognizes CD19 AND NOT |
| | CD33 based on PTPN6 phosphatase; Identifier 2 in WO2015075468 |
| | which recognizes CD19 AND NOT CD33 and recruits a |
| | PTPN6/CD148 fusion protein to an ITIM containing endodomain |
| TOSO | Identifier 5, 4 in WO2015075468 |
| Trophoblast | Identifier 6 in WO2015075468; Identifier 4 in US20160347854A1; |
| Glycoprotein 5T4 | Identifier 4 in EP3098237A1; Identifier 19, 20, 21, 22, 23, 24, 25, 26, |
| | 27, 28, 29, 30, 31, 32, 33, 34, 35, 36, 37, 38, 39 in |
| | WO2016034666A1 |
| TSLPR | Identifier 40, 41, 42 in WO2016034666A1; Identifier 39, 40, 41, 42, |
| | 43, 44, 45, 46 in WO2015084513; Identifier 39, 40, 41, 42, 43 in |
| | US20160311910A1 |
| VEGFR2 | Identifier 44, 45, 46 in US20160311910A1; Identifier 10, 11, 12 in |
| | US20120213783 |
| VNAR | Identifier 105, 106, 107, 108, 109, 110 in US20160333094A1 |

In various embodiments, the scFv fragment used in the arCAR system of the present disclosure may include a linker between the VH and VL domains. The linker can be a peptide linker and may include any naturally occurring amino acid. Exemplary amino acids that may be included into the linker are Gly, Ser Pro, Thr, Glu, Lys, Arg, Ile, Leu, His and The. The linker should have a length that is adequate to link the VH and the VL in such a way that they form the correct conformation relative to one another so that they retain the desired activity, such as binding to an antigen. The linker may be about 5-50 amino acids long. In some embodiments, the linker is about 10-40 amino acids long. In some embodiments, the linker is about 10-35 amino acids long. In some embodiments, the linker is about 10-30 amino acids long. In some embodiments, the linker is about 10-25 amino acids long. In some embodiments, the linker is about 10-20 amino acids long. In some embodiments, the linker is about 15-20 amino acids long. Exemplary linkers that may be used are Gly rich linkers, Gly and Ser containing linkers, Gly and Ala containing linkers, Ala and Ser containing linkers, and other flexible linkers.

In one embodiment, the linker is a Whitlow linker. In one embodiment, the Whitlow linker comprises the amino acid sequence set forth in SEQ ID NO: 3, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 3. In another embodiment, the linker is a (G₄S)₃ linker. In one embodiment, the (G₄S)₃ linker comprises the amino acid sequence set forth in SEQ ID NO: 25, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 25.

Other linker sequences may include portions of immunoglobulin hinge area, CL or CH1 derived from any immunoglobulin heavy or light chain isotype. Exemplary linkers that may be used include any of SEQ ID NOs: 26-56 in Table 1. Additional linkers are described for example in Int. Pat. Publ. No. WO2019/060695, incorporated by reference herein in its entirety.

In some embodiments, the second polypeptide comprises the antigen-binding domain at the N-terminus and the tag at the C-terminus. In some embodiments, the second polypeptide comprises the antigen-binding domain at the C-terminus and the tag at the N-terminus.

In some embodiments, the second polypeptide is a soluble polypeptide.

In some embodiments, the arCAR system further comprises one or more additional polypeptides each comprising (a) an antigen-binding domain that binds to a unique antigen and (b) a tag that is recognized by the tag-binding domain of the first polypeptide.

In another aspect, provided herein is an arCAR system comprising two or more arCARs described herein, wherein each arCAR comprises a unique pair of tag and tag-binding domain.

In further aspects, provided herein is a polypeptide of an arCAR described herein. In one aspect, provided herein is a first polypeptide of an arCAR described herein. In one aspect, provided herein is a second polypeptide of an arCAR described herein.

Without wishing to be bound by theory, the arCAR systems of the present disclosure may have various advantages including:
The high degree of specificity that the anti-ID molecule/antibody fragment minimizes off-target interactions, particularly in combination with the non-human protein target for the antibody specificity.
Specificity to the binding loops on the tag avoids undesirable interacting with similar framework/scaffold proteins. True-IDs, which neutralize the binding to target, can also be employed.
Inherent ability to develop a range of affinities to optimize and modulate the “tag” polypeptide and CAR interaction.
The system is unlimited in possible pairs such that numerous optimized CAR/“tag” polypeptide pairs can be developed, each with a unique specificity to enable fit-for-purpose therapy including modulation of binding affinities, logic-gating, multiple specificities per cell and multiple soluble drugs per treatment.
The approach is “platform agnostic” which allows one to incorporate a different scaffold (for example, replace an scFv with a VHH), if immunogenicity or other limitations towards one scaffold develop.

### Polynucleotides and Vectors

In another aspect, provided herein are polynucleotides encoding one or more polypeptides in an arCAR system of the present disclosure.

In some embodiments, provided herein is a polynucleotide encoding the first polypeptide of an arCAR system of the present disclosure. In some embodiments, provided herein is a polynucleotide encoding the first polypeptides of two or more arCAR systems of the present disclosure. In some embodiments, the two or more arCAR systems each comprise a unique pair of tag and tag-binding domain.

In some embodiments, provided herein is a polynucleotide encoding the second polypeptide of an arCAR system of the present disclosure. In some embodiments, provided herein is a polynucleotide encoding the second polypeptides of two or more arCAR systems of the present disclosure. In some embodiments, the two or more arCAR systems each comprise a unique pair of tag and tag-binding domain.

In some embodiments, provided herein is a polynucleotide encoding both polypeptides of an arCAR system of the present disclosure. In some embodiments, the polynucleotide encodes both polypeptides of two or more arCAR systems of the present disclosure. In some embodiments, the two or more arCAR systems each comprise a unique pair of tag and tag-binding domain.

The polynucleotide can comprise any type of nucleotides, including, but not limited to DNA and RNA, which can be single-stranded or double-stranded, synthesized or obtained in part from natural sources, and which can contain natural, non-natural or altered nucleotides. The polynucleotide can comprise naturally-occurring or non-naturally-occurring internucleotide linkages, or both types of linkages.

In some embodiments, the polynucleotide described herein is a DNA molecule. In various embodiments, the polynucleotide described herein is an RNA molecule.

In one aspect, the present disclosure provides recombinant vectors comprising a polynucleotide described herein.

In some embodiments, the recombinant vector comprises a polynucleotide encoding the first polypeptide of an arCAR system of the present disclosure. In some embodiments, the recombinant vector comprises a polynucleotide encoding the first polypeptides of two or more arCARs of the present disclosure. In some embodiments, the two or more arCARs each comprise a unique pair of tag and tag-binding domain.

In some embodiments, the recombinant vector comprises a polynucleotide encoding the second polypeptide of an arCAR system of the present disclosure. In some embodiments, the recombinant vector comprises a polynucleotide encoding the second polypeptides of two or more arCARs of the present disclosure. In some embodiments, the two or more arCARs each comprise a unique pair of tag and tag-binding domain.

In some embodiments, the recombinant vector comprises a polynucleotide encoding both polypeptides of an arCAR system of the present disclosure. In some embodiments, the recombinant vector comprises a polynucleotide encoding both polypeptides of two or more arCARs of the present disclosure. In some embodiments, the two or more arCARs each comprise a unique pair of tag and tag-binding domain.

A recombinant vector can be any suitable recombinant expression vector. Suitable vectors include those designed for propagation and expansion or for expression or both, such as plasmids and viruses. For example, a vector can be selected from the pUC series (Fermentas Life Sciences, Glen Burnie, Md.), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.). Bacteriophage vectors, such as λGT10, λGT11, λZapII (Stratagene), λEMBL4, and λNM1149, can also be used. Examples of plant expression vectors useful in the context of the disclosure include pBI01, pBI101.2, pBI101.3, pBI121 and pBIN19 (Clontech). Examples of animal expression vectors useful in the context of the disclosure include pcDNA, pEUK-Cl, pMAM, and pMAMneo (Clontech). In some embodiments, a bicistronic IRES vector (e.g., from Clontech) can be used to include both a polynucleotide encoding the first polypeptide and the second polypeptide of an arCAR system described herein.

In some embodiments, the recombinant vector is a non-viral vector. The viral vector may be a plasmid or a transposon (such as a PiggyBac- or a Sleeping Beauty transposon). In one embodiment, the vector is a plasmid.

In some embodiments, the recombinant vector is a viral vector Suitable viral vectors include, without limitation, retroviral vectors, lentiviral vectors, alphaviral vectors, adenoviral vectors, adeno-associated viral vectors (AAVs), herpes viral vectors, vaccinial vectors, and fowl pox viral vectors. In some embodiments, the viral vectors have a native or engineered capacity to transform a host cell (e.g., T cell).

Recombinant vectors can be prepared using standard recombinant DNA techniques described in, for example, Sambrook et al., Molecular Cloning: A Laboratory Manual, 3rd ed., Cold Spring Harbor Press, Cold Spring Harbor, N.Y. 2001; and Ausubel et al., Current Protocols in Molecular Biology, Greene Publishing Associates and John Wiley & Sons, NY, 1994. Constructs of expression vectors, which are circular or linear, can be prepared to contain a replication system functional in a prokaryotic or eukaryotic host cell. Replication systems can be derived, e.g., from ColE1, 2μ plasmid, λ, SV40, bovine papilloma virus, and the like.

A recombinant vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the recombinant expression vectors include, for instance, neomycin/G418 resistance genes, puromycin resistance genes, hygromycin resistance genes, histidinol resistance genes, tetracycline resistance genes, and ampicillin resistance genes.

Vectors useful in the context of the disclosure can be “naked” nucleic acid vectors (i.e., vectors having little or no proteins, sugars, and/or lipids encapsulating them), or vectors complexed with other molecules. Other molecules that can be suitably combined with the vectors include without limitation viral coats, cationic lipids, liposomes, polyamines, gold particles, and targeting moieties such as ligands, receptors, or antibodies that target cellular molecules.

Vector DNA can be introduced into a host cell, e.g., an immune effector cell, via conventional transformation or transfection techniques. As used herein, the terms “transformation” and “transfection” are intended to refer to a variety of art-recognized techniques for introducing foreign nucleic acid (e.g., DNA) into a cell, including calcium phosphate or calcium chloride co-precipitation, DEAE-dextran-mediated transfection, lipofection, gene gun, or electroporation.

In various embodiments, the polynucleotide encoding the arCAR polypeptide (e.g., first polypeptide or second polypeptide) is operably linked to at least a regulatory element. The regulatory element can be capable of mediating expression of the arCAR polypeptide (e.g., first polypeptide or second polypeptide) in the host cell. Regulatory elements include, but are not limited to, promoters, enhancers, initiation sites, polyadenylation (polyA) tails, IRES elements, response elements, and termination signals. In certain embodiments, the regulatory element regulates arCAR polypeptide expression. In certain embodiments, the regulatory element increased the expression of the arCAR polypeptide (e.g., first polypeptide or second polypeptide). In certain embodiments, the regulatory element increased the expression of the arCAR polypeptide (e.g., first polypeptide or second polypeptide) once the host cell is activated. In certain embodiments, the regulatory element decreases expression of the arCAR polypeptide (e.g., first polypeptide or second polypeptide). In certain embodiments, the regulatory element decreases expression of the arCAR polypeptide (e.g., first polypeptide or second polypeptide) once the host cell is activated.

### Modified Host Cells

In one aspect, provided herein are host cells modified to express one or more of the polypeptides of the arCARs of the present disclosure.

In some embodiments, provided herein is a host cell which is an immune effector cell comprising the first polypeptide of an arCAR system of the present disclosure. In some embodiments, provided herein is a host cell comprising the first polypeptides of two or more arCARs of the present disclosure. In some embodiments, the two or more arCARs each comprise a unique pair of tag and tag-binding domain.

In some embodiments, provided herein is a host cell comprising the second polypeptide of an arCAR of the present disclosure. In some embodiments, provided herein is a host cell comprising the second polypeptides of two or more arCARs of the present disclosure. In some embodiments, the two or more arCARs each comprise a unique pair of tag and tag-binding domain.

In some embodiments, provided herein is a host cell comprising both the first and the second polypeptides of an arCAR of the present disclosure.

In some embodiments, provided herein is a host cell comprising a polynucleotide, or a recombinant vector described herein.

In various embodiments, the cell having the chimeric antigen receptor comprising the first polypeptide is an immune-effector cell. In some embodiments, the cell is a T cell, a Natural Killer (NK) cell, a cytotoxic T lymphocyte (CTL), a regulatory T cell, a tumor-infiltrating lymphocyte (TIL), a dendritic cell or a macrophage.

In some embodiments, the immune effector cell is derived from a stem cell. In some embodiments, the cell is an induced pluripotent stem cell (iPSC), a hematopoietic stem cells (HSCs), or an embryonic stem cell (ESC). In some embodiments, the cell is an iPSC. In one embodiment, the cell is a T cell derived from an iPSC. In one embodiment, the cell is a NK cell derived from an iPSC.

In one aspect, provided herein are methods of preparing the modified host cells described herein.

In one embodiment, the method of preparing the modified host cell comprises introducing the polynucleotide encoding the first polypeptide of an arCAR of the present disclosure, or a recombinant vector comprising the polynucleotide, into the cell.

In one embodiment, the method of preparing the modified host cell comprises introducing the polynucleotide encoding the second polypeptide of an arCAR of the present disclosure, or a recombinant vector comprising the polynucleotide, into the cell.

In one embodiment, the method of preparing the modified host cell comprises introducing the polynucleotide encoding both the first polypeptide and the second polypeptide of an arCAR of the present disclosure, or a recombinant vector comprising the polynucleotide, into the cell.

In various embodiments, the modified cell constitutively expresses the first polypeptide of an arCAR described herein. In various embodiments, the modified cell inducibly expresses the first polypeptide of an arCAR described herein.

In various embodiments, the modified cell constitutively expresses the second polypeptide of an arCAR described herein. In various embodiments, the modified cell inducibly expresses the second polypeptide of an arCAR described herein.

In various embodiments, the host cells may be autologous/autogenic (“self”) or non-autologous (“non-self,” e.g., allogeneic, syngeneic or xenogeneic). In some embodiments, the host cells are obtained from a mammalian subject. In some embodiments, the host cells are obtained from a primate subject. In some embodiments, the host cells are obtained from a human subject.

In certain embodiments, immune cells such as lymphocytes are used. Lymphocytes can be obtained from sources such as, but not limited to, peripheral blood mononuclear cells, bone marrow, lymph nodes tissue, cord blood, thymus issue, tissue from a site of infection, ascites, pleural effusion, spleen tissue, and tumors. Lymphocytes may also be generated by differentiation of stem cells. In certain embodiments, lymphocytes can be obtained from blood collected from a subject using techniques generally known to the skilled person, such as sedimentation, e.g., FICOLL™ separation.

In certain embodiments, immune cells from the circulating blood of a subject are obtained by apheresis. An apheresis device typically contains lymphocytes, including T cells, monocytes, granulocytes, B cells, other nucleated white blood cells, red blood cells, and platelets. In certain embodiments, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. A washing step may be accomplished by methods known to those in the art, such as, but not limited to, using a semiautomated flowthrough centrifuge (e.g., Cobe 2991 cell processor, or the Baxter CytoMate). After washing, the cells may be resuspended in a variety of biocompatible buffers, cell culture medias, or other saline solution with or without buffer.

In certain embodiments, T lymphocytes can be isolated from peripheral blood mononuclear cells (PBMCs) by lysing the red blood cells and depleting the monocytes. As an example, the cells can be sorted by centrifugation through a PERCOLL™ gradient. In certain embodiments, after isolation of PBMC, both cytotoxic and helper T lymphocytes can be sorted into naive, memory, and effector T-cell subpopulations either before or after activation, expansion, and/or genetic modification.

In certain embodiments, T lymphocytes can be enriched. For example, a specific subpopulation of T lymphocytes, expressing one or more markers such as, but not limited to, CD3, CD4, CD8, CD14, CD15, CD16, CD19, CD27, CD28, CD34, CD36, CD45RA, CD45RO, CD56, CD62, CD62L, CD122, CD123, CD127, CD235a, CCR7, HLA-DR or a combination thereof using either positive or negative selection techniques. In certain embodiments, the T lymphocytes for use in the compositions of the disclosure do not express or do not substantially express one or more of the following markers: CD57, CD244, CD160, PD-1, CTLA4, TIM3, and LAG3.

In certain embodiments, NK cells can be enriched. For example, a specific subpopulation of T lymphocytes, expressing one or more markers such as, but not limited to, CD2, CD16, CD56, CD57, CD94, CD122 or a combination thereof using either positive or negative selection techniques.

In certain embodiments, pluripotent stem cells (PSCs) such as induced pluripotent stem cells (iPSCs) and embryonic stem cells (ESCs) are used to generate the host cells such as NK cells or T lymphocytes. Human iPSCs and human ESCs can be produced by various methods known in the art. PSCs (e.g., iPSCs or ESCs) can be modified by an arCAR of the present disclosure by, e.g., contacting the cells with a polynucleotide or recombinant vector encoding a polypeptide of the arCAR, and the engineered PSC can be used to produce or generate immune cells (e.g., T cells) comprising the arCAR of the present disclosure.

iPSCs can be generated directly from adult cells (e.g., somatic cells). iPSCs can be derived or generated by introducing a specific set of pluripotency-associated genes, or “reprogramming factors”, into a given cell type. Reprogramming factors include, but are not limited to, OCT4 (also known as “POU5FL”), SOX2, cMYC, and KLF4, which are also known as Yamanaka factors. See Takahashi, K; Yamanaka, S (2006). Cell 126 (4): 663-76. Each of the reprogramming factors can be functionally replaced by related transcription factors, miRNAs, small molecules, or even non-related genes such as lineage specifiers. Upon introduction of reprogramming factors, cells begin to form colonies that resemble PSCs, which can be isolated based on their morphology, conditions that select for their growth, or through expression of surface markers or reporter genes. In certain embodiments, the PSCs used in the methods of the present invention are subject-specific.

There are known technologies for producing PSCs from various types of somatic cells by reprogramming using the Yamanaka factors (OCT4, SOX2, KLF4, and cMYC). For example, reprogramming of mature lymphocytes into iPSCs was accomplished for murine B cells (Hanna et al., (2008) Cell, 133, pp. 250-264; Wada et al., (2011) Int. Immunol., 23, pp. 65-74), for murine T cells and mature NK T cells (Watarai et al., (2010) J. Clin. Invest., 120, pp. 2610-2618), and for human T cells (Loh et al., (2010) Cell Stem Cell, 7, pp. 15-19; Seki et al., (2010) Cell Stem Cell, 7, pp. 11-14). iPSCs can be produced from human T cells by using whole peripheral mononuclear cells (PBMCs) or CD3 cells as a source cell population (Loh et al., (2010) Cell Stem Cell, 7, pp. 15-19; Seki et al., (2010) Cell Stem Cell, 7, pp. 11-14; Staerk et al. (2010) Cell Stem Cell, 7, pp. 20-24; Brown et al, (2010) PloS One 5, e11373).

In order to reach sufficient therapeutic doses of host cell compositions, host cells are often subjected to one or more rounds of stimulation/activation. In certain embodiments, a method of producing host cells for administration to a subject comprises stimulating the host cells to become activated in the presence of one or more stimulatory signals or agents (e.g., compound, small molecule, e.g., small organic molecule, nucleic acid, polypeptide, or a fragment, isoform, variant, analog, or derivative thereof). In certain embodiments, a method of producing host cells for administration to a subject comprises stimulating the host cells to become activated and to proliferate in the presence of one or more stimulatory signals or agents.

Immune cells (e.g., T lymphocytes and NK cells) can be activated by inducing a change in their biologic state by which the cells express activation markers, produce cytokines, proliferate and/or become cytotoxic to target cells. All these changes can be produced by primary stimulatory signals. Co-stimulatory signals amplify the magnitude of the primary signals and suppress cell death following initial stimulation resulting in a more durable activation state and thus a higher cytotoxic capacity.

T cells can be activated generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety. In certain embodiments, the T cells are activated by binding to an agent that activates CD3ζ.

In some embodiments, a CD2-binding agent may be used to provide a primary stimulation signal to the T cells. For example, and not by limitation, CD2 agents include, but are not limited to, CD2 ligands and anti-CD2 antibodies, e.g., the Tl 1.3 antibody in combination with the Tl 1.1 or Tl 1.2 antibody (Meuer, S. C. et al. (1984) Cell 36:897-906) and the 9.6 antibody (which recognizes the same epitope as Tl 1.1) in combination with the 9-1 antibody (Yang, S. Y. et al. (1986) J. Immunol. 137:1097-1100). Other antibodies which bind to the same epitopes as any of the above described antibodies can also be used.

In some embodiments, the immune cells are activated by administering phorbol myristate acetate (PMA) and ionomycine. In certain embodiments, the host cells are activated by administering an appropriate antigen that induces activation and then expansion. In certain embodiments, PMA, ionomycin, and/or appropriate antigen are administered with CD3 to induce activation and/or expansion.

In general, the activating agents used in the present disclosure includes, but is not limited to, an antibody, a fragment thereof and a proteinaceous binding molecule with antibody-like functions. Examples of (recombinant) antibody fragments are Fab fragments, Fv fragments, single-chain Fv fragments (scFv), a divalent antibody fragment such as an (Fab)2′-fragment, diabodies, triabodies (Iliades, P., et al., FEBS Lett (1997) 409, 437-441), decabodies (Stone, E., et al., Journal of Immunological Methods (2007) 318, 88-94) and other domain antibodies (Holt, L. J., et al., Trends Biotechnol. (2003), 21, 11, 484-490). The divalent antibody fragment may be an (Fab)2′-fragment, or a divalent single-chain Fv fragment while the monovalent antibody fragment may be selected from the group consisting of a Fab fragment, a Fv fragment, and a single-chain Fv fragment (scFv).

In certain embodiments, one or more binding sites of the CD3ζ agents may be a bivalent proteinaceous artificial binding molecule such as a dimeric lipocalin mutein (i.e., duocalin). In certain embodiments the receptor binding reagent may have a single second binding site, (i.e., monovalent). Examples of monovalent agents include, but are not limited to, a monovalent antibody fragment, a proteinaceous binding molecule with antibody-like binding properties or an MHC molecule. Examples of monovalent antibody fragments include, but are not limited to a Fab fragment, a Fv fragment, and a single-chain Fv fragment (scFv), including a divalent single-chain Fv fragment.

The agent that specifically binds CD3 includes, but is not limited to, an anti-CD3-antibody, a divalent antibody fragment of an anti-CD3 antibody, a monovalent antibody fragment of an anti-CD3-antibody, and a proteinaceous CD3-binding molecule with antibody-like binding properties. A proteinaceous CD3-binding molecule with antibody-like binding properties can be an aptamer, a mutein based on a polypeptide of the lipocalin family, a glubody, a protein based on the ankyrin scaffold, a protein based on the crystalline scaffold, an adnectin, and an avimer. It also can be coupled to a bead.

In certain embodiments, the activating agent (e.g., CD3-binding agents) can be present in a concentration of about 0.1 to about 10 μg/ml. In certain embodiments, the activating agent (e.g., CD3-binding agents) can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In certain embodiments, the activating agent (e.g., CD3-binding agents) is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μM, about 0.9 μg/ml, about 1 μg/ml, about 2 μg/ml, about 3 μg/ml, about 4 μM, about 5 μg/ml, about 6 μg/ml, about 7 μg/ml, about 8 μg/ml, about 9 μg/ml, or about 10 μg/ml. In certain embodiments, the CD3-binding agents can be present in a concentration of 1 μg/ml.

NK cells can be activated generally using methods as described, for example, in U.S. Pat. Nos. 7,803,376, 6,949,520, 6,693,086, 8,834,900, 9,404,083, 9,464,274, 7,435,596, 8,026,097, 8,877,182; U.S. Patent Applications US2004/0058445, US2007/0160578, US2013/0011376, US2015/0118207, US2015/0037887; and PCT Patent Application WO2016/122147, each of which is incorporated herein by reference in its entirety.

In some embodiments, the NK cells are activated by, for example and not limitation, inhibition of inhibitory receptors on NK cells (e.g., KIR2DL1, KIR2DL2/3, KIR2DL4, KIR2DL5A, KIR2DL5B, KIR3DL1, KIR3DL2, KIR3DL3, LILRB1, NKG2A, NKG2C, NKG2E or LILRB5 receptor).

In certain embodiments, the NK cells are activated by, for example and not limitation, feeder cells (e.g., native K562 cells or K562 cells that are genetically modified to express 4-1BBL and cytokines such as IL15 or IL21).

In other embodiments, interferons or macrophage-derived cytokines can be used to activate NK cells. For example, such interferons include but are not limited to interferon alpha and interferon gamma, and such cytokines include but are not limited to IL-15, IL-2, IL-21.

In certain embodiments, the NK activating agent can be present in a concentration of about 0.1 to about 10 μg/ml. In certain embodiments, the NK activating agent can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 μg/ml. In certain embodiments, the NK activating agent is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μM, about 0.9 μg/ml, about 1 μg/ml, about 2 μg/ml, about 3 μg/ml, about 4 μM, about 5 μg/ml, about 6 μg/ml, about 7 μg/ml, about 8 μg/ml, about 9 μg/ml, or about 10 μg/ml. In certain embodiments, the NK activating agent can be present in a concentration of 1 μg/ml.

In certain embodiments, the activating agent is attached to a solid support such as, but not limited to, a bead, an absorbent polymer present in culture plate or well or other matrices such as, but not limited to, Sepharose or glass; may be expressed (such as in native or recombinant forms) on cell surface of natural or recombinant cell line by means known to those skilled in the art.

In certain embodiments, the host cells can be genetically modified after stimulation/activation. In certain embodiments, the host cells are modified within 12 hours, 16 hours, 24 hours, 36 hours, or 48 hours of stimulation/activation. In certain embodiments, the cells are modified within 16 to 24 hours after stimulation/activation. In certain embodiments, the host cells are modified within 24 hours. In certain embodiments, the host cells can be genetically modified before stimulation/activation.

In order to genetically modify the host cell to express the arCAR of the present disclosure, the arCAR polynucleotides or recombinant vectors must be transferred into the host cell. Polynucleotide transfer may be conducted via viral or non-viral delivery methods. Suitable methods for polynucleotide delivery for use with the current methods include any method known by those of skill in the art, by which a polynucleotide can be introduced into an organelle, cell, tissue or organism.

In various embodiments, genetic modification is conducted ex vivo. Various methods are available for transfecting cells and tissues removed from a subject via ex vivo modification. For example, retroviral gene transfer in vitro can be used to genetically modified cells removed from the subject and the cell transferred back into the subject. See e.g., Wilson et al., Science, 244:1344-1346, 1989 and Nabel et al., Science, 244(4910): 1342-1344, 1989, both of which are incorporated herein by reference in their entity. In certain embodiments, the host cells may be removed from the subject and transfected ex vivo using the polynucleotides or recombinant vectors of the disclosure. In certain embodiments, the host cells obtained from the subject can be transfected or transduced with the polynucleotides or recombinant vectors of the disclosure and then administered back to the subject.

In some embodiments, the host cells can be transduced via retroviral transduction. References describing retroviral transduction of genes are Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358; and Kuo et al., Blood 82:845 (1993), each of which is incorporated herein by reference in its entirety.

Another suitable method of gene transfer includes injection. In certain embodiments, a polynucleotide or recombinant vector may be delivered to a cell, tissue, or organism via one or more injections (e.g., a needle injection). Non-limiting methods of injection include injection of a composition (e.g., a saline based composition). Polynucleotides or recombinant vectors can also be introduced by direct microinjection. Non-limiting sites of injection include, subcutaneous, intradermal, intramuscular, intranodal (allows for direct delivery of antigen to lymphoid tissues). intravenous, intraprotatic, intratumor, intralymphatic (allows direct administration of DCs) and intraperitoneal. It is understood that proper site of injection preparation is necessary (e.g., shaving of the site of injection to observe proper needle placement).

Electroporation is another suitable method of gene transfer. See e.g., Potter et al., (1984) Proc. Nat'l Acad. Sci. USA, 81, 7161-7165 and Tur-Kaspa et al., (1986) Mol. Cell Biol., 6, 716-718, both of which are incorporated herein in their entirety for all purposes. Electroporation involves the exposure of a suspension of cells and DNA to a high-voltage electric discharge. In certain embodiments, cell wall-degrading enzymes, such as pectin-degrading enzymes, can be employed to render the host cells more susceptible to genetic modification by electroporation than untreated cells. See e.g., U.S. Pat. No. 5,384,253, incorporated herein by reference in its entirety for all purposes.

Methods of electroporation for use with this disclosure include, for example, Sardesai, N. Y., and Weiner, D. B., Current Opinion in Immunotherapy 23:421-9 (2011) and Ferraro, B. et al., Human Vaccines 7:120-127 (2011), both of which are hereby incorporated by reference herein in their entirety for all purposes.

Nucleic acid vaccines can be used to transfer arCAR polynucleotides or vectors into the host cells. Such vaccines include, but are not limited to non-viral vectors, “naked” DNA and RNA, and viral vectors. Methods of genetically modifying cells with these vaccines, and for optimizing the expression of genes included in these vaccines are known to those of skill in the art.

Additional methods of gene transfer include liposome-mediated transfection (e.g., polynucleotide entrapped in a lipid complex suspended in an excess of aqueous solution. See e.g., Ghosh and Bachhawat, (1991) In: Liver Diseases, Targeted Diagnosis and Therapy Using Specific Receptors and Ligands. pp. 87-104). Also contemplated is a polynucleotide complexed with Lipofectamine, or Superfect); DEAE-dextran (e.g., a polynucleotide is delivered into a cell using DEAE-dextran followed by polyethylene glycol. See e.g., Gopal, T. V., Mol Cell Biol. 1985 May; 5(5): 1188-90); calcium phosphate (e.g., polynucleotide is introduced to the cells using calcium phosphate precipitation. See e.g., Graham and van der Eb, (1973) Virology, 52, 456-467; Chen and Okayama, Mol. Cell Biol., 7(8):2745-2752, 1987), and Rippe et al., Mol. Cell Biol., 10:689-695, 1990); sonication loading (introduction of a polynucleotide by direct sonic loading. See e.g., Fechheimer et al., (1987) Proc. Nat'l Acad. Sci. USA, 84, 8463-8467); microprojectile bombardment (e.g., one or more particles may be coated with at least one polynucleotide and delivered into cells by a propelling force. See e.g., U.S. Pat. Nos. 5,550,318; 5,538,880; 5,610,042; and PCT Application WO 94/09699; Klein et al., (1987) Nature, 327, 70-73, Yang et al., (1990) Proc. Nat'l Acad. Sci. USA, 87, 9568-9572); and receptor-mediated transfection (e.g., selective uptake of macromolecules by receptor-mediated endocytosis that will be occurring in a target cell using cell type-specific distribution of various receptors. See e.g., Wu and Wu, (1987) J. Biol. Chem., 262, 4429-4432; Wagner et al., Proc. Natl. Acad. Sci. USA, 87(9):3410-3414, 1990; Perales et al., Proc. Natl. Acad. Sci. USA, 91:4086-4090, 1994; Myers, EPO 0273085; Wu and Wu, Adv. Drug Delivery Rev., 12:159-167, 1993; Nicolau et al., (1987) Methods Enzymol., 149, 157-176), each reference cited here is incorporated by reference in their entirety for all purposes.

In further embodiments, host cells are genetically modified using gene editing with homology-directed repair (HDR). Homology-directed repair (HDR) is a mechanism used by cells to repair double strand DNA breaks. In HDR, a donor polynucleotide with homology to the site of the double strand DNA break is used as a template to repair the cleaved DNA sequence, resulting in the transfer of genetic information from the donor polynucleotide to the DNA. As such, new nucleic acid material may be inserted or copied into a target DNA cleavage site. Double strand DNA breaks in host cells may be induced by a site-specific nuclease. The term “site-specific nuclease” as used herein refers to a nuclease capable of specifically recognizing and cleaving a nucleic acid (DNA or RNA) sequence. Suitable site-specific nucleases for use in the present disclosure include, but are not limited to, RNA-guided endonucleases (e.g., CRISPR-associated (Cas) proteins), zinc finger nucleases, TALEN nucleases, or mega-TALEN nucleases. For example, a site-specific nuclease (e.g., a Cas9+ guide RNA) capable of inducing a double strand break in a target DNA sequence can be introduced to a host cell, along with a donor polynucleotide encoding a polypeptide of an arCAR of the present disclosure.

After the host cells are activated and transduced, the cells are cultured to proliferate.

T cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of T cells can include interleukins, such as IL-2, IL-7, IL-15, or IL-21 (see for example Cornish et al. 2006, Blood. 108(2):600-8, Bazdar and Sieg, 2007, Journal of Virology, 2007, 81(22): 12670-12674, Battalia et al, 2013, Immunology, 139(1): 109-120). Other illustrative examples for agents that may be used for the expansion of T cells are agents that bind to CD8, CD45 or CD90, such as αCD8, αCD45 or αCD90 antibodies. Illustrative examples of T-cell population including antigen-specific T cells, T helper cells, cytotoxic T cells, memory T-cell (an illustrative example of memory T cells are CD62L|CD8| specific central memory T cells) or regulatory T cells (an illustrative example of Treg are CD4+CD25+CD45RA+ Treg cells).

Additional agents that can be used to expand T cells includes methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In certain embodiments, the agent(s) used for expansion (e.g., IL-2) are administered at about 20 units/ml to about 200 units/ml. In certain embodiments, the agent(s) used for expansion (e.g., IL-2) are administered at about 25 units/ml to about 190 units/ml, about 30 units/ml to about 180 units/ml, about 35 units/ml to about 170 units/ml, about 40 units/ml to about 160 units/ml, about 45 units/ml to about 150 units/ml, about 50 units/ml to about 140 units/ml, about 55 units/ml to about 130 units/ml, about 60 units/ml to about 120 units/ml, about 65 units/ml to about 110 units/ml, about 70 units/ml to about 100 units/ml, about 75 units/ml to about 95 units/ml, or about 80 units/ml to about 90 units/ml. In certain embodiments, the agent(s) used for expansion (e.g., IL-2) are administered at about 20 units/ml, about 25 units/ml, about 30 units/ml, 35 units/ml, 40 units/ml, 45 units/ml, about 50 units/ml, about 55 units/ml, about 60 units/ml, about 65 units/ml, about 70 units/ml, about 75 units/ml, about 80 units/ml, about 85 units/ml, about 90 units/ml, about 95 units/ml, about 100 units/ml, about 105 units/ml, about 110 units/ml, about 115 units/ml, about 120 units/ml, about 125 units/ml, about 130 units/ml, about 135 units/ml, about 140 units/ml, about 145 units/ml, about 150 units/ml, about 155 units/ml, about 160 units/ml, about 165 units/ml, about 170 units/ml, about 175 units/ml, about 180 units/ml, about 185 units/ml, about 190 units/ml, about 195 units/ml, or about 200 units/ml. In certain embodiments, the agent(s) used for expansion (e.g., IL-2) are administered at about 5 mg/ml to about 10 ng/ml. In certain embodiments, the agent(s) used for expansion (e.g., IL-2) are administered at about 5.5 ng/ml to about 9.5 ng/ml, about 6 ng/ml to about 9 ng/ml, about 6.5 ng/ml to about 8.5 ng/ml, or about 7 ng/ml to about 8 ng/ml. In certain embodiments, the agent(s) used for expansion (e.g., IL-2) are administered at about 5 ng/ml, 6 ng/ml, 7 ng/ml, 8 ng/ml, 9, ng/ml, or 10 ng/ml.

NK cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of NK cells can include agents that bind to CD16 or CD56, such as for example αCD16 or αCD56 antibodies. In certain embodiments, the binding agent includes antibodies (see for example Hoshino et al, Blood. 1991 Dec. 15; 78(12):3232-40.). Other agents that may be used for expansion of NK cells may be IL-15 (see for example Vitale et al. 2002. The Anatomical Record. 266:87-92, which is hereby incorporated by reference in its entirety for all purposes).

Conditions appropriate for T-cell culture include an appropriate media (e.g., Minimal Essential Media (MEM), RPMI Media 1640, Lonza RPMI 1640, Advanced RPMI, Clicks, AIM-V, DMEM, a-MEM, F-12, TexMACS, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion).

Examples of other additives for host cell expansion include, but are not limited to, surfactant, piasmanate, pH buffers such as HEPES, and reducing agents such as N-acetyl-cysteine and 2-mercaptoethanol, Antibiotics (e.g., penicillin and streptomycin), are included only in experimental cultures, not in cultures of cells that are to be infused into a subject. The target cells are maintained under conditions necessary to support growth, for example, an appropriate temperature (e.g., 37)° ° C. and atmosphere (e.g., air plus 5% CO₂).

In certain embodiments where PSCs (e.g., iPSCs or ESCs) are used as the starting cell population, the method may further comprise differentiating the PSCs into an immune cell such as a T cell. Applicable differentiation methods and compositions for obtaining iPSC derived hematopoietic cell lineages include those described in the art, for example, in International Patent Publication No. WO2017078807 and WO2019112899, the disclosure of which is incorporated herein by reference. The iPSC derived hematopoietic lineage cells may include, but not limited to, definitive hemogenic endothelium, hematopoietic multipotent progenitor cells, hematopoietic stem and progenitor cells, T cell progenitors, NK cell progenitors, T cells, NK cells, NKT cells, B cells, macrophages, and neutrophils.

In some embodiments, the present disclosure also provides methods to purify a polypeptide of an arCAR of the present disclosure. In some embodiments, the method involves purifying the second polypeptide of an arCAR from a host cell modified to express the second polypeptide of the arCAR. In some embodiments, the second polypeptide of an arCAR is a soluble protein.

### Pharmaceutical Compositions

In one aspect, the compositions comprise one or more polypeptides of the arCARs described herein, polynucleotides, vectors comprising same, and cell compositions, as disclosed herein. In some embodiments, compositions of the present disclosure include, but are not limited to pharmaceutical compositions.

In one aspect, the present disclosure provides a pharmaceutical composition comprising a polypeptide of an arCAR described herein, and a pharmaceutically accepted carrier and/or excipient. In some embodiments, the pharmaceutical composition comprises the second polypeptide of an arCAR described herein, and a pharmaceutically accepted carrier and/or excipient. In some embodiments, the pharmaceutical composition comprises the second polypeptides of two or more arCARs described herein, and a pharmaceutically accepted carrier and/or excipient.

In another aspect, the present disclosure provides a pharmaceutical composition comprising a polynucleotide or a recombinant vector described herein, and a pharmaceutically accepted carrier and/or excipient. In some embodiments, the pharmaceutical composition comprises a polynucleotide encoding the second polypeptide of an arCAR described herein, and a pharmaceutically accepted carrier and/or excipient. In some embodiments, the pharmaceutical composition comprises a polynucleotide encoding the second polypeptides of two or more arCARs described herein, and a pharmaceutically accepted carrier and/or excipient. In some embodiments, the pharmaceutical composition comprises two or more polynucleotides each encoding the second polypeptides of an arCARs described herein, and a pharmaceutically accepted carrier and/or excipient. In some embodiments, the pharmaceutical composition comprises a recombinant vector comprising a polynucleotide encoding the second polypeptide of an arCAR described herein, and a pharmaceutically accepted carrier and/or excipient. In some embodiments, the pharmaceutical composition comprises a recombinant vector comprising a polynucleotide encoding the second polypeptides of two or more arCARs described herein, and a pharmaceutically accepted carrier and/or excipient. In various embodiments, the two or more arCARs each comprise a unique pair of tag and tag-binding domain.

In another aspect, the present disclosure provides pharmaceutical composition comprising the modified host cells described herein and a pharmaceutically acceptable carrier and/or excipient.

Examples of pharmaceutical carriers include but are not limited to sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

Compositions comprising modified host cells disclosed herein may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions comprising modified host cells disclosed herein may comprise one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

In some embodiments, the compositions are formulated for parenteral administration, e.g., intravascular (intravenous or intraarterial), intraperitoneal, intratumoral, intraventricular, intrapleural or intramuscular administration. The parenteral preparation can be enclosed in ampoules, disposable syringes or multiple dose vials made of glass or plastic. An injectable pharmaceutical composition is preferably sterile. In some embodiments, the composition is reconstituted from a lyophilized preparation prior to administration.

In some embodiments, the modified host cells may be mixed with substances that adhere or penetrate then prior to their administration, e.g., but not limited to, nanoparticles.

### Treatment Methods

In some aspects, provided herein are methods of using the arCARs of the present disclosure for treatment of a disease.

In one aspect, provided herein is a method of treating a disease in a subject in need thereof, comprising administering to the subject a therapeutically effective amount of
(i) an immune effector cell comprising a chimeric antigen receptor comprising the first polypeptide of an arCAR described herein, and
(ii) the second polypeptide of said arCAR, or a polynucleotide encoding said second polypeptide, or a host cell comprising said second polypeptide.

In some embodiments, the method further comprises administering one or more additional polypeptides each comprising (a) an antigen-binding domain that binds to a unique antigen and (b) a tag that is recognized by the tag-binding domain of the first polypeptide.

In another aspect, provided herein is a method of treating a disease in a subject in need thereof, comprising administering to the subject a therapeutically effective amount of
(i) an immune effector cell comprising chimeric antigen receptors comprising the first polypeptides of two or more arCARs, wherein the two or more arCARs are selected from the arCAR system described herein, and
(ii) the second polypeptides of said two or more arCARs, or one or more polynucleotide encoding said second polypeptides, or one or more host cells comprising said second polypeptides.

In various embodiments of the treatment methods described herein, the cell is an immune effector cell. In some embodiments, the cell is a T cell, a Natural Killer (NK) cell, a cytotoxic T lymphocyte (CTL), a regulatory T cell or a tumor-infiltrating lymphocyte (TIL). In some embodiments, the cell is a T cell. In some embodiments, the cell is derived from an iPSC. In various embodiments, the cell constitutively expresses the first polypeptide.

In various embodiments of the treatment methods described herein, the host cell and/or the polypeptide(s) are administered as a pharmaceutical composition which also comprises a pharmaceutically accepted carrier and/or excipient as described herein.

In various embodiments of the treatment methods described herein, the disease is a cancer. The terms “cancer”, “malignancy”, “neoplasm”, “tumor”, and “carcinoma”, are used interchangeably herein to refer to cells that exhibit relatively abnormal, uncontrolled, and/or autonomous growth, so that they exhibit an aberrant growth phenotype characterized by a significant loss of control of cell proliferation. In general, cells of interest for treatment in the present application include precancerous (e.g., benign), malignant, pre-metastatic, metastatic, and non-metastatic cells. The teachings of the present disclosure may be relevant to any and all cancers. To give but a few, non-limiting examples, in some embodiments, teachings of the present disclosure are applied to one or more cancers such as, for example, hematopoietic cancers including leukemias, lymphomas (Hodgkin's and non-Hodgkin's), myelomas and myeloproliferative disorders; sarcomas, melanomas, adenomas, carcinomas of solid tissue, squamous cell carcinomas of the mouth, throat, larynx, and lung, liver cancer, genitourinary cancers such as prostate, cervical, bladder, uterine, and endometrial cancer and renal cell carcinomas, bone cancer, pancreatic cancer, skin cancer, cutaneous or intraocular melanoma, cancer of the endocrine system, cancer of the thyroid gland, cancer of the parathyroid gland, head and neck cancers, breast cancer, gastro-intestinal cancers and nervous system cancers, benign lesions such as papillomas, and the like. In some embodiments, the cancer treated by methods of the present disclosure is a solid tumor. In some embodiments, the cancer treated by methods of the present disclosure is a hematologic malignancy.

In some embodiments, the cancer treated by methods of the present disclosure is a glioblastoma, ovarian cancer, cervical cancer, head and neck cancer, liver cancer, prostate cancer, pancreatic cancer, renal cell carcinoma, bladder cancer, or hematologic malignancy. In some embodiment, the hematologic malignancy is a leukemia (e.g., acute lymphocytic (ALL), chronic lymphocytic (CLL), acute myeloid (AML), chronic myeloid (CML)), myeloma, or lymphoma (e.g., Hodgkin's or non-Hodgkin's (NHL)).

In various embodiments of the treatment methods described herein, the disease is autoimmune disease or disorder. In some embodiments, the autoimmune disease or disorder is Rheumatoid arthritis (RA), multiple sclerosis (MS), Sjögren's syndrome, Systemic lupus erythematosus, sarcoidosis, Type 1 diabetes mellitus, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Myasthenia gravis, Hashimoto's thyroiditis, Graves' disease, chronic inflammatory demyelinating polyneuropathy, Guillain-Barre syndrome, Crohn's disease or ulcerative colitis.

When more than one polypeptides with unique antigen-binding specificities are administered, the methods can be used to target multiple antigens (or multiple epitopes in the same antigen) in the same disease (e.g., tumor or autoimmune disease), or multiple antigens in different diseases (e.g., tumor or autoimmune disease).

In some embodiments, the modified immune effector cell and the second polypeptide(s), or polynucleotide(s) encoding said second polypeptide(s), or host cell(s) comprising said second polypeptide(s), are administered simultaneously.

In some embodiments, the modified immune effector cell and the second polypeptide(s), or polynucleotide(s) encoding said second polypeptide(s), or host cell(s) comprising said second polypeptide(s), are administered sequentially. For example, the second polypeptide(s), or polynucleotide(s) encoding said second polypeptide(s), or host cell(s) comprising said second polypeptide(s), may be administered before or after the administration of the modified host cell.

In some embodiments, the immune effector cell is an autologous cell with respect to the subject receiving the treatment. In some embodiments, the immune effector cell is an allogeneic cell with respect to the subject receiving the treatment. When the immune effector cell is isolated from a donor, the method may further include a method to prevent graft vs host disease (GVHD) and the host cell rejection.

In some embodiments, additional steps can be performed prior to administration to a subject. For instance, an immune effector cell can be expanded in vitro after contacting (e.g., transducing or transfecting) the immune effector cell with a polynucleotide or recombinant vector described herein (e.g., a polynucleotide or recombinant vector encoding a first polypeptide of an arCAR), but prior to the administration to a subject. In vitro expansion can be carried out for 1 day or more, e.g., 2 days or more, 3 days or more, 4 days or more, 6 days or more, or 8 days or more, prior to the administration to a subject. Alternatively, or in addition, in vitro expansion can proceed for 21 days or less, e.g., 18 days or less, 16 days or less, 14 days or less, 10 days or less, 7 days or less, or 5 days or less, prior to administration to a subject. For example, in vitro expansion can be carried out for 1-7 days, 2-10 days, 3-5 days, or 8-14 days prior to the administration to a subject.

In some embodiments, during in vitro expansion, an immune effector host cell can be stimulated with an antigen (e.g., a TCR antigen). Antigen specific expansion optionally can be supplemented with expansion under conditions that non-specifically stimulate lymphocyte proliferation such as, for example, anti-CD3 antibody, anti-Tac antibody, anti-CD28 antibody, or phytohemagglutinin (PHA). The expanded host cells can be directly administered into a subject or can be frozen for future use, i.e., for subsequent administrations to a subject.

In some embodiments, an immune effector host cell is treated ex vivo with interleukin-2 (IL-2) prior to infusion into a subject, and/or the subject is treated with IL-2 after infusion. Furthermore, in some embodiments, a patient can undergo preparative lymphodepletion—the temporary ablation of the immune system—prior to administration of a modified host cell. A combination of IL-2 treatment and preparative lymphodepletion can enhance persistence of modified host cell.

In some embodiments, an immune effector host cell is transduced or transfected with a nucleic acid encoding a cytokine, which nucleic acid can be engineered to provide for constitutive, regulatable, or temporally-controlled expression of the cytokine. Suitable cytokines include, for example, cytokines which act to enhance the survival of T lymphocytes during the contraction phase, which can facilitate the formation and survival of memory T lymphocytes.

In some embodiments, a composition of the present disclosure (e.g., host cell or polypeptide) is administered in a therapeutically effective amount. The dosages of the composition administered in the methods of the disclosure will vary widely, depending upon the subject's physical parameters, the frequency of administration, the manner of administration, the clearance rate, and the like. The initial dose may be larger, and might be followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve in vivo persistence of modified host cells. It is also contemplated that a variety of doses will be effective to improve in vivo effector function of modified host cells.

In some embodiments, a composition comprising the modified host cells prepared by the methods described herein may be administered at a dosage of 10² to 10¹⁰ cells/kg body weight, 10⁵ to 10⁹ cells/kg body weight, 10⁵ to 10⁸ cells/kg body weight, 10⁵ to 10⁷ cells/kg body weight, 10⁷ to 10⁹ cells/kg body weight, or 10⁷ to 10⁸, cells/kg body weight, including all integer values within those ranges. The number of modified host cells will depend on the therapeutic use for which the composition is intended for.

Modified host cells may be administered multiple times at dosages listed above. The modified host cells may be allogeneic, syngeneic, xenogeneic, or autologous to the patient undergoing therapy.

It is also contemplated that when used to treat various diseases/disorders, the compositions and methods of the present disclosure can be utilized with other therapeutic methods/agents suitable for the same or similar diseases/disorders. Such other therapeutic methods/agents can be co-administered (simultaneously or sequentially) to generate additive or synergistic effects. Suitable therapeutically effective dosages for each agent may be lowered due to the additive action or synergy.

In certain embodiments, a composition of the present disclosure (e.g., host cell or polypeptide) is administered prior to, substantially simultaneously with, or after the administration of an additional therapeutic agent such as a cancer therapeutic agent. The additional cancer therapeutic agent can be, e.g., a chemotherapeutic agent, a biological agent, surgery, gene therapy or radiation treatment. In some embodiments, a subject receiving a composition is not administered a treatment which is sufficient to cause a depletion of immune cells, such as lymphodepleting chemotherapy or radiation therapy.

In some embodiments of any of the above therapeutic methods, the method further comprises administering to the subject one or more additional compounds selected from the group consisting of immuno-suppressives, biologicals, probiotics, prebiotics, and cytokines (e.g., IFN or IL-2).

In some embodiments, the methods and compositions of the disclosure can be combined with other therapies that block inflammation (e.g., via blockage of IL1, INFα/β, IL6, TNF, IL23, etc.).

The methods and compositions of the disclosure can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 4-1BB, OX40, etc.). The methods of the disclosure can be also combined with other treatments that possess the ability to modulate NKT function or stability, including but not limited to CD1d, CD1d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e). The methods of the disclosure can also be combined with other treatments such as midostaurin, enasidenib, or a combination thereof.

Therapeutic methods of the disclosure can also be combined with additional immunotherapies and therapies. For example, when used for treating tumors, the compositions of the disclosure can be used in combination with conventional therapies, such as, e.g., surgery, radiotherapy, chemotherapy or combinations thereof, depending on type of the tumor, patient condition, other health issues, and a variety of factors. In certain aspects, other therapeutic agents useful for combination tumor therapy with the inhibitors of the disclosure include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In one embodiment, the modified host cells of the disclosure can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

Non-limiting examples of chemotherapeutic compounds which can be used in combination treatments of the present disclosure include, for example, aminoglutethimide, amsacrine, anastrozole, asparaginase, azacitidine, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, decitabine, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-tumor agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In various embodiments of the methods described herein, the subject is a mammal. In some embodiments, the subject is a human. The subject may be a juvenile or an adult, of any age or sex.

### EXAMPLES

The following examples are provided to further describe some of the embodiments disclosed herein. The examples are intended to illustrate the disclosed embodiments. It should be understood, however, that the application is not limited to the exemplary embodiments illustrated below.

### Example 1. Developing arCARs with Novel VHH Tags

Novel VHHs having specificity to a non-human protein are identified using a phage display technique or by immunization. Candidate VHHs are selected for production and quality control analysis. Candidate VHHs are further tested for specificity and cross-reactivity. Lead VHHs are used for producing anti-VHHs.

Anti-VHHs having specificity to the lead tag VHHs are identified using a phage display technique or generated by immunization in llamas or other camelids. Several commercially available phage campaigns may be employed including Tungsten, VHH and SuperHuman, and scFv. Candidate anti-VHHs are selected that bind to CDRs or other non-framework region of the tag VHHs.

Soluble tagged VHHs are expressed and produced in CHO cells. Soluble tagged VHHs are purified via Protein A chromatography. The anti-VHHs are incorporated as an extracellular tag-binding domain in a CAR construct. T cells are transduced with the lentiviral construct to express the CAR containing the anti-VHH domain. The anti-VHH is also tested for specificity and cross-reactivity.

In vitro assays are established to evaluate T cell activation through soluble, tagged VHH and anti-tag CAR by assessing cytokine production and cytotoxicity, respectively. Lead arCAR constructs are selected for further analysis.

### Example 2. Developing arCARs with Pre-Existing VHH Tags

Pre-existing VHHs that bind non-human proteins are selected. Non-limiting examples of VHHs that can be used in constructing the arCARs of the present disclosure include VHHs that target RSV F-protein, Listeria internalin, Cobra phospholipase A2, Ebola, nucleoprotein, HSV Glycoprotein D, Lacotococcal phage RBP, Geobacillus stearothermophilus, Ricin (e.g., V5E4), and chicken egg white lysozyme. The VHHs may also be engineered to eliminate binding by scrambling CDRs. Candidate VHHs are selected for production and quality control analysis. Selected VHHs are used for producing anti-VHHs.

Anti-VHHs having specificity to the lead tag VHHs are identified using phage display technique or by immunization in Llama. Several commercially available phage campaigns may be employed including Tungsten, VHH and SuperHuman, and scFv. Candidate anti-VHHs are selected that bind to CDRs or other non-framework region of the tag VHHs.

Soluble tagged VHHs are expressed and produced in CHO cells. Soluble tagged VHHs are purified via Protein A chromatography. The anti-VHHs are incorporated as an extracellular tag-binding domain in a lentiviral CAR construct. T cells are transduced with the lentiviral construct to express the CAR containing the anti-VHH domain. The anti-VHH is also tested for specificity and cross-reactivity.

In vitro assays are established to evaluate T cell activation through soluble, tagged VHH and anti-tag CAR by assessing cytokine production and cytotoxicity, respectively. Lead arCAR constructs are selected for further analysis.

### Example 3. arCARs with BCMA/anti-BCMA VHH

To test the arCAR concept, several approaches are used as listed below.

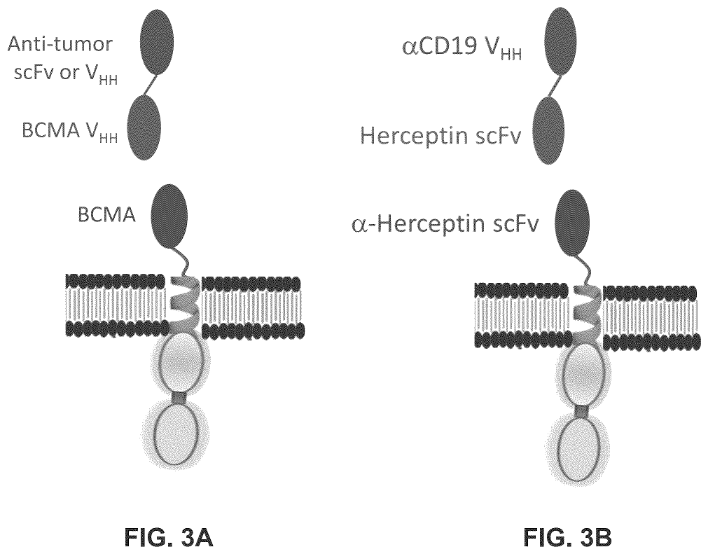

To test the initial concept of the protein: protein interaction in the arCAR platform, an anti-BCMA antibody is fused to a tumor targeting domain antibody. BCMA is fused to the transmembrane domain of the CAR. The exemplary arCAR incorporating the BCMA/anti-BCMA antibody is shown in FIG. 3A.

In another set of experiments, an scFv is developed which is derived from the Herceptin mAb sequence. The sequence identified for an anti-Herceptin idiotype mAb is converted to an scFv format. These known sequences are engineered into the arCAR platform to demonstrate the concept. Herceptin scFv is fused to the CAR and the Herceptin anti-ID scFv is fused to a soluble tumor targeting domain antibody (e.g., anti-CD19 scFv or VHH). The exemplary arCAR incorporating the Herceptin/Herceptin scFvs is shown in FIG. 3B. This arCAR is tested for cytotoxicity potential in vitro.

Several antibodies with specificity for non-human proteins have been identified in the literature. Such examples include Hodyx, hP3-3, hAnti-Z, hHSV GD, hLP RBP, hVHH4, hSbsB, and hV5E4. These are constructed in an scFv or VHH format for characterization. Candidates are selected based on biophysical properties. An anti-ID campaign is then carried out against these binders to develop a diverse affinity panel that enables proof-of-concept around advantages for modulating the arCAR affinity.

### Example 4. arCARs with Herceptin/anti-Herceptin scFvs

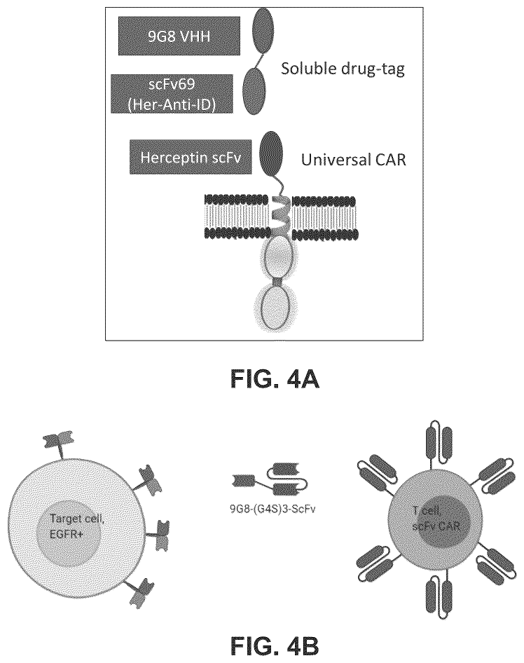

In a Proof-of-Concept study, an scFv was developed which is derived from Herceptin (Trastuzumab) mAb. Another scFv (scFv69) was developed from an anti-Herceptin idiotype mAb. These sequences were assembled into different CAR expression constructs (Table 5), each of which was then packaged into a lentiviral vector and expressed via viral transduction in either Jurkat cells or primary T-cells. CAR expression was detected by flow cytometry and binding to EGFR soluble protein. The CAR expressing cells were used in combination with CHO-cells over-expressing EGFR or cell lines that have native cell surface expression of EGFR to demonstrate EGFR-specific cytotoxicity. FIG. 4A shows an exemplary arCAR where Herceptin scFv is fused to the CAR and the Herceptin anti-ID scFv is fused to an EGFR-targeting VHH (9G8). FIG. 4B shows that a T cell expressing the Herceptin scFv CAR, coupled with the 9G8-scFv69 fusion protein, is capable of targeting an EGFR+ cell.

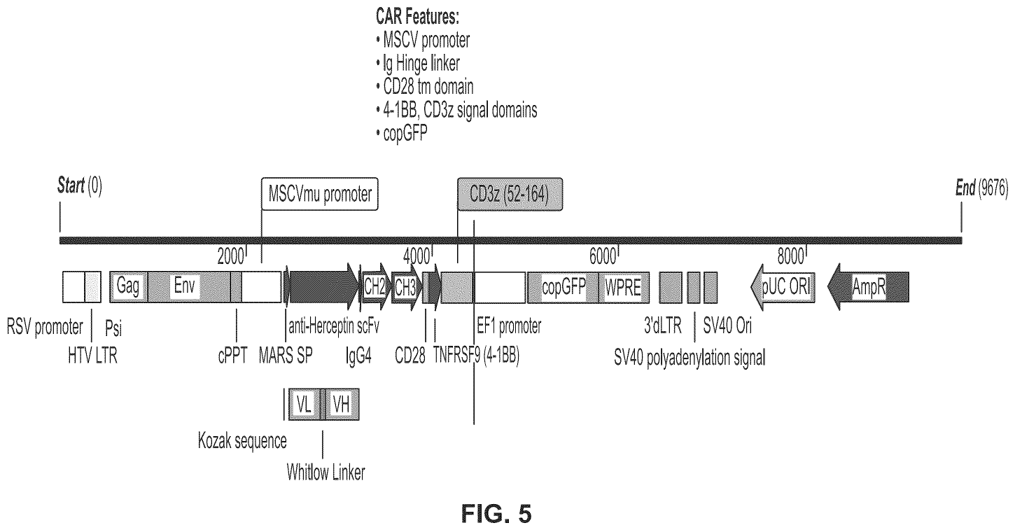

Table 5 presents the CAR expression constructs generated which comprise either the Herceptin scFv or the anti-Herceptin idiotype scFv69 as the extracellular tag-binding domain. FIG. 5 shows a vector map of the lentiviral vector used to deliver the CAR constructs. Table 6 presents the Herceptin scFv and scFv69 (Herceptin anti-ID).

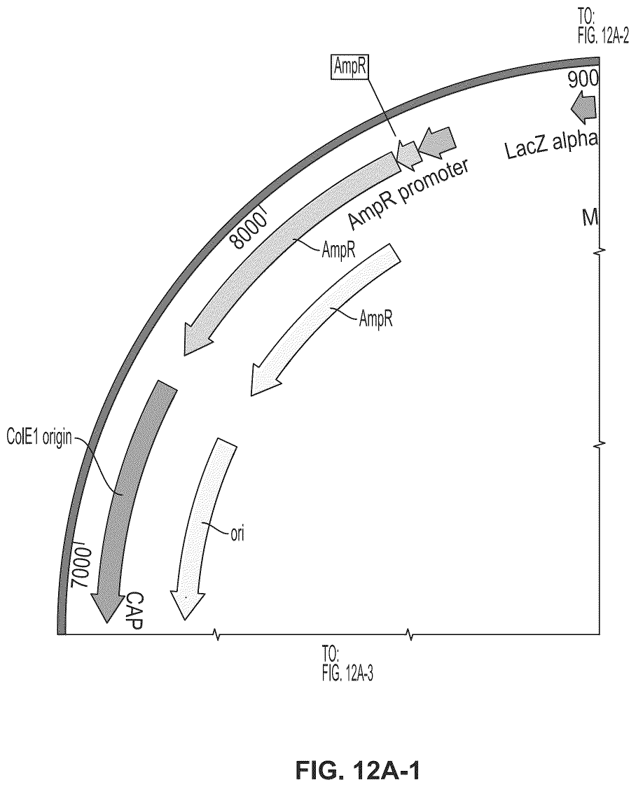

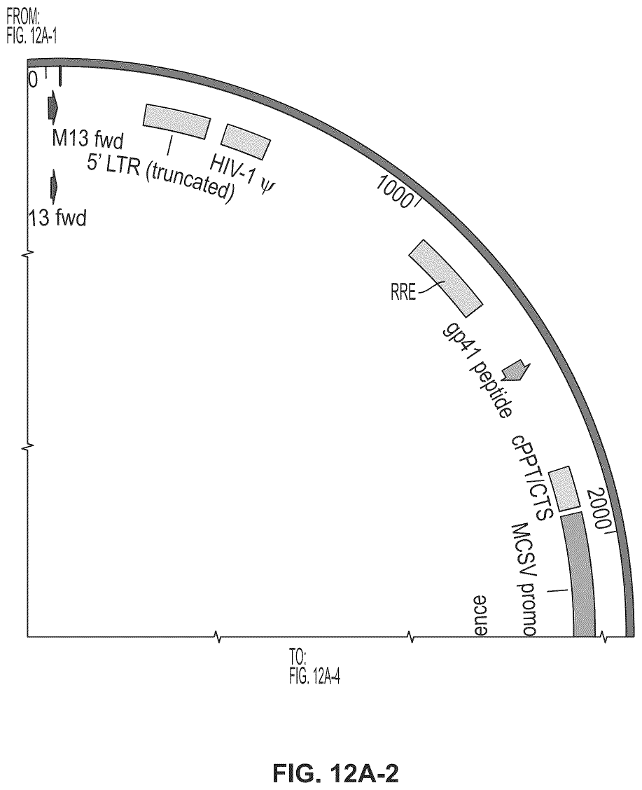

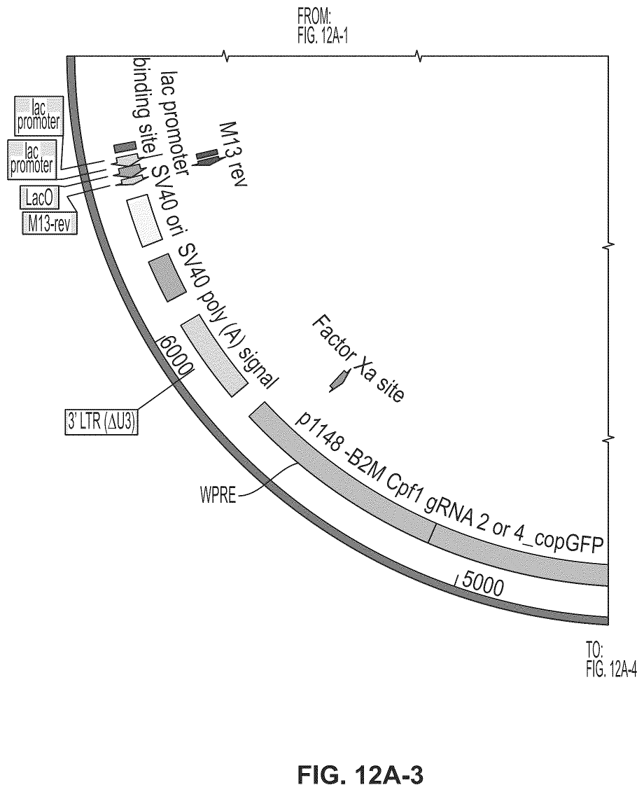

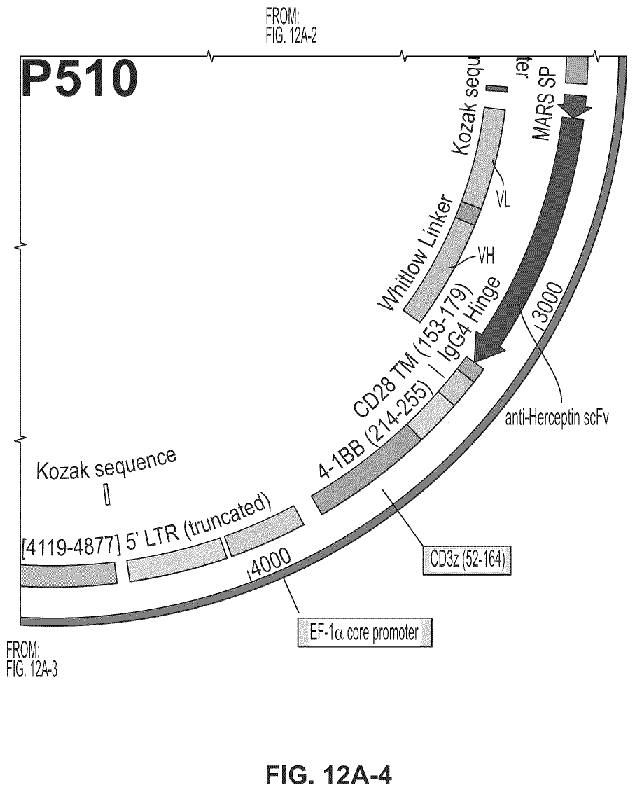

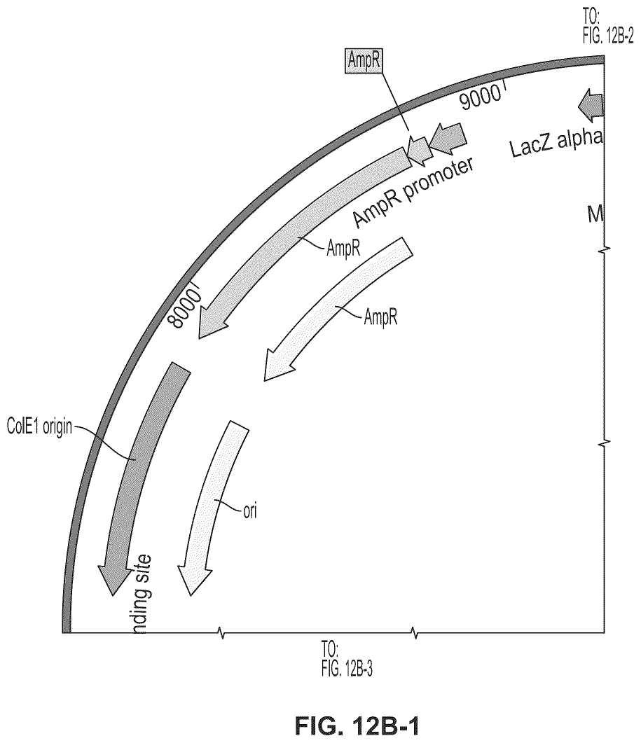

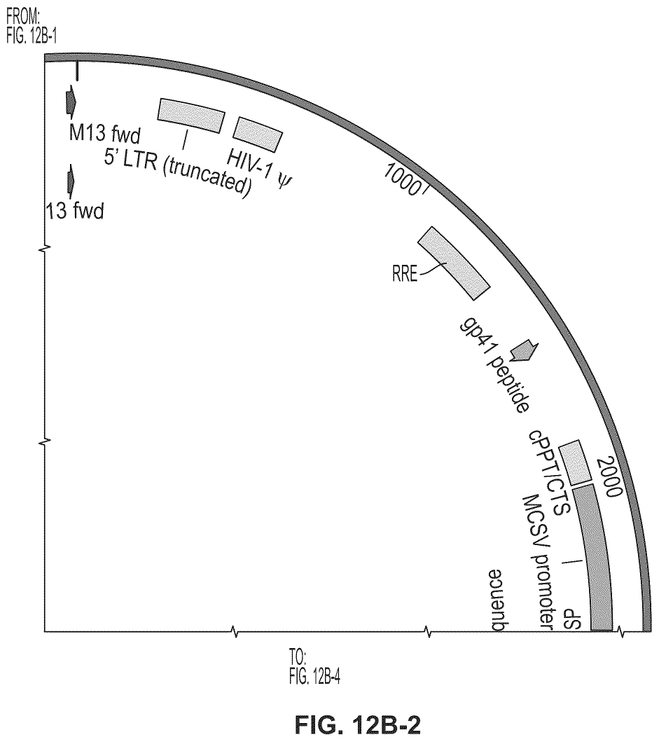

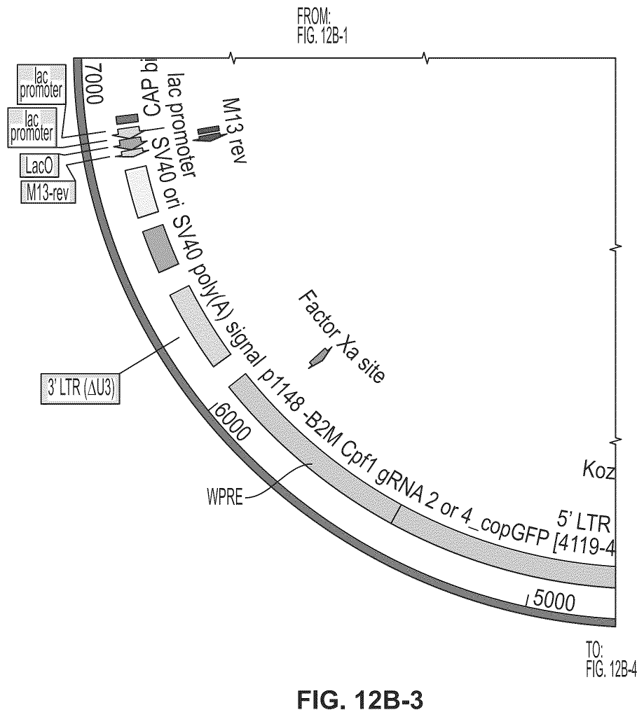

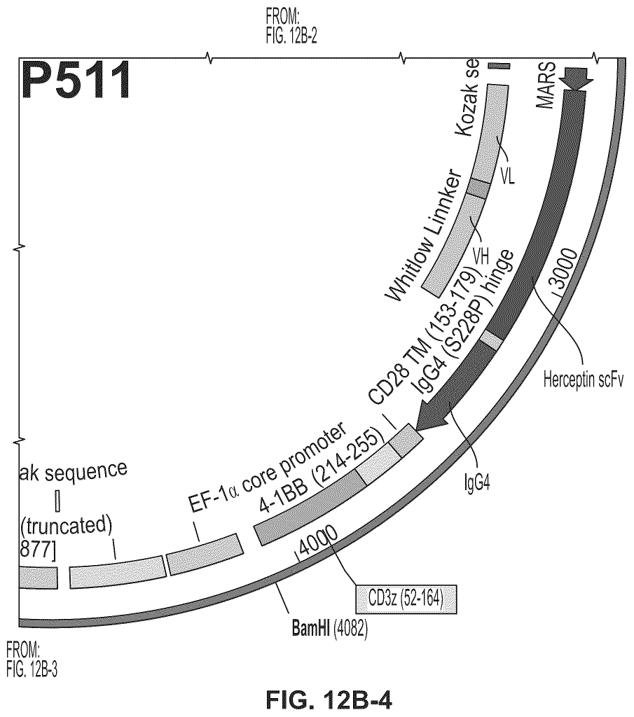

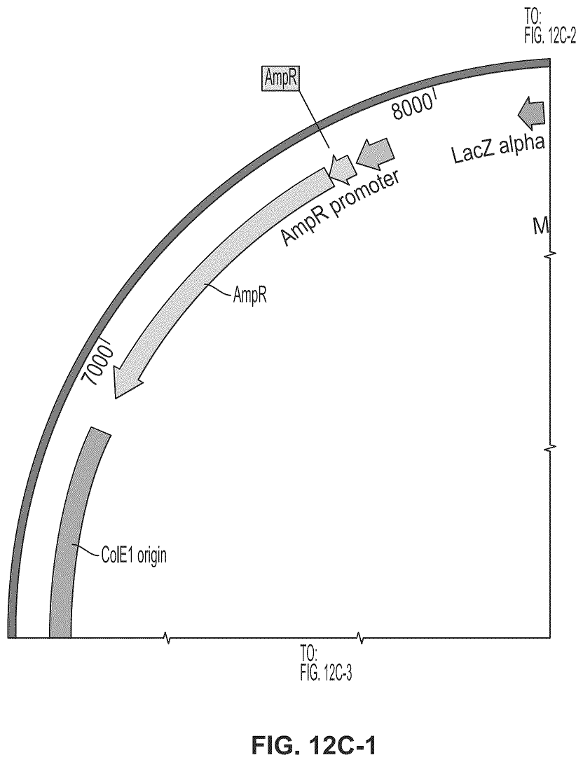

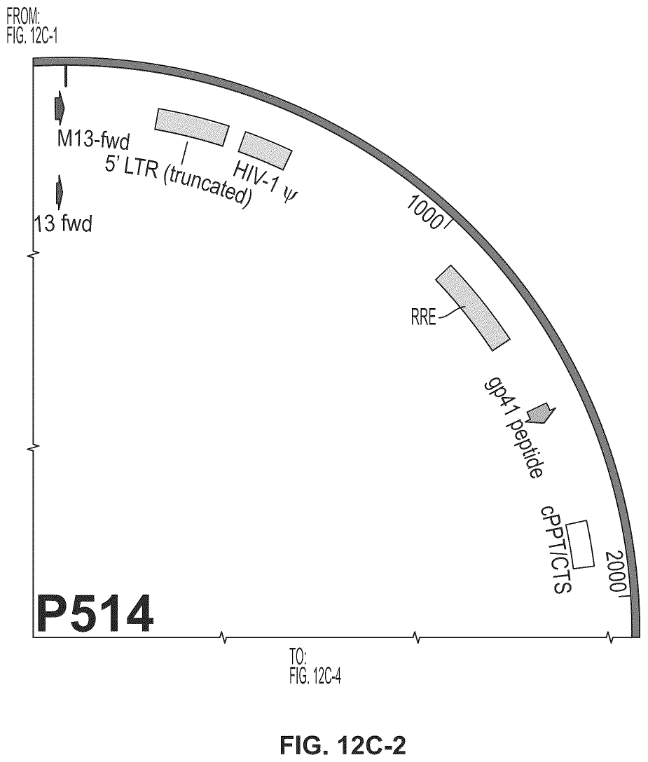

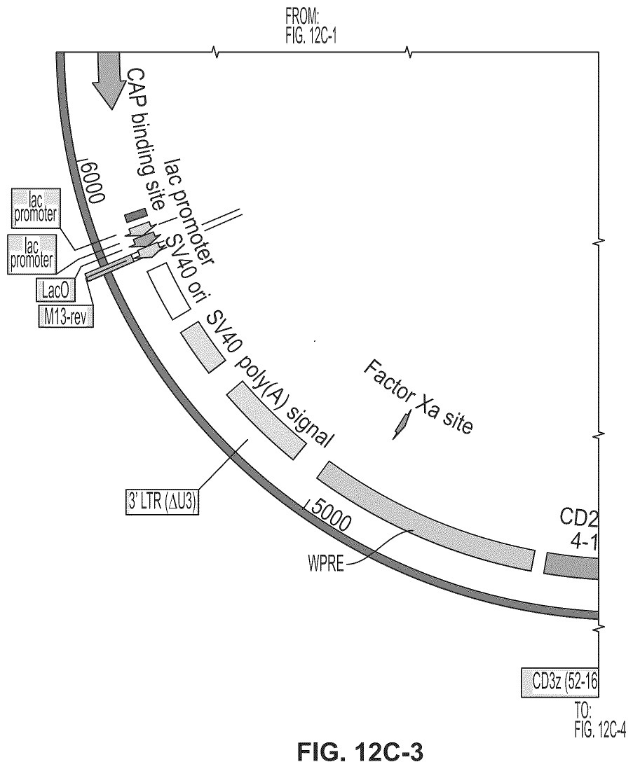

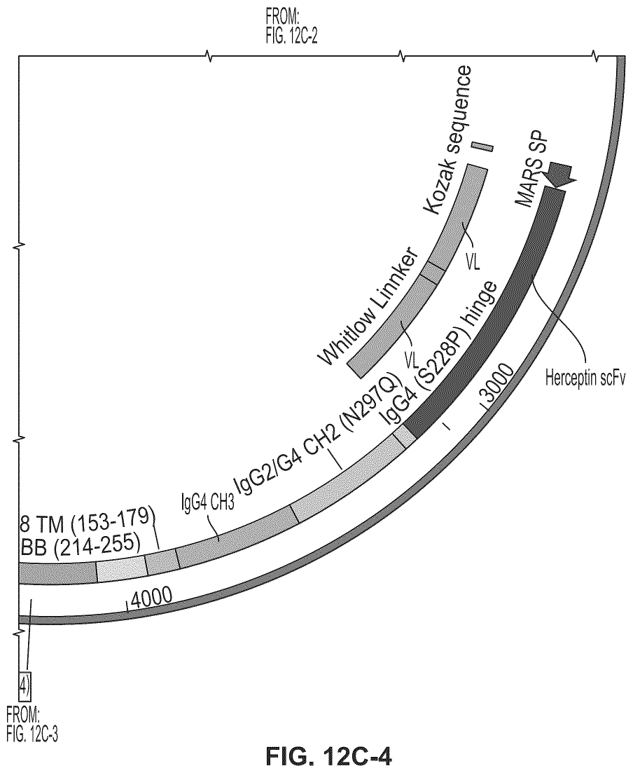

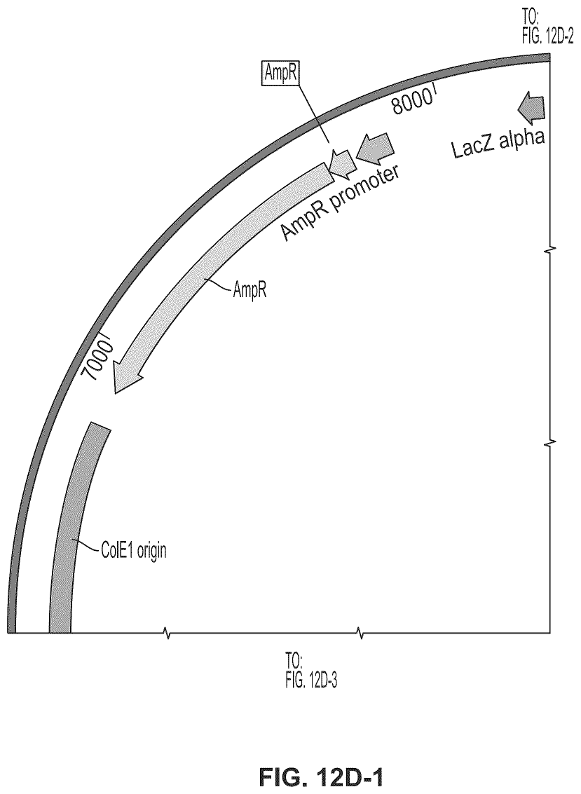

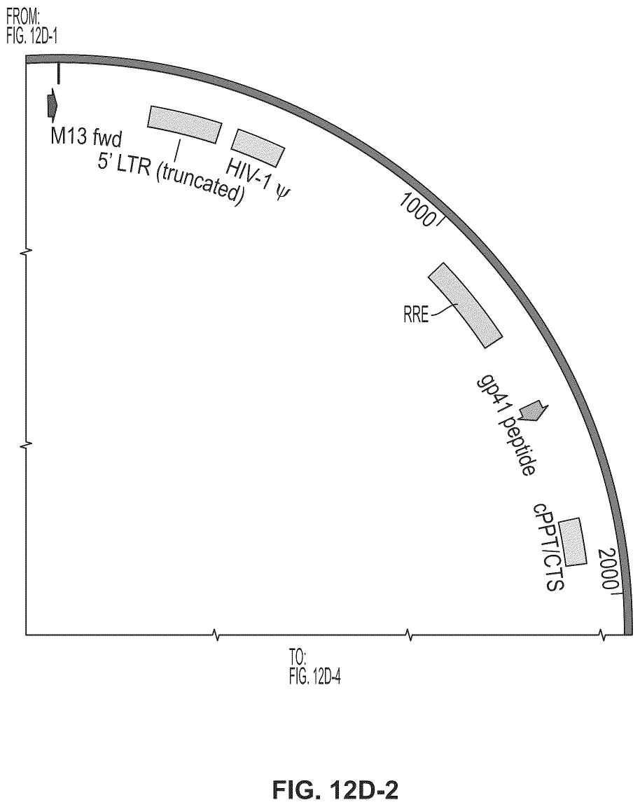

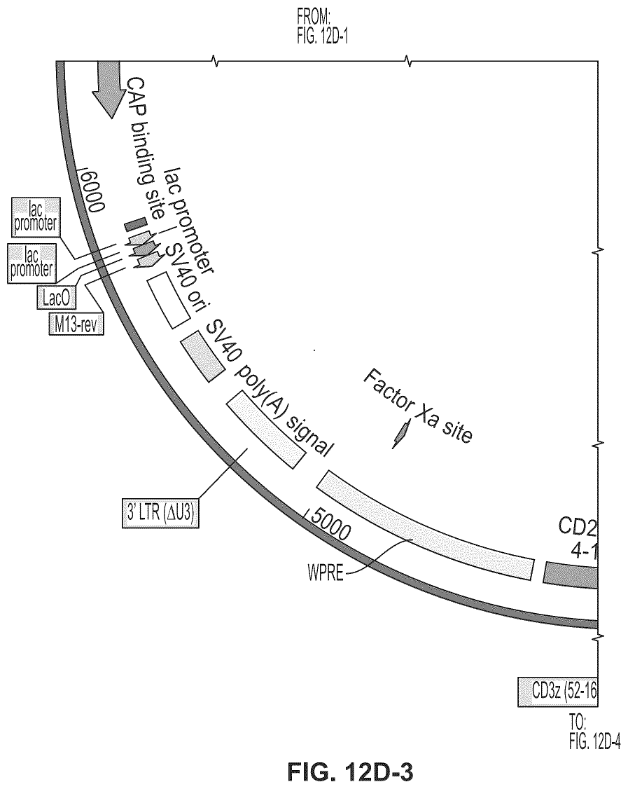

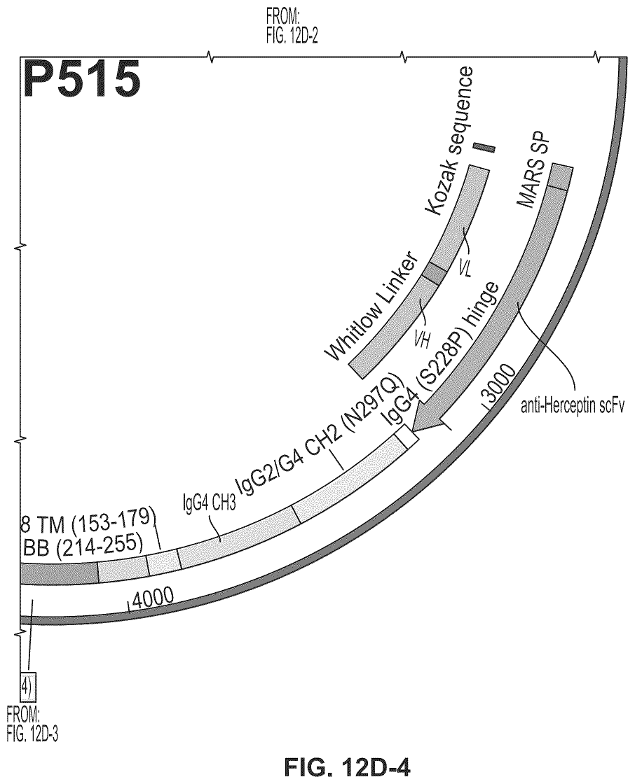

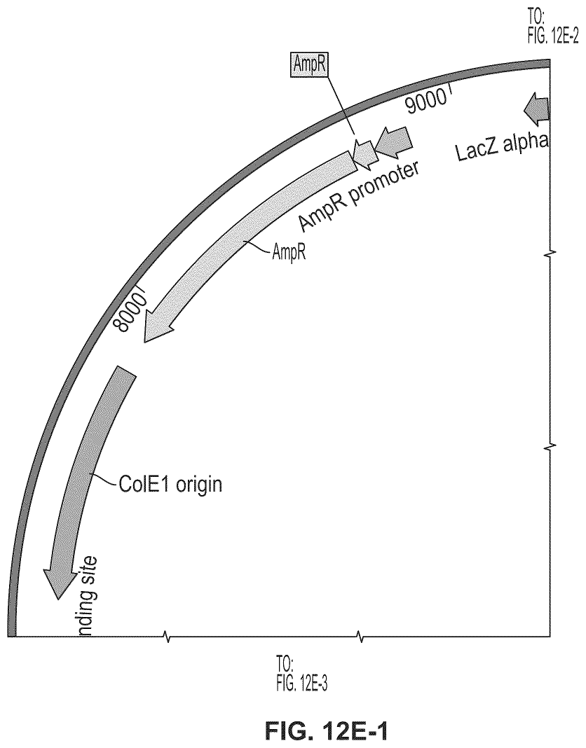

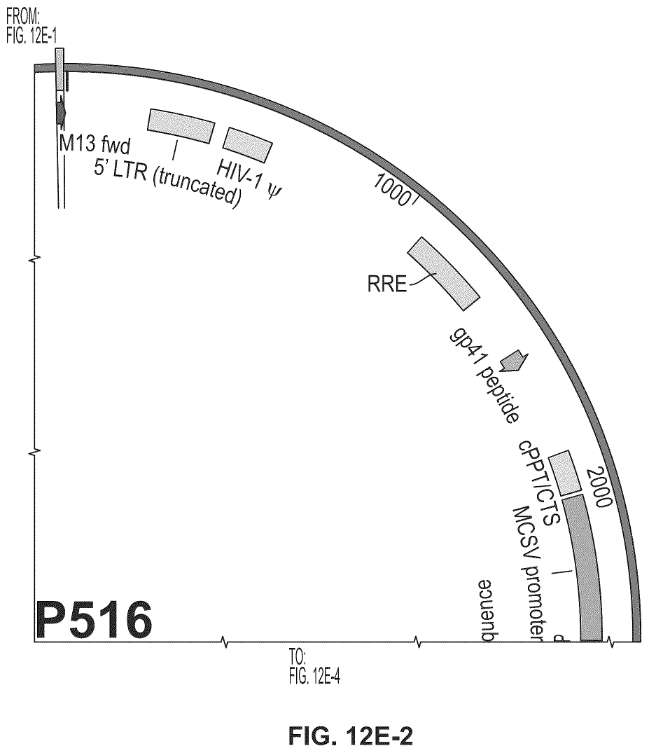

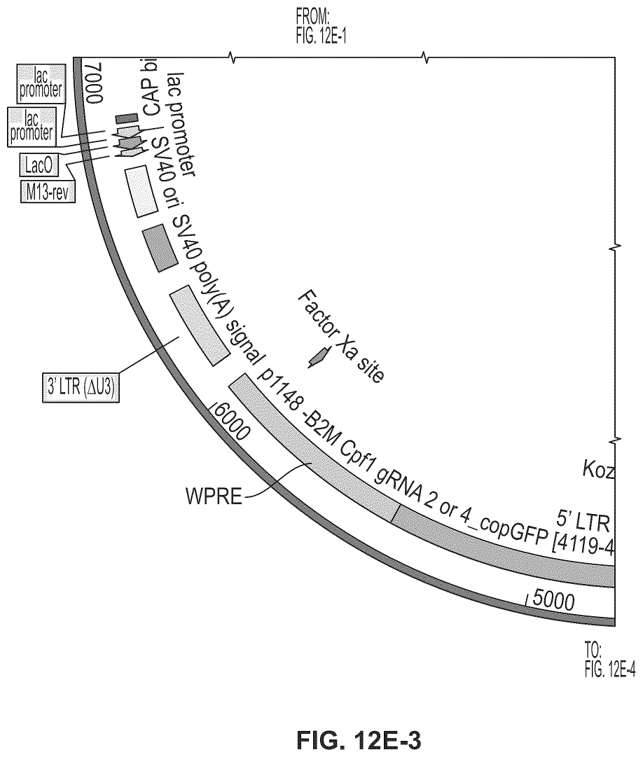

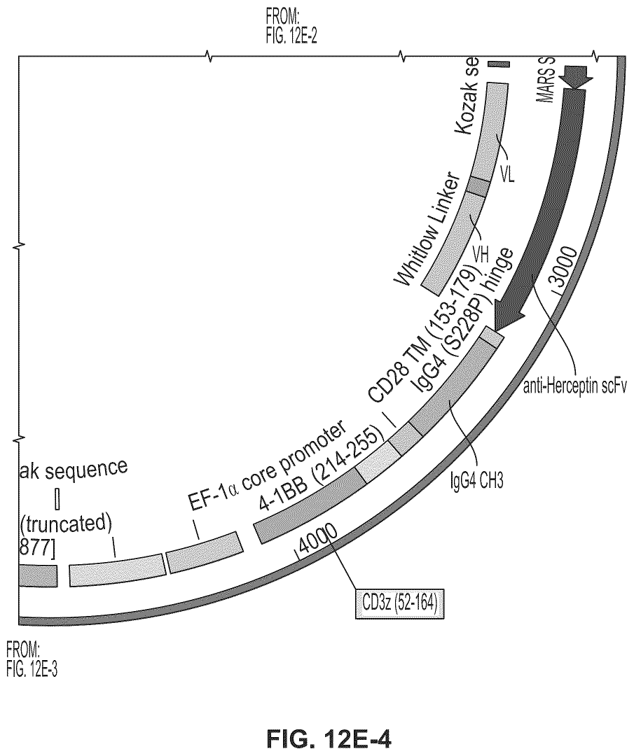

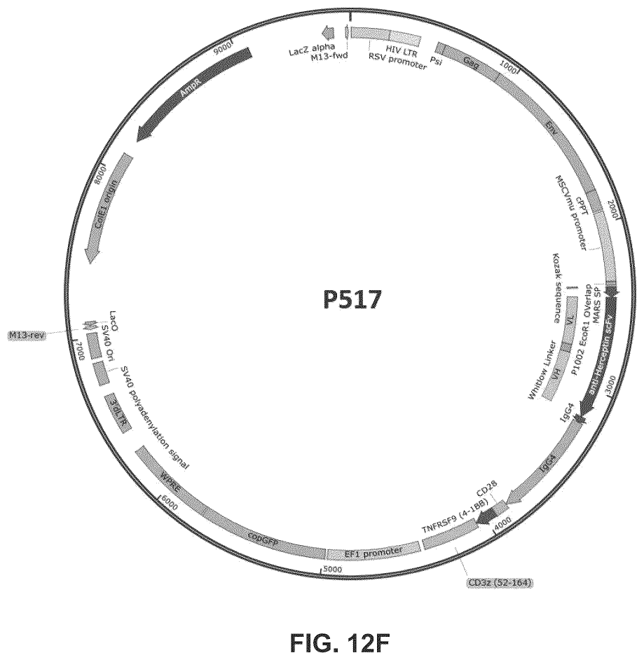

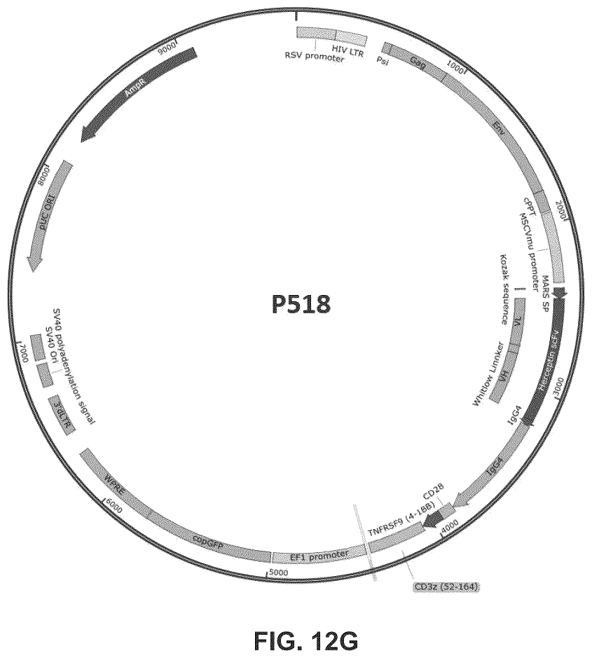

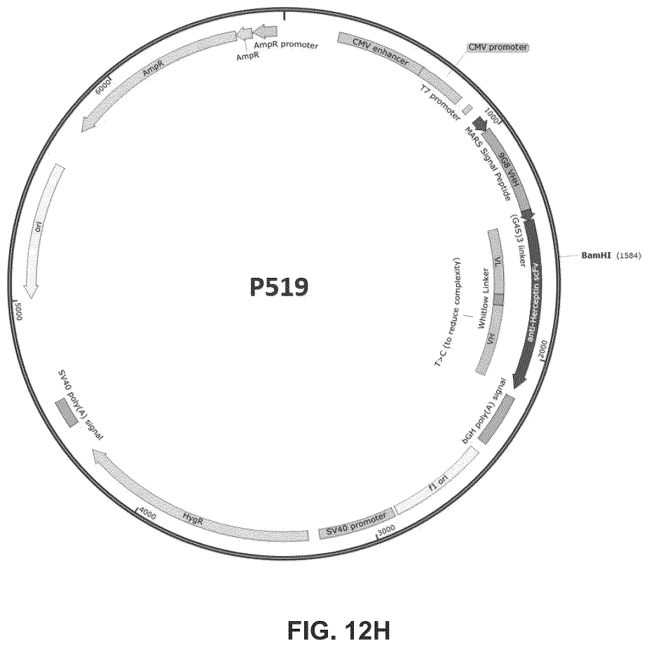

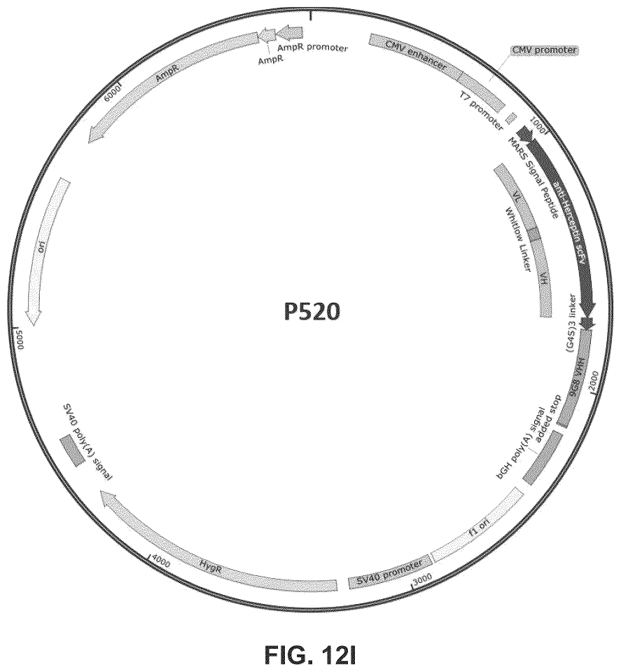

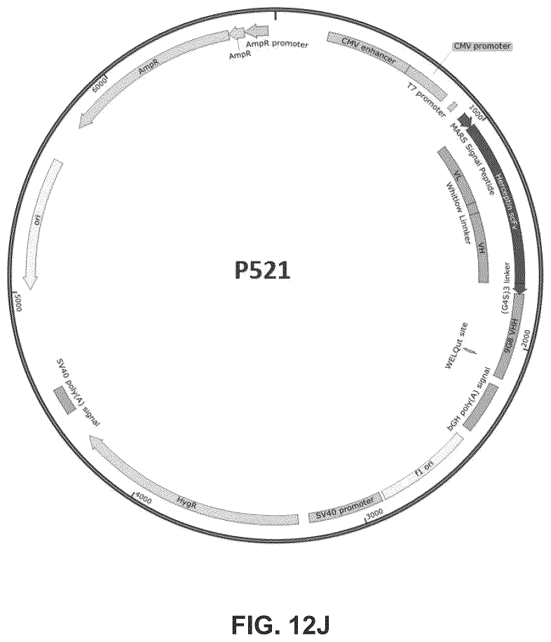

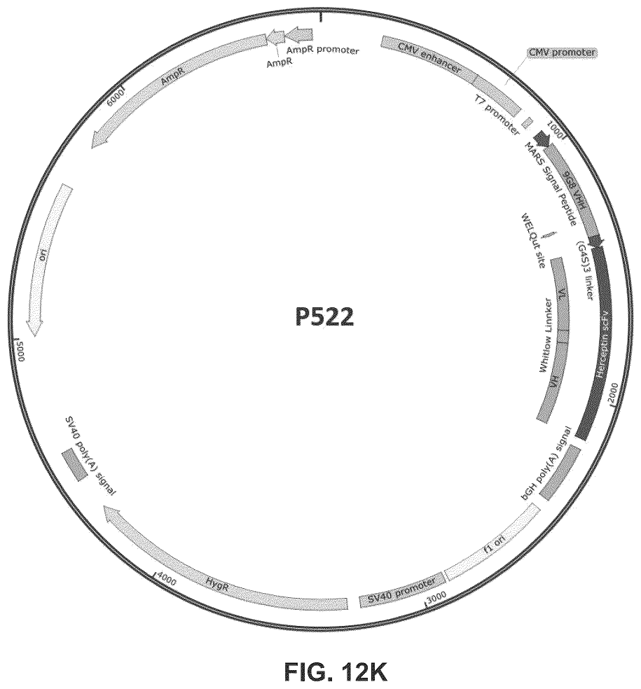

Jurkat cells were transduced with a lentiviral vector carrying a CAR construct described in Table 5 to evaluate CAR expression and CD69 activation. To measure CD69 regulation as a readout for activation, the CAR expressing Jurkat cells were co-cultured with EGFR positive cells for 24 and 48 hours. After the co-culture, the cells were stained for CD69 levels using an anti-Human CD69-BV421 antibody and measured on an Intellicyte flow cytometer. The CAR constructs were also expressed in T cells to evaluate cytotoxicity. To perform this assay, T-cells were isolated using the Pan T cell Isolation Kit (Miltenyi Cat #130-096-535) according to the manufacturer's directions. Cells were resuspended in RPMI, 10% FBS with 30 units/ml IL2 and activated overnight with Dynabeads™ Human T-Activator CD3/CD28 (ThermoFisher, Cat #11132D). The next day, the T-cells were transduced with Lentivirus plasmids containing the CAR expression cassette using TransDux™ MAX Lentivirus Transduction Reagent (SBI, Cat #LV860A-1) (FIGS. 12A-12K). The T-cells were expanded for seven days and on the seventh day, the cytotox assay was set up. Target cells labeled with Recon cell trace violet were co-cultured with the CAR positive T-cells at various ratios for 24 or 48 hours. After 48 hours, the viability of the cells were checked on the Intellicyte Flow Cytometer.

TABLE 5
CAR expression constructs

| CAR Format | Antibody | PLASMID |
|---|---|---|
| Long Hinge, CD28 tm 4-1BB, CD3z | Herceptin | p518 |
| Long Hinge, CD28 tm 4-1BB, CD3z | scFv69 | p517 |
| Med Hinge, CD28 tm 4-1BB, CD3z | Herceptin | p511 |
| Med Hinge, CD28 tm 4-1BB, CD3z | scFv69 | p516 |
| Long Hinge, CD28 tm 4-1BB, CD3z | Herceptin | p514 |
| Long Hinge, CD28 tm 4-1BB, CD3z | scFv69 | p515 |
| Short Hinge, CD28 tm 4-1BB, CD3z | scFv69 | p510 |
| | 9G8 | |

TABLE 6
Herceptin, scFv69, and 9G8 CAR associated sequences

| CAR regoins | Sequence | SEQ ID NO |
|---|---|---|
| Herceptin scFv | GACATCCAGATGACTCAGTCACCATCAAGCCTGAGTGCA | 69 |
| (nucleotide | TCCGTGGGCGATCGAGTGACAATAACATGTAGAGCGAGC | |
| sequence) | CAGGATGTAAATACGGCAGTAGCGTGGTACCAACAGAAA | |
| | CCCGGCAAGGCTCCTAAGCTGTTAATCTACAGCGCCAGC | |
| | TTCCTTTATAGTGGAGTGCCTTCAAGGTTCTCAGGATCT | |
| | AGGTCCGGTACTGACTTCACGCTGACAATCTCGAGCCTA | |
| | CAACCCGAGGACTTCGCCACTTATTACTGCCAGCAGCAT | |
| | TACACTACTCCTCCCACATTCGGACAGGGAACCAAAGTC | |
| | GAGATCAAAGGATCAACCTCTGGATCTGGCAAGCCCGGG | |
| | AGCGGGGAAGGCTCTACTAAGGGTGAGGTGCAACTAGTG | |
| | GAGAGTGGCGGAGGGCTCGTCCAGCCAGGAGGTTCCCTG | |
| | AGGCTGAGTTGCGCTGCAAGCGGATTCAATATCAAGGAC | |
| | ACGTACATACACTGGGTGCGCCAGGCCCCCGGAAAGGGA | |
| | CTGGAGTGGGTCGCCCGAATCTATCCTACTAATGGCTAC | |
| | ACCAGGTATGCTGATTCAGTGAAAGGAAGGTTTACAATC | |
| | TCTGCCGATACTTCAAAGAATACAGCTTATCTACAGATG | |
| | AATTCACTTAGAGCCGAGGATACAGCCGTGTATTATTGC | |
| | TCCCGATGGGGAGGAGATGGGTTCTACGCTATGGACTAC | |
| | TGGGGTCAAGGAACCCTGGTGACCGTTAGTTCA | |
| Herceptin scFv | DIQMTQSPSSLSASVGDRVTITCRASQDVNTAVAWYQQK | 70 |
| (amino acid | PGKAPKLLIYSASFLYSGVPSRFSGSRSGTDFTLTISSL | |
| sequence) | QPEDFATYYCQQHYTTPPTFGQGTKVEIKGSTSGSGKPG | |
| | SGEGSTKGEVQLVESGGGLVQPGGSLRLSCAASGFNIKD | |
| | TYIHWVRQAPGKGLEWVARIYPTNGYTRYADSVKGRFTI | |
| | SADTSKNTAYLQMNSLRAEDTAVYYCSRWGGDGFYAMDY | |
| | WGQGTLVTVSS | |
| Herceptin scFv | DIQMTQSPSSLSASVGDRVTITCRASQDVNTAVAWYQQK | 71 |
| VL | PGKAPKLLIYSASFLYSGVPSRFSGSRSGTDFTLTISSL | |
| | QPEDFATYYCQQHYTTPPTFGQGTKVEIK | |
| Herceptin scFv | EVQLVESGGGLVQPGGSLRLSCAASGFNIKDTYIHWVRQ | 72 |
| VH | APGKGLEWVARIYPTNGYTRYADSVKGRFTISADTSKNT | |
| | AYLQMNSLRAEDTAVYYCSRWGGDGFYAMDYWGQGTLVT | |
| | VSS | |
| Herceptin scFv | GSTSGSGKPGSGEGSTKG | 3 |
| Linker | | |
| scFv69 | TCTTCTGAGCTGACTCAGGACCCTGCTGTGTCTGTGGCC | 73 |
| (Herceptin | TTGGGACAGACAGTCAGGATCACATGCCAAGGAGACAGC | |
| anti-ID) | CTCAGAAGCTATTATGCAAGCTGGTACCAGCAGAAGCCA | |
| (nucleic acid) | GGACAGGCCCCTGTACTTGTCATCTATGGTAAAAACAAC | |
| | CGGCCCTCAGGGATCCCAGACCGATTCTCTGGCTCCAGC | |
| | TCAGGAAACACAGCTTCCTTGACCATCACTGGGGCTCAG | |
| | GCGGAAGATGAGGCTGACTATTACTGTAACAGCAGTGAA | |
| | CCAACCCCACCAAGAGTGGTCTTCGGCGGCGGAACAAAA | |
| | CTGACAGTGCTGGGCTCTACAAGCGGCAGCGGCAAACCT | |
| | GGATCTGGCGAGGGATCTACCAAGGGCGAGGTGCAACTA | |
| | TTGGAAAGTGGTGGCGGGCTGGTCCAACCGGGCGGGTCC | |
| | TTGAGGCTGTCCTGTGCAGCCAGCGGGTTTACTTTTTCT | |
| | TCCTACGCCATGTCTTGGGTACGACAGGCTCCCGGAAAA | |
| | GGGCTCGAGTGGGTGAGTGCAATCTCCGGGAGTGGGGGC | |
| | TCTACCTACTACGCCGATTCTGTCAAGGGTAGGTTCACT | |
| | ATCTCCAGGGATAATTCAAAGAATACTTTATACCTGCAG | |
| | ATGAATTCACTGCGAGCGGAAGATACAGCAGTGTACTAT | |
| | TGTGCCAAGAACGTGCACATCCAGCCCTTTGATTACTGG | |
| | GGCCAGGGCACCAGCGTGACCGTGTCTAGC | |
| scFv69 | SSELTQDPAVSVALGQTVRITCQGDSLRSYYASWYQQKP | 74 |
| (Herceptin | GQAPVLVIYGKNNRPSGIPDRFSGSSSGNTASLTITGAQ | |
| anti-ID) | AEDEADYYCNSSEPTPPRVVFGGGTKLTVLGSTSGSGKP | |
| (amino acid | GSGEGSTKGEVQLLESGGGLVQPGGSLRLSCAASGFTFS | |
| sequence) | SYAMSWVRQAPGKGLEWVSAISGSGGSTYYADSVKGRFT | |
| | ISRDNSKNTLYLQMNSLRAEDTAVYYCAKNVHIQPFDYW | |
| | GQGTSVTVSS | |
| scFv69 VL | SSELTQDPAVSVALGQTVRITCQGDSLRSYYASWYQQKP | 75 |
| | GQAPVLVIYGKNNRPSGIPDRFSGSSSGNTASLTITGAQ | |
| | AEDEADYYCNSSEPTPPRVVFGGGTKLTVL | |
| scFv69 VH | EVQLLESGGGLVQPGGSLRLSCAASGFTFSSYAMSWVRQ | 76 |
| | APGKGLEWVSAISGSGGSTYYADSVKGRFTISRDNSKNT | |
| | LYLQMNSLRAEDTAVYYCAKNVHIQPFDYWGQGTSVTVS | |
| | S | |
| scFv69 Linker | GSTSGSGKPGSGEGSTKG | 3 |
| 9G8 VHH | GAAGTGCAGCTTGTGGAGAGTGGCGGTGGTTTAGTTCAA | 77 |
| (anti-EGFR) | CCAGGGGGCAGCCTGCGCTTGAGTTGTGCCGCATCGGGT | |
| (nucleotide | CGTACCTTTTCTAGCTACGCAATGGGGTGGTTTCGTCAA | |
| sequence) | GCTCCTGGGAAGGAACGTGAGTTTGTGGTCGCCATTAAT | |
| | TGGTCATCAGGGAGTACATACTACGCTGATTCCGTCAAA | |
| | GGGCGCTTTACAATCTCACGCGATAACAGCAAGAATACC | |
| | CTTTATTTACAAATGAATAGTCTGCGTGCAGAAGATACG | |
| | GCTGTGTATTACTGCGCTGCGGGGTACCAAATCAACTCT | |
| | GGGAATTACAACTTTAAGGACTACGAGTATGATTATTGG | |
| | GGCCAGGGCACTCAGGTTACAGTCTCGAGC | |
| 9G8 VHH | EVQLVESGGGLVQAGGSLRLSCAASGRTFSSYAMGWFRQ | 78 |
| (anti-EGFR) | APGKEREFVVAINWSSGSTYYADSVKGRFTISRDNAKNT | |
| (amino acid | MYLQMNSLKPEDTAVYYCAAGYQINSGNYNFKDYEYDYW | |
| sequence) | GQGTQVTVSS | |
| T2A sequence | GGATCCGGCGCCACAAACTTCAGCCTGCTGAAACAGGCC | 79 |
| (nucleotide | GGCGACGTGGAGGAAAACCCAGGCCCA | |
| sequence) | | |
| T2A sequence | GSGATNFSLLKQAGDVEENPGP | 80 |
| (amino acid | | |
| sequence) | | |
| GFP (for | GTGTCCAAGGGCGAAGAACTGTTCACCGGCGTGGTGCCC | 81 |
| expression | ATTCTGGTGGAACTGGACGGGGATGTGAACGGCCACAAG | |
| detection) | TTCAGCGTTAGAGGCGAAGGCGAAGGGGATGCCACAAAC | |
| (nucleotide | GGCAAGCTGACCCTGAAGTTCATCTGCACCACCGGAAAG | |
| sequence) | CTGCCCGTGCCTTGGCCTACACTGGTCACCACACTGACA | |
| | TACGGCGTGCAGTGCTTCAGCAGATACCCCGACCATATG | |
| | AAGCAGCACGACTTCTTCAAGAGCGCCATGCCTGAGGGC | |
| | TACGTGCAAGAGAGAACCATCACCTTCAAGGACGACGGC | |
| | ACCTACAAGACCAGAGCCGAAGTGAAGTTCGAGGGCGAC | |
| | ACCCTGGTCAACCGGATCGAGCTGAAGGGCATCGACTTC | |
| | AAAGAGGACGGCAACATCCTGGGCCACAAACTTGAGTAC | |
| | AACTTCAACAGCCACAACGTGTAtATCACCGCCGACAAG | |
| | CAGAAGAACGGCATCAAGGCCAACTTCAAGATCCGGCAC | |
| | AACGTGGAAGATGGCAGCGTGCAGCTGGCCGATCACTAC | |
| | CAGCAGAACACACCCATCGGAGATGGCCCTGTGCTGCTG | |
| | CCCGATAACCACTACCTGAGCACCCAGAGCAAGCTGAGC | |
| | AAGGACCCCAACGAGAAGCGGGACCACATGGTGCTGCTG | |
| | GAATTTGTGACAGCCGCCGGAATCACCCACGGCATGGAT | |
| | GAGCTGTACAAG | |
| 8GFP (for | VSKGEELFTGVVPILVELDGDVNGHKFSVRGEGEGDATN | 82 |
| expression | GKLTLKFICTTGKLPVPWPTLVTTLTYGVQCFSRYPDHM | |
| detection) | KQHDFFKSAMPEGYVQERTITFKDDGTYKTRAEVKFEGD | |
| (amino acid | TLVNRIELKGIDFKEDGNILGHKLEYNFNSHNVYITADK | |
| sequence) | QKNGIKANFKIRHNVEDGSVQLADHYQQNTPIGDGPVLL | |
| | PDNHYLSTQSKLSKDPNEKRDHMVLLEFVTAAGITHGMD | |
| | ELYK | |

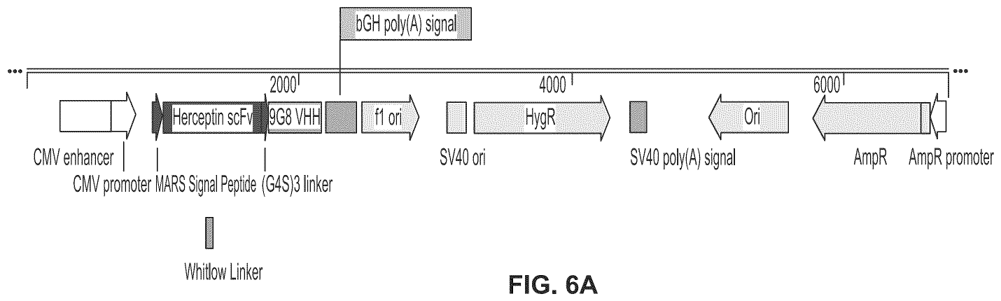

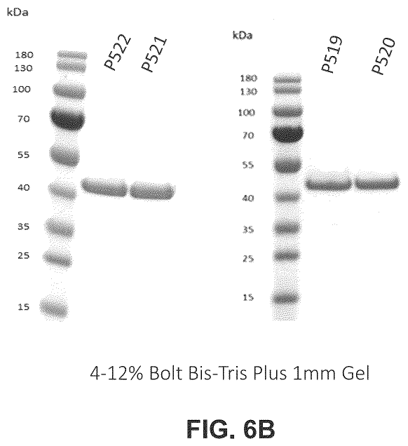

Four fusion protein (or bridge protein) constructs were assembled (Table 7) comprising the 9G8 VHH (Table 6) fused with either the Herceptin scFv or the anti-Herceptin idiotype scFv69 via a (G₄S)₃ linker (SEQ ID NO: 25). FIG. 6A shows a vector map of the plasmid used for cloning the bridge constructs described in Table 7.

EXPi-293 cells were transfected with a plasmid carrying a bridge construct described in Table 6 for protein expression using Expifectamine according to manufacturer's directions. Soluble bridge proteins were purified. Clarified supernatant was loaded over 1 ml HiTrap MabSelect SuRe column at 1 ml/min. The column was washed with 20 column volumes of PBS to remove unbound protein. The column was eluted with 10 cV of 0.1 M NaAcetate pH 3.5 and the sample was neutralized with 2.5 M Tris pH 6.5. The protein samples were polished by SEC to remove high molecular weight species. FIG. 6B shows a gel electrophoresis image demonstrating the purity of the bridge proteins purified from each construct.

TABLE 7
Bridge protein constructs

| Bridge Format | PLASMID |
|---|---|
| 9G8-Herceptin | p522 |
| Herceptin-9G8 | p521 |
| 9G8-scFv69 | p519 |
| scFv69-9G8 | p520 |

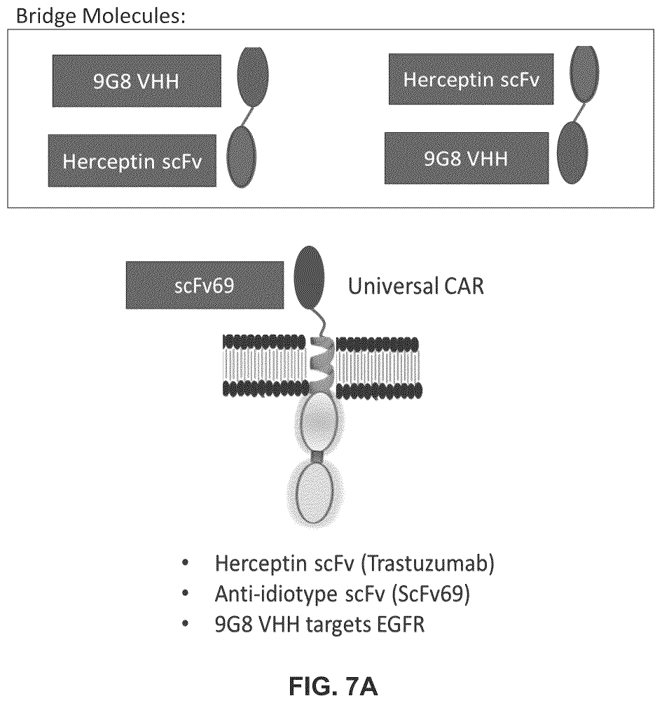

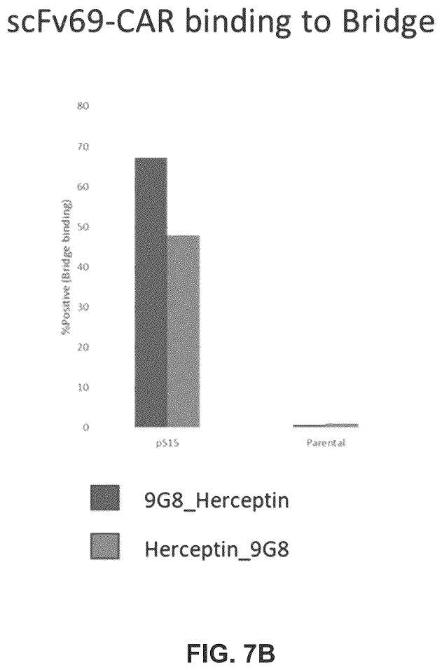

Binding assays for bridge protein binding to CAR transduced Jurkat cells were carried out to detect binding between Jurkat cells transduced with scFv69-CAR and two variants of the bridge protein: 9G8_Herceptin scFv and Herceptin scFv 9G8, as shown in FIG. 7A. Bridge protein binding was detected on the Intellicyte via after incubating the cell staining assay was performed as follows. The CAR transduced Jurkat cells were incubated for 30 minutes with bridge protein. Following the incubation, the cells were washed and then incubated for 30 minutes with Biotinylated EGFR. After a wash step, PE-labeled Streptavidin was incubated with the cells. The bridge protein binding, CAR transduced Jurkat cells were determined by flow cytometry to be 65% positive for 9G8_Herceptin scFv and 45% positive for Herceptin scFv 9G8 (FIG. 7B). Bridge protein binding was also detected in the opposite orientation: Herceptin CAR with scFv69_9 G8 bridge and Herceptin CAR with 9G8_scFv69 bridge (data not shown).

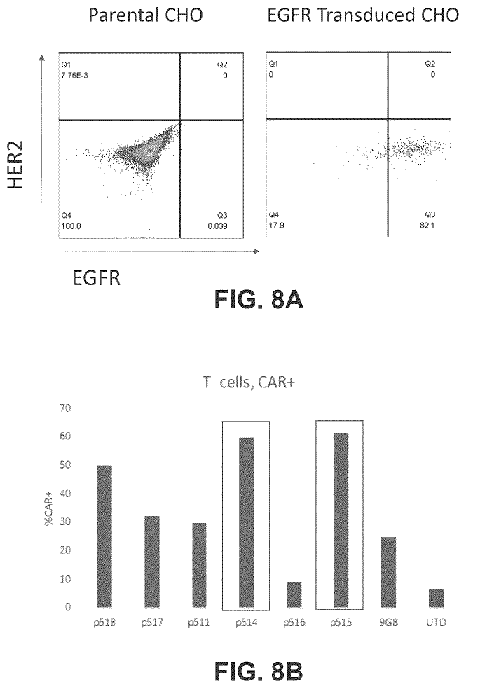

Cytotoxicity activity was evaluated between target cells and CAR expressing T cells. As assessed by flow cytometry, expression of EGFR was detected in EGFR transduced CHO cells (FIG. 8A). CAR expression was also evaluated in T cells. Highest expression was observed in CARs with the Long Ig-Hinge format for both HER-CAR (p514) and scFv69-CAR (p515) (FIG. 8B).

To set up the cytotoxicity activity assay, target cells (EGFR transduced CHO and parental CHO) were plated at 10,000 cells/well, labeled with CellTrace™ Violet (CTV). CAR T cells were plated at 100,000 cells/well, for approximate CAR+ effector to target cell ratio (E:T) of ˜5:1. CAR-T cells were co-cultured with CHO-EGFR+ cells for 48 hours. The controls used in the assay are shown in Table 8. 10, 1, 0.1, and 0 nM bridge proteins 9G8-Herceptin (p522) and 9G8-scFv69 (p519) were used.

TABLE 8
Cytotoxicity Activity Assay Controls

| Control | Testing |
|---|---|
| 9G8-CAR T | Positive control, target-specific CAR-mediated |
| | killing |
| Untransfected T cell | Negative control, non-specific T cell-mediated |
| | killing |
| No T cell | Negative control, baseline cell death |
| Herceptin CAR and | Negative control, specificity of CAR-Bridge- |
| Herceptin Bridge | killing mechanism |
| scFv69 CAR and | Negative control, specificity of CAR-Bridge- |
| scFv69 bridge | killing mechanism |

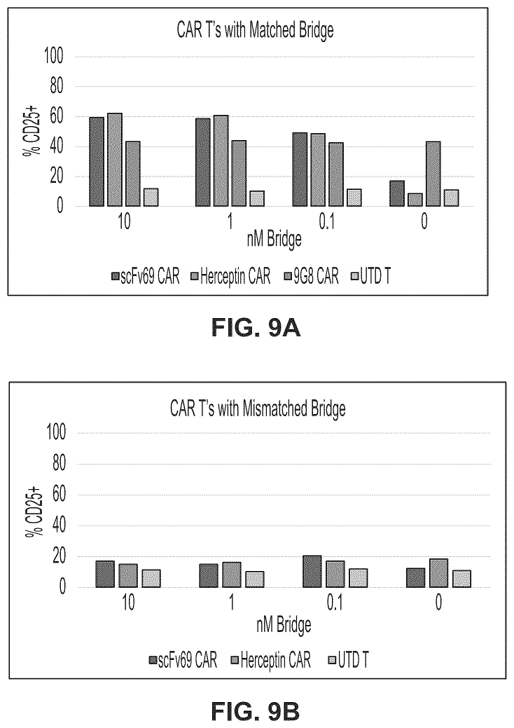

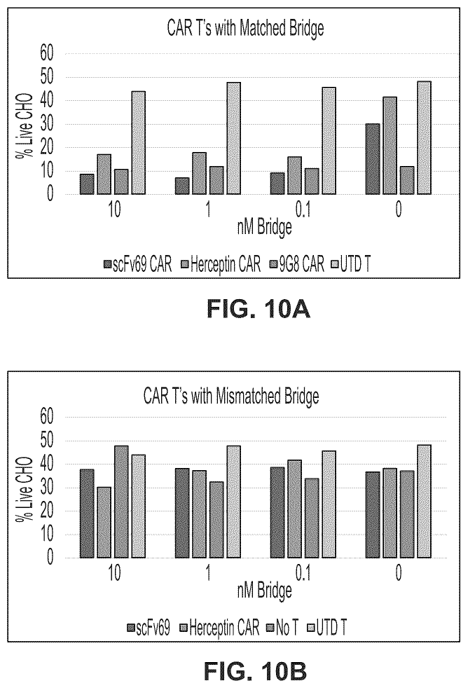

Universal CAR-T cells demonstrated CD25 activation when paired with the correct bridge protein but not when paired with a mis-matched bridge protein (FIGS. 9A, 9B). Further, universal CAR-T cells demonstrated cytotoxic activity when paired with the correct bridge protein but not when paired with a mis-matched bridge protein (FIGS. 10A, 10B).

### Example 5. Identification of VHH for Universal CAR Platform

VHH binders were selected as potential targets for discovery of anti-idiotypes to form a specific and unique binding pair which may serve as the “tag” and “anti-tag”, interchangeably. A list of VHHs were identified which are known to bind to non-human proteins. Additional VHHs that bind to Hen Egg Lysozyme (HEL) were also obtained. These VHHs and the generated plasmids are provided in Table 9 and sequences are provided in Table 10 (underlined sequences denote CDR locations).

TABLE 9
Candidate VHHs

| | | Corresponding Candidate |
|---|---|---|
| Non-human protein target | Plasmid | VVH Polypeptide |
| RSV F Protein | p243 | SEQ ID NO.: 94 |
| Listeria Internalin | p279 | SEQ ID NO.: 84 |
| Cobra Phospholipase A2 | p247 | SEQ ID NO.: 86 |
| Ebola Nucleoprotein | p246 | SEQ ID NO.: 88 |
| HSV Glycoprotein D | p249 | SEQ ID NO.: 90 |
| Lacotococcal phage RBP | p251 | SEQ ID NO.: 92 |
| Geobacillus stearothermophilus | p250 | SEQ ID NO.: 96 |
| Ricin (V5E4) | p252 | SEQ ID NO.: 98 |
| LYSO_CW_P01_B10 | p245 | SEQ ID NO.: 100 |
| LYSO_CW_P01_B11 | p242 | SEQ ID NO.: 102 |
| LYSO_CW_P01_D04 | p244 | SEQ ID NO.: 104 |
| LYSO_CW_P01_F11 | p248 | SEQ ID NO.: 106 |
| LYSO_CW_P01_F09 | p278 | SEQ ID NO.: 108 |
| LYSO_CW_P01_C04 | p253 | SEQ ID NO.: 110 |

TABLE 10
Candidate VHH sequences

| CAR regions | Sequence | SEQ ID NO |
|---|---|---|
| Listeria: | CAGGTGAAGTTGGAAGAGTCTGGTGGTGGCCTCGTCCAGGC | 83 |
| internalin 6dyx | TGGGGGGAGTCTTCGCCTTAGTTGCGCAGCTTCAGGTCGGA | |
| (nucleotide | CATACAGCACTTACGCGATGGGGTGGTTTCGCCAGACTCCG | |
| sequence) | GGGAAGGAGAGAGAATTGGTGGCAGCAATAAATTGGTCAGG | |
| | GGGCAACACGCACTATGCAGACTCTGTAAAAGGCCGGTTCA | |
| | CGATCAGTAGAGATAACGCCAAGAGCACCGTCTACCTCCAG | |
| | ATGAATTCCCTGAAACCTGAGGACACAGCAGTTTATTACTG | |
| | TGCCGCTCCGAAAGGCCACACAGGGGATCATTACTGGGGAC | |
| | CCGGCACCCAAGTGACTGTGAGCTCG | |
| Listeria: | QVKLEESGGGLVQAGGSLRLSCAASGRTYSTYAMGWFRQTP | 84 |
| internalin 6dyx | GKERELVAAINWSGGNTHYADSVKGRFTISRDNAKSTVYLQ | |
| (amino acid | MNSLKPEDTAVYYCAAPKGHTGDHYWGPGTQVTVSS | |
| sequence) | | |
| Naja kaouthia | CAGGTGCAACTCGTTGAAAGTGGAGGCGGTAGCGTTCAGGC | 85 |
| (cobra): | AGGTGGAAGCCTCAGGCTGTCCTGTGCGGCCAGTAGAGACA | |
| Phospholipase A2 | CGTATGATTCACACTGTATGGGGTGGTTCCGGCAAGCGCCC | |
| P3-3 | GGAAAAGAGAGGGAACAGGTGGCGGCACATAACGGTGGCCG | |
| (nucleotide | AAACACATATTACGCAGATAGCGTTAAAGGACGATTTACAA | |
| sequence) | TATCTCAGGACAATGCTAAGAATACGATGTATCTCCAAATG | |
| | AATAGCCTTAAACCTGAAGATACAGCCATTTACTACTGCGC | |
| | CGCGGACATGTCCGCGAGAAGGGTCGCAAACACAGGATGCA | |
| | GATACAATTATTGGGGCCAAGGCACTTTGGTAACTGTGAGC | |
| | TCG | |
| Naja kaouthia | QVQLVESGGGSVQAGGSLRLSCAASRDTYDSHCMGWFRQAP | 86 |
| (cobra): | GKEREQVAAHNGGRNTYYADSVKGRFTISQDNAKNTMYLQM | |
| Phospholipase A2 | NSLKPEDTAIYYCAADMSARRVANTGCRYNYWGQGTLVTVS | |
| P3-3 | S | |
| (amino acid | | |
| sequence) | | |
| Ebola: | CAGGTGCAACTTCAACAAAGCGGCGGGGGATCCGTAACGCC | 87 |
| nucleoprotein | AGGTGGATCACTGCGGCTTTCTTGTGCTGCTTCCGGATCCA | |
| Anti-Z C | TTTCAGACTTTGCTGCTATGGCATGGTATCGGCAGGCCCCT | |
| (nucleotide | GGCAAAGAAAGGGATTGGGTTTTTGGAACCATATTCTCAGC | |
| sequence) | AGGTGCTTTGTTGTACGCAGAACCAGTTAAAGGTCGGTTTA | |
| | CCATATCAAGGGATAACGCTAAGAATACTGTATATCTTCAA | |
| | ATGAACAGTTTGAAACCAGAGGATACTGCCGTATACTATTG | |
| | TCGCCTCTATGCTAAGGCAATTTATTGGGGTCAGGGCACAC | |
| | AGGTAACGGTGAGCTCG | |
| Ebola: | KVQLQQSGGGSVTPGGSLRLSCAASGSISDFAAMAWYRQAP | 88 |
| nucleoprotein | GKERDWVFGTIFSAGALLYAEPVKGRFTISRDNAKNTVYLQ | |
| Anti-Z C | MNSLKPEDTAVYYCRLYAKAIYWGQGTQVTVSS | |
| (amino acid | | |
| sequence) | | |
| Herpes simplex | CAGGTGCAACTTCAGGCTAGTGGAGGGGGACTGGTGCAAGC | 89 |
| virus: | TGGCGGGTCTTTGCGACTGTCCTGTGCAGCCTCAGGCCGAG | |
| Glycoprotein D | CTACAGGCAATTATCCCATGGGATGGTTCCGCCAGGCCCCT | |
| (nucleotide | GGAAAAGAACGCGAGTTCGTTGCAGCCATCAGTCGGGACGG | |
| sequence) | CGACAGTACATACTACCGGGATAGTGTTAAAGGCCGATTTA | |
| | CCATATCCCGAGACAATACGAAAAATACGGCATATCTTCAG | |
| | ATGAACAGTCTTAAGCCGGAGGACACAGCCGTCTACTATTG | |
| | TGCAGCTGACCGGCTGACAGCATATCGATACAATCCAGGGC | |
| | AGATTGACTATTGGGGACAGGGTACACAAGTTACGGTGAGC | |
| | TCG | |
| Herpes simplex | EVQLQASGGGLVQAGGSLRLSCAASGRATGNYPMGWFRQAP | 90 |
| virus: | GKEREFVAAISRDGDSTYYRDSVKGRFTISRDNTKNTAYLQ | |
| Glycoprotein D | MNSLKPEDTAVYYCAADRLTAYRYNPGQIDYWGQGTQVTVS | |
| (amino acid | S | |
| sequence) | | |
| Lactococcal | CAGGTGCAACTGGTCGAAAGTGGTGGGGGGCTCGTTCAAGC | 91 |
| phage: RBP | CGGTGGCAGTTTGCGCTTGTCATGCGCCGCTAGTGAAAGTA | |
| (nucleotide | CCTTCTCTAACTACGCGATGGGATGGTTCAGGCAAGCACCA | |
| sequence) | GGCCCTGAAAGGGAATTTGTGGCTACGATTTCTCAAACAGG | |
| | GTCCCACACCTACTACCGCAATTCTGTGAAGGGACGCTTCA | |
| | CGATTAGTCGGGATAACGCCAAGAACACAGTGTACCTTCAA | |
| | ATGAACAATATGAAGCCTGAAGACACGGCCGTGTATTATTG | |
| | TGCAGCCGGAGACAACTATTACTATACCAGAACTTATGAGT | |
| | ACGACTACTGGGGCCAGGGTACTCAGGTCACTGTGAGCTCG | |
| Lactococcal | QVQLVESGGGLVQAGGSLRLSCAASESTFSNYAMGWFRQAP | 92 |
| phage: RBP | GPEREFVATISQTGSHTYYRNSVKGRFTISRDNAKNTVYLQ | |
| (amino acid | MNNMKPEDTAVYYCAAGDNYYYTRTYEYDYWGQGTQVTVSS | |
| sequence) | | |
| RSV: F-protein | CAGGTGCAGTTGGTCGAGTCAGGTGGAGGGTCAGTGCAGCC | 93 |
| (nucleotide | AGGGGGGTCCCTTCGACTTAGTTGTGCAGCCAGTGGTTTTA | |
| sequence) | CACTGGATTACTATTACATTGGGTGGTTTCGACAGGCCCCC | |
| | GGGAAGGAACGCGAGGGTGTTTCTTGTATTTCCAGCTCACA | |
| | TGGCTCAACCTATTATGCTGACTCAGTAAAAGGTCGGTTTA | |
| | CGATAAGCCGGGATAATGCAAAGAATACCGTGTATCTTCAA | |
| | ATGAATAGCCTTAAACCAGAAGATACCGCTGTGTACTATTG | |
| | TGCCACTATACGCTCTAGCTCATGGGGGGGCTGTGTCCACT | |
| | ACGGGATGGATTATTGGGGGAAGGGCACGCAAGTCACGGTG | |
| | AGCTCG | |
| RSV: F-protein | QVQLVESGGGSVQPGGSLRLSCAASGFTLDYYYIGWFRQAP | 94 |
| (amino acid | GKEREGVSCISSSHGSTYYADSVKGRFTISRDNAKNTVYLQ | |
| sequence) | MNSLKPEDTAVYYCATIRSSSWGGCVHYGMDYWGKGTQVTV | |
| | SS | |
| Geobacillus | CAGGTGCAGTTGCAAGAAAGTGGAGGGGGATTGGTGCAGGC | 95 |
| stearothermophilus: | AGGTGGATCTTTGAGGCTGTCCTGTGCAGCCTCTGGTCGCA | |
| ilus: SbsB | CTAGCTCTGCGTACGCTATGGGTTGGTTTCGACAGGCCCCT | |
| (nucleotide | GGGAAAGAACGCGAGTTCGTTGCCGGCATTTCAAGCAAAGG | |
| sequence) | CGGTAGCACGTATTATGGTGCCAGCATGAAAGGACGCTTTA | |
| | CGATATCACGGGATAACGCGAAAAATACGGTCTACTTGCAG | |
| | ATGAACGGTCTGGCCCCAGAAGACACGGCAGTGTACTACTG | |
| | CGCTGCGAGCGACAAGTATAATTTCGACACCAGCCATGCGG | |
| | GATACGGCTATTGGGGCCAAGGGACCCAGGTTACAGTGAGC | |
| | TCG | |
| Geobacillus | QVQLQESGGGLVQAGGSLRLSCAASGRTSSAYAMGWFRQAP | 96 |
| stearothermophilus: | GKEREFVAGISSKGGSTYYGASMKGRFTISRDNAKNTVYLQ | |
| ilus: SbsB | MNGLAPEDTAVYYCAASDKYNFDTSHAGYGYWGQGTQVTVS | |
| (amino acid | S | |
| sequence) | | |
| Ricin (V5E4) | CAGGTGCAACTCGTGGAGACGGGTGGAGGACTTGTGCAAGC | 97 |
| (amino acid | GGGCGGAAGCCTTAGGTTGAGCTGTGCTGCGTCTGGATTTA | |
| sequence) | CATTTAGTAGCTATGCAATGGGCTGGTTTCGCCAGGCGCCG | |
| | GGGAAGGAACGCGACTTCGTTGCGGGTATCTCACTTAGCGG | |
| | CGCCGGGACGTACTATGTAAAAGGAAGGTTCACCATTTCAC | |
| | GCGATAACGCTAAAAACACTGTCTATTTGCAGATGAACAGC | |
| | CTCAAACCAGAGGATACTGCAGTATATTACTGTAAGGCCAC | |
| | AGGAGAAAGGGGGTATGGAGATCAGGGATATCTTGAAGTCT | |
| | GGGGGAGAGGGACGCTGGTTACCGTGAGCTCG | |
| Ricin (V5E4) | QVQLVETGGGLVQAGGSLRLSCAASGFTFSSYAMGWFRQAP | 98 |
| (amino acid | GKERDFVAGISLSGAGTYYVKGRFTISRDNAKNTVYLQMNS | |
| sequence) | LKPEDTAVYYCKATGERGYGDQGYLEVWGRGTLVTVSS | |
| Lyso CW P01 | CAGGTGCAACTTGTGGAGAGCGGGGGTGGTCTCGTCCAGGC | 99 |
| B10 | TGGTGGCTCACTTCGCCTCTCCTGCGCGGCTAGCGGCGGGA | |
| (nucleotide | TATTCTCAGGTGGAAGAATGGGCTGGTTCAGACAAGCTCCA | |
| sequence) | GGTAAAGAGAGAGAGTTCGTAGCGGCGGTAATCACTCGCGG | |
| | GGGGTCTACTTATTATGCGGACTCTGTGAAGGGTAGATTTA | |
| | CAATTTCACGGGACAACGCAAAGAATACTGTGTACCTTCAA | |
| | ATGAACTCACTTAAACCTGAGGACACGGCGGTTTATTACTG | |
| | CGCTGCAAGCGAGGTAACCTATGACGAGGGACATTACATCG | |
| | GAACCAAATCCACTTACGACACTTGGGGACAGGGCACGCAG | |
| | GTAACTGTGAGCTCG | |
| Lyso CW P01 | QVQLVESGGGLVQAGGSLRLSCAASGGIFSGGRMGWFRQAP | 100 |
| B10 | GKEREFVAAVITRGGSTYYADSVKGRFTISRDNAKNTVYLQ | |
| (amino acid | MNSLKPEDTAVYYCAASEVTYDEGHYIGTKSTYDTWGQGTQ | |
| sequence) | VTVSS | |
| Lyso CW P01 | CAGGTGCAGCTCGTAGAGTCCGGGGGAGGTCTGGTGCAAGC | 101 |
| B11 | TGGAGGTTCCTTGCGATTGTCATGTGCGGCAAGCGGAGGAA | |
| (nucleotide | TTTTTTCTGGAGGACGAATGGGGTGGTTCCGACAGGCACCG | |
| sequence) | GGAAAAGAGAGGGAGTTCGTAGCTGCCGTGATTACAAGGGG | |
| | TGGTAGCACATACTATGCAGATAGCGTAAAGGGTAGGTTTA | |
| | CGATATCCAGGGATAACGCAAAGAACACGGTCTACCTGCAG | |
| | ATGAACTCCCTTAAACCAGAAGATACTGCCGTTTATTATTG | |
| | CGCCGCATCAGAGGTAACTTACGATGAAGGTCGATACATTG | |
| | GGACGAAGAGCACCTATGACACATGGGGGCAGGGTACTCAA | |
| | GTGACCGTGAGCTCG | |
| Lyso CW P01 | QVQLVESGGGLVQAGGSLRLSCAASGGIFSGGRMGWFRQAP | 102 |
| B11 | GKEREFVAAVITRGGSTYYADSVKGRFTISRDNAKNTVYLQ | |
| (amino acid | MNSLKPEDTAVYYCAASEVTYDEGRYIGTKSTYDTWGQGTQ | |
| sequence) | VTVSS | |
| Lyso CW P01 | CAGGTGCAACTGGTGGAATCCGGGGGTGGACTGGTACAAGC | 103 |
| D04 | CGGAGGGAGTTTGAGACTCTCTTGCGCTGCCTCCGGTAGAA | |
| (nucleotide | TTTTTTCAATATACGACACCATAGGATGGTTTCGACAAGCG | |
| sequence) | CCTGGAAAGGAAAGAGAGTTTGTAGCGGCCACTATCACAAC | |
| | GGCGGGTATTACGACATATGATGATAGCGTTAAAGGACGGT | |
| | TCACGATAAGCCGCGACAATGCCAAGAACACAGTGTACCTC | |
| | CAAATGAATAGCCTTAAGCCCGAGGATACAGCCGTTTACTA | |
| | TTGTTACGTCCGAGTTGGTCGCGGTGACTACTGGGGTCAAG | |
| | GTACTCAGGTGACTGTAAGCTCG | |
| Lyso CW P01 | QVQLVESGGGLVQAGGSLRLSCAASGRIFSIYDTIGWFRQA | 104 |
| D04 | PGKEREFVAATITTAGITTYDDSVKGRFTISRDNAKNTVYL | |
| (amino acid | QMNSLKPEDTAVYYCYVRVGRGDYWGQGTQVTVSS | |
| sequence) | | |
| Lyso CW P01 | CAGGTGCAACTGGTTGAATCTGGTGGTGGGCTGGTCCAAGC | 105 |
| F11 | GGGAGGCAGTCTTCGACTTTCCTGCGCAGCCTCAGGGAGTA | |
| (nucleotide | TATTCAGCTTTTACGACGTAGGTTGGTTCCGCCAAGCGCCC | |
| sequence) | GGTAAAGAACGAGAGTTCGTCGCTGCCAGTATAACGAAGGG | |
| | AGGCGGGACGTACTACGTAGATTCAGTAAAAGGGAGATTTA | |
| | CCATAAGTAGGGACAATGCAAAGAACACGGTCTACCTCCAG | |
| | ATGAATAGCCTTAAACCAGAAGATACGGCAGTCTATTACTG | |
| | CGCCCTGGCAACCCCCCACGGATATGACTTTTGGGGCCAAG | |
| | GTACGCAAGTCACGGTGAGCTCG | |
| Lyso CW P01 | QVQLVESGGGLVQAGGSLRLSCAASGSIFSFYDVGWFRQAP | 106 |
| F11 | GKEREFVAASITKGGGTYYVDSVKGRFTISRDNAKNTVYLQ | |
| (amino acid | MNSLKPEDTAVYYCALATPHGYDFWGQGTQVTVSS | |
| sequence) | | |
| Lyso CW P01 | CAGGTGCAGCTCGTTGAAAGCGGGGGTGGCTTGGTCCAAGC | 107 |
| F09 | GGGAGGTTCCCTGAGACTGTCCTGCGCTGCGTCAGGATCAA | |
| (nucleotide | TAATCACGATTTATGTAGTCGGATGGTTCCGACAAGCCCCA | |
| sequence) | GGTAAAGAGCGGGAATTTGTGGCCTCCGACATAGGCTCTGG | |
| | TGGGTCAACTTATTACAGTGACTCCGTAAAAGGTCGGTTCA | |
| | CAATCTCAAGGGATAACGCAAAGAATACAGTCTACTTGCAA | |
| | ATGAATTCATTGAAGCCTGAGGATACAGCAGTGTACTACTG | |
| | CGTTACTGGAGATCCCTCTACTCCGTATTCATACTGGGGTC | |
| | AAGGCACACAGGTTACAGTGAGCTCG | |
| Lyso CW P01 | QVQLVESGGGLVQAGGSLRLSCAASGSIITIYVVGWFRQAP | 108 |
| F09 | GKEREFVASDIGSGGSTYYSDSVKGRFTISRDNAKNTVYLQ | |
| (amino acid | MNSLKPEDTAVYYCVTGDPSTPYSYWGQGTQVTVSS | |
| sequence) | | |
| Lyso CW P01 | CAGGTGCAACTGGTCGAGAGTGGTGGTGGACTGGTTCAAGC | 109 |
| C04 | TGGGGGCAGCCTGAGATTGTCCTGCGCCGCATCTGGCTCCT | |
| (nucleotide | CCTTCTCAATTTACGACGTGGGCTGGTTCCGCCAAGCACCT | |
| sequence) | GGAAAGGAAAGAGAGTTTGTTGCTGCGACAATTGAGACTGG | |
| | GGGACACACGTCTTACGCCGACTCAGTGAAAGGTAGATTTA | |
| | CAATCTCAAGGGATAACGCTAAAAACACCGTTTATCTGCAA | |
| | ATGAACTCCCTGAAACCGGAGGATACAGCTGTGTACTATTG | |
| | TTATGCGAAGATTGTCTACGACCAGGGCCCGAGCTACTACT | |
| | ATTGGGGCCAGGGGACACAGGTTACCGTGAGCTCG | |
| Lyso CW P01 | QVQLVESGGGLVQAGGSLRLSCAASGSSFSIYDVGWFRQAP | 110 |
| C04 | GKEREFVAATIETGGHTSYADSVKGRFTISRDNAKNTVYLQ | |
| (amino acid | MNSLKPEDTAVYYCYAKIVYDQGPSYYYWGQGTQVTVSS | |
| sequence) | | |

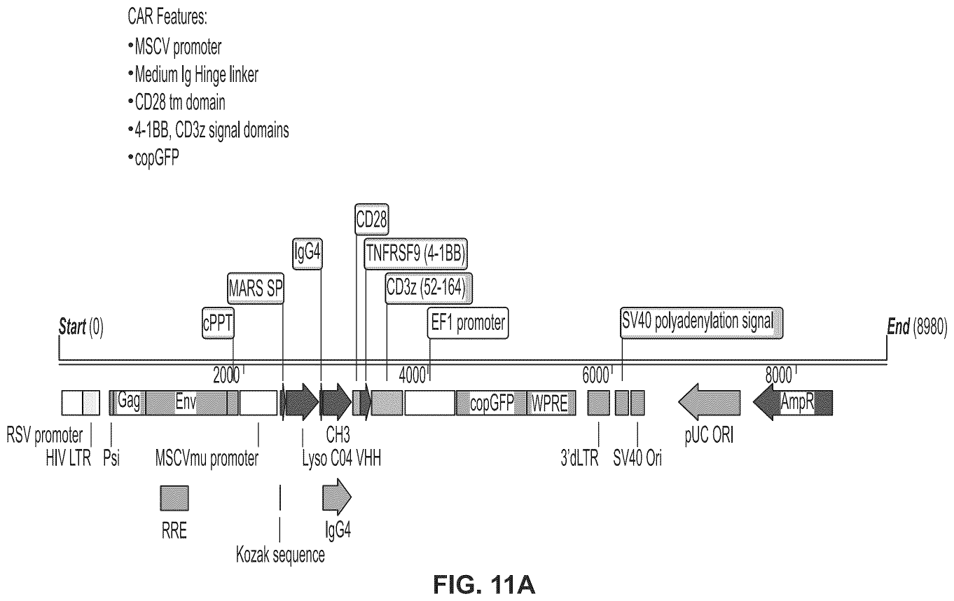

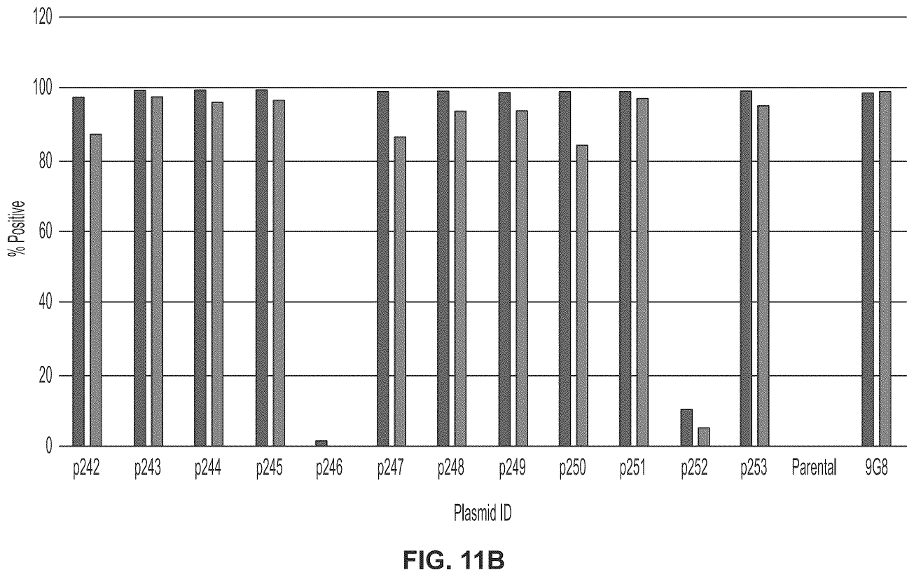

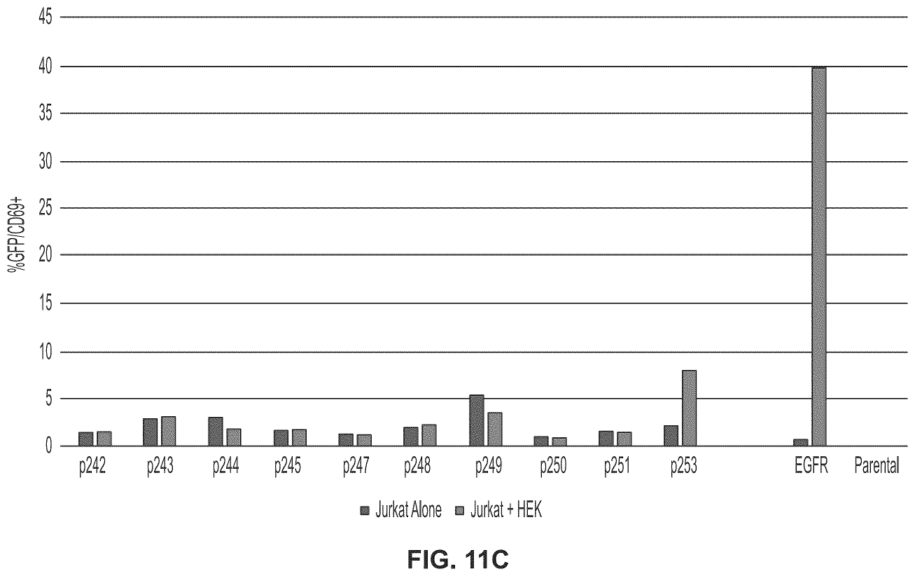

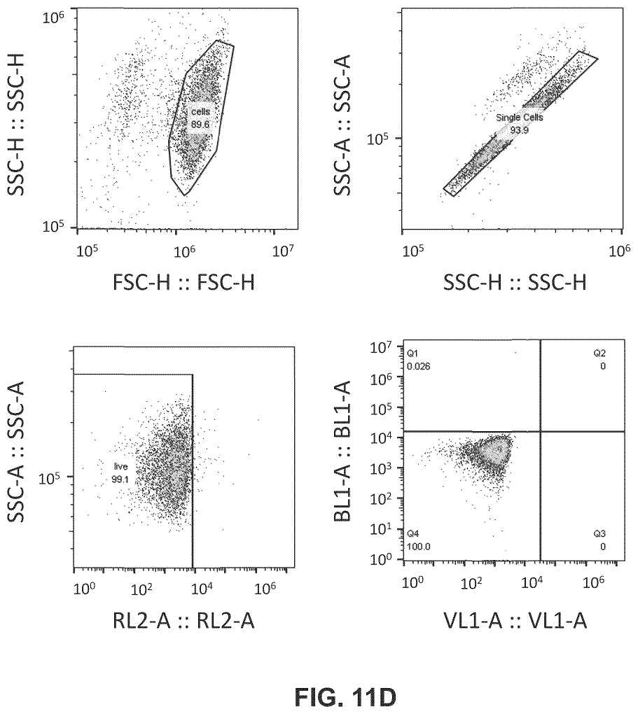

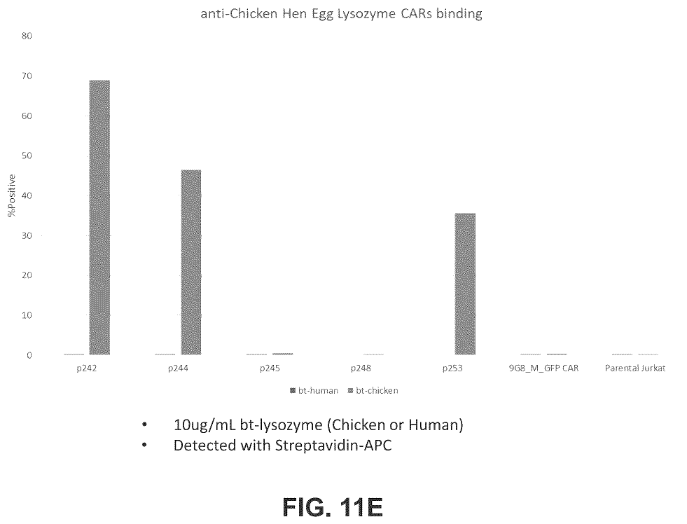

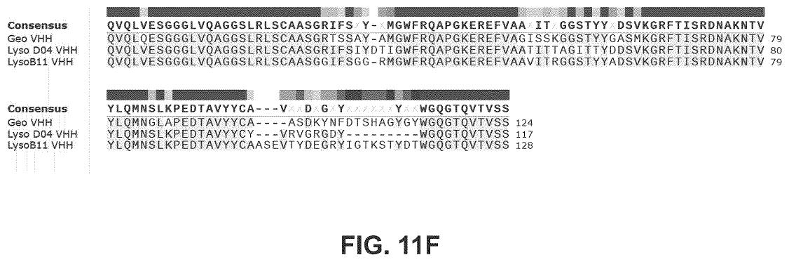

Lentiviral CAR expression constructs comprising a VHH described in Table 9 were assembled. FIG. 11A shows a vector map of the lentiviral vector used to deliver the VHH CARs. The lentiviral vectors were then used to transduce Jurkat cells. Robust CAR expression was observed for 10/14 VHH CAR on Jurkat cells (FIG. 11B). All candidate VHH expressed at very high levels in Jurkat cells as a CAR. To determine if there was any tonic signaling or nonspecific activation of these VHH when present on a CAR, transduced Jurkat cells were incubated with human embryonic kidney cells (HEK) overnight and assessed the next day for the T cell activation marker CD69. Low or no non-specific activation on Jurkat cells was observed for all VHH CAR constructs (FIGS. 11C, 11D) indicating that there was little or no signaling through the CAR without target-driven activation. Three anti-lysozyme VHH CARs bind to chicken lysozyme and are not cross-reactive to human (FIG. 11E), demonstrating specificity for the chicken protein.

Based on these data, the LYSO_CW_P01_B11 (B11) and LYSO_CW_P01_D04 (D04) VHHs were selected for further consideration owing to their high expression as CARs, low to no tonic signaling and nonspecific activity, and ideal biophysical characteristics (indicated by their strong expression as soluble proteins). These VHHs had good expression either in the CAR or in the bridge protein.

TABLE 11
Purification data for B11 and D04

| DNA | Protein | | Construct | Conc. | Vol | Yield | % |
|---|---|---|---|---|---|---|---|
| ID | ID | VHH | Format | (mg/mL) | (uL) | (ug) | Monomer |
| P1184 | PROT951 | LYSO_CW_P01_B11 | VHH_IgG1.Fc | 1.53 | 200 | 306.7 | 98.50 |
| P1186 | PROT949 | LYSO_CW_P01_D04 | VHH_IgG1.Fc | 1.94 | 200 | 387.1 | >99 |

FIG. 11F shows alignment of the amino acid sequences of VHHs targeting Geobacillus stearothermophilus, LYSO_CW_P01_B11 and LYSO_CW_P01_D04. A consensus sequence was generated based on the aligned sequences.

As a final characterization of the specificity of these VHH, CAR Jurkat cells transduced with B11 and D04 VHH were tested, as well as the other anti-HEL VHH of the panel of candidates, for binding to biotinylated chicken and human lysozyme, to ensure there was no binding to human lysozyme protein. Both the B11(p242) and D04 (p244) VHH show binding to chicken lysozyme with no cross-reactivity to human lysozyme protein (FIG. 11E).

### Example 6. Humanization of the B11 and D04 VHH

The B11 and D04 proteins were derived from the camelid VHH phage display library at Distributed Bio (San Francisco, CA). To limit the possibility of immunogenicity of these VHH, they were humanized in the framework regions of the VHH. From this Example onward, references to B11 and D04 are to the humanized VHH.

TABLE 12
Humanized versions of the originally camelid VHH B11 and D04

| | CAR | | |
|---|---|---|---|
| | Vector | | SEQ |
| VHH | Number | Amino Acid Sequence | ID NO |
| hyLYSO_ | P1649 | EVQLLESGGGLVQPGGSLRLSCAASGGIFSGGRMGWFRQAPGKERE | 111 |
| CW_P01_B11 | | FVAAVITRGGSTYYADSVKGRFTISRDNSKNTLYLQMNSLRAEDTA | |
| | | VYYCAASEVTYDEGRYIGTKSTYDTWGQGTQVTVSS | |
| hyLYSO_ | P1650 | EVQLLESGGGLVQPGGSLRLSCAASGRIFSIYDTIGWFRQAPGKER | 112 |
| CW_P01_D04 | | EFVAATITTAGITTYDDSVKGRFTISRDNSKNTLYLQMNSLRAEDT | |
| | | AVYYCYVRVGRGDYWGQGTQVTVSS | |

The humanized B11 and D04 went through similar characterization steps as the camelid versions to ensure that humanization of the framework regions did not compromise their biophysical characteristics, ability to express as CARs, or their properties when present on a CAR.

TABLE 13
Purification data for humanized B11 and D04

| DNA | PROT | | Conc. | Volume | Yield | % | Endotoxin |
|---|---|---|---|---|---|---|---|
| ID | ID | VHH | (mg/mL) | (mL) | (mg) | Monomer | units/mL |
| P1376 | PROT1045 | huLysoB11 | 4.21 | 2.85 | 12.0 | >99 | <1 |
| P1377 | PROT1046 | huLysoD04 | 3.17 | 4.20 | 13.3 | >99 | <1 |

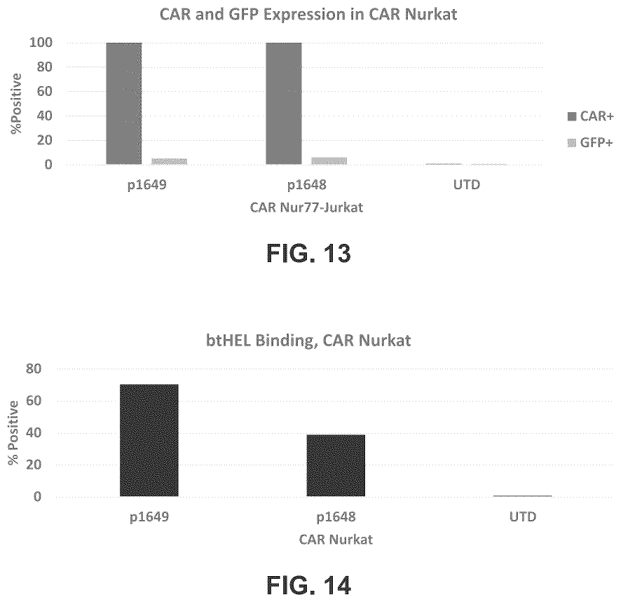

As before, each VHH CAR was transduced into Jurkat cells. Additionally, each VHH CAR was transduced into Nurkat cells, the Nur77 reporter line of Jurkat cells (Nurkat). This cell line contains a GFP reporter that is responsive to Nur77, a transcription factor that is activated by CD3z stimulation and serves as a reporter for CAR activation. Thus, activation via a CAR in Nurkat cells causes the cells to express GFP. FIG. 13 shows CAR and GFP expression of Nurkat cells transduced with lentivirus containing the humanized B11 (p1649) and D04 (p1648) CARs. UTD cells are untransduced Nurkat cells. CAR Expression was measured using the polyclonal anti-camelid VHH cocktail (Genscript). These results indicated that the expression of the B11 and D04 VHH as CARs was not impacted by the humanization of their sequences. Additionally, the lack of GFP expression in these CAR Nurkats show that there is very little tonic signaling mediated by the humanized versions of the CARs.

To ensure that there was no change in the binding characteristics of each VHH as a result of humanization, we tested whether these humanized VHH CARs could still bind HEL. FIG. 14 shows the results of an assay in which B11 (p1649) and D04 (p1648) CAR Nurkats were stained with biotinylated HEL and detected with streptavidin-APC via flow cytometry. UTD are untransduced Nurkats as a negative control. Both B11 and D04 retained their ability to bind HEL after humanization.

### Example 7. Phage Display Panning for Anti-Idiotype VHH Against B11 and D04

Humanized B11 and D04 VHH were used in phage library panning to identify specific, anti-ID VHH against each specifically. The synthetic VHH phage library consisted of four sub-libraries that are differentiated primarily by the length of their CDR3 sequence, with library 1 (CNTY1) having the shortest CDR length, and libraries 3 and 4 (CNTY3 and CNTY4) having the longest CDR lengths.

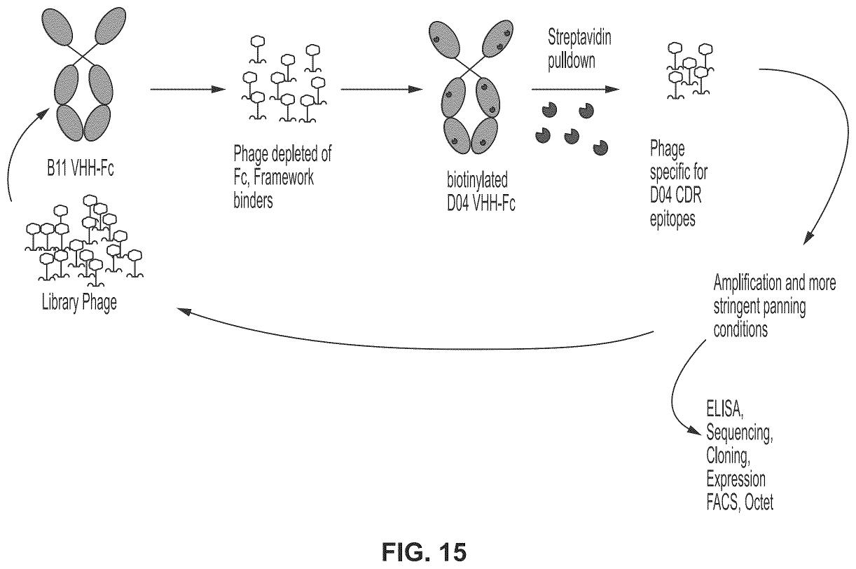

One panning schema used is shown in FIG. 15. In this method, the anti-idiotype VHH were discovered against D04 by first depleting the library against the B11-Fc protein. This was done to remove any phage that bind the framework regions of the VHH and the Fc portion of the protein. This depleted phage output was then panned on biotinylated D04-Fc protein. The biotinylated D04, with any phage binders could be isolated via streptavidin. The output phage were amplified in bacteria and used in further rounds of the panning with increasingly stringent panning conditions. Alternatively, the VHH displayed by the phage could be tested by ELISA for binding to D04 or B11 protein, sequenced, and cloned into expression vectors for more rigorous testing. This phage library panning approach was used to identify VHH binders to both D04 and B11.

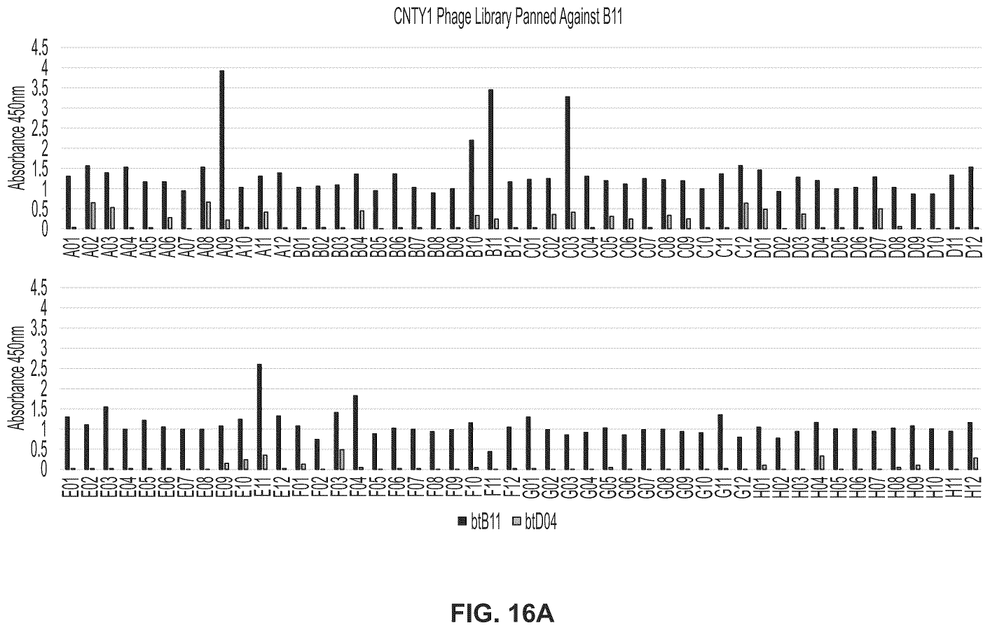

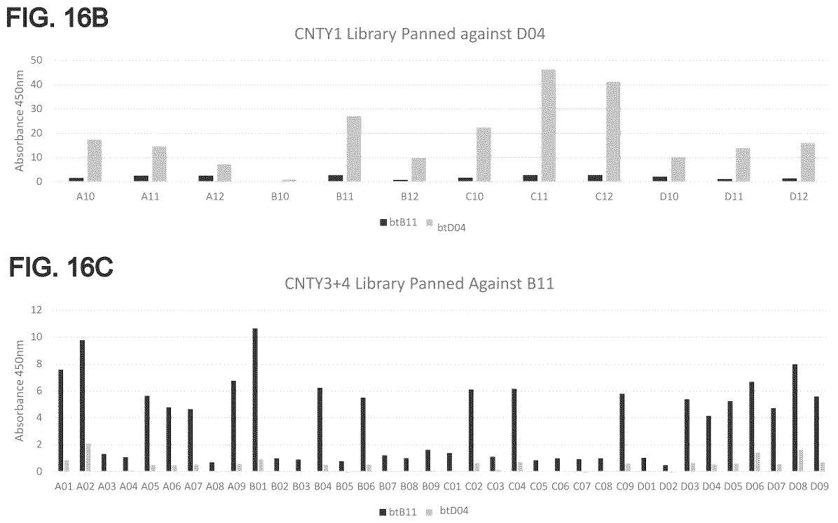

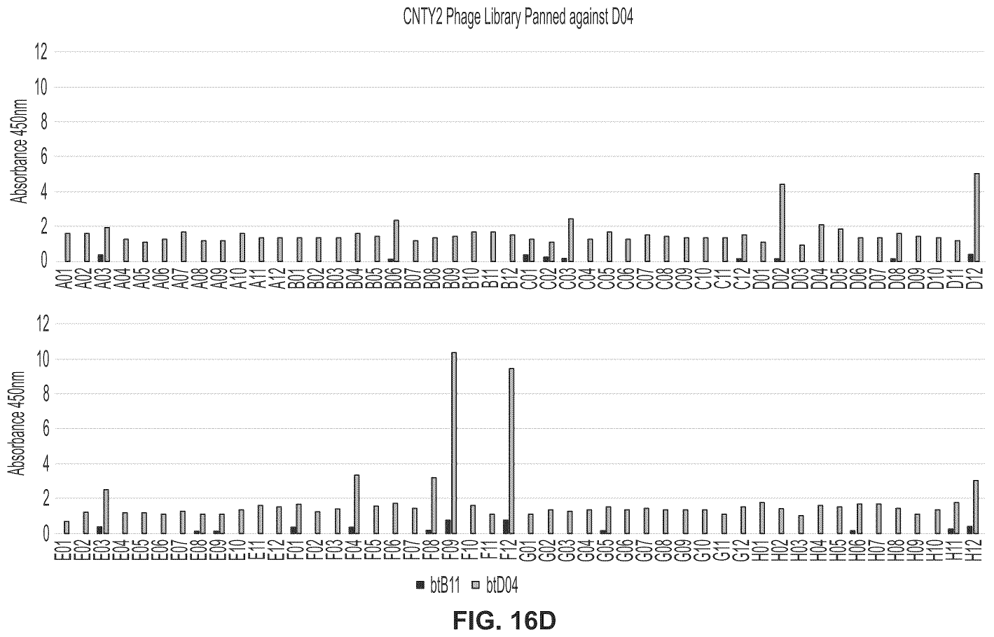

This panning schema was iterated 4 times with progressively decreasing target antigen concentration to select for high affinity binders (200 nM for the first and second rounds, 50 nM for the third round, and 10 nM for the fourth round). The output phage of the final round of panning was used to transform bacteria to single colonies, which were screened as periplasmic extract (PPE) in an ELISA against both the target VHH and off target VHH (FIG. 16).

PPE samples that showed specific responses to their target protein and little response to the off target proteins were selected for additional characterization. 6 unique B11 binders and 18 unique D04 binders were identified. These unique sequences were cloned into Fc-fusion expression vectors for further testing. The sequences for these proteins and identifiers are shown in Table 14.

TABLE 14
Anti-idiotypes expressed as IgG1-Fc fusions

| | | | |
|---|---|---|---|
| Fc | | | |
| Fusion | Anti-ID | | SEQ |
| Prot ID | VHH | Amino Acid Sequence of anti-Idiotype VHH | ID NO |

Anti-idiotypes that arose from panning that targeted D04

| | | | |
|---|---|---|---|
| AMD67 | huD04_ | EVQLLESGGGLVQPGGSLRLSCAASGRTFSSGAMGWYRQAPGKERE | 113 |
| | CNTY2_F09 | LVSAISNRGSTVYADSVKGRFTISRDNSKNTLYLQMNSLRAEDTAV | |
| | | YYCAADRNPSSAGAGVAAYRLIARFNYWGQGTQVTVSS | |
| AMD68 | huD04_ | EVQLLESGGGLVQPGGSLRLSCAASGFTLSSYAMGWYRQAPGKERE | 114 |
| | CNTY2_F08 | GVSSISYAGVTNYADSVKGRFTISRDNSKNTLYLQMNSLRAEDTAV | |
| | | YYCAAALGTGYYAYRRLWSYRIGSQAYDYWGQGTQVTVSS | |
| AMD69 | huD04_ | EVQLLESGGGLVQPGGSLRLSCAASGLTYTGYAMGWYRQAPGKERE | 115 |
| | CNTY2_E03 | LVSAISSRSSIYYADSVKGRFTISRDNSKNTLYLQMNSLRAEDTAV | |
| | | YYCAAGRPVALGSWRRTATWSAGLGAEYAYWGQGTQVTVSS | |
| AMD70 | huD04_ | EVQLLESGGGLVQPGGSLRLSCAASGRTFSSYGMGWYRQAPGKERE | 116 |
| | CNTY2_F04 | LVSAISNRGSTYYADSVKGRFTISRDNSKNTLYLQMNSLRAEDTAV | |
| | | YYCAASPARVGVSGHSSSRRSYYGYSYDYWGQGTQVTVSS | |
| ABD72 | huD04_ | EVQLLESGGGLVQPGGSLRLSCAASGSIFSSDVMGWYRQAPGKERE | 117 |
| | CNTY2_H12 | LVSAISSGGSTRYADSVKGRFTISRDNSKNTLYLQMNSLRAEDTAV | |
| | | YYCAATMGKSKTNRRNYGTWRYGAYAYWGQGTQVTVSS | |
| AMD74 | huD04_ | EVQLLESGGGLVQPGGSLRLSCAASGFTFSSNAMGWYRQAPGKERE | 118 |
| | CNTY2_F12 | FVSSISSGRSTTYADSVKGRFTISRDNSKNTLYLQMNSLRAEDTAV | |
| | | YYCAAHGTKYKWTRARLRSARQKQLETYRYWGQGTQVTVSS | |
| AMD77 | huD04_ | EVQLLESGGGLVQPGGSLRLSCAASGFTSSNYVMGWYRQAPGKERE | 119 |
| | CNTY1_A11 | FVSAIGSGGSTYYADSVKGRFTISRDNSKNTLYLQMNSLRAEDTAV | |
| | | YYCAAEIYTSGARDYWGQGTQVTVSS | |
| AMD78 | huD04_ | EVQLLESGGGLVQPGGSLRLSCAASGLTFSRYAMGWYRQAPGKERE | 120 |
| | CNTY2_C03 | LVSAISSRRSTYYADSVKGRFTISRDNSKNTLYLQMNSLRAEDTAV | |
| | | YYCAADYRLTSLERARYASASITYDYWGQGTQVTVSS | |
| AMD79 | huD04_ | EVQLLESGGGLVQPGGSLRLSCAASGFTFNRYSMGWYRQAPGKERE | 121 |
| | CNTY2_B06 | LVSAISSGGSGYYADSVKGRFTISRDNSKNTLYLQMNSLRAEDTAV | |
| | | YYCAIQDYSRIWEYIALDRRRSYYGMDYWGQGTQVTVSS | |
| AMD80 | huD04_ | EVQLLESGGGLVQPGGSLRLSCAASGFTFRSYAMGWYRQAPGKERE | 122 |
| | CNTY2_D02 | GVSVISSGGTPSYADSVKGRFTISRDNSKNTLYLQMNSLRAEDTAV | |
| | | YYCDAIMGLVEADYVSTGTYEYTAYAYWGQGTQVTVSS | |
| AMD81 | huD04_ | EVQLLESGGGLVQPGGSLRLSCAASGLTFSGVCMGWFRQAPGKERE | 123 |
| | CNTY2_D04 | GVSAIYSSGSTYYADSVKGRFTISRDNSKNTLYLQMNSLRAEDTAV | |
| | | YYCAADRIPDLGEPCIGTTNLARTYNYWGQGTQVTVSS | |
| AMD82 | huD04_ | EVQLLESGGGLVQPGGSLRLSCAASGRTSSNYVMGWYRQAPGKERE | 124 |
| | CNTY1_B12 | GVSAITSGGSTYYADSVKGRFTISRDNSKNTLYLQMNSLRAEDTAV | |
| | | YYCAANLYSRTGAYDYWGQGTQVTVSS | |
| AMD84 | huD04_ | EVQLLESGGGLVQPGGSLRLSCAASGYIYSSYSMGWYRQAPAKNAS | 125 |
| | CNTY2_D05 | LSAITSSGETYYADSVKGRFTISRDNSKNTLYLQMNSLRAEDTAVY | |
| | | YCAADALDAPIAGDRYYRGSGAGYAYWGQGTQVTVSS | |
| AMD85 | huD04_ | EVQLLESGGGLVQPGGSLRLSCAASGSAFSSNAMGWYRQAPGKERE | 126 |
| | CNTY2_D12 | LVSAISSGGSTNYADSVKGRFTISRDNSKNTLYLQMNSLRAEDTAV | |
| | | YYCAARNPLTYTALVSNAPSGDYYLFEYRLWGQGTQVTVSS | |
| AMD86 | huD04_ | EVQLLESGGGLVQPGGSLRLSCAASGFTSSNYVMGWYRQAPGKERE | 127 |
| | CNTY1_D12 | GVSAISSSGRTYYADSVKGRFTISRDNSKNTLYLQMNSLRAEDTAV | |
| | | YYCAAAYYSGYGETDYWGQGTQVTVSS | |
| AMD87 | huD04_ | EVQLLESGGGLVQPGGSLRLSCAASGSTFSYYYMGWYRQAPGKERE | 128 |
| | CNTY1_D11 | LVSAISSGGSIYYADSVKGRFTISRDNSKNTLYLQMNSLRAEDTAV | |
| | | YYCARRTYYGDEADYWGQGTQVTVSS | |
| AMD88 | huD04_ | EVQLLESGGGLVQPGGSLRLSCAASGRTSSNYVIGWYRQAPGKERE | 129 |
| | CNTY1_D10 | FVSAISGGGSTYYADSVKGRFTISRDNSKNTLYLQMNSLRAEDTAV | |
| | | YYCATRIYTARGAGDYWGQGTQVTVSSS | |

Anti-idiotypes that arose from panning that targeted B11

| | | | |
|---|---|---|---|
| AMD71 | huB11_ | EVQLLESGGGLVQPGGSLRLSCAASGRTYTGSAMGWYRQAPGKERE | 130 |
| | CNTY3 + | LVSAISTGGETYYADSVKGRFTISRDNSKNTLYLQMNSLRAEDTAV | |
| | 4_A02 | YYCAAGYVGLPYTYRPATSRRGTYTWGQGTQVTVSS | |
| AMD73 | huB11_ | EVQLLESGGGLVQPGGSLRLSCAASGFTFSSYSIGWYRAPGGKERE | 131 |
| | CNTY3 + | LVSVISSGGSTYYADSVKGRFTISRDNSKNTLYLQMNSLRAEDTAV | |
| | 4_A01 | YYCAASDWSYSWITYTGTWRLYEYWGQGTQVTVSS | |
| AMD75 | huB11_ | EVQLLESGGGLVQPGGSLRLSCAASGFAFSDYVMGWYRQAPGKERE | 132 |
| | CNTY1_E11 | FVSAIRSTTSTYYADSVKGRFTISRDNSKNTLYLQMNSLRAEDTAV | |
| | | YYCAADVWSGYEYDWYGQGTQVTVSS | |
| AMD76 | huB11_ | EVQLLESGGGLVQPGGSLRLSCAASGLAFSSYAMGWYRQAPGKERE | 133 |
| | CNTY1_B11 | FVSVISGTGETLYADSVKGRFTISRDNSKNTLYLQMNSLRAEDTAV | |
| | | YYCAADVWSGYEYDYWGQGTQVTVSS | |

### Example 8. Specificity and Affinity Testing of Anti-Idiotype VHH

To determine the specificity and binding of anti-idiotypes, the Fc fusion proteins were tested via FACS against Nurkat cells expressing either B11 or D04 VHH CARs.

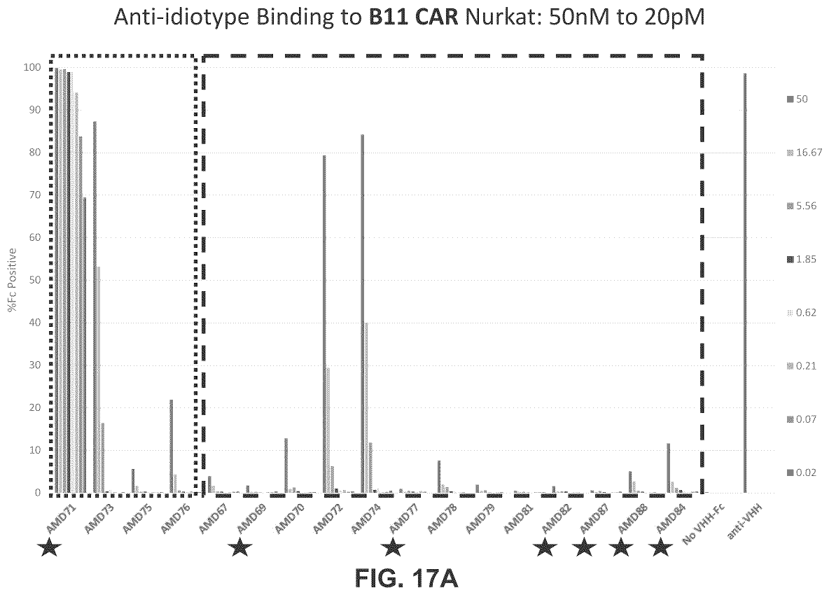

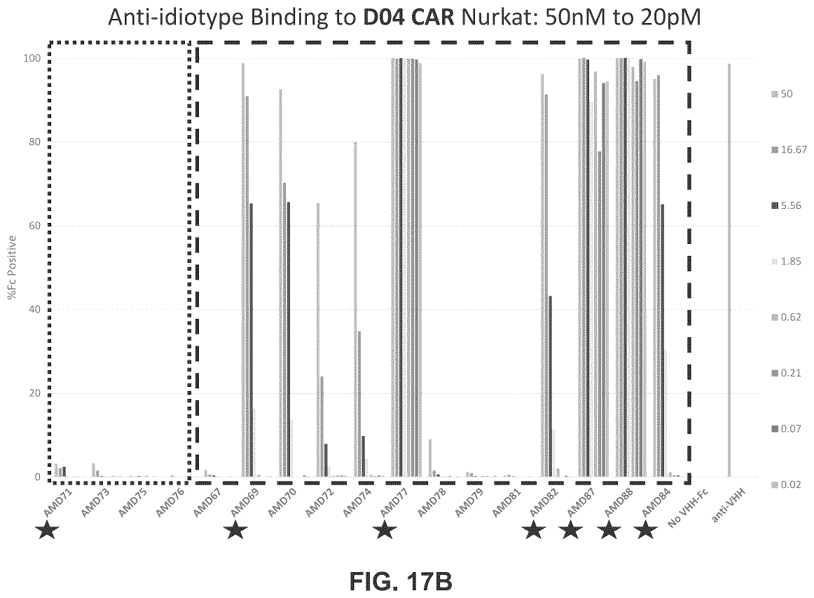

As shown in FIGS. 17A-17B, several of the anti-idiotypes showed robust and specific binding to the VHH on the CAR in a range of concentrations with negligible binding to the off-target CAR (e.g., AMD 71 (to B11) and AMD 69, AMD 77, AMD 82, AMD 84 AMD 87, and AMD 88 (to D04)). Also noted were a few binders that showed nonspecific binding, indicated by binding to both cell lines (AMD 72 and AMD 74). Of these, AMD 71, AMD 77, AMD 82, AMD 84, AMD 87, and AMD 88 were chosen for further specificity testing. Biotinylated anti-Idiotype VHH-Fc fusion proteins were arrayed against a variety of other VHH CAR-Nurkat, and any binding to those cell lines via FACS was subsequently detected.

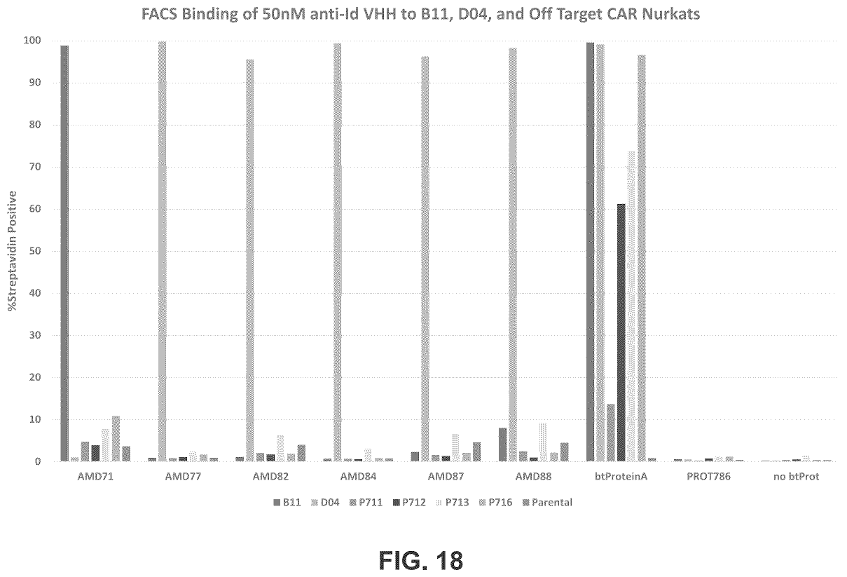

FIG. 18 shows binding of the lead anti-idiotype biotinylated VHH-Fc to target and off target CAR Nurkat cells detected via FACS with streptavidin-APC. The cells lines that were tested include B11, D04, P711, P712, P713, P716, and parental cells. B11 and D04 cells are CAR Nurkat cells expressing B11 and D04 VHH CARs, respectively. P711, 712, 713, 716 cells are off-target CAR Nurkat cells. Parental cells are untransduced, non-CAR-expressing Nurkat cells. Controls include biotinylated Protein A, which nonspecifically binds most VHH, PROT786, an unrelated biotinylated VHH-Fc protein, and no biotinylated protein added. The lead VHH demonstrated negligible binding to off-target VHH CARs and Parental Nurkat while still having robust binding to the target VHH.

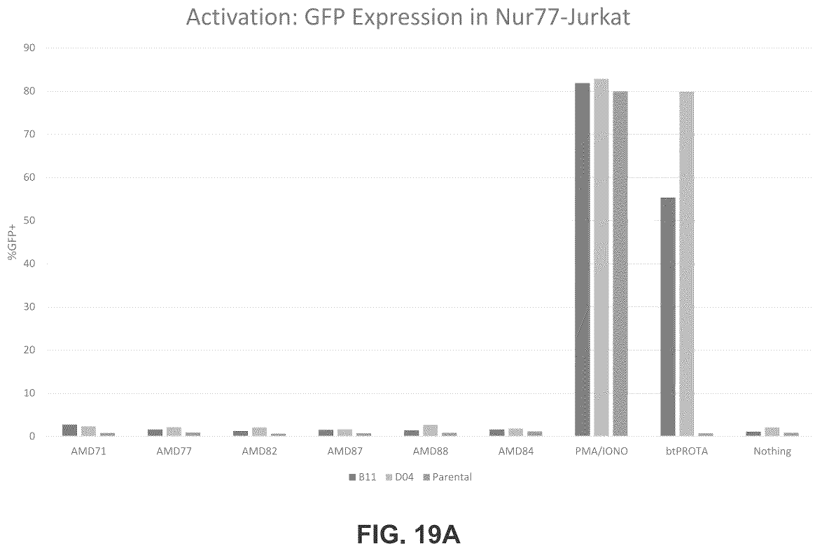

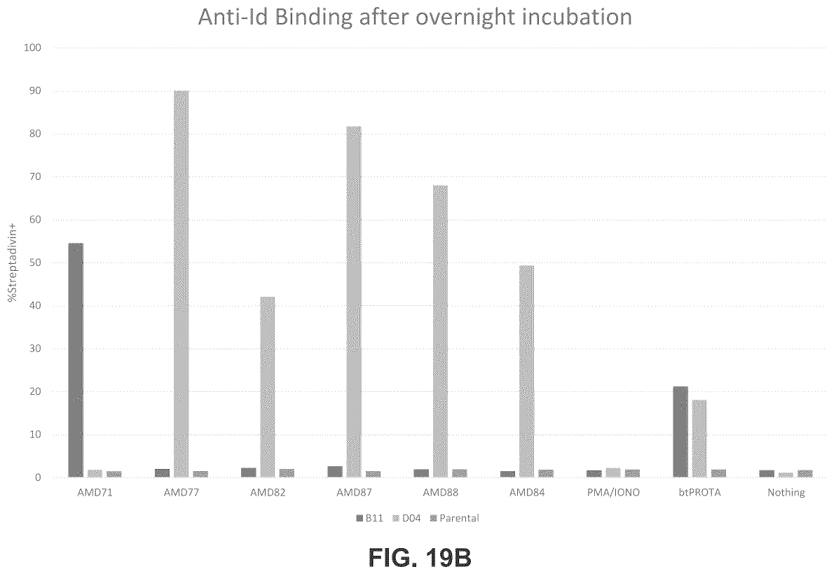

In some instances, the specific binding event that forms the basis of the “tag” and “anti-tag” interaction of the universal CAR is an important aspect to the safety profile of the platform. In addition, the binding event of the lysozyme VHH and an anti-idiotype alone should not be sufficient to cause CAR-induced activation. To determine whether the binding of B11 or D04 to their soluble anti-idiotype(s) causes CAR-induced activation absent the fusion of a tumor-antigen-targeting VHH to the soluble protein, an overnight stimulation assay in which the lead soluble anti-Idiotype Fc-fusion proteins were incubated with their target and off target CAR Nurkats as well as parental Nurkat. FIGS. 19A-19B show results of an overnight stimulation assay with Nurkat cells incubated with 10 nM anti-idiotype VHH Fc-fusion via FACS. FIG. 19A shows GFP expression of B11 and D04 CAR Nurkat cells, as well as parental Nurkat cells after overnight coculture with anti-idiotype Fc-fusion proteins. FIG. 19B shows detection of biotinylated protein via streptavidin-APC after the overnight incubation. Controls include PMA/Ionomycin (a TCR-crosslinker that results in robust Nurkat cell activation), biotinylated protein A, and a sample with no protein added. None of the anti-idiotype leads caused observable activation in the Nurkats as measured by GFP expression, compared to PMA/ionomycin induction. The anti-idiotypes were also still detectable after the overnight incubation, indicating that, if the anti-idiotypes did trigger CAR activation, the concentration of antigen used in the assay was sufficient to cause activation over the time course of the experiment that would have been observable.

Affinity measurements were obtained by Octet for the anti-ID binders to both the B11-his tagged VHH and D04-his tagged VHH.

TABLE 15
Octet results of anti-idiotype VHH Fc-fusion proteins
tested against B11 his-tagged proteins

| Sample ID | KD (M) | kon(1/Ms) | kdis(1/s) | Response |
|---|---|---|---|---|
| AMD71 | <1.0E−12 | 2.16E+03 | <1.0E−07 | 0.0062 |
| AMD77 | 2.08E−09 | 6.96E+05 | 1.45E−03 | 0.497 |
| AMD82 | 1.25E−07 | 6.97E+05 | 8.68E−02 | 0.1718 |
| AMD87 | 3.69E−06 | 1.76E+04 | 6.49E−02 | 0.1306 |
| AMD88 | 7.23E−08 | 5.88E+05 | 4.25E−02 | 0.2893 |
| AMD84 | 1.85E−07 | 7.90E+05 | 1.47E−01 | 0.0991 |
| PROT253 | NA | NA | NA | −0.05 |

Shown are affinity values (KD), on rates (kon), off rates (kdis), and response for anti-idiotype VHH fc-fusion proteins against B11-his protein

TABLE 16
Octet results of anti-idiotype VHH Fc-fusion proteins
tested against D04 his-tagged proteins

| Sample ID | KD (M) | kon(1/Ms) | kdis(1/s) | Response |
|---|---|---|---|---|
| AMD71 | 5.15E−07 | 2.73E+05 | 1.40E−01 | 0.0566 |
| AMD77 | 7.96E−08 | 3.54E+03* | 2.82E−04 | 0.1785 |
| AMD82 | NA | NA | NA | −0.0225 |
| AMD87 | NA | NA | NA | −0.0301 |
| AMD88 | NA | NA | NA | −0.0183 |
| AMD84 | NA | NA | NA | −0.0341 |
| PROT253 | NA | NA | NA | −0.0648 |

Shown are affinity values (KD), on rates (kon), off rates (kdis), and response for anti-idiotype VHH fc-fusion proteins against B11-his protein.

The anti-idiotype proteins demonstrated a range of affinities from 2 nM (AMD77 to D04) to 3.7 μM (AMD87 to D04). To verify the specificity of the two highest affinity binders, AMD77 (2 nM) and AMD88 (72 nM) via Octet, we tested each protein for binding against itself, its target VHH, off-target VHH, total human IgG from sera, and Fc-y fragment.

TABLE 17
Octet specificity analysis of anti-idiotypes AMD77 and AMD88

| Sample ID | Analyte | Response |
|---|---|---|
| AMD77 | LysoD04.His | 0.3374 |
| AMD77 | AMD77 | 0.0493 |
| AMD77 | AMD88 | 0.2773 |
| AMD77 | IgG | −0.0155 |
| AMD77 | Fc | −0.009 |
| AMD77 | PROT939 | −0.0053 |
| AMD77 | LysoD04.Fc | 2.6741 |
| AMD88 | LysoD04.His | 0.0923 |
| AMD88 | AMD77 | 0.0229 |
| AMD88 | AMD88 | 0.1492 |
| AMD88 | IgG | −0.0338 |
| AMD88 | Fc | −0.0261 |
| AMD88 | PROT939 | −0.024 |
| AMD88 | LysoD04.Fc | 2.1784 |

Each sample (either AMD77 or AMD88) was tested against the analytes D04-his, AMD77, AMD88, IgG from sera, Fc-y fragment, PROT939 (an off target VHH-fc soluble), and D04-fc. Response values are shown in Table 17. AMD77 and AMD88 both showed minimal responses to human IgG, Fc, and the off target VHH-fc PROT939, while maintaining strong responses to D04-his and D04-Fc. AMD88 did show a response to itself and to AMD77. AMD77 did not associate with itself.

### Example 9. Bridge Protein Designs

Bridge proteins contain the VHH arCAR binder and a tumor targeting VHH, specifically in this example anti-CD70 and anti-EGFR. These were constructed with and without an Fc domain that would function in vivo as a half-life extender. To achieve the specificity of the universal CAR, bridge proteins were designed that incorporated selected anti-idiotypes to D04 partnered with the D04 CAR or D04 on the soluble bridge protein partnered with an anti-ID to D04 as the CAR.

TABLE 18
Bridge Proteins without Fc: VHH1-linker-VHH2

| Protein | | | | Tumor |
|---|---|---|---|---|
| ID | VHH1 | VHH2 | Linker | Target |
| AMD1 | CD70W_DB02_D08 | huLysoB11 | (G4S)3 | CD70 |
| AMD2 | CD70W_DB02_D08 | huLysoB11 | WHITLOW | CD70 |
| AMD3 | CD70W_DB02_G07 | huLysoB11 | (G4S)3 | CD70 |
| AMD4 | CD70W_DB02_G07 | huLysoB11 | WHITLOW | CD70 |
| AMD5 | CD70W_DB02_G08 | huLysoB11 | (G4S)3 | CD70 |
| AMD6 | CD70W_DB02_G08 | huLysoB11 | WHITLOW | CD70 |
| AMD7 | CD70W_DB02_G09 | huLysoB11 | (G4S)3 | CD70 |
| AMD8 | CD70W_DB02_G09 | huLysoB11 | WHITLOW | CD70 |
| AMD9 | CD70W_DB02_E01 | huLysoB11 | (G4S)3 | CD70 |
| AMD10 | CD70W_DB02_E01 | huLysoB11 | WHITLOW | CD70 |
| AMD11 | CD70W_DB02_E06 | huLysoB11 | (G4S)3 | CD70 |
| AMD12 | CD70W_DB02_E06 | huLysoB11 | WHITLOW | CD70 |
| AMD13 | CD70W_DB02_D08 | huLysoB11 | (G4S)3 | CD70 |
| AMD14 | CD70W_DB02_D08 | huLysoB11 | WHITLOW | CD70 |
| AMD15 | CD70W_DB02_G07 | huLysoB11 | (G4S)3 | CD70 |
| AMD16 | CD70W_DB02_G07 | huLysoB11 | WHITLOW | CD70 |
| AMD17 | CD70W_DB02_G08 | huLysoB11 | (G4S)3 | CD70 |
| AMD18 | CD70W_DB02_G08 | huLysoB11 | WHITLOW | CD70 |
| AMD19 | CD70W_DB02_G09 | huLysoB11 | (G4S)3 | CD70 |
| AMD20 | CD70W_DB02_G09 | huLysoB11 | WHITLOW | CD70 |
| AMD21 | CD70W_DB02_E01 | huLysoB11 | (G4S)3 | CD70 |
| AMD22 | CD70W_DB02_E01 | huLysoB11 | WHITLOW | CD70 |
| AMD23 | CD70W_DB02_E06 | huLysoB11 | (G4S)3 | CD70 |
| AMD24 | CD70W_DB02_E06 | huLysoB11 | WHITLOW | CD70 |
| AMD25 | CD70W_DB02_D08 | huLysoD04 | (G4S)3 | CD70 |
| AMD26 | CD70W_DB02_D08 | huLysoD04 | WHITLOW | CD70 |
| AMD27 | CD70W_DB02_G07 | huLysoD04 | (G4S)3 | CD70 |
| AMD28 | CD70W_DB02_G07 | huLysoD04 | WHITLOW | CD70 |
| AMD29 | CD70W_DB02_G08 | huLysoD04 | (G4S)3 | CD70 |
| AMD30 | CD70W_DB02_G08 | huLysoD04 | WHITLOW | CD70 |
| AMD31 | CD70W_DB02_G09 | huLysoD04 | (G4S)3 | CD70 |
| AMD32 | CD70W_DB02_G09 | huLysoD04 | WHITLOW | CD70 |
| AMD33 | CD70W_DB02_E01 | huLysoD04 | (G4S)3 | CD70 |
| AMD34 | CD70W_DB02_E01 | huLysoD04 | WHITLOW | CD70 |
| AMD35 | CD70W_DB02_E06 | huLysoD04 | (G4S)3 | CD70 |
| AMD36 | CD70W_DB02_E06 | huLysoD04 | WHITLOW | CD70 |
| AMD37 | CD70W_DB02_D08 | huLysoD04 | (G4S)3 | CD70 |
| AMD38 | CD70W_DB02_D08 | huLysoD04 | WHITLOW | CD70 |
| AMD39 | CD70W_DB02_G07 | huLysoD04 | (G4S)3 | CD70 |
| AMD40 | CD70W_DB02_G07 | huLysoD04 | WHITLOW | CD70 |
| AMD41 | CD70W_DB02_G08 | huLysoD04 | (G4S)3 | CD70 |
| AMD42 | CD70W_DB02_G08 | huLysoD04 | WHITLOW | CD70 |
| AMD43 | CD70W_DB02_G09 | huLysoD04 | (G4S)3 | CD70 |
| AMD44 | CD70W_DB02_G09 | huLysoD04 | WHITLOW | CD70 |
| AMD45 | CD70W_DB02_E01 | huLysoD04 | (G4S)3 | CD70 |
| AMD46 | CD70W_DB02_E01 | huLysoD04 | WHITLOW | CD70 |
| AMD47 | CD70W_DB02_E06 | huLysoD04 | (G4S)3 | CD70 |
| AMD48 | CD70W_DB02_E06 | huLysoD04 | WHITLOW | CD70 |

TABLE 19
Bridge proteins with VHH1-whitlow-VHH2-IgG1 Fc constructs

| Protein | | | Tumor |
|---|---|---|---|
| ID | VHH1 | VHH2 | Target |
| AMD99 | huD04_CNTY1_A11 | CD70W_DB02_G08 | CD70 |
| AMD100 | huD04_CNTY1_B12 | CD70W_DB02_G08 | CD70 |
| AMD101 | huD04_CNTY1_D10 | CD70W_DB02_G08 | CD70 |
| AMD102 | CD70W_DB02_G08 | huD04_CNTY1_A11 | CD70 |
| AMD103 | CD70W_DB02_G08 | huD04_CNTY1_B12 | CD70 |
| AMD104 | CD70W_DB02_G08 | huD04_CNTY1_D10 | CD70 |
| AMD105 | huLysoD04 | CD70W_DB02_G08 | CD70 |
| AMD106 | CD70W_DB02_G08 | huLysoD04 | CD70 |
| AMD107 | huD04_CNTY1_A11 | 9G8 | EGFR |
| AMD108 | huD04_CNTY1_B12 | 9G8 | EGFR |
| AMD109 | huD04_CNTY1_D10 | 9G8 | EGFR |
| AMD110 | 9G8 | huD04_CNTY1_A11 | EGFR |
| AMD111 | 9G8 | huD04_CNTY1_B12 | EGFR |
| AMD112 | 9G8 | huD04_CNTY1_D10 | EGFR |
| AMD113 | huLysoD04 | 9G8 | EGFR |
| AMD114 | 9G8 | huLysoD04 | EGFR |

TABLE 20
Bridge proteins with VHH-IgG1 Fc-(G4S)1-VHH constructs

| Protein | | | Tumor |
|---|---|---|---|
| ID | VHH1 | VHH2 | Target |
| AMD115 | huD04_CNTY1_A11 | CD70W_DB02_G08 | CD70 |
| AMD116 | huD04_CNTY1_B12 | CD70W_DB02_G08 | CD70 |
| AMD117 | huD04_CNTY1_D10 | CD70W_DB02_G08 | CD70 |
| AMD118 | CD70W_DB02_G08 | huD04_CNTY1_A11 | CD70 |
| AMD119 | CD70W_DB02_G08 | huD04_CNTY1_B12 | CD70 |
| AMD120 | CD70W_DB02_G08 | huD04_CNTY1_D10 | CD70 |
| AMD121 | huLysoD04 | CD70W_DB02_G08 | CD70 |
| AMD122 | CD70W_DB02_G08 | huLysoD04 | CD70 |
| AMD123 | huD04_CNTY1_A11 | 9G8 | EGFR |
| AMD124 | huD04_CNTY1_B12 | 9G8 | EGFR |
| AMD125 | huD04_CNTY1_D10 | 9G8 | EGFR |
| AMD126 | 9G8 | huD04_CNTY1_A11 | EGFR |
| AMD127 | 9G8 | huD04_CNTY1_B12 | EGFR |
| AMD128 | 9G8 | huD04_CNTY1_D10 | EGFR |
| AMD129 | huLysoD04 | 9G8 | EGFR |
| AMD130 | 9G8 | huLysoD04 | EGFR |

### Example 10. T Cell Cytotoxicity with D04 CAR and huD04_CNTY1 D10 Bridge

To determine if the universal platform could mediate cytotoxicity by T cells, we created bridge proteins that consisted of the VHH from the AMD88 protein, (huD04_CNTY1_D10) linked in tandem by a Whitlow linker to the EGFR targeting VHH 9G8, fused to an IgG1 Fc fragment (Table 19). Each bridge protein was tested in a T cell cytotoxicity assay with primary T cells expressing the D04 CAR.

TABLE 21
Protein concentration and percent monomer
for AMD109 and AMD112 bridge proteins

| DNA ID | Concentration (mg/mL) | % Monomer |
|---|---|---|
| AMD109 | 0.35 | 97.40 |
| AMD112 | 0.46 | 97.34 |

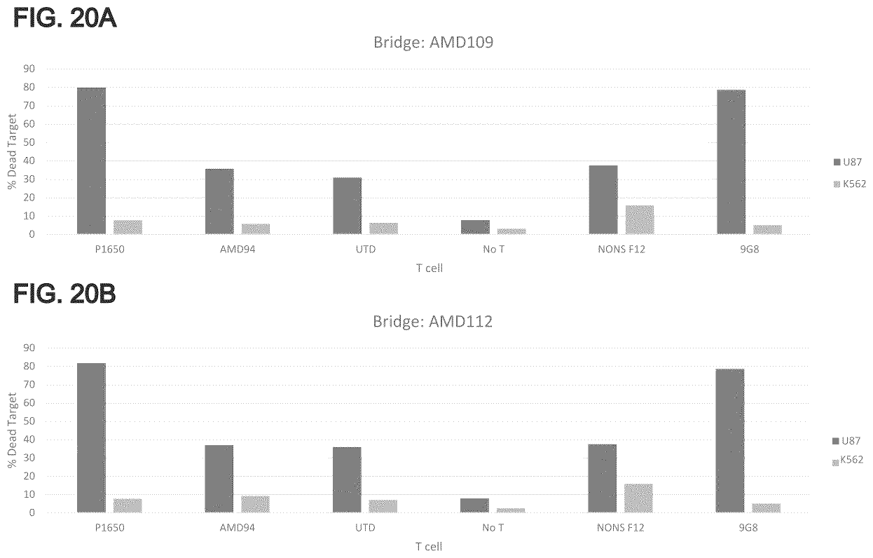

The killing assay was performed by coculturing CAR T cells, with EGFR positive cells labeled with CellTrace Violet at an effector to target ratio of 1:2, adjusted for the percent CAR positivity of the T cells, for 48 hours. 5 nM of the bridge, either AMD109 (FIG. 20A) or AMD112 (FIG. 20B).

As shown in FIGS. 20A-20B, when the bridge and CAR are ‘matched’ (e.g., an anti-idiotype VHH is present on the bridge protein while its target VHH is present on the CAR) robust cytotoxicity of EGFR positive cells is observed as in the case of P1650 CAR expressing T cells, which have D04 on their CAR (huD04_CNTY1_D10, an anti-idiotype to D04 on the soluble bridge protein). This cytotoxic activity is comparable to the 9G8 CAR. When the CAR and bridge are mismatched, as with the CAR AMD94 or the NONS F12 CAR, no cytotoxicity is observed.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A universal chimeric antigen receptor system comprising two or more adaptable receptor specificity components (arCARs), comprising
(i) an immune effector cell comprising two or more chimeric antigen receptors each comprising a first polypeptide comprising:
(a) an extracellular tag-binding domain, wherein each of the two or more chimeric antigen receptors comprises a unique extracellular tag-binding domain,
(b) a transmembrane domain, and
(c) at least one intracellular signaling domain; and
(ii) two or more second polypeptides comprising:
(a) an antigen-binding domain that binds to at least one antigen on a target cell, and
(b) a tag that is recognized by the extracellular tag-binding domain, wherein each of the two or more second polypeptides comprises a unique tag recognized by the unique extracellular tag-binding domain;
wherein:
(i) the tag comprises an antibody, an antigen-binding fragment thereof, or an alternative scaffold, and
the extracellular tag-binding domain comprises an anti-idiotype molecule that binds to the tag; or
(ii) the tag comprises an anti-idiotype molecule that binds to the extracellular tag-binding domain, and
the extracellular tag-binding domain comprises an antibody, or antigen-binding fragment thereof, or an alternative scaffold.

2. The arCAR system of claim 1, wherein the anti-idiotype molecule binds to at least one antigen binding region of the antibody, antigen-binding fragment thereof, or alternative scaffold.

3. The arCAR system of claim 1, wherein the anti-idiotype molecule binds to at least one complementarity determining region (CDR) of the antibody, or antigen-binding fragment thereof.

4. The arCAR system of claim 1, wherein the antigen-binding domain of the two or more second polypeptides each comprises an antibody, or antigen-binding fragment thereof, or an alternative scaffold.

5. The arCAR system of claim 1, wherein the anti-idiotype molecule is an anti-idiotype antibody, or antigen-binding fragment thereof, or an anti-idiotype alternative scaffold.

6-10. (canceled)

11. The arCAR system of claim 1, wherein one or more of the antigen-binding fragment, the extracellular tag-binding domain, the antigen-binding domain, and the tag comprise a polypeptide sequence having at least 90%, 91%, 92%, 93%, 94%, 95%, 96%, 97%, 98%, 99% or 100% sequence identity to a sequence selected from the group consisting of SEQ ID NOs.: 84, 86, 88, 90, 92, 94, 96, 98, 100, 102, 104, 106, 108, and 110-133.

12-16. (canceled)

17. The arCAR system of claim 1, wherein the antigen-binding domain binds to at least one tumor antigen or autoimmune antigen.

18. The arCAR system of claim 17, wherein each of the at least one tumor antigen or autoimmune antigen is associated with the same tumor or autoimmune disease.

19. The arCAR system of claim 17, wherein the at least one tumor antigen is associated with glioblastoma, ovarian cancer, cervical cancer, head and neck cancer, liver cancer, prostate cancer, pancreatic cancer, renal cell carcinoma, bladder cancer, or hematologic malignancy.

20-31. (canceled)

32. The arCAR system of claim 1, wherein the two or more second polypeptides is each separately a soluble polypeptide.

33. (canceled)

34. The arCAR system of claim 1, wherein the immune effector cell is derived from an induced pluripotent stem cell (iPSC).

35. The arCAR system of claim 34, wherein the immune effector cell is a T cell or NK cell derived from an induced pluripotent stem cell (iPSC).

36-52. (canceled)

53. A method of treating a disease in a subject in need thereof, comprising administering to the subject a therapeutically effective amount of:
(i) an immune effector cell comprising the first polypeptides of the two or more chimeric antigen receptors of the two or more arCARs of claim 1, and
(ii) the two or more second polypeptides of said two or more arCARs, or one or more polynucleotides encoding said two or more second polypeptides, or one or more host cells comprising said two or more second polypeptides.

54. The method of claim 53, wherein the immune effector cell is a T cell, a Natural Killer (NK) cell, a cytotoxic T lymphocyte (CTL), a regulatory T cell or a tumor-infiltrating lymphocyte (TIL), a dendritic cell, or a macrophage.

55. The method of claim 53, wherein the immune effector cell is derived from an iPSC.

56. The method of claim 53, wherein the immune effector cell constitutively expresses the first polypeptide.

57. The method of claim 53, wherein the disease is a cancer or autoimmune disease.

58. The method of claim 57, wherein the cancer is glioblastoma, ovarian cancer, cervical cancer, head and neck cancer, liver cancer, prostate cancer, pancreatic cancer, renal cell carcinoma, bladder cancer, or hematologic malignancy.

59-61. (canceled)

62. The method of claim 53, wherein the immune effector cell is an allogeneic cell.

63. The method of claim 53, wherein the subject is a human.
